SEQUENCE LISTING (96 sequences)

SEQ ID NO: 1 - DNA, 1707 bp, Homo sapiens
atgaaggaagcagagatggacggtgaggcagtccgcttctgcacagataaccagtgtgtctccctgcacccccaagaggtggactctgtggcaatggctcctgcagcccccaagataccgaggctcgttcaggctaccccggcatttatggctgtgaccttggtcttctctcttgtgactctctttgtagtgggtaagcccccagttcaacagcagacaagacctgttccgaagcctgtgcaagccgtaattctgggagacaacattactgggcatttaccttttgaacccaacaatcatcaccactttggcagggaggcagaaatgcgagagcttatccagacatttaaaggccacatggagaattccagtgcctgggtagtagaaatccagatgttgaagtgcagagtggacaatgtcaattcgcagctccaggtgctcggtgatcatctgggaaacaccaatgctgacatccagatggtaaaaggagttctaaaggatgccactacattgagtttgcagacacagatgttaaggagttccctggagggaaccaatgctgagatccagaggctcaaggaagaccttgaaaaggcagatgctttaactttccagacgctgaatttcttaaaaagcagtttagaaaacaccagcattgagctccacgtgctaagcagaggcttagaaaatgcaaactctgaaattcagatgttgaatgccagagccaatgctgagatccagggactaaaggaaaatttgcagaacacaaatgctttaaactcccagacccaggcctttataaaaagcagttttgacaacactagtgctgagatccagttcttaagaggtcatttggaaagagctggtgatgaaattcacgtgttaaaaagggatttgaaaatggtcacagcccagacccaaaaagcaaatggccgtctggaccagacagatactcagattcaggtattcaagtcagagatggaaaatgtgaataccttaaatgcccagattcaggtcttaaatggtcatatgaaaaatgccagcagagagatacagaccctaaaacaaggaatgaagaatgcttcagccttaacttcccagacccagatgttagacagcaatctgcagaaggccagtgccgagatccagaggttaagaggggatctagagaacaccaaagctctaaccatggaaatccagcaggagcagagtcgcctgaagaccctccatgtggtcattacttcacaggaacagctacaaagaacccaaagtcagcttctccagatggtcctgcaaggctggaagttcaatggtggaagcttatattatttttctagtgtcaagaagtcttggcatgaggctgagcagttctgcgtgtcccagggagcccatctggcatctgtggcctccaaggaggagcaggcatttctggtagagttcacaagtaaagtgtactactggatcggtctcactgacaggggcacagagggctcctggcgctggacagatgggacaccattcaacgccgcccagaacaaagcccctgttgtcttcgggttttgggaaaagaatcagtctgacaactggcggcacaagaatgggcagactgaagactgtgtccaaattcagcagaagtggaatgacatgacctgtgacaccccctatcagtgggtgtgcaagaagcccatgggccagggtgtggcctga

SEQ ID NO: 2 - DNA, 1095 bp, Homo sapiens
atgtcgagacaaggaaaattattttctgcttttggagtgggttgttgtgtaacggcaggcttgccaaaggacgataacactcctagcaccattgcggatgtgcacaatggttatacgatgaatgttgtagagcaagttctaaaggatagttttgtgttatttttcccaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactatgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatga

SEQ ID NO: 3 - DNA, 984 bp, Homo sapiens
atgttgctcttattcagtgtaatcctaatctcatgggtatccactgttgggggagaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactatgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatga

SEQ ID NO: 4 - DNA, 465 bp, Homo sapiens
atggcaccggccagagcaggatgctgccccctgctgctgctgcttctggggctgtgggtggcagaggtcctagtcagagccaagcccaaggacatgacatcatctcagtggtttaaaactcagcatgtgcagcccagccctcaagcatgcaactcagccatgagcatcatcaataagtacacagaacggtgcaaagacctcaacaccttcctgcacgagcccttctccagtgtggccatcacctgccagacccccaacatagcctgcaagaatagctgtaaaaactgccaccagagccacgggcccatgtccctgaccatgggtgagctcacctcagggaagtacccaaactgcaggtacaaagagaagcacctgaacacaccttacatagtggcctgtgaccctccacaacagggtgacccagggtacccacttgttcctgtgcacttggataaagttgtctaa

SEQ ID NO: 5 - DNA, 1509 bp, Homo sapiens
atgcgccagctgggagggtctctccgccccccgcgcgcggcccacggggccgagcctctccccagtgcgctggggccctgcgctgggggtgaccgggacctgggtcggggaacccccggatgggagcccaggcgcgccagggtccccatccacgagcaggtcgaccccccgcgagaggggccccacctcttccagaacctcctgctcttcctgtgggccctgctgaactgtggtttgggggtcagtgctcagggtccgggcgagtggaccccgtgggtgtcctggacccgctgctccagctcctgcgggcgtggcgtctctgtgcgcagccggcgctgcctccggcttcctggggaagaaccgtgctggggagactcccatgagtaccgcctctgccagttgccagactgccccccaggggctgtgcccttccgagacctacagtgtgccctgtacaatggccgccctgtcctgggcacccagaagacctaccagtgggtgcccttccatggggcgcccaaccagtgcgacctcaactgcctggctgaggggcacgccttctaccacagcttcggccgcgtcctggacggcaccgcctgcagcccgggtgcccagggggtctgcgtggctggccgctgccttagcgccggctgtgatgggttgttgggctcgggtgccctcgaggaccgctgtggccgctgcggaggcgccaacgactcgtgccttttcgtgcagcgcgtgtttcgtgacgccggtgccttcgctgggtactggaacgtgaccctgatccccgagggcgccagacacatccgcgtggaacacaggagccgcaaccacctgggtatcctaggatcactgatggggggcgatgggcgctacgtgcttaatgggcactgggtggtcagcccaccagggacctacgaggcggccggcacgcatgtggtctacacccgagacacagggccccaggagacattgcaagcagccgggcccacctcccatgacctgctcctacaggtcctcctgcaggagcccaaccctggcatcgagtttgagttctggctccctcgggagcgctacagccccttccaggctcgtgtgcaggccctgggctggcccctgaggcagcctcagccccggggggtggagcctcagccccccgcagcccctgctgtcacccctgcacagaccccaacgctggccccagtgttccaggcccgagtgctgggccaccaccaccaggcccaggagacccgctatgaggtgcgcatccagctcgtctacaagaaccgctcgccactgcgggcacgcgagtacgtgtgggcgccaggccactgcccctgcccgatgctggcaccccaccgggactacctgatggctgtccagcgtcttgtcagccccgacggcacacaggaccagctgctgctgccccacgccggctacgcccggccctggagccctgcggaggacagccgcatacgcctgactgcccggcgctgtcctggctga

SEQ ID NO: 6 - DNA, 1356 bp, Homo sapiens
atggactcggcccctctgttccccaggccccacctcttccagaacctcctgctcttcctgtgggccctgctgaactgtggtttgggggtcagtgctcagggtccgggcgagtggaccccgtgggtgtcctggacccgctgctccagctcctgcgggcgtggcgtctctgtgcgcagccggcgctgcctccggcttcctggggaagaaccgtgctggggagactcccatgagtaccgcctctgccagttgccagactgccccccaggggctgtgcccttccgagacctacagtgtgccctgtacaatggccgccctgtcctgggcacccagaagacctaccagtgggtgcccttccatggggcgcccaaccagtgcgacctcaactgcctggctgaggggcacgccttctaccacagcttcggccgcgtcctggacggcaccgcctgcagcccgggtgcccagggggtctgcgtggctggccgctgccttagcgccggctgtgatgggttgttgggctcgggtgccctcgaggaccgctgtggccgctgcggaggcgccaacgactcgtgccttttcgtgcagcgcgtgtttcgtgacgccggtgccttcgctgggtactggaacgtgaccctgatccccgagggcgccagacacatccgcgtggaacacaggagccgcaaccacctgggtatcctaggatcactgatggggggcgatgggcgctacgtgcttaatgggcactgggtggtcagcccaccagggacctacgaggcggccggcacgcatgtggtctacacccgagacacagggccccaggagacattgcaagcagccgggcccacctcccatgacctgctcctacaggtcctcctgcaggagcccaaccctggcatcgagtttgagttctggctccctcgggagcgctacagccccttccaggctcgtgtgcaggccctgggctggcccctgaggcagcctcagccccggggggtggagcctcagccccccgcagcccctgctgtcacccctgcacagaccccaacgctggccccagtgttccaggcccgagtgctgggccaccaccaccaggcccaggagacccgctatgaggtgcgcatccagctcgtctacaagaaccgctcgccactgcgggcacgcgagtacgtgtgggcgccaggccactgcccctgcccgatgctggcaccccaccgggactacctgatggctgtccagcgtcttgtcagccccgacggcacacaggaccagctgctgctgccccacgccggctacgcccggccctggagccctgcggaggacagccgcatacgcctgactgcccggcgctgtcctggctga

SEQ ID NO: 7 - DNA, 1296 bp, Homo sapiens
atgatccggaccccattgtcggcctctgcccatcgcctgctcctcccaggctcccgcggccgacccccgcgcaacatgcagcccacgggccgcgagggttcccgcgcgctcagccggcggtatctgcggcgtctgctgctcctgctactgctgctgctgctgcggcagcccgtaacccgcgcggagaccacgccgggcgcccccagagccctctccacgctgggctcccccagcctcttcaccacgccgggtgtccccagcgccctcactaccccaggcctcactacgccaggcacccccaaaaccctggaccttcggggtcgcgcgcaggccctgatgcggagtttcccactcgtggacgggtatgtaggtctgaacagctctcaaaagctggcctgcctcattggcgtggagggtggtcactcactggacagcagcctctctgtgctgcgcagtttctatgtgctgggggtgcgctacctgacacttaccttcacctgcagtacaccatgggcagagagttccaccaagttcagacaccacatgtacaccaacgtcagcggattgacaagctttggtgagaaagtagtagaggagttgaaccgcctgggcatgatgatagatttgtcctatgcatcggacaccttgataagaagggtcctggaagtgtctcaggctcctgtgatcttctcccactcagctgccagagctgtgtgtgacaatttgttgaatgttcccgatgatatcctgcagcttctgaagaagaacggtggcatcgtgatggtgacactgtccatgggggtgctgcagtgcaacctgcttgctaacgtgtccactgtggcagatgattcgaatcgatgctcggtacccgtcattggatctgagttcatcgggattggtggaaattatgacgggactggccggttccctcaggggctggaggatgtgtccacatacccagtcctgatagaggagttgctgagtcgtagctggagcgaggaagagcttcaaggtgtccttcgtggaaacctgctgcgggtcttcagacaagtggaaaaggtgagagaggagagcagggcgcagagccccgtggaggctgagtttccatatgggcaactgagcacatcctgccactcccacctcgtgcctcagaatggacaccaggctactcatctggaggtgaccaagcagccaaccaatcgggtcccctggaggtcctcaaatgcctccccataccttgttccaggccttgtggctgctgccaccatcccaaccttcacccagtggctctgctga

SEQ ID NO: 8 - DNA, 1443 bp, Homo sapiens
atgcagccctccggcctcgagggtcccggcacgtttggtcggtggcctctgctgagtctgctgctcctgctgctgctgctccagcctgtaacctgtgcctacaccacgccaggcccccccagagccctcaccacgctgggcgcccccagagcccacaccatgccgggcacctacgctccctcgaccacactcagtagtcccagcacccagggcctgcaagagcaggcacgggccctgatgcgggacttcccgctcgtggacggccacaacgacctgcccctggtcctaaggcaggtttaccagaaagggctacaggatgttaacctgcgcaatttcagctacggccagaccagcctggacaggcttagagatggcctcgtgggcgcccagttctggtcagcctatgtgccatgccagacccaggaccgggatgccctgcgcctcaccctggagcagattgacctcatacgccgcatgtgtgcctcctattctgagctggagcttgtgacctcggctaaaggtctgaacagctctcaaaagctggcctgcctcattggcgtggagggtggtcactcactggacagcagcctctctgtgctgcgcagtttctatgtgctgggggtgcgctacctgacacttaccttcacctgcagtacaccatgggcagagagttccaccaagttcagacaccacatgtacaccaacgtcagcggattgacaagctttggtgagaaagtagtagaggagttgaaccgcctgggcatgatgatagatttgtcctatgcatcggacaccttgataagaagggtcctggaagtgtctcaggctcctgtgatcttctcccactcagctgccagagctgtgtgtgacaatttgttgaatgttcccgatgatatcctgcagcttctgaagaagaacggtggcatcgtgatggtgacactgtccatgggggtgctgcagtgcaacctgcttgctaacgtgtccactgtggcagatcactttgaccacatcagggcagtcattggatctgagttcatcgggattggtggaaattatgacgggactggccggttccctcaggggctggaggatgtgtccacatacccagtcctgatagaggagttgctgagtcgtagctggagcgaggaagagcttcaaggtgtccttcgtggaaacctgctgcgggtcttcagacaagtggaaaaggtgagagaggagagcagggcgcagagccccgtggaggctgagtttccatatgggcaactgagcacatcctgccactcccacctcgtgcctcagaatggacaccaggctactcatctggaggtgaccaagcagccaaccaatcgggtcccctggaggtcctcaaatgcctccccataccttgttccaggccttgtggctgctgccaccatcccaaccttcacccagtggctctgctga

SEQ ID NO: 9 - DNA, 1116 bp, Homo sapiens
atggatgcagccacagctccaaagcaagcctggcccccatggcccccgctccttttcctcctcctcctacctggagggagcggtggcagctgccctgctgtgtgtgactgcacctcccagccccaggctgtgctctgtggccacaggcaactggaggctgtacctggaggactcccactggacactgagctcctggacctgagtgggaaccgcctcccgaaggctcagcccctggtgcggctccaggagctacgcctgtcaggggcatgcctcacctccattgctgcccatgccttccatggcttgactgccttccacctcctggatgtggcagataacgcccttcagacactagaggaaacagctttcccttctccagacaaactggtcaccttgaggctgtctggcaaccccctaacctgtgactgccgcctcctctggctgctccggctccgccacctggactttggcatgtccccccctgcctgtgctggcccccatcatgtccaggggaagagcctgaaggagttttcagacatcctgcctccagggcacttcacctgcaaaccagccctgatccgaaagtcggggcctcgatgggtcattgcagaggagggcgggcatgcggttttctcctgctctggagatggagacccagcccccactgtctcctggatgaggcctcatggggcttggctgggcagggctgggagagtaagggtcctagaggatgggacactggagatccgctcagtgcagctacgggacagaggggcctatgtctgtgtggttagcaatgtcgctgggaatgactccctgaggacctggctggaagtcatccaggtggaaccaccaaacggcacactttctgaccccaacatcaccgtgccagggatcccagggcctttttttctggatagcagaggtgtggccatggtgctggcagtcggcttcctccccttcctcacctcagtgaccctctgctttggcctgattgccctttggagcaagggcaaaggtcgggtcaaacatcacatgacctttgactttgtggcacctcggccctctggggataaaaactctgggggtaaccgggtcactgccaagctcttctga

SEQ ID NO: 10 - DNA, 1779 bp, Homo sapiens
atggatgcagccacagctccaaagcaagcctggcccccatggcccccgctccttttcctcctcctcctacctggagggagcggtggcagctgccctgctgtgtgtgactgcacctcccagccccaggctgtgctctgtggccacaggcaactggaggctgtacctggaggactcccactggacactgagctcctggacctgagtgggaaccgcctgtgggggctccagcagggaatgctctcccgcctgagcctgctccaggaattggacctcagctacaaccagctctcaacccttgagcctggggccttccatggcctacaaagcctactcaccctgaggctgcagggcaatcggctcagaatcatggggcctggggtcttctcaggcctctctgctctgaccctgctggacctccgcctcaaccagattgttctcttcctagatggagcttttggggagctaggcagcctccagaagctggaggttggggacaaccacctggtatttgtggctccgggggcctttgcagggctagccaagttgagcaccctcaccctggagcgctgcaacctcagcacagtgcctggcctagcccttgcccgtctcccggcactagtggccctaaggcttagagaactggatattgggaggctgccagctggggccctgcgggggctggggcagctcaaggagctggagatccacctctggccatctctggaggctctggaccctgggagcctggttgggctcaatctcagcagcctggccatcactcgctgcaatctgagctcggtgcccttccaagcactgtaccacctcagcttcctcagggtcctggatctgtcccagaatcccatctcagccatcccagcccgaaggctcagccccctggtgcggctccaggagctacgcctgtcaggggcatgcctcacctccattgctgcccatgccttccatggcttgactgccttccacctcctggatgtggcagataacgcccttcagacactagaggaaacagctttcccttctccagacaaactggtcaccttgaggctgtctggcaaccccctaacctgtgactgccgcctcctctggctgctccggctccgccacctggactttggcatgtccccccctgcctgtgctggcccccatcatgtccaggggaagagcctgaaggagttttcagacatcctgcctccagggcacttcacctgcaaaccagccctgatccgaaagtcggggcctcgatgggtcattgcagaggagggcgggcatgcggttttctcctgctctggagatggagacccagcccccactgtctcctggatgaggcctcatggggcttggctgggcagggctgggagagtaagggtcctagaggatgggacactggagatccgctcagtgcagctacgggacagaggggcctatgtctgtgtggttagcaatgtcgctgggaatgactccctgaggacctggctggaagtcatccaggtggaaccaccaaacggcacactttctgaccccaacatcaccgtgccagggatcccagggcctttttttctggatagcagaggtgtggccatggtgctggcagtcggcttcctccccttcctcacctcagtgaccctctgctttggcctgattgccctttggagcaagggcaaaggtcgggtcaaacatcacatgacctttgactttgtggcacctcggccctctggggataaaaactctgggggtaaccgggtcactgccaagctcttctga

SEQ ID NO: 11 - DNA, 429 bp, Homo sapiens
atggccaggtacatgctgctgctgctcctggcggtatgggtgctgaccggggagctgtggccgggagctgaggcccgggcagcgccttacggggtcaggctttgcggccgagaattcatccgagcagtcatcttcacctgcgggggctcccggtggagacgatcagacatcctggcccacgaggctatgggagataccttcccggatgcagatgctgatgaagacagtctggcaggcgagctggatgaggccatggggtccagcgagtggctggccctgaccaagtcaccccaggccttttacagggggcgacccagctggcaaggaacccctggggttcttcggggcagccgagatgtcctggctggcctttccagcagctgctgcaagtgggggtgtagcaaaagtgaaatcagtagcctttgctag

SEQ ID NO: 12 - DNA, 692 bp, Homo sapiens
atgtctgaagaagtgacctacgcgacactcacatttcaggattctgctggagcaaggaataaccgagatggaaataacctaagaaaaagagggcatccagctccatctcccatttggcgtcatgctgctctgggtctggtaactctttgcctgatgttgctgattgggctggtgacgttggggatgatgtttttgcagatatctaatgacattaactcagattcagagaaattgagtcaacttcagaaaaccatccaacagcagcaggataacttatcccagcaactgggcaactccaacaacttgtccatggaggaggaatttctcaagtcacagatctccagtctactgaagaggcaggaacaaatggccatcaaactgtgccaagagctaatcattcatacttcagaccacagatgtaatccatgtcctaagatgtggcaatggtaccaaaatagttgctactattttacaacaaatgaggagaaaacctgggctaacagtagaaaggactgcatagacaagaactccaccctagtgaagatagacagtttggaagaaaaggattttcttatgtcacagccattactcatgttttcgttcttttggctgggattatcatgggactcctctggcagaagttggttctgggaagatggctctgttccctctccatccttgtacgtctctaa

SEQ ID NO: 13 - DNA, 585 bp, Homo sapiens
atgtggctgtccccagctctgctgcttctcatcctcccaggttactccattgccgctaaaatcactggtccaacaacagtgaatggctcggagcagggctcattgactgtgcagtgtgcttatggctcaggctgggagacctacttgaagtggcggtgtcaaggagctgattggaattactgtaacatccttgttaaaacaaatggatcagagcaggaggtaaagaagaatcgagtttccatcagggacaatcagaaaaaccacatgttcaccgtgaccatggagaatctcaaaagagatgatgctgacagttattggtgtgggactgagagacctggaattgatcttggggtcaaagttcaagtgaccattaacccaggcacacaaactgcagtctcagaatggacaaccacaacagcaagcctggctttcacagctgcagccacccagaagaccagcagccccctcaccaggtccccgctcaagagcacccacttcctgttcctgttcctcctggagctgcctctgctcctgagcatgctggggaccgtcctctgggtgaacagaccacaaagaaggtcttga

SEQ ID NO: 14 - DNA, 1002 bp, Homo sapiens
atgaggatctggtggcttctgcttgccattgaaatctgcacagggaacataaactcacaggacacctgcaggcaagggcaccctggaatccctgggaaccccggtcacaatggtctgcctggaagagatggacgagacggagcgaagggtgacaaaggcgatgcaggagaaccaggacgtcctggcagcccggggaaggatgggacgagtggagagaagggagaacgaggagcagatggaaaagttgaagcaaaaggcatcaaaggtgatcaaggctcaagaggatccccaggaaaacatggccccaaggggcttgcagggcccatgggagagaagggcctccgaggagagactgggcctcaggggcagaaggggaataagggtgacgtgggtcccactggtcctgaggggccaaggggcaacattgggcctttgggcccaactggtttaccgggccccatgggccctattggaaagcctggtcccaaaggagaagctggacccacggggccccagggtgagccaggagtccggggaataagaggctggaaaggagatcgaggagagaaagggaaaatcggtgagactctagtcttgccaaaaagtgctttcactgtggggctcacggtgctgagcaagtttccttcttcagatatgcccattaaatttgataagatcctgtataacgaattcaaccattatgatacagcagcggggaaattcacgtgccacattgctggggtctattacttcacctaccacatcactgttttctccagaaatgttcaggtgtctttggtcaaaaatggagtaaaaatactgcacaccaaagatgcttacatgagctctgaggaccaggcctctggcggcattgtcctgcagctgaagctcggggatgaggtgtggctgcaggtgacaggaggagagaggttcaatggcttgtttgctgatgaggacgatgacacaactttcacagggttccttctgttcagcagcccgtga

SEQ ID NO: 15 - DNA, 678 bp, Homo sapiens
atgaggatctggtggcttctgcttgccattgaaatctgcacagggaacataaactcacaggacacctgcaggcaagggcaccctggaatccctgggaaccccggtcacaatggtctgcctggaagagatggacgagacggagcgaagggtgacaaaggcgatgcaggagaagctggacccacggggccccagggtgagccaggagtccggggaataagaggctggaaaggagatcgaggagagaaagggaaaatcggtgagactctagtcttgccaaaaagtgctttcactgtggggctcacggtgctgagcaagtttccttcttcagatatgcccattaaatttgataagatcctgtataacgaattcaaccattatgatacagcagcggggaaattcacgtgccacattgctggggtctattacttcacctaccacatcactgttttctccagaaatgttcaggtgtctttggtcaaaaatggagtaaaaatactgcacaccaaagatgcttacatgagctctgaggaccaggcctctggcggcattgtcctgcagctgaagctcggggatgaggtgtggctgcaggtgacaggaggagagaggttcaatggcttgtttgctgatgaggacgatgacacaactttcacagggttccttctgttcagcagcccgtga

SEQ ID NO: 16 - DNA, 618 bp, Homo sapiens
atgatgagaactctcatcaccacacacccactgcccctgcttctattgccgcagcagctgctgcagctggtgcagtttcaagaggtggatacagattttgatttcccagaagaagataaaaaagaagaatttgaagagtgtttggaaaaattttttagtacagggcccgccagaccacctaccaaagaaaaagtcaaaagacgtgtccttattgaacctggaatgccactaaatcatatagagtactgtaaccatgaaatcatgggaaaaaatgtttactacaaacaccgttgggtggcagaacattacttccttcttatgcaatatgacgagctccaaaaaatctgttacaacagatttgtgccatgtaagaatggaattaggaaatgtaacaggagcaaaggtcttgtagaaggagtgtattgtaatttaacagaagcatttgaaataccagcgtgtaaatacgaatcactttataggaagggctacgtccttatcacttgttcatggcaaaatgaaatgcaaaaacgtattcctcatactataaatgatctcgtggagccacctgaacacagaagtttcctcagtgaggatggtgtctttgtcatatcgccctag

SEQ ID NO: 17 - DNA, 288 bp, Homo sapiens
atggaggtggtgctgatctttctatgcagcctgttggcccacattgtcctggccgatgcagttgagagggagaagcaaattgacccttttcattatgactaccagaccctgaggattagggggttggtatgtgctgtggtcctcttctccattgggatcctccttatcctaggttgcagatgcaagtgcagtttcaatcagaagcccaggaccccaggagaggaggaagcccaggtggagaacctcatcactgcaaatgcaacaaagctccagaaagcagagagctga

SEQ ID NO: 18 - DNA, 1788 bp, Homo sapiens
atggaggtgggaatgggatgctgggcccgggaggtgctggtccccgaggggcccttgtaccgcgtggctggcacagctgtctccatctcctgcaatgtgaccggctatgagggccctgcccagcagaacttcgagtggttcctgtataggcccgaggccccagatactgcactgggcattgtcagtaccaaggatacccagttctcctatgctgtcttcaagtcccgagtggtggcgggtgaggtgcaggtgcagcgcctacaaggtgatgccgtggtgctcaagattgcccgcctgcaggcccaggatgccggcatttatgagtgccacaccccctccactgatacccgctacctgggcagctacagcggcaaggtggagctgagagttcttccagatgtcctccaggtgtctgctgcccccccagggccccgaggccgccaggccccaacctcacccccacgcatgacggtgcatgaggggcaggagctggcactgggctgcctggcgaggacaagcacacagaagcacacacacctggcagtgtcctttgggcgatctgtgcccgaggcaccagttgggcggtcaactctgcaggaagtggtgggaatccggtcagacttggccgtggaggctggagctccctatgctgagcgattggctgcaggggagcttcgtctgggcaaggaagggaccgatcggtaccgcatggtagtagggggtgcccaggcaggggacgcaggcacctaccactgcactgccgctgagtggattcaggatcctgatggcagctgggcccagattgcagagaaaagggccgtcctggcccacgtggatgtgcagacgctgtccagccagctggcagtgacagtggggcctggtgaacgtcggatcggcccaggggagcccttggaactgctgtgcaatgtgtcaggggcacttcccccagcaggccgtcatgctgcatactctgtaggttgggagatggcacctgcggggcacctggggcccggccgcctggtagcccagctggacacagagggtgtgggcagcctgggccctggctatgagggccgacacattgccatggagaaggtggcatccagaacataccggctacggctagaggctgccaggcctggtgatgcgggcacctaccgctgcctcgccaaagcctatgttcgagggtctgggacccggcttcgtgaagcagccagtgcccgttcccggcctctccctgtacatgtgcgggaggaaggtgtggtgctggaggctgtggcatggctagcaggaggcacagtgtaccgcggggagactgcctccctgctgtgcaacatctctgtgcggggtggccccccaggactgcggctggccgccagctggtgggtggagcgaccagaggacggagagctcagctctgtccctgcccagctggtgggtggcgtaggccaggatggtgtggcagagctgggagtccggcctggaggaggccctgtcagcgtagagctggtggggccccgaagccatcggctgagactacacagcttggggcccgaggatgaaggcgtgtaccactgtgcccccagcgcctgggtgcagcatgccgactacagctggtaccaggcgggcagtgcccgctcagggcctgttacagtctacccctacatgcatgccctggacaccctatttgtgcctctgctggtgggtacaggggtggccctagtcactggtgccactgtccttggtaccatcacttgctgcttcatgaagaggcttcgaaaacggtga

SEQ ID NO: 19 - DNA, 1842 bp, Homo sapiens
atgggcgccctcaggcccacgctgctgccgccttcgctgccgctgctgctgctgctaatgctaggaatgggatgctgggcccgggaggtgctggtccccgaggggcccttgtaccgcgtggctggcacagctgtctccatctcctgcaatgtgaccggctatgagggccctgcccagcagaacttcgagtggttcctgtataggcccgaggccccagatactgcactgggcattgtcagtaccaaggatacccagttctcctatgctgtcttcaagtcccgagtggtggcgggtgaggtgcaggtgcagcgcctacaaggtgatgccgtggtgctcaagattgcccgcctgcaggcccaggatgccggcatttatgagtgccacaccccctccactgatacccgctacctgggcagctacagcggcaaggtggagctgagagttcttccagatgtcctccaggtgtctgctgcccccccagggccccgaggccgccaggccccaacctcacccccacgcatgacggtgcatgaggggcaggagctggcactgggctgcctggcgaggacaagcacacagaagcacacacacctggcagtgtcctttgggcgatctgtgcccgaggcaccagttgggcggtcaactctgcaggaagtggtgggaatccggtcagacttggccgtggaggctggagctccctatgctgagcgattggctgcaggggagcttcgtctgggcaaggaagggaccgatcggtaccgcatggtagtagggggtgcccaggcaggggacgcaggcacctaccactgcactgccgctgagtggattcaggatcctgatggcagctgggcccagattgcagagaaaagggccgtcctggcccacgtggatgtgcagacgctgtccagccagctggcagtgacagtggggcctggtgaacgtcggatcggcccaggggagcccttggaactgctgtgcaatgtgtcaggggcacttcccccagcaggccgtcatgctgcatactctgtaggttgggagatggcacctgcgggggcacctgggcccggccgcctggtagcccagctggacacagagggtgtgggcagcctgggccctggctatgagggccgacacattgccatggagaaggtggcatccagaacataccggctacggctagaggctgccaggcctggtgatgcgggcacctaccgctgcctcgccaaagcctatgttcgagggtctgggacccggcttcgtgaagcagccagtgcccgttcccggcctctccctgtacatgtgcgggaggaaggtgtggtgctggaggctgtggcatggctagcaggaggcacagtgtaccgcggggagactgcctccctgctgtgcaacatctctgtgcggggtggccccccaggactgcggctggccgccagctggtgggtggagcgaccagaggacggagagctcagctctgtccctgcccagctggtgggtggcgtaggccaggatggtgtggcagagctgggagtccggcctggaggaggccctgtcagcgtagagctggtggggccccgaagccatcggctgagactacacagcttggggcccgaggatgaaggcgtgtaccactgtgcccccagcgcctgggtgcagcatgccgactacagctggtaccaggcgggcagtgcccgctcagggcctgttacagtctacccctacatgcatgccctggacaccctatttgtgcctctgctggtgggtacaggggtggccctagtcactggtgccactgtccttggtaccatcacttgctgcttcatgaagaggcttcgaaaacggtga

SEQ ID NO: 20 - DNA, 1791 bp, Homo sapiens
atggccgccaactccaccagcgacctccacactcccgggacgcagctgagcgtggctgacatcatcgtcatcactgtgtattttgctctgaacgtggccgtgggcatatggtcctcttgtcgggccagtaggaacacggtgaatggctacttcctggcaggccgggacatgacgtggtggccgattggagcctccctcttcgccagcagcgagggctctggcctcttcattggactggcgggctcaggcgcggcaggaggtctggccgtggcaggcttcgagtggaatgccacgtacgtgctgctggcactggcatgggtgttcgtgcccatctacatctcctcagagatcgtcaccttacctgagtacattcagaagcgctacgggggccagcggatccgcatgtacctgtctgtcctgtccctgctactgtctgtcttcaccaagatatcgctggacctgtacgcgggggctctgtttgtgcacatctgcctgggctggaacttctacctctccaccatcctcacgctcggcatcacagccctgtacaccatcgcagggggcctggctgctgtaatctacacggacgccctgcagacgctcatcatggtggtgggggctgtcatcctgacaatcaaagcttttgaccagatcggtggttacgggcagctggaggcagcctacgcccaggccattccctccaggaccattgccaacaccacctgccacctgccacgtacagacgccatgcacatgtttcgagacccccacacaggggacctgccgtggaccgggatgacctttggcctgaccatcatggccacctggtactggtgcaccgaccaggtcatcgtgcagcgatcactgtcagcccgggacctgaaccatgccaaggcgggctccatcctggccagctacctcaagatgctccccatgggcctgatcataatgccgggcatgatcagccgcgcattgttcccagatgatgtgggctgcgtggtgccgtccgagtgcctgcgggcctgcggggccgaggtcggctgctccaacatcgcctaccccaagctggtcatggaactgatgcccatcggtctgcgggggctgatgatcgcagtgatgctggcggcgctcatgtcgtcgctgacctccatcttcaacagcagcagcaccctcttcactatggacatctggaggcggctgcgtccccgctccggcgagcgggagctcctgctggtgggacggctggtcatagtggcactcatcggcgtgagtgtggcctggatccccgtcctgcaggactccaacagcgggcaactcttcatctacatgcagtcagtgaccagctccctggccccaccagtgactgcagtctttgtcctgggcgtcttctggcgacgtgccaacgagcagggggccttctggggcctgatagcagggctggtggtgggggccacgaggctggtcctggaattcctgaacccagccccaccgtgcggagagccagacacgcggccagccgtcctggggagcatccactacctgcacttcgctgtcgccctctttgcactcagtggtgctgttgtggtggctggaagcctgctgaccccacccccacagagtgtccagattgagaaccttacctggtggaccctggctcaggatgtgcccttgggaactaaagcaggtgatggccaaacaccccagaaacacgccttctgggcccgtgtctgtggcttcaatgccatcctcctcatgtgtgtcaacatattcttttatgcctacttcgcctga

SEQ ID NO: 21 - DNA, 1182 bp, Homo sapiens
atggattccttaaagaatgagaactatgatctggtatttgttgaagcatttgatttctgttctttcctgattgctgagaagcttgtgaaaccatttgtggccattcttcccaccacattcggctctttggattttgggctaccaagccccttgtcttatgttccagtattcccttccttgctgactgatcacatggacttctggggccgagtgaagaattttctgatgttctttagtttctccaggagccaatgggacatgcagtctacatttgacaacaccatcaaggagcatttcccagaaggctctaggccagttttgtctcatcttctactgaaagcagagttgtggtttgttaactctgattttgcctttgattttgcccggcccctgcttcccaacactgtttatattggaggcttgatggaaaaacctattaaaccagtaccacaagacttggacaacttcattgccaactttggggatgcagggtttgtccttgtggcctttggctccatgttgaacacccatcagtcccaggaagtcctcaagaagatgcacaatgcctttgcccacctccctcaaggagtgatatggacatgtcagagttctcattggcccagagatgttcatttggccacaaatgtgaaaattgtggactggcttcctcagagtgacctcctggctcaccccagcatccgtctttttgtcactcatggtgggcagaacagcgtaatggaggccatccgtcatggtgtgcccatggtgggattaccagtcaatggagaccagcatggaaacatggtccgagtagtagccaaaaattatggtgtctctatccggttgaatcaggtcacagccgacacactgacacttacaatgaaacaagtcatagaagacaagaggtacaagtcggcagtggtggcagccagtgtcatcctgcactctcagcccctgagccccgcacagcggctggtgggctggatcgaccacatcctccagactgggggagcgacgcacctcaagccctatgccttccagcagccttggcatgagcagtacctcattgatgtctttgtgtttctgctggggctcactctgggcactatgtggctttgtgggaagctgctgggtgtggtggccaggtggctgcgtggggccaggaaggtgaagaagacatga

SEQ ID NO: 22 - DNA, 1572 bp, Homo sapiens
atggttgggcagcgggtgctgcttctagtggccttccttctttctggggtcctgctctcagaggctgccaaaatcctgacaatatctacactgggtggaagccattacctactgttggaccgggtgtctcagattcttcaagagcatggtcataatgtgactatgcttcatcagagtggaaagtttttgatcccagatattaaagaggaggaaaaatcataccaagttatcaggtggttttcacctgaagatcatcaaaaaagaattaagaagcattttgatagctacatagaaacagcattggatggcagaaaagaatctgaagcccttgtaaagctaatggaaatatttgggactcaatgtagttatttgctaagcagaaaggatataatggattccttaaagaatgagaactatgatctggtatttgttgaagcatttgatttctgttctttcctgattgctgagaagcttgtgaaaccatttgtggccattcttcccaccacattcggctctttggattttgggctaccaagccccttgtcttatgttccagtattcccttccttgctgactgatcacatggacttctggggccgagtgaagaattttctgatgttctttagtttctccaggagccaatgggacatgcagtctacatttgacaacaccatcaaggagcatttcccagaaggctctaggccagttttgtctcatcttctactgaaagcagagttgtggtttgttaactctgattttgcctttgattttgcccggcccctgcttcccaacactgtttatattggaggcttgatggaaaaacctattaaaccagtaccacaagacttggacaacttcattgccaactttggggatgcagggtttgtccttgtggcctttggctccatgttgaacacccatcagtcccaggaagtcctcaagaagatgcacaatgcctttgcccacctccctcaaggagtgatatggacatgtcagagttctcattggcccagagatgttcatttggccacaaatgtgaaaattgtggactggcttcctcagagtgacctcctggctcaccccagcatccgtctttttgtcactcatggtgggcagaacagcgtaatggaggccatccgtcatggtgtgcccatggtgggattaccagtcaatggagaccagcatggaaacatggtccgagtagtagccaaaaattatggtgtctctatccggttgaatcaggtcacagccgacacactgacacttacaatgaaacaagtcatagaagacaagaggtacaagtcggcagtggtggcagccagtgtcatcctgcactctcagcccctgagccccgcacagcggctggtgggctggatcgaccacatcctccagactgggggagcgacgcacctcaagccctatgccttccagcagccttggcatgagcagtacctcattgatgtctttgtgtttctgctggggctcactctgggcactatgtggctttgtgggaagctgctgggtgtggtggccaggtggctgcgtggggccaggaaggtgaagaagacatga

SEQ ID NO: 23 - DNA, 759 bp, Homo sapiens
atgagctgcgtcctgggtggtgtcatccccttggggctgctgttcctggtctgcggatcccaaggctacctcctgcccaacgtcactctcttagaggagctgctcagcaaataccagcacaacgagtctcactcccgggtccgcagagccatccccagggaggacaaggaggagatcctcatgctgcacaacaagcttcggggccaggtgcagcctcaggcctccaacatggagtacatgacctgggatgacgaactggagaagtctgctgcagcgtgggccagtcagtgcatctgggagcacgggcccaccagtctgctggtgtccatcgggcagaacctgggcgctcactggggcaggtatcgctctccggggttccatgtgcagtcctggtatgacgaggtgaaggactacacctacccctacccgagcgagtgcaacccctggtgtccagagaggtgctcggggcccatgtgcacgcactacacacagatagtttgggccaccaccaacaagatcggttgtgctgtgaacacctgccggaagatgactgtctggggagaagtttgggagaacgcggtctactttgtctgcaattattctccaaaggggaactggattggagaagccccctacaagaatggccggccctgctctgagtgcccacccagctatggaggcagctgcaggaacaacttgtgttaccgaggtaggaaatttactcccaacacttttgcaatgaatttgccctcagtctga

SEQ ID NO: 24 - DNA, 1494 bp, Homo sapiens
atgagctgcgtcctgggtggtgtcatccccttggggctgctgttcctggtctgcggatcccaaggctacctcctgcccaacgtcactctcttagaggagctgctcagcaaataccagcacaacgagtctcactcccgggtccgcagagccatccccagggaggacaaggaggagatcctcatgctgcacaacaagcttcggggccaggtgcagcctcaggcctccaacatggagtacatgacctgggatgacgaactggagaagtctgctgcagcgtgggccagtcagtgcatctgggagcacgggcccaccagtctgctggtgtccatcgggcagaacctgggcgctcactggggcaggtatcgctctccggggttccatgtgcagtcctggtatgacgaggtgaaggactacacctacccctacccgagcgagtgcaacccctggtgtccagagaggtgctcggggcccatgtgcacgcactacacacagatagtttgggccaccaccaacaagatcggttgtgctgtgaacacctgccggaagatgactgtctggggagaagtttgggagaacgcggtctactttgtctgcaattattctccaaaggggaactggattggagaagccccctacaagaatggccggccctgctctgagtgcccacccagctatggaggcagctgcaggaacaacttgtgttaccgagaagaaacctacactccaaaacctgaaacggacgagatgaatgaggtggaaacggctcccattcctgaagaaaaccatgtttggctccaaccgagggtgatgagacccaccaagcccaagaaaacctctgcggtcaactacatgacccaagtcgtcagatgtgacaccaagatgaaggacaggtgcaaagggtccacgtgtaacaggtaccagtgcccagcaggctgcctgaaccacaaggcgaagatctttggaactctgttctatgaaagctcgtctagcatatgccgcgccgccatccactacgggatcctggatgacaagggaggcctggtggatatcaccaggaacgggaaggtccccttcttcgtgaagtctgagagacacggcgtgcagtccctcagcaaatacaaaccttccagctcattcatggtgtcaaaagtgaaagtgcaggatttggactgctacacgaccgttgctcagctgtgcccgtttgaaaagccagcaactcactgcccaagaatccattgtccggcacactgcaaagacgaaccttcctactgggctccggtgtttggaaccaacatctatgcagatacctcaagcatctgcaagacagccgtgcacgcgggagtcatcagcaacgagagtgggggtgacgtggacgtgatgcccgtggataaaaagaagacctacgtgggctcgctcaggaatggagttcagtctgaaagcctggggactcctcgggatggaaaggccttccggatctttgctgtcaggcagtga

SEQ ID NO: 25 - DNA, 1315 bp, Homo sapiens
batgctgcacaacaagcttcggggccaggtgcagcctcaggcctccaacatggagtacatgacctgggatgacgaactggagaagtctgctgcagcgtgggccagtcagtgcatctgggagcacgggcccaccagtctgctggtgtccatcgggcagaacctgggcgctcactggggcaggtatcgctctccggggttccatgtgcagtcctggtatgacgaggtgaaggactacacctacccctacccgagcgagtgcaacccctggtgtccagagaggtgctcggggcccatgtgcacgcactacacacagatagtttgggccaccaccaacaagatcggttgtgctgtgaacacctgccggaagatgactgtctggggagaagtttgggagaacgcggtctactttgtctgcaattattctccaaaggggaactggattggagaagccccctacaagaatggccggccctgctctgagtgcccacccagctatggaggcagctgcaggaacaacttgtgttaccgagaagaaacctacactccaaaacctgaaacggacgagatgaatgaggtggaaacggctcccattcctgaagaaaaccatgtttggctccaaccgagggtgatgagacccaccaagcccaagaaaacctctgcggtcaactacatgacccaagtcgtcagatgtgacaccaagatgaaggacaggtgcaaagggtccacgtgtaacaggtaccagtgcccagcaggctgcctgaaccacaaggcgaagatctttggaactctgttctatgaaagctcgtctagcatatgccgcgccgccatccactacgggatcctggatgacaagggaggcctggtggatatcaccaggaacgggaaggtccccttcttcgtgaagtctgagagacacggcgtgcagtccctcagcaaatacaaaccttccagctcattcatggtgtcaaaagtgaaagtgcaggatttggactgctacacgaccgttgctcagctgtgcccgtttgaaaagccagcaactcactgcccaagaatccattgtccggcacactgcaaagacgaaccttcctactgggctccggtgtttggaaccaacatctatgcagatacctcaagcatctgcaagacagccgtgcacgcgggagtcatcagcaacgagagtgggggtgacgtggacgtgatgcccgtggataaaaagaagacctacgtgggctcgctcaggaatggagttcagtctgaaagcctggggactcctcgggatggaaaggccttccggatctttgctgtcaggcagtga

SEQ ID NO: 26 - DNA, 927 bp, Homo sapiens
atggttgggggcgtcctcgcctcgctgggcttcgtcttctcggctttcgccagcgatctgctgcatctctacctcggcctgggcctcctcgctggctttggttgggccctggtgttcgcccccgccctaggcaccctctcgcgttacttctcccgccgtcgagtcttggcggtggggctggcgctcaccggcaacggggcctcctcgctgctcctggcgcccgccttgcagcttcttctcgatactttcggctggcggggcgctctgctcctcctcggcgcgatcaccctccacctcaccccctgtggcgccctgctgctacccctggtccttcctggagaccccccagccccaccgcgtagtcccctagctgccctcggcctgagtctgttcacacgccgggccttctcaatctttgctctaggcacagccctggttgggggcgggtacttcgttccttacgtgcacttggctccccacgctttagaccggggcctggggggatacggagcagcgctggtggtggccgtggctgcgatgggggatgcgggcgcccggctggtctgcgggtggctggcagaccaaggctgggtgcccctcccgcggctgctggccgtattcggggctctgactgggctggggctgtgggtggtggggctggtgcccgtggtgggcggcgaagagagctgggggggtcccctgctggccgcggctgtggcctatgggctgagcgcggggagttacgccccgctggttttcggtgtactccccgggctggtgggcgtcggaggtgtggtgcaggccacagggctggtgatgatgctgatgagcctcggggggctcctgggccctcccctgtcaggtaaggacctgagctcacagatctgcctacaactatcctctgcccctggggttcgaggcttctaa

SEQ ID NO: 27 - DNA, 1344 bp, Homo sapiens
atgaccccccagcccgccggacccccggatgggggctggggctgggtggtggcggccgcagccttcgcgataaacgggctgtcctacgggctgctgcgctcgctgggccttgccttccctgaccttgccgagcactttgaccgaagcgcccaggacactgcgtggatcagcgccctggccctggccgtgcagcaggcagccagccccgtgggcagcgccctgagcacgcgctggggggcccgccccgtggtgatggttgggggcgtcctcgcctcgctgggcttcgtcttctcggctttcgccagcgatctgctgcatctctacctcggcctgggcctcctcgctggctttggttgggccctggtgttcgcccccgccctaggcaccctctcgcgttacttctcccgccgtcgagtcttggcggtggggctggcgctcaccggcaacggggcctcctcgctgctcctggcgcccgccttgcagcttcttctcgatactttcggctggcggggcgctctgctcctcctcggcgcgatcaccctccacctcaccccctgtggcgccctgctgctacccctggtccttcctggagaccccccagccccaccgcgtagtcccctagctgccctcggcctgagtctgttcacacgccgggccttctcaatctttgctctaggcacagccctggttgggggcgggtacttcgttccttacgtgcacttggctccccacgctttagaccggggcctggggggatacggagcagcgctggtggtggccgtggctgcgatgggggatgcgggcgcccggctggtctgcgggtggctggcagaccaaggctgggtgcccctcccgcggctgctggccgtattcggggctctgactgggctggggctgtgggtggtggggctggtgcccgtggtgggcggcgaagagagctgggggggtcccctgctggccgcggctgtggcctatgggctgagcgcggggagttacgccccgctggttttcggtgtactccccgggctggtgggcgtcggaggtgtggtgcaggccacagggctggtgatgatgctgatgagcctcggggggctcctgggccctcccctgtcaggcttcctaagggatgagacaggagacttcaccgcctctttcctcctgtctggttctttgatcctctccggcagcttcatctacatagggttgcccagggcgctgccctcctgtggtccagcctcccctccagccacgcctcccccagagacgggggagctgcttcccgctccccaggcagtcttgctgtccccaggaggccctggctccactctggacaccacttgttga

SEQ ID NO: 28 - DNA, 1375 bp, Homo sapiens
batggcgcgcaggacagagccccccgacgggggctggggatgggtggtggtgctctcagcgttcttccagtcggcgcttgtgtttggggtgctccgctcctttggggtcttcttcgtggagtttgtggcggcgtttgaggagcaggcagcgcgcgtctcctggatcgcctccataggaatcgcggtgcagcagtttgggagcccggtaggcagtgccctgagcacgaagttcgggcccaggcccgtggtgatgactggaggcatcttggctgcgctggggatgctgctcgcctcttttgctacttccttgacccacctatacctgagtattgggttgctgtcaggctctggctgggctttgaccttcgctccgaccctggcctgcctgtcctgttatttctctcgccgacgatccctggccaccgggctggcactgacaggcgtgggcctctcctccttcacatttgccccctttttccagtggctgctcagccactacgcctggagggggtccctgctgctggtgtctgccctctccctccacctagtggcctgtggtgctctcctccgcccaccctccctggctgaggaccctgctgtgggtggtcccagggcccaactcacctctctcctccatcatggccccttcctccgttacactgttgccctcaccctgatcaacactggctacttcattccctacctccacctggtggcccatctccaggacctggattgggacccactacctgctgccttcctactctcagttgttgctatttctgacctcgtggggcgtgtggtctccggatggctgggagatgcagtcccagggcctgtgacacgactcctgatgctctggaccaccttgactggggtgtcactagccctgttccctgtagctcaggctcccacagccctggtggctctggctgtggcctacggcttcacatcaggggctctggccccactggccttctccgtgctgcctgaactaatagggactagaaggatttactgtggcctgggactgttgcagatgatagagagcatcggggggctgctggggcctcctctctcaggctacctccgggatgtgtcaggcaactacacggcttcttttgtggtggctggggccttccttctttcagggagtggcattctcctcaccctgccccacttcttctgcttctcaactactacctccgggcctcaggaccttgtaacagaagcactagatactaaagttcccctacccaaggaggggctggaaggaggactgaactccacagagtcaggcccagaaagccaaagcttgacagctccaggtcttctcttgccacgtcttggtctccacagaaccacagtgccttaa

SEQ ID NO: 29 - DNA, 510 bp, Homo sapiens
atgaccatgaagacctctggggccacttgtgatgcaaacagtgtgatgaactgcgggatccgcgggtctgaaatgtttgctgagatggatttgagggccataaaaccttaccagactctgatcaaaaaagtcgggcagagacattgcgtggaccctgctgtcatcgcagccatcatctccagggaaagccatgggggatctgtcctgcaagacggctgggaccacaggggacttaaatttggcttgatgcagcttgataaacaaacgtaccaccctgtcggtgcctgggatagcaaagagcacctttcacaggctactgggattctaacagagagaattaaggcaatccagaaaaaattccccacgtggagtgttgctcagcacctcaaaggtggtctctcagcttttaagtcaggaattgaagcgattgccaccccatcggacatagacaatgacttcgtcaatgatatcattgctcgagctaagttctataaaagacaaagcttctag

SEQ ID NO: 30 - DNA, 561 bp, Homo sapiens
atgaagcctcacctacatccacgcctgtaccacggctgctatggggacatcatgaccatgaagacctctggggccacttgtgatgcaaacagtgtgatgaactgcgggatccgcgggtctgaaatgtttgctgagatggatttgagggccataaaaccttaccagactctgatcaaaaaagtcgggcagagacattgcgtggaccctgctgtcatcgcagccatcatctccagggaaagccatgggggatctgtcctgcaagacggctgggaccacaggggacttaaatttggcttgatgcagcttgataaacaaacgtaccaccctgtcggtgcctgggatagcaaagagcacctttcacaggctactgggattctaacagagagaattaaggcaatccagaaaaaattccccacgtggagtgttgctcagcacctcaaaggtggtctctcagcttttaagtcaggaattgaagcgattgccaccccatcggacatagacaatgacttcgtcaatgatatcattgctcgagctaagttctataaaagacaaagcttctag

SEQ ID NO: 31 - DNA, 2028 bp, Homo sapiens
atggagagcggcaccagcagccctcagcctccacagttagatcccctggatgcgtttccccagaagggcttggagcctggggacatcgcggtgctagttctgtacttcctctttgtcctggctgttggactatggtccacagtgaagaccaaaagagacacagtgaaaggctacttcctggctggaggggacatggtgtggtggccagtgggtgcatccttgtttgccagcaatgttggaagtggacatttcattggcctggcagggtcaggtgctgctacgggcatttctgtatcagcttatgaacttaatggcttgttttctgtgctgatgttggcctggatcttcctacccatctacattgctggtcaggtcaccacgatgccagaatacctacggaagcgcttcggtggcatcagaatccccatcatcctggctgtactctacctatttatctacatcttcaccaagatctcggtagacatgtatgcaggtgccatcttcatccagcagtctttgcacctggatctgtacctggccatagttgggctactggccatcactgctgtatacacggttgctggtggcctggctgctgtgatctacacggatgccctgcagacgctgatcatgcttataggagcgctcaccttgatgggctacagtttcgccgcggttggtgggatggaaggactgaaggagaagtacttcttggccctggctagcaaccggagtgagaacagcagctgcgggctgccccgggaagatgccttccatattttccgagatccgctgacatctgatctcccgtggccgggggtcctatttggaatgtccatcccatccctctggtactggtgcacggatcaggtgattgtccagcggactctggctgccaagaacctgtcccatgccaaaggaggtgctctgatggctgcatacctgaaggtgctgcccctcttcataatggtgttccctgggatggtcagccgcatcctcttcccagatcaagtggcctgtgcagatccagagatctgccagaagatctgcagcaacccctcaggctgttcggacatcgcgtatcccaaactcgtgctggaactcctgcccacagggctccgtgggctgatgatggctgtgatggtggcggctctcatgtcctccctcacctccatctttaacagtgccagcaccatcttcaccatggacctctggaatcacctccggcctcgggcatctgagaaggagctcatgattgtgggcagggtgtttgtgctgctgctggtcctggtctccatcctctggatccctgtggtccaggccagccagggcggccagctcttcatctatatccagtccatcagctcctacctgcagccgcctgtggcggtggtcttcatcatgggatgtttctggaagaggaccaatgaaaagggtgccttctggggcctgatctcgggcctgctcctgggcttggttaggctggtcctggactttatttacgtgcagcctcgatgcgaccagccagatgagcgcccggtcctggtgaagagcattcactacctctacttctccatgatcctgtccacggtcaccctcatcactgtctccaccgtgagctggttcacagagccaccctccaaggagatggtcagccacctgacctggtttactcgtcacgaccccgtggtccagaaggaacaagcaccaccagcagctcccttgtctcttaccctctctcagaacgggatgccagaggccagcagcagcagcagcgtccagttcgagatggttcaagaaaacacgtctaaaacccacagctgtgacatgaccccaaagcagtccaaagtggtgaaggccatcctgtggctctgtggaatacaggagaagggcaaggaagagctcccggccagagcagaagccatcatagtttccctggaagaaaaccccttggtgaagaccctcctggacgtcaacctcattttctgcgtgagctgcgccatctttatctggggctattttgcttag

SEQ ID NO: 32 - DNA, 1458 bp, Homo sapiens
atggagccctgttggggagaaggcttgtttcatctagcacccccacgccaccatccccagaaggctgactggcatttctgtccacagcacattcaggaattcaccaatgagacatggcaggcgcgtactggagagccactgcccgatcacctagtcctgcttatgtggtccctcatcgtgtctctgtatcccctgggaggcctctttggagcactgcttgcaggtcccttggccatcacgctgggaaggaagaagtccctcctggtgaataacatctttgtggtgtcagcagcaatcctgtttggattcagccgcaaagcaggctcctttgagatgatcatgctgggaagactgctcgtgggagtcaatgcaggtgtgagcatgaacatccagcccatgtacctgggggagagcgcccctaaggagctccgaggagctgtggccatgagctcagccatctttacggctctggggatcgtgatgggacaggtggtcggactcagggagctcctaggtggccctcaggcctggcccctgctgctggccagctgcctggtgcccggggcgctccagctcgcctccctgcctctgctccctgaaagcccgcgctacctcctcattgactgtggagacaccgaggcctgcctggcagcactacggcggctccggggctccggggacttggcaggggagctggaggagctggaggaggagcgcgctgcctgccagggctgccgtgcccggcgcccatgggagctgttccagcatcgggccctgaggagacaggtgacaagcctcgtggttctgggcagtgccatggagctctgcgggaatgactcggtgtacgcctacgcctcctccgtgttccggaaggcaggagtgccggaagcgaagatccagtacgcgatcatcgggactgggagctgcgagctgctcacggcggttgttagttgtgtggtaatcgagagggtgggtcggcgcgtgctgctcatcggtgggtacagcctgatgacctgctgggggagcatcttcactgtggccctgtgcctgcagagctccttcccctggacactctacctggccatggcctgcatctttgccttcatcctcagctttggcattggccctgccggagtgacggggatcctggccacagagctgtttgaccagatggccaggcctgctgcctgcatggtctgcggggcgctcatgtggatcatgctcatcctggtcggcctgggatttccctttatcatggaggccttgtcccacttcctctatgtccctttccttggtgtctgtgtctgtggggccatctacactggcctgttccttcctgagaccaaaggcaagaccttccaagagatctccaaggaattacacagactcaacttccccaggcgggcccagggccccacgtggaggagcctggaggttatccagtcaacagaactctag

SEQ ID NO: 33 - DNA, 1491 bp, Homo sapiens
atgagagcgctccgaagactgattcagggcaggatcctgctcctgaccatctgcgctgccggcattggtgggacttttcagtttggctataacctctctatcatcaatgccccgaccttgcacattcaggaattcaccaatgagacatggcaggcgcgtactggagagccactgcccgatcacctagtcctgcttatgtggtccctcatcgtgtctctgtatcccctgggaggcctctttggagcactgcttgcaggtcccttggccatcacgctgggaaggaagaagtccctcctggtgaataacatctttgtggtgtcagcagcaatcctgtttggattcagccgcaaagcaggctcctttgagatgatcatgctgggaagactgctcgtgggagtcaatgcaggtgtgagcatgaacatccagcccatgtacctgggggagagcgcccctaaggagctccgaggagctgtggccatgagctcagccatctttacggctctggggatcgtgatgggacaggtggtcggactcagggagctcctaggtggccctcaggcctggcccctgctgctggccagctgcctggtgcccggggcgctccagctcgcctccctgcctctgctccctgaaagcccgcgctacctcctcattgactgtggagacaccgaggcctgcctggcagcactacggcggctccggggctccggggacttggcaggggagctggaggagctggaggaggagcgcgctgcctgccagggctgccgtgcccggcgcccatgggagctgttccagcatcgggccctgaggagacaggtgacaagcctcgtggttctgggcagtgccatggagctctgcgggaatgactcggtgtacgcctacgcctcctccgtgttccggaaggcaggagtgccggaagcgaagatccagtacgcgatcatcgggactgggagctgcgagctgctcacggcggttgttagttgtgtggtaatcgagagggtgggtcggcgcgtgctgctcatcggtgggtacagcctgatgacctgctgggggagcatcttcactgtggccctgtgcctgcagagctccttcccctggacactctacctggccatggcctgcatctttgccttcatcctcagctttggcattggccctgccggagtgacggggatcctggccacagagctgtttgaccagatggccaggcctgctgcctgcatggtctgcggggcgctcatgtggatcatgctcatcctggtcggcctgggatttccctttatcatggaggccttgtcccacttcctctatgtccctttccttggtgtctgtgtctgtggggccatctacactggcctgttccttcctgagaccaaaggcaagaccttccaagagatctccaaggaattacacagactcaacttccccaggcgggcccagggccccacgtggaggagcctggaggttatccagtcaacagaactctag

SEQ ID NO: 34 - DNA, 1501 bp, Homo sapiens
batgctccacgccctcctgcgatctagaatgattcagggcaggatcctgctcctgaccatctgcgctgccggcattggtgggacttttcagtttggctataacctctctatcatcaatgccccgaccttgcacattcaggaattcaccaatgagacatggcaggcgcgtactggagagccactgcccgatcacctagtcctgcttatgtggtccctcatcgtgtctctgtatcccctgggaggcctctttggagcactgcttgcaggtcccttggccatcacgctgggaaggaagaagtccctcctggtgaataacatctttgtggtgtcagcagcaatcctgtttggattcagccgcaaagcaggctcctttgagatgatcatgctgggaagactgctcgtgggagtcaatgcaggtgtgagcatgaacatccagcccatgtacctgggggagagcgcccctaaggagctccgaggagctgtggccatgagctcagccatctttacggctctggggatcgtgatgggacaggtggtcggactcagggagctcctaggtggccctcaggcctggcccctgctgctggccagctgcctggtgcccggggcgctccagctcgcctccctgcctctgctccctgaaagcccgcgctacctcctcattgactgtggagacaccgaggcctgcctggcagcactacggcggctccggggctccggggacttggcaggggagctggaggagctggaggaggagcgcgctgcctgccagggctgccgtgcccggcgcccatgggagctgttccagcatcgggccctgaggagacaggtgacaagcctcgtggttctgggcagtgccatggagctctgcgggaatgactcggtgtacgcctacgcctcctccgtgttccggaaggcaggagtgccggaagcgaagatccagtacgcgatcatcgggactgggagctgcgagctgctcacggcggttgttagttgtgtggtaatcgagagggtgggtcggcgcgtgctgctcatcggtgggtacagcctgatgacctgctgggggagcatcttcactgtggccctgtgcctgcagagctccttcccctggacactctacctggccatggcctgcatctttgccttcatcctcagctttggcattggccctgccggagtgacggggatcctggccacagagctgtttgaccagatggccaggcctgctgcctgcatggtctgcggggcgctcatgtggatcatgctcatcctggtcggcctgggatttccctttatcatggaggccttgtcccacttcctctatgtccctttccttggtgtctgtgtctgtggggccatctacactggcctgttccttcctgagaccaaaggcaagaccttccaagagatctccaaggaattacacagactcaacttccccaggcgggcccagggccccacgtggaggagcctggaggttatccagtcaacagaactctag

SEQ ID NO: 35 - DNA, 4077 bp, Homo sapiens
atggtagtggtgaagcccatgaacacaatggctccggtggtcacccggaataccggtcttattctctatgagggtcagtctcggcccctcacaggccctgcaggcagtggtccgcaaaacttggtcatcagcgatgaggatgacctagaagcagtgcggctagaggtggtggctgggctccggcatggtcaccttgtcattctgggtgcttccagtggcagctctgctcccaagagctttacagtggctgagctggcagccggccaggtggtctaccagcatgatgacagagacggctcgctgagcgacaacctggtgcttcgcatggtggatggaggaggcaggcaccaggtacagtttctgttccccatcaccttagtgcctgtggatgaccagccacctgttctcaatgccaacacggggctgacactggcagagggtgaaacagtgcccatcctgcccctttccctgagtgcaactgacatggattcagatgattctctgctgctttttgtgctggagtcacccttcttaactacggggcatctgcttctccgccaaactcaccctccccatgagaagcaggaacttctcagaggcctttggaggaaggagggggcattttatgagcgaacagtgacagagtggcagcagcaggacataacagagggcaggctgttctatagacactctgggccccatagtcctgggccagtcacagaccagttcacatttagagtccaggataaccatgaccctcctaatcagtccgggctacagcggtttgtgattcgtatccatcctgtggatcgcctccctccggagctgggcagtggctgtccccttcgtatggtggtacaggaatcccagctcacaccactgaggaagaagtggctgcgctacactgacctggacacagatgaccgagaactacgttacacagtgactcagtcccccacagacacagacgaaaatcacctgccagccccactgggtaccttggtcttgactgacaacccctcagtcgtggtgacccattttacccaagcccagatcaaccatcataaaattgcttacagacccccgggtcaagaactgggcgtggctactcgagtggcccagttccagttccaggtggaagaccgagctgggaatgtggctccaggtacctttaccctttacttgcatcccgtggacaaccagccacctgagatcctcaacaccggcttcactattcaggagaagggtcaccacatcctgagtgagacagagttgcacgtgaatgatgtagacactgatgttgcccatatctctttcactctcactcaggcacccaaacatggccacatgagagtgtctggacagatcctgcatgtagggggtctcttccacttggaggacataaaacagggccgagtttcctatgcccataatggggacaagtccctgactgatagctgctccttggaagtcagtgacagacatcatgtggtgcccatcactctcagagtaaatgtccggccagtggatgatgaagtgcccatactgagccatcctactggcactctggagtcctatctagatgtcttagaaaatggggctactgaaatcactgccaatgttattaaggggaccaatgaggaaactgatgacttgatgttgactttcctcttggaagatccacctttgtatggggaaatcttggtcaatggcattccagcagagcagtttactcaaagggacatcttggagggctctgttgtatatacccacaccagtggtgagataggcctattgcctaaagcggattcttttaacctgagtctgtcagatatgtctcaagaatggagaattggtggcaatactatccaaggagttactatatgggtgaccatcctgcctgttgatagccaggccccagaaatctttgtaggtgaacagttgatagtaatggaaggtgataaaagtgttataacatcagtgcatataagtgctgaagatgtcgactccctgaatgatgacatcttgtgcactatagttattcagcctacttcaggttatgttgaaaacatttctccagcaccaggctctgagaaatcaagagcagggattgccataagtgctttcaacttgaaagatctcaggcagggccacataaactatgtccagagtgtccataaaggggtggaacctgtggaggaccgatttgtatttcgttgttctgatggcattaacttttcagagagacagttcttccccattgtaatcattcccaccaatgatgaacagccagagatgtttatgagagaatttatggtgatggaaggcatgagtctggtaattgatacacccattctcaatgctgctgatgctgatgttcccctggatgatttaactttcactattacccaattccccactcatggtcacatcatgaatcagctgataaatggcacggttttggtcgaaagcttcaccttggatcagatcatagagagttccagcattatttatgagcatgatgactccgagacccaggaagacagttttgtgattaaactaacagatgggaagcactctgtggaaaagacggtcctcattatagttatccctgttgatgatgagacgcccagaatgactatcaataatggactagaaatagaaattggggataccaagattatcaacaacaaaatattaatggcaacagatttagattcagaagacaaatctttggtttatattattcgttatgggccaggacatggcttattacagagacgaaaacctactggtgcctttgaaaatatcacactgggcatgaattttacccaggatgaagtagacagaaacttaattcagtatgtccatttggggcaagagggcattcgggacctaattaaatttgatgtgactgatggaataaatcccctcatagatcgttacttttatgtgtccatcgggagcattgacattgtcttccctgatgtgataagtaagggagtgtccttgaaagaaggtggcaaagtcactcttacaacagacctactaagcactagtgacttgaacagtcctgatgaaaacttggtttttaccatcaccagggctcccatgcgaggtcacctggaatgcacggatcagcctggtgtgtccatcacgtctttcactcagctgcaactggctggaaacaaaatctactacatccacacagctgatgatgaagtgaaaatggacagttttgagtttcaagtcaccgatggacgtaaccctgtctttcggacattccgtatctccattagcgatgtggacaataaaaagccagtggtcaccatccacaagctggttgtcagtgaaagtgaaaacaagctgattactccttttgagctcactgtcgaagacagagatactcctgacaagctcctgaaattcactatcacccaggtgcctattcatggccatctcctattcaacaataccagacctgtcatggtttttaccaagcaagacttgaatgaaaacttaatcagctacaaacatgatggcactgagtcaagtgaagatagcttctccttcacagtgactgatggcacccatacagacttctatgtttttcctgatacggtgtttgaaacaaggagaccccaagtgatgaagatccaggtcttggctgttgacaacagtgtcccccaaatcgcagtgaataagggggcctctacacttcgcactctagccactggccacttggggttcatgatcacaagcaaaatattgaaagtggaggacagagacagcttacacatttctcttagatttatcgtgacagaggcccctcaacatggatatcttctcaacctggacaaaggcaaccacagcatcactcagttcacacaagctgacattgatgacatgaaaatatgctatgtcttaagagaaggggctaatgccacaagtgatatgttctattttgcagttgaagatggtggtaagtattcccctctcctggtagtgaccgcaaggagagacgcttttcttggttgctctctgatgacattattacaagaagtcttcatcaagtaa

SEQ ID NO: 36 - DNA, 9318 bp, Homo sapiens
atggcacgatcttggctcactgcaacctccacctcccggcccgctgccttcggcagggcgttgctgtcccctggtctcgcgggggctgcaggggtccctgctgaggaggccatagtgctggcgaaccgcggactccgggtgcctttcggccgtgaagtctggctggatcccctgcatgacctggtgttgcaggtgcagcccggggaccgctgcgcggtttcggtactagacaacgacgcactggcccagcgaccgggccgcctgagtcccaagcgcttcccgtgcgactttggccctggcgaggtgcgctactctcacctgggcgcgcgcagcccgtctcgggaccgcgtccggctgcagctgcgctatgacgcgcccggaggggcagtagtgctaccactggtactggaggtggaggtggtcttcacccagctggaggttgtgactcggaacttgcctctggtcgtggaagaactgctggggaccagcaatgccctggacgcgcggagcctggagttcgccttccagcccgagacagaggagtgccgcgtgggcatcctgtccggcttgggcgcgctgcctcgctatggagaactcctccactacccgcaggtccctggaggagccagagagggaggcgccccggagactctcctgatggactgcaaagctttccaggaactaggcgtgcgctatcgccacacagccgccagtcgctcaccaaacagggactggatacccatggtggtggagctgcgttcacgaggggctcctgtgggcagccctgctttgaaacgcgagcacttccaggttctggtgaggatccgaggaggggccgagaacactgcacccaagcccagtttcgtggccatgatgatgatggaggtggaccagtttgtactgacggccctgaccccagacatgctggcagccgaggatgctgagtctccctctgacctgttgatcttcaaccttacttctccattccagcctggccagggctacttggtgagcaccgatgatcgcagcctgcccctttcctccttcactcagagggatctgcggctcctgaagattgcctaccagcccccttctgaagactctgaccaggagcgcctctttgaactggaattggaggtagtggatctagaaggagcagcttcagacccttttgccttcatggtagtggtgaagcccatgaacacaatggctccggtggtcacccggaataccggtcttattctctatgagggtcagtctcggcccctcacaggccctgcaggcagtggtccgcaaaacttggtcatcagcgatgaggatgacctagaagcagtgcggctagaggtggtggctgggctccggcatggtcaccttgtcattctgggtgcttccagtggcagctctgctcccaagagctttacagtggctgagctggcagccggccaggtggtctaccagcatgatgacagagacggctcgctgagcgacaacctggtgcttcgcatggtggatggaggaggcaggcaccaggtacagtttctgttccccatcaccttagtgcctgtggatgaccagccacctgttctcaatgccaacacggggctgacactggcagagggtgaaacagtgcccatcctgcccctttccctgagtgcaactgacatggattcagatgattctctgctgctttttgtgctggagtcacccttcttaactacggggcatctgcttctccgccaaactcaccctccccatgagaagcaggaacttctcagaggcctttggaggaaggagggggcattttatgagcgaacagtgacagagtggcagcagcaggacataacagagggcaggctgttctatagacactctgggccccatagtcctgggccagtcacagaccagttcacatttagagtccaggataaccatgaccctcctaatcagtccgggctacagcggtttgtgattcgtatccatcctgtggatcgcctccctccggagctgggcagtggctgtccccttcgtatggtggtacaggaatcccagctcacaccactgaggaagaagtggctgcgctacactgacctggacacagatgaccgagaactacgttacacagtgactcagccccccacagacacagacgaaaatcacctgccagccccactgggtaccttggtcttgactgacaacccctcagtcgtggtgacccattttacccaagcccagatcaaccatcataaaattgcttacagacccccgggtcaagaactgggcgtggctactcgagtggcccagttccagttccaggtggaagaccgagctgggaatgtggctccaggtacctttaccctttacttgcatcccgtggacaaccagccacctgagatcctcaacaccggcttcactattcaggagaagggtcaccacatcctgagtgagacagagttgcacgtgaatgatgtagacactgatgttgcccatatctctttcactctcactcaggcacccaaacatggccacatgagagtgtctggacagatcctgcatgtagggggtctcttccacttggaggacataaaacagggccgagtttcctatgcccataatggggacaagtccctgactgatagctgctccttggaagtcagtgacagacatcatgtggtgcccatcactctcagagtaaatgtccggccagtggatgatgaagtgcccatactgagccatcctactggcactctggagtcctatctagatgtcttagaaaatggggctactgaaatcactgccaatgttattaaggggaccaatgaggaaactgatgacttgatgttgactttcctcttggaagatccacctttgtatggggaaatcttggtcaatggcattccagcagagcagtttactcaaagggacatcttggagggctctgttgtatatacccacaccagtggtgagataggcctattgcctaaagcggattcttttaacctgagtctgtcagataagtctcaagaatggagaattggtggcaatactatccaaggagttactatatgggtgaccatcctgcctgttgatagccaggccccagaaatctctgtaggtgaacagttgatagtaatggaaggtgataaaagtgttataacatcagtgcatataagtgctgaagatgtcgactccctgaatgatgacatcttgtgcactatagttattcagcctacttcaggttatgttgaaaacatttctccagcaccaggctctgagaaatcaagagcagggattgccataagtgctttcaacttgaaagatctcaggcagggccacataaactatgtccagagtgtccataaaggggtggaacctgtggaggaccgatttgtatttcgttgttctgatggcattaacttttcagagagacagttcttccccattgtaatcattcccaccaatgatgaacagccagagatgtttatgagagaatttatggtgatggaaggcatgagtctggtaattgatacacccattctcaatgctgctgatgctgatgttcccctggatgatttaactttcactattacccaattccccactcatggtcacatcatgaatcagctgataaatggcacggttttggtcgaaagcttcaccttggatcagatcatagagagttccagcattatttatgagcatgatgactccgagacccaggaagacagttttgtgattaaactaacagatgggaagcactctgtggaaaagacggtcctcattatagttatccctgttgatgatgagacgcccagaatgactatcaataatggactagaaatagaaattggggataccaagattatcaacaacaaaatattaatggcaacagatttagattcagaagacaaatctttggtttatattattcgttatgggccaggacatggcttattacagagacgaaaacctactggtgcctttgaaaatatcacactgggcatgaattttacccaggatgaagtagacagaaacttaattcagtatgtccatttggggcaagagggcattcgggacctaattaaatttgatgtgactgatggaataaatcccctcatagatcgttacttttatgtgtccatcgggagcattgacattgtcttccctgatgtgataagtaagggagtgtccttgaaagaaggtggcaaagtcactcttacaacagacctactaagcactagtgacttgaacagtcctgatgaaaacttggtttttaccatcaccagggctcccatgcgaggtcacctggaatgcacggatcagcctggtgtgtccatcacgtctttcactcagctgcaactggctggaaacaaaatctactacatccacacagctgatgatgaagtgaaaatggacagttttgagtttcaagtcaccgatggacgtaaccctgtctttcggacattccgtatctccattagcgatgtggacaataaaaagccagtggtcaccatccacaagctggttgtcagtgaaagtgaaaacaagctgattactccttttgagctcactgtcgaagacagagatactcctgacaagctcctgaaattcactatcacccaggtgcctattcatggccatctcctattcaacaataccagacctgtcatggtttttaccaagcaagacttgaatgaaaacttaatcagctacaaacatgatggcactgagtcaagtgaagatagcttctccttcacagtgactgatggcacccatacagacttctatgtttttcctgatacggtgtttgaaacaaggagaccccaagtgatgaagatccaggtcttggctgttgacaacagtgtcccccaaatcgcagtgaataagggggcctctacacttcgcactctagccactggccacttggggttcatgatcacaagcaaaatattgaaagtggaggacagagacagcttacacatttctcttagatttatcgtgacagaggcccctcaacatggatatcttctcaacctggacaaaggcaaccacagcatcactcagttcacacaagctgacattgatgacatgaaaatatgctatgtcttaagagaaggggctaatgccacaagtgatatgttctattttgcagttgaagatggtggtggaaacaagttaacgtaccagaattttcgtctgaattgggcatggatctcctttgaaaaggaatattacctggtcaatgaggactccaaatttctagatgttgttcttaaacgtagaggttacttgggagaaacttcttttataagtattggcacaagagacagaactgcagaaaaagacaaagacttcaagggcaaagcacagaaacaagtgcagttcaacccaggccagaccagggccacatggcgagtgcggatcctgagtgatggggagcatgagcagtctgaaacctttcaggtggtactctcagagcccgtgctggctgccttggaattccccacagtcgccactgttgagatcgttgatccaggagatgagccaactgtgtttattccccagtccaaatactccgttgaagaagatgttggtgagctgttcattcccatcaggaggagcggagatgtgagccaggagttgatggtggtctgttatacccaacaaggaacagcaactggaactgtgccgacttccgtgttgtcttactctgattacatatccaggcctgaggaccacaccagtgttgtccgctttgacaaagatgaacgggagaaactgtgtcggatagtcataattgatgactctttgtacgaggaggaggaaaccttccatgtccttctgagcatgcccatggggggaagaatcggatcagagttcccaggggctcaagttacaatcgttcctgacaaagatgatggtccatctgattccaaatttaatgttgctgagaactacagtttactgcctttcacctgttttcaaggtagcatagccacggcagaggcagccacccagggagggggacgcagcaccagacaagtagctgcagtcaaaaaagacaaagacttcaagggcaaagcacagaaacaagtgcagttcaacccaggccagaccagggccacatggcgagtgcggatcctgagtgatggggagcatgagcagtctgaaacctttcaggtggtactctcagagcccgtgctggctgccttggaattccccacagtcgccactgttgagatcgttgatccaggagatgcatgcccatggggggaagaatcggatcagagttcccaggggctcaagttacaatcgttcctgacaaagatgatggtgagtactaatttacttgaaaattctttttcccgggaagatcaacatcaggaacaactttctagacagaaaaaatgggaatctaaaacaatgataatatacacatttattttatgtgagacagaaaagccctgcattcttgagctgatggacgatgtgctctatgaggaggtagaggagctccgcctggtactcggcactccacaaagcaactctccctttggggctgcagttggtgaacaaaatgaaactctcataaggatccgagatgatgctgataagactgttattaaatttggagaaaccaaatttagtgtcactgaacccaaagaacctggagagtcggtggttataagaattccagtgattcgccaaggagacacttcaaaggtttccattgtgagagtccacaccaaggatggctcggccacctctggagaagactaccaccctgtgtcagaagaaattgagtttaaggaaggggaaacccagcacgtggttgaaatcgaagttacctttgacggggtgagagagatgagagaggccttcactgttcacctaaaacctgatgaaaatatgatagcagagatgcagctttccaactttgagctcaccctcagccctgatggcacaagagttggaaaccacaagtgctccaacctcctggattatactgaagtgaagactcattatggtttcttgactgatgctaccaaaaatccagaaataattggagagacatatccttaccagtacagcttgtccatcagaggttccactaccttgcgcttctaccggaacctgaacctagaggcctgtttatgggagttcgttagctactatgacatgtcagaactccttgctgactgcagatcagtgttgaatgcaagcatatttcatgagatggcacctgaaggaaagcaaagtaaatgcttggtaaattctactttatactctattttggaatgtcatgagagccttccaaacttttgcatttcagcattaaggatgggaaaatggagaaaaataaaaagcaaaccatcagcacagactccatgtgctcagaggcttcgtggttttattgaccaccccaggaaacagcccctgcagcaagccagtgctgacccaggcatgctccccgtgatctccactagagagctttccaactttgagctcaccctcagccctgatggcacaagagttggaaaccacaagtgctccaacctcctggattatactgaagtgaagactcattatggtttcttgactgatgctaccaaaaatccagaaataattggagagacatatccttaccagtacagcttgtccatcagaggttccactaccttgcgcttctaccggaacctgaacctagaggcctgtttatgggagttcgttagctactatgacatgtcagaactccttgctgactgtggtggcaccattggaacagatggacaggtcctaaacctagtgcagtcctatgtgacccttcgagtccctctgtatgtttcctacgtgttccattcccccgtgggggtaggaggctggcagcattttgacttgaagtcagagcttcgtctaacttttgtgtacgacaccgccatcttgtggaatgatggaattggcagccccccagaggctgaacttcaaggttctctctatccaaccagcatgcgcatcggtgatgaggggcgcttggccgtgcacttcaagacagaggctcagttccatggcttatttgtgctgtcacatcccgcatcctttacaagctcagtgatcatgtcagctgatcatccaggcctgacattttccctccgcctcataaggagtgaaccaacctataaccagccagtacagcagtggagctttgtctctgactttgccgtacgtgactactcagggacctatactgtgaagctggtgccatgcactgccccatcacatcaggaataccgcctgccagtcacctgcaaccccagagaacctgtcacctttgaccttgacatccgattccaacaggtcagtgatccagtggctgctgagtttagcttgaacacccaaatgtacctgctctctaagaagagtctctggttgtctgatggatccatgggattcgggcaagagagtgatgttgcttttgcagaaggtgatataatttatggtcgtgtcatggtggatcctgtccagaatctgggtgactccttttactgcagcattgagaaggtgtttctatgcactggagctgatggctatgttcccaagtatagtccaatgaatgcagaatatggctgcttagccgactctccttcactcttatatagatttaaaattgtggacaaagctcagccagagacacaagcgaccagttttggaaatgtcctatttaatgccaaactagcagtggatgaccctgaagccattctcttagtgaatcagcctggatctgatggatttaaagtcgactcaacaccactctttcaggtcgctctaggccgagaatggtatatacatacgatctatacagtgagatcgaaagacaatgccaatcgaggtattggcaaaagaagtgtggagtaccattctctggtgagtcaaggaaagccccaatccaccaccaagagccggaagaagagagagatcaggagcacaccctcactggcatgggagattggtgctgaaaacagtcgaggaacaaacatccagcacattgccctggaccgcaccaagaggcagatcccccatgggagagcacctccagatggcatcctcccctgggagctcaacagccccagctctgcagtcagcctggtcactgtggtgggaggcaccacggtagggttactcaccatctgcctcactgtcattgcagtgctgatgtgcaggggcaaggaaagtttcagggggaaggatgccccgaaaggctccagcagcagtgagcccatggtgcccccacagagccatcacaatgacagctcagaagtttga

SEQ ID NO: 37 - DNA, 1374 bp, Homo sapiens
atggggtgtagtggagcctggggcttgagctgcccttgtccacagacgccatcctgggcctggaggaagatgcggacgccatccatgaggaaccggagtggcgccgtgtggagcagggcctctgtccccttctctgcctgggaagtagaggtgcagatgagggtgacgggactggggcgccggggagcccagggcatggccgtgtggtacacccggggcaggggccatgtaggctctgtccttggggggctggcttcgtgggacggcatcgggatcttctttgactctccggcagaggatactcaggacagtcctgccatccgtgtgctggccagcgacgggcacatcccctctgagcagcctggggatggagctagccaagggctgggctcctgtcattgggacttccggaaccggccacaccccttcagagcacggatcacctactgggggcagaggctgcgcatgtccttgaacagtggcctcactcccagtgatccaggtgagttctgtgtggatgtggggcccctgcttttggtccctggaggtttctttggggtctcagcagccaccggcaccctggcagatgatcatgatgtcctgtccttcctgaccttcagcctgagtgagcccagcccagaggttccccctcagcccttcctggagatgcagcacgtccgcctggcgaggcagctggaagggctgtgggcaaggctgggcttgggcaccagggaggatgtaactccaaaatcagactctgaagctcaaggagaaggggaaaggctctttgacctggaggagacgctgggcagacaccgccggatcctgcaggctctgcggggtctctccaagcagctggcccaggctgagagacaatggaagaagcagctgggcccccaggccaagccaggcctgacggaggctggggatgcagctgtccgcatggctgcagaagcccaggtctcctacctgcctgtgggcattgagcatcatttcttagagctggaccacatcctgggcctcctgcaggaggagcttcggggcccggcgaaggcagcagccaaggccccccgcccacctggccagcccccaagggcctcctcgtgcctgcagcctggcatcttcctgttctacctcctcattcagactgtaggcttcttcggctacgtgcacttcaggcgcccggtgccccggccggcgaagaccatggcgttcatggtgaagaccatggtgggcggccagctgaagaacctcactgggagcctgggaggcggcgaggataagggagatggggacaagtcggcagccgaagctcagggcatgagccgggaggagtacgaggagtatcagaagcaactcgtggaagagaagtga

SEQ ID NO: 38 - DNA, 1581 bp, Homo sapiens
atgccggcggtcagtggtccaggtcccttattctgccttctcctcctgctcctggacccccacagccctgagacggggtgtcctcctctacgcaggtttgagtacaagctcagcttcaaaggcccaaggctggcattgcctggggctggaatacccttctggagccatcatggagacgccatcctgggcctggaggaagtgcggctgacgccatccatgaggaaccggagtggcgccgtgtggagcagggcctctgtccccttctctgcctgggaagtagaggtgcagatgagggtgacgggactggggcgccggggagcccagggcatggccgtgtggtacacccggggcaggggccatgtaggctctgtccttggggggctggcttcgtgggacggcatcgggatcttctttgactctccggcagaggatactcaggacagtcctgccatccgtgtgctggccagcgacgggcacatcccctctgagcagcctggggatggagctagccaagggctgggctcctgtcattgggacttccggaaccggccacaccccttcagagcacggatcacctactgggggcagaggctgcgcatgtccttgaacagtggcctcactcccagtgatccaggtgagttctgtgtggatgtggggcccctgcttttggtccctggaggtttctttggggtctcagcagccaccggcaccctggcagatgatcatgatgtcctgtccttcctgaccttcagcctgagtgagcccagcccagaggttccccctcagcccttcctggagatgcagcacgtccgcctggcgaggcagctggaagggctgtgggcaaggctgggcttgggcaccagggaggatgtaactccaaaatcagactctgaagctcaaggagaaggggaaaggctctttgacctggaggagacgctgggcagacaccgccggatcctgcaggctctgcggggtctctccaagcagctggcccaggctgagagacaatggaagaagcagctggggcccccaggccaagccaggcctgacggaggctgggccctggatgcttcctgccagattccatccaccccagggaggggtggccacctctccatgtcactcaataaggactctgccaaggtcggtgccctgctccatggacagtggactctgctccaggccctgcaagagatgagggatgcagctgtccgcatggctgcagaagcccaggtctcctacctgcctgtgggcattgagcatcatttcttagagctggaccacatcctgggcctcctgcaggaggagcttcggggcccggcgaaggcagcagccaaggccccccgcccacctggccagcccccaagggcctcctcgtgcctgcagcctggcatcttcctgttctacctcctcattcagactgtaggcttcttcggctacgtgcacttcaggcaggagctgaacaagagccttcaggagtgtctgtccacaggcagccttcctctgggtcctgcaccacacacccccagggccctggggattctgaggaggcagcctctccctgccagcatgcctgcctga

SEQ ID NO: 39 - DNA, 2715 bp, Homo sapiens
atgctcttggctctatgttcatctctggctcttatttttgcagctcctgtcagtggacagcttgagcattcagggaactactactgcacagctgacaatggctttggcccccagcacagtgaggtggtgagtctctctgtcactgtccctgtgtctcatcctgtcctcaccctcagctctgctgaggccctgacttttgaaggagccactgtgacacttcactcttcgctgatcctgcaagctccactttctgtgtttgaaggagacttcgtggttctgaggtgccgggcaaaggcggaagtaacactgaatactatgtacaagaatggtaacgtcctgacattccttaataaaagctctgacttccatattcatcacgcaagtctcaaagacaatggtgcatatcactttactggatttaacggaagtaatttctctgtttcttccaacatagtcaaaatccaagtccaagagcatctcctgccccagtggttcctgaaggcacctgatcccactgtggcccttagtgagatattctcagtcaatagaggtccacttctgacaggcacaggctcccgagttatgactccctggatttacttccctactgaggactggaacttcactcttattcccattactgtggacgatgcttgcaaaaggccttgtgctcctcgggctccttgggaagttggaagtctaacaccaggaaagagtttccagcagaaggggaacccagacctgctcatgggcccttttgatgttcttcacttgtccctcatcacacttttttgcaacatggcaggttctttgttttattatatggaacttggagaaaagaaagatgcattatttgttacaggaaatgaaagcagaagctatcattggttccagaagttcagccttgctggtgggcaagaagcagatcaaaagttgtggtttccccctggactgatgagtcaggatattttcatatatatcagacaaggtgagagttgtcttgtgattgagatgtttcatcaggtctacagacggcctgctggaggtgttccagtagagcatatgagtgtatgggctctcagggtgaatagttcgggactcttcgcctacgctgatggctggctctttactctctttcagctccagtctgtggacaatctgagcccatcaactcagtctcaaacacacgaaggccatgaaaagttatgtcccttcctggccatagggccacctgcacatagcaggggcagcttcctcagagcaaagaagagtctagttgcatacatcaaaggaaatcaactctccttccctccagtagaagacctattgcctaagtcccaatggccctgcatccaggtgcttcccaggaatatagctgcagctgcacacaaacctgtgatttccgtccatcctccatggaccacattcttcaaaggagagagagtgactctgacttgcaatggatttcagttctatgcaacagagaaaacaacatggtatcatcggcactactggggagaaaagttgaccctgaccccaggaaacaccctcgaggttcgggaatctggactgtacagatgccaggcccggggctccccacgaagtaaccctgtgcgcttgctcttttcttcaggtgagaaagaagactgtgtctctgacaagaatcctgctacaaatcacactccaatgtccaggaagagatcctcatgtagttctctaagtgccacttctttcatagctcctgctctgagactcgactccttaatcctgcaggcaccatattctgtgtttgaaggtgacacattggttctgagatgccacagaagaaggaaagagaaattgactgctgtgaaatatacttggaatggaaacattctttccatttctaataaaagctgggatcttcttatcccacaagcaagttcaaataacaatggcaattatcgatgcattggatatggagacgagaatgatgtatttagatcaaatttcaaaataattaaaattcaagggatccctgtgtctggggtgctcctggagacccagccctcagggggccaggctgttgaaggggagatgctggtccttgtctgctccgtggctgaaggcacaggggataccacattctcctggcaccgagaggacatgcaggagagtctggggaggaaaactcagcgttccctgagagcagagctggagctccctgccatcagacagagccatgcagggggatactactgtacagcagacaacagctacggccctgtccagagcatggtgctgaatgtcactgtgagagagaccccaggcaacagagatggccttgtcgccgcgggagccactggagggctgctcagtgctcttctcctggctgtggccctgctgtttcactgctggcgtcggaggaagtcagtacaccccaaaaagggagatttggtatactctgagatccagactactcagctgggagaagaagaggaaggaaatcatggcaataaaaatcaagaactagaacttgttaatgtaggagagagtttttctcacagggcatgcatatggagtactctaatgggaacttgccaaacaatcgggggtgctaatacctccaggacacttctagaggataaggatgtctcagttgtctactctgaggtaaagacacaacacccagataactcagctggaaagatcagctctaaggatgaagaaagttaa

SEQ ID NO: 40 - DNA, 1548 bp, Homo sapiens
atgctgctgtgggcgtccttgctggcctttgctccagtctgtggacaatctgcagctgcacacaaacctgtgatttccgtccatcctccatggaccacattcttcaaaggagagagagtgactctgacttgcaatggatttcagttctatgcaacagagaaaacaacatggtatcatcggcactactggggagaaaagttgaccctgaccccaggaaacaccctcgaggttcgggaatctggactgtacagatgccaggcccggggctccccacgaagtaaccctgtgcgcttgctcttttcttcagactccttaatcctgcaggcaccatattctgtgtttgaaggtgacacattggttctgagatgccacagaagaaggaaagagaaattgactgctgtgaaatatacttggaatggaaacattctttccatttctaataaaagctgggatcttcttatcccacaagcaagttcaaataacaatggcaattatcgatgcattggatatggagatgagaatgatgtatttagatcaaatttcaaaataattaaaattcaagaactatttccacatccagagctgaaagctacagactctcagcctacagaggggaattctgtaaacctgagctgtgaaacacagcttcctccagagcggtcagacaccccacttcacttcaacttcttcagagatggcgaggtcatcctgtcagactggagcacgtacccggaactccagctcccaaccgtctggagagaaaactcaggatcctattggtgtggtgctgaaacagtgaggggtaacatccacaagcacagtccctcgctacagatccatgtgcagcggatccctgtgtctggggtgctcctggagacccagccctcagggggccaggctgttgaaggggagatgctggtccttgtctgctccgtggctgaaggcacaggggataccacattctcctggcaccgagaggacatgcaggagagtctggggaggaaaactcagcgttccctgagagcagagctggagctccctgccatcagacagagccatgcagggggatactactgtacagcagacaacagctacggccctgtccagagcatggtgctgaatgtcactgtgagagagaccccaggcaacagagatggccttgtcgccgcgggagccactggagggctgctcagtgctcttctcctggctgtggccctgctgtttcactgctggcgtcggaggaagtcaggagttggtttcttgggagacgaaaccaggctccctcccgctccaggcccaggagagtcctcccattccatctgccctgcccaggtggagcttcagtcgttgtatgttgatgtacaccccaaaaagggagatttggtatactctgagatccagactactcagctgggagaagaagaggaagctaatacctccaggacacttctagaggataaggatgtctcagttgtctactctgaggtaaagacacaacacccagataactcagctggaaagatcagctctaaggatgaagaaagttaa

SEQ ID NO: 41 - DNA, 2494 bp, Homo sapiens
batggctgtgggaggaggacacaaaggagttagatgtcctccctctgctcgcagctcccaggtgcccacgtgctgcgctggctggaggcagcaaggggacgagtgtgggattgcggtgtgcgaaggcaactccacgtgctcagagaacgaggtgtgcgtgaggcctggcgagtgccgctgccgccacggctacttcggtgccaactgcgacaccaagtgcccgcgccagttctggggccccgactgcaaggagctgtgtagctgccacccacacgggcagtgcgaggacgtgacaggccagtgtacttgtcacgcgcggcgctggggcgcgcgctgcgagcatgcgtgccagtgccagcacggcacgtgccacccccggagcggcgcgtgccgctgtgagtccggctggtggggcgcgcagtgcgccagcgcgtgctactgcagcgccacgtcgcgctgcgacccacagaccggcgcctgcctgtgccacgcaggctggtggggccgcagctgcaacaaccagtgcgcctgcaactcgtctccctgcgagcagcagagcggccgctgtcagtgccgcgagcgtacgttcggcgcgcgctgcgatcgctactgccagtgcttccgcggccgctgccaccctgtggacggcacgtgtgcctgcgagccgggctaccgcggcaagtactgtcgcgagccgtgccccgccggcttctacggcttgggctgtcgccgccggtgtggccagtgcaagggccagcagccgtgcacggtggccgagggccgctgcttgacgtgcgagcccggctggaacggaaccaagtgcgaccagccttgcgccaccggtttctatggcgagggctgcagccaccgctgtccgccatgccgcgacgggcatgcctgtaaccatgtcaccggcaagtgtacgcgctgcaacgcgggctggatcggcgaccggtgcgagaccaagtgtagcaatggcacttacggcgaggactgcgccttcgtgtgcgccgactgcggcagcggacactgcgacttccagtcggggcgctgcctgtgcagccctggcgtccacgggccccactgtaacgtgacgtgcccgcccggactccacggcgcggactgtgctcaggcctgcagctgccacgaggacacgtgcgacccggtcactggtgcctgccacctagaaaccaaccagcgcaagggcgtgatgggcgcgggcgcgctgctcgtcctgctcgtctgcctgctgctctcgctgctcggctgctgctgcgcttgccgcggcaaggaccctacgcgccgaccccgcccccgcagggagctttcgcttgggaggaagaaggcgccgcaccgactatgcgggcgcttcagtcgcatcagcatgaagctgccccggatcccgctccggaggcagaaactacccaaagtcgtagtggcccaccacgacctggataacacactcaactgcagcttcctggagccaccctcagggctggagcagccctcaccatcctggtcctctcgggcctccttctcctcgtttgacaccactgatgaaggccctgtgtactgtgtaccccatgaggaggcaccagcggagagccgggaccccgaagtccccactgtccctgccgaggcgccggcgccgtcccctgtgcccttgaccacgccagcctccgccgaggaggcgatacccctccccgcgtcctccgacagcgagcggtcggcgtccagcgtggaggggcccggaggggctctgtacgcgcgcgtggcccgacgcgaggcccggccggcccgggcccggggcgagattgggggcctgtcgctgtcgccatcgcccgagcgcaggaaaccgccgccacctgaccccgccaccaagcctaaggtgtcctggatccacggcaagcacagcgccgctgcagctggccgtgcgccctcaccaccgccgccaggctccgaggccgcgcccagccccagcaagaggaaacggacgcccagcgacaaatcggcgcatacggtcgaacacggcagcccccggacccgcgacccaacgccgcgccccccggggctgcccgaggaggcgacagccctcgctgcgccctcgccgcccagggcccgagcgcgcgccgcgccccggcctcttggagcccacggacgccggcggtcccccgcgaagcgcgccgaggctgcctccatgttggccgctgacgtgcgcggcaagactcgcagcctgggccgcgccgaggtggccctgggcgcgcagggccccagggaaaagccggcgcccccacagaaagccaagcgctccgtgccgccagcctcgcccgcccgcgcgcccccagcgaccgaaaccccggggcctgagaaggcggcgaccgacttgcccgcgcctgagaccccccggaagaagacccccatccagaagccgccgcgcaagaagagccgggaggcggcgggcgagctgggcagggcgggcgcacccaccctgtag

SEQ ID NO: 42 - DNA, 2614 bp, Homo sapiens
batggagggcgcagggccccggggggccgggccggcgcggcgccggggagccggggggccgccgtcaccgctgctgccgtcgctgctgctgctgctgctgctctggatgctgccggacaccgtggcgcctcaggaactgaaccctcgcggccgcaacgtgtgccgtgctcccggctcccaggtgcccacgtgctgcgctggctggaggcagcaaggggacgagtgtgggattgcggtgtgcgaaggcaactccacgtgctcagagaacgaggtgtgcgtgaggcctggcgagtgccgctgccgccacggctacttcggtgccaactgcgacaccaagtgcccgcgccagttctggggccccgactgcaaggagctgtgtagctgccacccacacgggcagtgcgaggacgtgacaggccagtgtacttgtcacgcgcggcgctggggcgcgcgctgcgagcatgcgtgccagtgccagcacggcacgtgccacccccggagcggcgcgtgccgctgtgagtccggctggtggggcgcgcagtgcgccagcgcgtgctactgcagcgccacgtcgcgctgcgacccacagaccggcgcctgcctgtgccacgcaggctggtggggccgcagctgcaacaaccagtgcgcctgcaactcgtctccctgcgagcagcagagcggccgctgtcagtgccgcgagcgtacgttcggcgcgcgctgcgatcgctactgccagtgcttccgcggccgctgccaccctgtggacggcacgtgtgcctgcgagccgggctaccgcggcaagtactgtcgcgagccgtgccccgccggcttctacggcttgggctgtcgccgccggtgtggccagtgcaagggccagcagccgtgcacggtggccgagggccgctgcttgacgtgcgagcccggctggaacggaaccaagtgcgaccagccttgcgccaccggtttctatggcgagggctgcagccaccgctgtccgccatgccgcgacgggcatgcctgtaaccatgtcaccggcaagtgtacgcgctgcaacgcgggctggatcggcgaccggtgcgagaccaagtgtagcaatggcacttacggcgaggactgcgccttcgtgtgcgccgactgcggcagcggacactgcgacttccagtcggggcgctgcctgtgcagccctggcgtccacgggccccactgtaacgtgacgtgcccgcccggactccacggcgcggactgtgctcaggcctgcagctgccacgaggacacgtgcgacccggtcactggtgcctgccacctagaaaccaaccagcgcaagggcgtgatgggcgcgggcgcgctgctcgtcctgctcgtctgcctgctgctctcgctgctcggctgctgctgcgcttgccgcggcaaggaccctacgcgccgaccccgcccccgcagggagctttcgcttgggaggaagaaggcgccgcaccgactatgcgggcgcttcagtcgcatcagcatgaagctgccccggatcccgctccggaggcagaaactacccaaagtcgtagtggcccaccacgacctggataacacactcaactgcagcttcctggagccaccctcagggctggagcagccctcaccatcctggtcctctcgggcctccttctcctcgtttgacaccactgatgaaggccctgtgtactgtgtaccccatgaggaggcaccagcggagagccgggaccccgaagtccccactgtccctgccgaggcgccggcgccgtcccctgtgcccttgaccacgccagcctccgccgaggaggcgatacccctccccgcgtcctccgacagcgagcggtcggcgtccagcgtggaggggcccggaggggctctgtacgcgcgcgtggcccgacgcgaggcccggccggcccgggcccggggcgagattgggggcctgtcgctgtcgccatcgcccgagcgcaggaaaccgccgccacctgaccccgccaccaagcctaaggtgtcctggatccacggcaagcacagcgccgctgcagctggccgtgcgccctcaccaccgccgccaggctccgaggccgcgcccagccccagcaagaggaaacggacgcccagcgacaaatcggcgcatacggtcgaacacggcagcccccggacccgcgacccaacgccgcgccccccggggctgcccgaggaggcgacagccctcgctgcgccctcgccgcccagggcccgagcgcgcgccgcgccccggcctcttggagcccacggacgccggcggtcccccgcgaagcgcgccgaggctgcctccatgttggccgctgacgtgcgcggcaagactcgcagcctgggccgcgccgaggtggccctgggcgcgcagggccccagggaaaagccggcgcccccacagaaagccaagcgctccgtgccgccagcctcgcccgcccgcgcgcccccagcgaccgaaaccccggggcctgagaaggcggcgaccgacttgcccgcgcctgagaccccccggaagaagacccccatccagaagccgccgcgcaagaagagccgggaggcggcgggcgagctgggcagggcgggcgcacccaccctgtag

SEQ ID NO: 43 - DNA, 402 bp, Homo sapiens
atcgactgccgcgcggagcagggccgtacgccgctcatggtggccgtggggctgccggaccccgcgctgcgcgcgcgcttcgtgcggctgctgctcgagcagggtgctgcagtgaacctgcgagacgagcgcggccgcaccgcactcagcctggcgtgcgagcgaggccacctggacgccgtgcagctgctggtgcagttcagcggtgaccccgaggcggccgactctgcgggcaacagcccggtgatgtgggcggcggcctgcggccacggggcggtgctcgagttcctggtgcggtccttccgccgcctaggcctgcgcctcgaccgcaccaaccgtgcggggctcaccgcgctgcaactggccgccgcccgcggccacgggacctgtgtgcaggccctc

SEQ ID NO: 44 - DNA, 975 bp, Homo sapiens
atgctcaaacccaaggacctgtgcccccgagcggggacgcgcaccttcctggaggccatgcaggcgggcaaagtgcacttggcccgcttcgtgttggatgcgctggaccgcagcatcatcgactgccgcgcggagcagggccgtacgccgctcatggtggccgtggggctgccggaccccgcgctgcgcgcgcgcttcgtgcggctgctgctcgagcagggtgctgcagtgaacctgcgagacgagcgcggccgcaccgcactcagcctggcgtgcgagcgaggccacctggacgccgtgcagctgctggtgcagttcagcggtgaccccgaggcggccgactctgcgggcaacagcccggtgatgtgggcggcggcctgcggccacggggcggtgctcgagttcctggtgcggtccttccgccgcctaggcctgcgcctcgaccgcaccaaccgtgcggggctcaccgcgctgcaactggccgccgcccgcggccacgggacctcggcgggcggccacggcggcgaggctggctcagcgggcaagaattcgggccggcaccgggcgcagggcagcgaacggcccgagctgggtcggagcatgagcctggctctaggtgcggtaaccgaggaggaggctgcccgcctgcgggctggggccctgatggccctaccaaactcgccccagtcttcggggactgggcggtggcgctcacaggaggtgctggagggagcgcccccaaccttagcgcaagcccccattggcctcagtccccacccggagggcggccccggctctggccgcctgggtttgcgccgacgctccacagccccagatatccccagcctggtcggggaggcgccagggcccgagagtggcccggagttagaggccaacgctctgtctgtctcggtgcctgggccgaacccttggcaggcgggcaccgaggctgtggtgctgcgtgctcagcggtaa

SEQ ID NO: 45 - protein, 568 aa, Homo sapiens
MKEAEMDGEAVRFCTDNQCVSLHPQEVDSVAMAPAAPKIPRLVQATPAFMAVTLVFSLVTLFVVGKPPVQQQTRPVPKPVQAVILGDNITGHLPFEPNNHHHFGREAEMRELIQTFKGHMENSSAWVVEIQMLKCRVDNVNSQLQVLGDHLGNTNADIQMVKGVLKDATTLSLQTQMLRSSLEGTNAEIQRLKEDLEKADALTFQTLNFLKSSLENTSIELHVLSRGLENANSEIQMLNARANAEIQGLKENLQNTNALNSQTQAFIKSSFDNTSAEIQFLRGHLERAGDEIHVLKRDLKMVTAQTQKANGRLDQTDTQIQVFKSEMENVNTLNAQIQVLNGHMKNASREIQTLKQGMKNASALTSQTQMLDSNLQKASAEIQRLRGDLENTKALTMEIQQEQSRLKTLHVVITSQEQLQRTQSQLLQMVLQGWKFNGGSLYYFSSVKKSWHEAEQFCVSQGAHLASVASKEEQAFLVEFTSKVYYWIGLTDRGTEGSWRWTDGTPFNAAQNKAPVVFGFWEKNQSDNWRHKNGQTEDCVQIQQKWNDMTCDTPYQWVCKKPMGQGVA

SEQ ID NO: 46 - protein, 364 aa, Homo sapiens
MSRQGKLFSAFGVGCCVTAGLPKDDNTPSTIADVHNGYTMNVVEQVLKDSFVLFFPGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTMKTCGYIPELEYGYVQPSVPPYQHGVSVEVNCRNEYAMIGNNMITCINGIWTELPMCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 47 - protein, 327 aa, Homo sapiens
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTMKTCGYIPELEYGYVQPSVPPYQHGVSVEVNCRNEYAMIGNNMITCINGIWTELPMCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 48 - protein, 154 aa, Homo sapiens
MAPARAGCCPLLLLLLGLWVAEVLVRAKPKDMTSSQWFKTQHVQPSPQACNSAMSIINKYTERCKDLNTFLHEPFSSVAITCQTPNIACKNSCKNCHQSHGPMSLTMGELTSGKYPNCRYKEKHLNTPYIVACDPPQQGDPGYPLVPVHLDKVV

SEQ ID NO: 49 - protein, 502 aa, Homo sapiens
MRQLGGSLRPPRAAHGAEPLPSALGPCAGGDRDLGRGTPGWEPRRARVPIHEQVDPPREGPHLFQNLLLFLWALLNCGLGVSAQGPGEWTPWVSWTRCSSSCGRGVSVRSRRCLRLPGEEPCWGDSHEYRLCQLPDCPPGAVPFRDLQCALYNGRPVLGTQKTYQWVPFHGAPNQCDLNCLAEGHAFYHSFGRVLDGTACSPGAQGVCVAGRCLSAGCDGLLGSGALEDRCGRCGGANDSCLFVQRVFRDAGAFAGYWNVTLIPEGARHIRVEHRSRNHLGILGSLMGGDGRYVLNGHWVVSPPGTYEAAGTHVVYTRDTGPQETLQAAGPTSHDLLLQVLLQEPNPGIEFEFWLPRERYSPFQARVQALGWPLRQPQPRGVEPQPPAAPAVTPAQTPTLAPVFQARVLGHHHQAQETRYEVRIQLVYKNRSPLRAREYVWAPGHCPCPMLAPHRDYLMAVQRLVSPDGTQDQLLLPHAGYARPWSPAEDSRIRLTARRCPG

SEQ ID NO: 50 - protein, 451 aa, Homo sapiens
MDSAPLFPRPHLFQNLLLFLWALLNCGLGVSAQGPGEWTPWVSWTRCSSSCGRGVSVRSRRCLRLPGEEPCWGDSHEYRLCQLPDCPPGAVPFRDLQCALYNGRPVLGTQKTYQWVPFHGAPNQCDLNCLAEGHAFYHSFGRVLDGTACSPGAQGVCVAGRCLSAGCDGLLGSGALEDRCGRCGGANDSCLFVQRVFRDAGAFAGYWNVTLIPEGARHIRVEHRSRNHLGILGSLMGGDGRYVLNGHWVVSPPGTYEAAGTHVVYTRDTGPQETLQAAGPTSHDLLLQVLLQEPNPGIEFEFWLPRERYSPFQARVQALGWPLRQPQPRGVEPQPPAAPAVTPAQTPTLAPVFQARVLGHHHQAQETRYEVRIQLVYKNRSPLRAREYVWAPGHCPCPMLAPHRDYLMAVQRLVSPDGTQDQLLLPHAGYARPWSPAEDSRIRLTARRCPG

SEQ ID NO: 51 - protein, 431 aa, Homo sapiens
MIRTPLSASAHRLLLPGSRGRPPRNMQPTGREGSRALSRRYLRRLLLLLLLLLLRQPVTRAETTPGAPRALSTLGSPSLFTTPGVPSALTTPGLTTPGTPKTLDLRGRAQALMRSFPLVDGYVGLNSSQKLACLIGVEGGHSLDSSLSVLRSFYVLGVRYLTLTFTCSTPWAESSTKFRHHMYTNVSGLTSFGEKVVEELNRLGMMIDLSYASDTLIRRVLEVSQAPVIFSHSAARAVCDNLLNVPDDILQLLKKNGGIVMVTLSMGVLQCNLLANVSTVADDSNRCSVPVIGSEFIGIGGNYDGTGRFPQGLEDVSTYPVLIEELLSRSWSEEELQGVLRGNLLRVFRQVEKVREESRAQSPVEAEFPYGQLSTSCHSHLVPQNGHQATHLEVTKQPTNRVPWRSSNASPYLVPGLVAAATIPTFTQWLC

SEQ ID NO: 52 - protein, 480 aa, Homo sapiens
MQPSGLEGPGTFGRWPLLSLLLLLLLLQPVTCAYTTPGPPRALTTLGAPRAHTMPGTYAPSTTLSSPSTQGLQEQARALMRDFPLVDGHNDLPLVLRQVYQKGLQDVNLRNFSYGQTSLDRLRDGLVGAQFWSAYVPCQTQDRDALRLTLEQIDLIRRMCASYSELELVTSAKGLNSSQKLACLIGVEGGHSLDSSLSVLRSFYVLGVRYLTLTFTCSTPWAESSTKFRHHMYTNVSGLTSFGEKVVEELNRLGMMIDLSYASDTLIRRVLEVSQAPVIFSHSAARAVCDNLLNVPDDILQLLKKNGGIVMVTLSMGVLQCNLLANVSTVADHFDHIRAVIGSEFIGIGGNYDGTGRFPQGLEDVSTYPVLIEELLSRSWSEEELQGVLRGNLLRVFRQVEKVREESRAQSPVEAEFPYGQLSTSCHSHLVPQNGHQATHLEVTKQPTNRVPWRSSNASPYLVPGLVAAATIPTFTQWLC

SEQ ID NO: 53 - protein, 371 aa, Homo sapiens
MDAATAPKQAWPPWPPLLFLLLLPGGSGGSCPAVCDCTSQPQAVLCGHRQLEAVPGGLPLDTELLDLSGNRLPKAQPLVRLQELRLSGACLTSIAAHAFHGLTAFHLLDVADNALQTLEETAFPSPDKLVTLRLSGNPLTCDCRLLWLLRLRHLDFGMSPPACAGPHHVQGKSLKEFSDILPPGHFTCKPALIRKSGPRWVIAEEGGHAVFSCSGDGDPAPTVSWMRPHGAWLGRAGRVRVLEDGTLEIRSVQLRDRGAYVCVVSNVAGNDSLRTWLEVIQVEPPNGTLSDPNITVPGIPGPFFLDSRGVAMVLAVGFLPFLTSVTLCFGLIALWSKGKGRVKHHMTFDFVAPRPSGDKNSGGNRVTAKLF

SEQ ID NO: 54 - protein, 592 aa, Homo sapiens
MDAATAPKQAWPPWPPLLFLLLLPGGSGGSCPAVCDCTSQPQAVLCGHRQLEAVPGGLPLDTELLDLSGNRLWGLQQGMLSRLSLLQELDLSYNQLSTLEPGAFHGLQSLLTLRLQGNRLRIMGPGVFSGLSALTLLDLRLNQIVLFLDGAFGELGSLQKLEVGDNHLVFVAPGAFAGLAKLSTLTLERCNLSTVPGLALARLPALVALRLRELDIGRLPAGALRGLGQLKELEIHLWPSLEALDPGSLVGLNLSSLAITRCNLSSVPFQALYHLSFLRVLDLSQNPISAIPARRLSPLVRLQELRLSGACLTSIAAHAFHGLTAFHLLDVADNALQTLEETAFPSPDKLVTLRLSGNPLTCDCRLLWLLRLRHLDFGMSPPACAGPHHVQGKSLKEFSDILPPGHFTCKPALIRKSGPRWVIAEEGGHAVFSCSGDGDPAPTVSWMRPHGAWLGRAGRVRVLEDGTLEIRSVQLRDRGAYVCVVSNVAGNDSLRTWLEVIQVEPPNGTLSDPNITVPGIPGPFFLDSRGVAMVLAVGFLPFLTSVTLCFGLIALWSKGKGRVKHHMTFDFVAPRPSGDKNSGGNRVTAKLF

SEQ ID NO: 55 - protein, 142 aa, Homo sapiens
MARYMLLLLLAVWVLTGELWPGAEARAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

SEQ ID NO: 56 - protein, 230 aa, Homo sapiens
MSEEVTYATLTFQDSAGARNNRDGNNLRKRGHPAPSPIWRHAALGLVTLCLMLLIGLVTLGMMFLQISNDINSDSEKLSQLQKTIQQQQDNLSQQLGNSNNLSMEEEFLKSQISSLLKRQEQMAIKLCQELIIHTSDHRCNPCPKMWQWYQNSCYYFTTNEEKTWANSRKDCIDKNSTLVKIDSLEEKDFLMSQPLLMFSFFWLGLSWDSSGRSWFWEDGSVPSPSLYVS

SEQ ID NO: 57 - protein, 194 aa, Homo sapiens
MWLSPALLLLILPGYSIAAKITGPTTVNGSEQGSLTVQCAYGSGWETYLKWRCQGADWNYCNILVKTNGSEQEVKKNRVSIRDNQKNHMFTVTMENLKRDDADSYWCGTERPGIDLGVKVQVTINPGTQTAVSEWTTTTASLAFTAAATQKTSSPLTRSPLKSTHFLFLFLLELPLLLSMLGTVLWVNRPQRRS

SEQ ID NO: 58 - protein, 333 aa, Homo sapiens
MRIWWLLLAIEICTGNINSQDTCRQGHPGIPGNPGHNGLPGRDGRDGAKGDKGDAGEPGRPGSPGKDGTSGEKGERGADGKVEAKGIKGDQGSRGSPGKHGPKGLAGPMGEKGLRGETGPQGQKGNKGDVGPTGPEGPRGNIGPLGPTGLPGPMGPIGKPGPKGEAGPTGPQGEPGVRGIRGWKGDRGEKGKIGETLVLPKSAFTVGLTVLSKFPSSDMPIKFDKILYNEFNHYDTAAGKFTCHIAGVYYFTYHITVFSRNVQVSLVKNGVKILHTKDAYMSSEDQASGGIVLQLKLGDEVWLQVTGGERFNGLFADEDDDTTFTGFLLFSSP

SEQ ID NO: 59 - protein, 225 aa, Homo sapiens
MRIWWLLLAIEICTGNINSQDTCRQGHPGIPGNPGHNGLPGRDGRDGAKGDKGDAGEAGPTGPQGEPGVRGIRGWKGDRGEKGKIGETLVLPKSAFTVGLTVLSKFPSSDMPIKFDKILYNEFNHYDTAAGKFTCHIAGVYYFTYHITVFSRNVQVSLVKNGVKILHTKDAYMSSEDQASGGIVLQLKLGDEVWLQVTGGERFNGLFADEDDDTTFTGFLLFSSP

SEQ ID NO: 60 - protein, 205 aa, Homo sapiens
MMRTLITTHPLPLLLLPQQLLQLVQFQEVDTDFDFPEEDKKEEFEECLEKFFSTGPARPPTKEKVKRRVLIEPGMPLNHIEYCNHEIMGKNVYYKHRWVAEHYFLLMQYDELQKICYNRFVPCKNGIRKCNRSKGLVEGVYCNLTEAFEIPACKYESLYRKGYVLITCSWQNEMQKRIPHTINDLVEPPEHRSFLSEDGVFVISP

SEQ ID NO: 61 - protein, 95 aa, Homo sapiens [sequence omitted]

SEQ ID NO: 62 - protein, 595 aa, Homo sapiens
MEVGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGHLGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 63 - protein, 613 aa, Homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 64 - protein, 596 aa, Homo sapiens
MAANSTSDLHTPGTQLSVADIIVITVYFALNVAVGIWSSCRASRNTVNGYFLAGRDMTWWPIGASLFASSEGSGLFIGLAGSGAAGGLAVAGFEWNATYVLLALAWVFVPIYISSEIVTLPEYIQKRYGGQRIRMYLSVLSLLLSVFTKISLDLYAGALFVHICLGWNFYLSTILTLGITALYTIAGGLAAVIYTDALQTLIMVVGAVILTIKAFDQIGGYGQLEAAYAQAIPSRTIANTTCHLPRTDAMHMFRDPHTGDLPWTGMTFGLTIMATWYWCTDQVIVQRSLSARDLNHAKAGSILASYLKMLPMGLIIMPGMISRALFPDDVGCVVPSECLRACGAEVGCSNIAYPKLVMELMPIGLRGLMIAVMLAALMSSLTSIFNSSSTLFTMDIWRRLRPRSGERELLLVGRLVIVALIGVSVAWIPVLQDSNSGQLFIYMQSVTSSLAPPVTAVFVLGVFWRRANEQGAFWGLIAGLVVGATRLVLEFLNPAPPCGEPDTRPAVLGSIHYLHFAVALFALSGAVVVAGSLLTPPPQSVQIENLTWWTLAQDVPLGTKAGDGQTPQKHAFWARVCGFNAILLMCVNIFFYAYFA

SEQ ID NO: 65 - protein, 393 aa, Homo sapiens
MDSLKNENYDLVFVEAFDFCSFLIAEKLVKPFVAILPTTFGSLDFGLPSPLSYVPVFPSLLTDHMDFWGRVKNFLMFFSFSRSQWDMQSTFDNTIKEHFPEGSRPVLSHLLLKAELWFVNSDFAFDFARPLLPNTVYIGGLMEKPIKPVPQDLDNFIANFGDAGFVLVAFGSMLNTHQSQEVLKKMHNAFAHLPQGVIWTCQSSHWPRDVHLATNVKIVDWLPQSDLLAHPSIRLFVTHGGQNSVMEAIRHGVPMVGLPVNGDQHGNMVRVVAKNYGVSIRLNQVTADTLTLTMKQVIEDKRYKSAVVAASVILHSQPLSPAQRLVGWIDHILQTGGATHLKPYAFQQPWHEQYLIDVFVFLLGLTLGTMWLCGKLLGVVARWLRGARKVKKT

SEQ ID NO: 66 - protein, 523 aa, Homo sapiens
MVGQRVLLLVAFLLSGVLLSEAAKILTISTLGGSHYLLLDRVSQILQEHGHNVTMLHQSGKFLIPDIKEEEKSYQVIRWFSPEDHQKRIKKHFDSYIETALDGRKESEALVKLMEIFGTQCSYLLSRKDIMDSLKNENYDLVFVEAFDFCSFLIAEKLVKPFVAILPTTFGSLDFGLPSPLSYVPVFPSLLTDHMDFWGRVKNFLMFFSFSRSQWDMQSTFDNTIKEHFPEGSRPVLSHLLLKAELWFVNSDFAFDFARPLLPNTVYIGGLMEKPIKPVPQDLDNFIANFGDAGFVLVAFGSMLNTHQSQEVLKKMHNAFAHLPQGVIWTCQSSHWPRDVHLATNVKIVDWLPQSDLLAHPSIRLFVTHGGQNSVMEAIRHGVPMVGLPVNGDQHGNMVRVVAKNYGVSIRLNQVTADTLTLTMKQVIEDKRYKSAVVAASVILHSQPLSPAQRLVGWIDHILQTGGATHLKPYAFQQPWHEQYLIDVFVFLLGLTLGTMWLCGKLLGVVARWLRGARKVKKT

SEQ ID NO: 67 - protein, 252 aa, Homo sapiens [sequence omitted]

SEQ ID NO: 68 - protein, 497 aa, Homo sapiens
MSCVLGGVIPLGLLFLVCGSQGYLLPNVTLLEELLSKYQHNESHSRVRRAIPREDKEEILMLHNKLRGQVQPQASNMEYMTWDDELEKSAAAWASQCIWEHGPTSLLVSIGQNLGAHWGRYRSPGFHVQSWYDEVKDYTYPYPSECNPWCPERCSGPMCTHYTQIVWATTNKIGCAVNTCRKMTVWGEVWENAVYFVCNYSPKGNWIGEAPYKNGRPCSECPPSYGGSCRNNLCYREETYTPKPETDEMNEVETAPIPEENHVWLQPRVMRPTKPKKTSAVNYMTQVVRCDTKMKDRCKGSTCNRYQCPAGCLNHKAKIFGTLFYESSSSICRAAIHYGILDDKGGLVDITRNGKVPFFVKSERHGVQSLSKYKPSSSFMVSKVKVQDLDCYTTVAQLCPFEKPATHCPRIHCPAHCKDEPSYWAPVFGTNIYADTSSICKTAVHAGVISNESGGDVDVMPVDKKKTYVGSLRNGVQSESLGTPRDGKAFRIFAVRQ

SEQ ID NO: 69 - protein, 438 aa, Homo sapiens
BMLHNKLRGQVQPQASNMEYMTWDDELEKSAAAWASQCIWEHGPTSLLVSIGQNLGAHWGRYRSPGFHVQSWYDEVKDYTYPYPSECNPWCPERCSGPMCTHYTQIVWATTNKIGCAVNTCRKMTVWGEVWENAVYFVCNYSPKGNWIGEAPYKNGRPCSECPPSYGGSCRNNLCYREETYTPKPETDEMNEVETAPIPEENHVWLQPRVMRPTKPKKTSAVNYMTQVVRCDTKMKDRCKGSTCNRYQCPAGCLNHKAKIFGTLFYESSSSICRAAIHYGILDDKGGLVDITRNGKVPFFVKSERHGVQSLSKYKPSSSFMVSKVKVQDLDCYTTVAQLCPFEKPATHCPRIHCPAHCKDEPSYWAPVFGTNIYADTSSICKTAVHAGVISNESGGDVDVMPVDKKKTYVGSLRNGVQSESLGTPRDGKAFRIFAVRQ

SEQ ID NO: 70 - protein, 308 aa, Homo sapiens
MVGGVLASLGFVFSAFASDLLHLYLGLGLLAGFGWALVFAPALGTLSRYFSRRRVLAVGLALTGNGASSLLLAPALQLLLDTFGWRGALLLLGAITLHLTPCGALLLPLVLPGDPPAPPRSPLAALGLSLFTRRAFSIFALGTALVGGGYFVPYVHLAPHALDRGLGGYGAALVVAVAAMGDAGARLVCGWLADQGWVPLPRLLAVFGALTGLGLWVVGLVPVVGGEESWGGPLLAAAVAYGLSAGSYAPLVFGVLPGLVGVGGVVQATGLVMMLMSLGGLLGPPLSGKDLSSQICLQLSSAPGVRGF

SEQ ID NO: 71 - protein, 447 aa, Homo sapiens
MTPQPAGPPDGGWGWVVAAAAFAINGLSYGLLRSLGLAFPDLAEHFDRSAQDTAWISALALAVQQAASPVGSALSTRWGARPVVMVGGVLASLGFVFSAFASDLLHLYLGLGLLAGFGWALVFAPALGTLSRYFSRRRVLAVGLALTGNGASSLLLAPALQLLLDTFGWRGALLLLGAITLHLTPCGALLLPLVLPGDPPAPPRSPLAALGLSLFTRRAFSIFALGTALVGGGYFVPYVHLAPHALDRGLGGYGAALVVAVAAMGDAGARLVCGWLADQGWVPLPRLLAVFGALTGLGLWVVGLVPVVGGEESWGGPLLAAAVAYGLSAGSYAPLVFGVLPGLVGVGGVVQATGLVMMLMSLGGLLGPPLSGFLRDETGDFTASFLLSGSLILSGSFIYIGLPRALPSCGPASPPATPPPETGELLPAPQAVLLSPGGPGSTLDTTC

SEQ ID NO: 72 - protein, 458 aa, Homo sapiens
BMARRTEPPDGGWGWVVVLSAFFQSALVFGVLRSFGVFFVEFVAAFEEQAARVSWIASIGIAVQQFGSPVGSALSTKFGPRPVVMTGGILAALGMLLASFATSLTHLYLSIGLLSGSGWALTFAPTLACLSCYFSRRRSLATGLALTGVGLSSFTFAPFFQWLLSHYAWRGSLLLVSALSLHLVACGALLRPPSLAEDPAVGGPRAQLTSLLHHGPFLRYTVALTLINTGYFIPYLHLVAHLQDLDWDPLPAAFLLSVVAISDLVGRVVSGWLGDAVPGPVTRLLMLWTTLTGVSLALFPVAQAPTALVALAVAYGFTSGALAPLAFSVLPELIGTRRIYCGLGLLQMIESIGGLLGPPLSGYLRDVSGNYTASFVVAGAFLLSGSGILLTLPHFFCFSTTTSGPQDLVTEALDTKVPLPKEGLEGGLNSTESGPESQSLTAPGLLLPRLGLHRTTVP

SEQ ID NO: 73 - protein, 169 aa, Homo sapiens
MTMKTSGATCDANSVMNCGIRGSEMFAEMDLRAIKPYQTLIKKVGQRHCVDPAVIAAIISRESHGGSVLQDGWDHRGLKFGLMQLDKQTYHPVGAWDSKEHLSQATGILTERIKAIQKKFPTWSVAQHLKGGLSAFKSGIEAIATPSDIDNDFVNDIIARAKFYKRQSF

SEQ ID NO: 74 - protein, 186 aa, Homo sapiens
MKPHLHPRLYHGCYGDIMTMKTSGATCDANSVMNCGIRGSEMFAEMDLRAIKPYQTLIKKVGQRHCVDPAVIAAIISRESHGGSVLQDGWDHRGLKFGLMQLDKQTYHPVGAWDSKEHLSQATGILTERIKAIQKKFPTWSVAQHLKGGLSAFKSGIEAIATPSDIDNDFVNDIIARAKFYKRQSF

SEQ ID NO: 75 - protein, 675 aa, Homo sapiens
MESGTSSPQPPQLDPLDAFPQKGLEPGDIAVLVLYFLFVLAVGLWSTVKTKRDTVKGYFLAGGDMVWWPVGASLFASNVGSGHFIGLAGSGAATGISVSAYELNGLFSVLMLAWIFLPIYIAGQVTTMPEYLRKRFGGIRIPIILAVLYLFIYIFTKISVDMYAGAIFIQQSLHLDLYLAIVGLLAITAVYTVAGGLAAVIYTDALQTLIMLIGALTLMGYSFAAVGGMEGLKEKYFLALASNRSENSSCGLPREDAFHIFRDPLTSDLPWPGVLFGMSIPSLWYWCTDQVIVQRTLAAKNLSHAKGGALMAAYLKVLPLFIMVFPGMVSRILFPDQVACADPEICQKICSNPSGCSDIAYPKLVLELLPTGLRGLMMAVMVAALMSSLTSIFNSASTIFTMDLWNHLRPRASEKELMIVGRVFVLLLVLVSILWIPVVQASQGGQLFIYIQSISSYLQPPVAVVFIMGCFWKRTNEKGAFWGLISGLLLGLVRLVLDFIYVQPRCDQPDERPVLVKSIHYLYFSMILSTVTLITVSTVSWFTEPPSKEMVSHLTWFTRHDPVVQKEQAPPAAPLSLTLSQNGMPEASSSSSVQFEMVQENTSKTHSCDMTPKQSKVVKAILWLCGIQEKGKEELPARAEAIIVSLEENPLVKTLLDVNLIFCVSCAIFIWGYFA

SEQ ID NO: 76 - protein, 485 aa, Homo sapiens
MEPCWGEGLFHLAPPRHHPQKADWHFCPQHIQEFTNETWQARTGEPLPDHLVLLMWSLIVSLYPLGGLFGALLAGPLAITLGRKKSLLVNNIFVVSAAILFGFSRKAGSFEMIMLGRLLVGVNAGVSMNIQPMYLGESAPKELRGAVAMSSAIFTALGIVMGQVVGLRELLGGPQAWPLLLASCLVPGALQLASLPLLPESPRYLLIDCGDTEACLAALRRLRGSGDLAGELEELEEERAACQGCRARRPWELFQHRALRRQVTSLVVLGSAMELCGNDSVYAYASSVFRKAGVPEAKIQYAIIGTGSCELLTAVVSCVVIERVGRRVLLIGGYSLMTCWGSIFTVALCLQSSFPWTLYLAMACIFAFILSFGIGPAGVTGILATELFDQMARPAACMVCGALMWIMLILVGLGFPFIMEALSHFLYVPFLGVCVCGAIYTGLFLPETKGKTFQEISKELHRLNFPRRAQGPTWRSLEVIQSTEL

SEQ ID NO: 77 - protein, 496 aa, Homo sapiens
MRALRRLIQGRILLLTICAAGIGGTFQFGYNLSIINAPTLHIQEFTNETWQARTGEPLPDHLVLLMWSLIVSLYPLGGLFGALLAGPLAITLGRKKSLLVNNIFVVSAAILFGFSRKAGSFEMIMLGRLLVGVNAGVSMNIQPMYLGESAPKELRGAVAMSSAIFTALGIVMGQVVGLRELLGGPQAWPLLLASCLVPGALQLASLPLLPESPRYLLIDCGDTEACLAALRRLRGSGDLAGELEELEEERAACQGCRARRPWELFQHRALRRQVTSLVVLGSAMELCGNDSVYAYASSVFRKAGVPEAKIQYAIIGTGSCELLTAVVSCVVIERVGRRVLLIGGYSLMTCWGSIFTVALCLQSSFPWTLYLAMACIFAFILSFGIGPAGVTGILATELFDQMARPAACMVCGALMWIMLILVGLGFPFIMEALSHFLYVPFLGVCVCGAIYTGLFLPETKGKTFQEISKELHRLNFPRRAQGPTWRSLEVIQSTEL

SEQ ID NO: 78 - protein, 500 aa, Homo sapiens
BMLHALLRSRMIQGRILLLTICAAGIGGTFQFGYNLSIINAPTLHIQEFTNETWQARTGEPLPDHLVLLMWSLIVSLYPLGGLFGALLAGPLAITLGRKKSLLVNNIFVVSAAILFGFSRKAGSFEMIMLGRLLVGVNAGVSMNIQPMYLGESAPKELRGAVAMSSAIFTALGIVMGQVVGLRELLGGPQAWPLLLASCLVPGALQLASLPLLPESPRYLLIDCGDTEACLAALRRLRGSGDLAGELEELEEERAACQGCRARRPWELFQHRALRRQVTSLVVLGSAMELCGNDSVYAYASSVFRKAGVPEAKIQYAIIGTGSCELLTAVVSCVVIERVGRRVLLIGGYSLMTCWGSIFTVALCLQSSFPWTLYLAMACIFAFILSFGIGPAGVTGILATELFDQMARPAACMVCGALMWIMLILVGLGFPFIMEALSHFLYVPFLGVCVCGAIYTGLFLPETKGKTFQEISKELHRLNFPRRAQGPTWRSLEVIQSTEL

SEQ ID NO: 79 - protein, 1358 aa, Homo sapiens
MVVVKPMNTMAPVVTRNTGLILYEGQSRPLTGPAGSGPQNLVISDEDDLEAVRLEVVAGLRHGHLVILGASSGSSAPKSFTVAELAAGQVVYQHDDRDGSLSDNLVLRMVDGGGRHQVQFLFPITLVPVDDQPPVLNANTGLTLAEGETVPILPLSLSATDMDSDDSLLLFVLESPFLTTGHLLLRQTHPPHEKQELLRGLWRKEGAFYERTVTEWQQQDITEGRLFYRHSGPHSPGPVTDQFTFRVQDNHDPPNQSGLQRFVIRIHPVDRLPPELGSGCPLRMVVQESQLTPLRKKWLRYTDLDTDDRELRYTVTQSPTDTDENHLPAPLGTLVLTDNPSVVVTHFTQAQINHHKIAYRPPGQELGVATRVAQFQFQVEDRAGNVAPGTFTLYLHPVDNQPPEILNTGFTIQEKGHHILSETELHVNDVDTDVAHISFTLTQAPKHGHMRVSGQILHVGGLFHLEDIKQGRVSYAHNGDKSLTDSCSLEVSDRHHVVPITLRVNVRPVDDEVPILSHPTGTLESYLDVLENGATEITANVIKGTNEETDDLMLTFLLEDPPLYGEILVNGIPAEQFTQRDILEGSVVYTHTSGEIGLLPKADSFNLSLSDMSQEWRIGGNTIQGVTIWVTILPVDSQAPEIFVGEQLIVMEGDKSVITSVHISAEDVDSLNDDILCTIVIQPTSGYVENISPAPGSEKSRAGIAISAFNLKDLRQGHINYVQSVHKGVEPVEDRFVFRCSDGINFSERQFFPIVIIPTNDEQPEMFMREFMVMEGMSLVIDTPILNAADADVPLDDLTFTITQFPTHGHIMNQLINGTVLVESFTLDQIIESSSIIYEHDDSETQEDSFVIKLTDGKHSVEKTVLIIVIPVDDETPRMTINNGLEIEIGDTKIINNKILMATDLDSEDKSLVYIIRYGPGHGLLQRRKPTGAFENITLGMNFTQDEVDRNLIQYVHLGQEGIRDLIKFDVTDGINPLIDRYFYVSIGSIDIVFPDVISKGVSLKEGGKVTLTTDLLSTSDLNSPDENLVFTITRAPMRGHLECTDQPGVSITSFTQLQLAGNKIYYIHTADDEVKMDSFEFQVTDGRNPVFRTFRISISDVDNKKPVVTIHKLVVSESENKLITPFELTVEDRDTPDKLLKFTITQVPIHGHLLFNNTRPVMVFTKQDLNENLISYKHDGTESSEDSFSFTVTDGTHTDFYVFPDTVFETRRPQVMKIQVLAVDNSVPQIAVNKGASTLRTLATGHLGFMITSKILKVEDRDSLHISLRFIVTEAPQHGYLLNLDKGNHSITQFTQADIDDMKICYVLREGANATSDMFYFAVEDGGKYSPLLVVTARRDAFLGCSLMTLLQEVFIK

SEQ ID NO: 80 - protein, 3105 aa, Homo sapiens
MARSWLTATSTSRPAAFGRALLSPGLAGAAGVPAEEAIVLANRGLRVPFGREVWLDPLHDLVLQVQPGDRCAVSVLDNDALAQRPGRLSPKRFPCDFGPGEVRYSHLGARSPSRDRVRLQLRYDAPGGAVVLPLVLEVEVVFTQLEVVTRNLPLVVEELLGTSNALDARSLEFAFQPETEECRVGILSGLGALPRYGELLHYPQVPGGAREGGAPETLLMDCKAFQELGVRYRHTAASRSPNRDWIPMVVELRSRGAPVGSPALKREHFQVLVRIRGGAENTAPKPSFVAMMMMEVDQFVLTALTPDMLAAEDAESPSDLLIFNLTSPFQPGQGYLVSTDDRSLPLSSFTQRDLRLLKIAYQPPSEDSDQERLFELELEVVDLEGAASDPFAFMVVVKPMNTMAPVVTRNTGLILYEGQSRPLTGPAGSGPQNLVISDEDDLEAVRLEVVAGLRHGHLVILGASSGSSAPKSFTVAELAAGQVVYQHDDRDGSLSDNLVLRMVDGGGRHQVQFLFPITLVPVDDQPPVLNANTGLTLAEGETVPILPLSLSATDMDSDDSLLLFVLESPFLTTGHLLLRQTHPPHEKQELLRGLWRKEGAFYERTVTEWQQQDITEGRLFYRHSGPHSPGPVTDQFTFRVQDNHDPPNQSGLQRFVIRIHPVDRLPPELGSGCPLRMVVQESQLTPLRKKWLRYTDLDTDDRELRYTVTQPPTDTDENHLPAPLGTLVLTDNPSVVVTHFTQAQINHHKIAYRPPGQELGVATRVAQFQFQVEDRAGNVAPGTFTLYLHPVDNQPPEILNTGFTIQEKGHHILSETELHVNDVDTDVAHISFTLTQAPKHGHMRVSGQILHVGGLFHLEDIKQGRVSYAHNGDKSLTDSCSLEVSDRHHVVPITLRVNVRPVDDEVPILSHPTGTLESYLDVLENGATEITANVIKGTNEETDDLMLTFLLEDPPLYGEILVNGIPAEQFTQRDILEGSVVYTHTSGEIGLLPKADSFNLSLSDKSQEWRIGGNTIQGVTIWVTILPVDSQAPEISVGEQLIVMEGDKSVITSVHISAEDVDSLNDDILCTIVIQPTSGYVENISPAPGSEKSRAGIAISAFNLKDLRQGHINYVQSVHKGVEPVEDRFVFRCSDGINFSERQFFPIVIIPTNDEQPEMFMREFMVMEGMSLVIDTPILNAADADVPLDDLTFTITQFPTHGHIMNQLINGTVLVESFTLDQIIESSSIIYEHDDSETQEDSFVIKLTDGKHSVEKTVLIIVIPVDDETPRMTINNGLEIEIGDTKIINNKILMATDLDSEDKSLVYIIRYGPGHGLLQRRKPTGAFENITLGMNFTQDEVDRNLIQYVHLGQEGIRDLIKFDVTDGINPLIDRYFYVSIGSIDIVFPDVISKGVSLKEGGKVTLTTDLLSTSDLNSPDENLVFTITRAPMRGHLECTDQPGVSITSFTQLQLAGNKIYYIHTADDEVKMDSFEFQVTDGRNPVFRTFRISISDVDNKKPVVTIHKLVVSESENKLITPFELTVEDRDTPDKLLKFTITQVPIHGHLLFNNTRPVMVFTKQDLNENLISYKHDGTESSEDSFSFTVTDGTHTDFYVFPDTVFETRRPQVMKIQVLAVDNSVPQIAVNKGASTLRTLATGHLGFMITSKILKVEDRDSLHISLRFIVTEAPQHGYLLNLDKGNHSITQFTQADIDDMKICYVLREGANATSDMFYFAVEDGGGNKLTYQNFRLNWAWISFEKEYYLVNEDSKFLDVVLKRRGYLGETSFISIGTRDRTAEKDKDFKGKAQKQVQFNPGQTRATWRVRILSDGEHEQSETFQVVLSEPVLAALEFPTVATVEIVDPGDEPTVFIPQSKYSVEEDVGELFIPIRRSGDVSQELMVVCYTQQGTATGTVPTSVLSYSDYISRPEDHTSVVRFDKDEREKLCRIVIIDDSLYEEEETFHVLLSMPMGGRIGSEFPGAQVTIVPDKDDGPSDSKFNVAENYSLLPFTCFQGSIATAEAATQGGGRSTRQVAAVKKDKDFKGKAQKQVQFNPGQTRATWRVRILSDGEHEQSETFQVVLSEPVLAALEFPTVATVEIVDPGDACPWGEESDQSSQGLKLQSFLTKMMVSTNLLENSFSREDQHQEQLSRQKKWESKTMIIYTFILCETEKPCILELMDDVLYEEVEELRLVLGTPQSNSPFGAAVGEQNETLIRIRDDADKTVIKFGETKFSVTEPKEPGESVVIRIPVIRQGDTSKVSIVRVHTKDGSATSGEDYHPVSEEIEFKEGETQHVVEIEVTFDGVREMREAFTVHLKPDENMIAEMQLSNFELTLSPDGTRVGNHKCSNLLDYTEVKTHYGFLTDATKNPEIIGETYPYQYSLSIRGSTTLRFYRNLNLEACLWEFVSYYDMSELLADCRSVLNASIFHEMAPEGKQSKCLVNSTLYSILECHESLPNFCISALRMGKWRKIKSKPSAQTPCAQRLRGFIDHPRKQPLQQASADPGMLPVISTRELSNFELTLSPDGTRVGNHKCSNLLDYTEVKTHYGFLTDATKNPEIIGETYPYQYSLSIRGSTTLRFYRNLNLEACLWEFVSYYDMSELLADCGGTIGTDGQVLNLVQSYVTLRVPLYVSYVFHSPVGVGGWQHFDLKSELRLTFVYDTAILWNDGIGSPPEAELQGSLYPTSMRIGDEGRLAVHFKTEAQFHGLFVLSHPASFTSSVIMSADHPGLTFSLRLIRSEPTYNQPVQQWSFVSDFAVRDYSGTYTVKLVPCTAPSHQEYRLPVTCNPREPVTFDLDIRFQQVSDPVAAEFSLNTQMYLLSKKSLWLSDGSMGFGQESDVAFAEGDIIYGRVMVDPVQNLGDSFYCSIEKVFLCTGADGYVPKYSPMNAEYGCLADSPSLLYRFKIVDKAQPETQATSFGNVLFNAKLAVDDPEAILLVNQPGSDGFKVDSTPLFQVALGREWYIHTIYTVRSKDNANRGIGKRSVEYHSLVSQGKPQSTTKSRKKREIRSTPSLAWEIGAENSRGTNIQHIALDRTKRQIPHGRAPPDGILPWELNSPSSAVSLVTVVGGTTVGLLTICLTVIAVLMCRGKESFRGKDAPKGSSSSEPMVPPQSHHNDSSEV

SEQ ID NO: 81 - protein, 457 aa, Homo sapiens
MGCSGAWGLSCPCPQTPSWAWRKMRTPSMRNRSGAVWSRASVPFSAWEVEVQMRVTGLGRRGAQGMAVWYTRGRGHVGSVLGGLASWDGIGIFFDSPAEDTQDSPAIRVLASDGHIPSEQPGDGASQGLGSCHWDFRNRPHPFRARITYWGQRLRMSLNSGLTPSDPGEFCVDVGPLLLVPGGFFGVSAATGTLADDHDVLSFLTFSLSEPSPEVPPQPFLEMQHVRLARQLEGLWARLGLGTREDVTPKSDSEAQGEGERLFDLEETLGRHRRILQALRGLSKQLAQAERQWKKQLGPQAKPGLTEAGDAAVRMAAEAQVSYLPVGIEHHFLELDHILGLLQEELRGPAKAAAKAPRPPGQPPRASSCLQPGIFLFYLLIQTVGFFGYVHFRRPVPRPAKTMAFMVKTMVGGQLKNLTGSLGGGEDKGDGDKSAAEAQGMSREEYEEYQKQLVEEK

SEQ ID NO: 82 - protein, 526 aa, Homo sapiens
MPAVSGPGPLFCLLLLLLDPHSPETGCPPLRRFEYKLSFKGPRLALPGAGIPFWSHHGDAILGLEEVRLTPSMRNRSGAVWSRASVPFSAWEVEVQMRVTGLGRRGAQGMAVWYTRGRGHVGSVLGGLASWDGIGIFFDSPAEDTQDSPAIRVLASDGHIPSEQPGDGASQGLGSCHWDFRNRPHPFRARITYWGQRLRMSLNSGLTPSDPGEFCVDVGPLLLVPGGFFGVSAATGTLADDHDVLSFLTFSLSEPSPEVPPQPFLEMQHVRLARQLEGLWARLGLGTREDVTPKSDSEAQGEGERLFDLEETLGRHRRILQALRGLSKQLAQAERQWKKQLGPPGQARPDGGWALDASCQIPSTPGRGGHLSMSLNKDSAKVGALLHGQWTLLQALQEMRDAAVRMAAEAQVSYLPVGIEHHFLELDHILGLLQEELRGPAKAAAKAPRPPGQPPRASSCLQPGIFLFYLLIQTVGFFGYVHFRQELNKSLQECLSTGSLPLGPAPHTPRALGILRRQPLPASMPA

SEQ ID NO: 83 - protein, 904 aa, Homo sapiens [sequence omitted]

SEQ ID NO: 84 - protein, 515 aa, Homo sapiens
MLLWASLLAFAPVCGQSAAAHKPVISVHPPWTTFFKGERVTLTCNGFQFYATEKTTWYHRHYWGEKLTLTPGNTLEVRESGLYRCQARGSPRSNPVRLLFSSDSLILQAPYSVFEGDTLVLRCHRRRKEKLTAVKYTWNGNILSISNKSWDLLIPQASSNNNGNYRCIGYGDENDVFRSNFKIIKIQELFPHPELKATDSQPTEGNSVNLSCETQLPPERSDTPLHFNFFRDGEVILSDWSTYPELQLPTVWRENSGSYWCGAETVRGNIHKHSPSLQIHVQRIPVSGVLLETQPSGGQAVEGEMLVLVCSVAEGTGDTTFSWHREDMQESLGRKTQRSLRAELELPAIRQSHAGGYYCTADNSYGPVQSMVLNVTVRETPGNRDGLVAAGATGGLLSALLLAVALLFHCWRRRKSGVGFLGDETRLPPAPGPGESSHSICPAQVELQSLYVDVHPKKGDLVYSEIQTTQLGEEEEANTSRTLLEDKDVSVVYSEVKTQHPDNSAGKISSKDEES

SEQ ID NO: 85 - protein, 831 aa, Homo sapiens
BMAVGGGHKGVRCPPSARSSQVPTCCAGWRQQGDECGIAVCEGNSTCSENEVCVRPGECRCRHGYFGANCDTKCPRQFWGPDCKELCSCHPHGQCEDVTGQCTCHARRWGARCEHACQCQHGTCHPRSGACRCESGWWGAQCASACYCSATSRCDPQTGACLCHAGWWGRSCNNQCACNSSPCEQQSGRCQCRERTFGARCDRYCQCFRGRCHPVDGTCACEPGYRGKYCREPCPAGFYGLGCRRRCGQCKGQQPCTVAEGRCLTCEPGWNGTKCDQPCATGFYGEGCSHRCPPCRDGHACNHVTGKCTRCNAGWIGDRCETKCSNGTYGEDCAFVCADCGSGHCDFQSGRCLCSPGVHGPHCNVTCPPGLHGADCAQACSCHEDTCDPVTGACHLETNQRKGVMGAGALLVLLVCLLLSLLGCCCACRGKDPTRRPRPRRELSLGRKKAPHRLCGRFSRISMKLPRIPLRRQKLPKVVVAHHDLDNTLNCSFLEPPSGLEQPSPSWSSRASFSSFDTTDEGPVYCVPHEEAPAESRDPEVPTVPAEAPAPSPVPLTTPASAEEAIPLPASSDSERSASSVEGPGGALYARVARREARPARARGEIGGLSLSPSPERRKPPPPDPATKPKVSWIHGKHSAAAAGRAPSPPPPGSEAAPSPSKRKRTPSDKSAHTVEHGSPRTRDPTPRPPGLPEEATALAAPSPPRARARAAPRPLGAHGRRRSPAKRAEAASMLAADVRGKTRSLGRAEVALGAQGPREKPAPPQKAKRSVPPASPARAPPATETPGPEKAATDLPAPETPRKKTPIQKPPRKKSREAAGELGRAGAPTL

SEQ ID NO: 86 - protein, 871 aa, Homo sapiens
BMEGAGPRGAGPARRRGAGGPPSPLLPSLLLLLLLWMLPDTVAPQELNPRGRNVCRAPGSQVPTCCAGWRQQGDECGIAVCEGNSTCSENEVCVRPGECRCRHGYFGANCDTKCPRQFWGPDCKELCSCHPHGQCEDVTGQCTCHARRWGARCEHACQCQHGTCHPRSGACRCESGWWGAQCASACYCSATSRCDPQTGACLCHAGWWGRSCNNQCACNSSPCEQQSGRCQCRERTFGARCDRYCQCFRGRCHPVDGTCACEPGYRGKYCREPCPAGFYGLGCRRRCGQCKGQQPCTVAEGRCLTCEPGWNGTKCDQPCATGFYGEGCSHRCPPCRDGHACNHVTGKCTRCNAGWIGDRCETKCSNGTYGEDCAFVCADCGSGHCDFQSGRCLCSPGVHGPHCNVTCPPGLHGADCAQACSCHEDTCDPVTGACHLETNQRKGVMGAGALLVLLVCLLLSLLGCCCACRGKDPTRRPRPRRELSLGRKKAPHRLCGRFSRISMKLPRIPLRRQKLPKVVVAHHDLDNTLNCSFLEPPSGLEQPSPSWSSRASFSSFDTTDEGPVYCVPHEEAPAESRDPEVPTVPAEAPAPSPVPLTTPASAEEAIPLPASSDSERSASSVEGPGGALYARVARREARPARARGEIGGLSLSPSPERRKPPPPDPATKPKVSWIHGKHSAAAAGRAPSPPPPGSEAAPSPSKRKRTPSDKSAHTVEHGSPRTRDPTPRPPGLPEEATALAAPSPPRARARAAPRPLGAHGRRRSPAKRAEAASMLAADVRGKTRSLGRAEVALGAQGPREKPAPPQKAKRSVPPASPARAPPATETPGPEKAATDLPAPETPRKKTPIQKPPRKKSREAAGELGRAGAPTL

SEQ ID NO: 87 - protein, 134 aa, Homo sapiens
IDCRAEQGRTPLMVAVGLPDPALRARFVRLLLEQGAAVNLRDERGRTALSLACERGHLDAVQLLVQFSGDPEAADSAGNSPVMWAAACGHGAVLEFLVRSFRRLGLRLDRTNRAGLTALQLAAARGHGTCVQAL

SEQ ID NO: 88 - protein, 324 aa, Homo sapiens
MLKPKDLCPRAGTRTFLEAMQAGKVHLARFVLDALDRSIIDCRAEQGRTPLMVAVGLPDPALRARFVRLLLEQGAAVNLRDERGRTALSLACERGHLDAVQLLVQFSGDPEAADSAGNSPVMWAAACGHGAVLEFLVRSFRRLGLRLDRTNRAGLTALQLAAARGHGTSAGGHGGEAGSAGKNSGRHRAQGSERPELGRSMSLALGAVTEEEAARLRAGALMALPNSPQSSGTGRWRSQEVLEGAPPTLAQAPIGLSPHPEGGPGSGRLGLRRRSTAPDIPSLVGEAPGPESGPELEANALSVSVPGPNPWQAGTEAVVLRAQR

SEQ ID NO: 89 - DNA, 1569 bp, Homo sapiens
atggatttcctgctgctcggtctctgtctatactggctgctgaggaggccctcgggggtggtcttgtgtctgctgggggcctgctttcagatgctgcccgccgcccccagcgggtgcccgcagctgtgccggtgcgaggggcggctgctgtactgcgaggcgctcaacctcaccgaggcgccccacaacctgtccggcctgctgggcttgtccctgcgctacaacagcctctcggagctgcgcgccggccagttcacggggttaatgcagctcacgtggctctatctggatcacaatcacatctgctccgtgcagggggacgcctttcagaaactgcgccgagttaaggaactcacgctgagttccaaccagatcacccaactgcccaacaccaccttccggcccatgcccaacctgcgcagcgtggacctctcgtacaacaagctgcaggcgctcgcgcccgacctcttccacgggctgcggaagctcaccacgctgcatatgcgggccaacgccatccagtttgtgcccgtgcgcatcttccaggactgccgcagcctcaagtttctcgacatcggatacaatcagctcaagagtctggcgcgcaactctttcgccggcttgtttaagctcaccgagctgcacctcgagcacaacgacttggtcaaggtgaacttcgcccacttcccgcgcctcatctccctgcactcgctctgcctgcggaggaacaaggtggccattgtggtcagctcgctggactgggtttggaacctggagaaaatggacttgtcgggcaacgagatcgagtacatggagccccatgtgttcgagaccgtgccgcacctgcagtccctgcagctggactccaaccgcctcacctacatcgagccccggatcctcaactcttggaagtccctgacaagcatcaccctggccgggaacctgtgggattgcgggcgcaacgtgtgtgccctagcctcgtggctcaacaacttccaggggcgctacgatggcaacttgcagtgcgccagcccggagtacgcacagggcgaggacgtcctggacgccgtgtacgccttccacctgtgcgaggatggggccgagcccaccagcggccacctgctctcggccgtcaccaaccgcagtgatctggggccccctgccagctcggccaccacgctcgcggacggcggggaggggcagcacgacggcacattcgagcctgccaccgtggctcttccaggcggcgagcacgccgagaacgccgtgcagatccacaaggtggtcacgggcaccatggccctcatcttctccttcctcatcgtggtcctggtgctctacgtgtcctggaagtgtttcccagccagcctcaggcagctcagacagtgctttgtcacgcagcgcaggaagcaaaagcagaaacagaccatgcatcagatggctgccatgtctgcccaggaatactacgttgattacaaaccgaaccacattgagggagccctggtgatcatcaacgagtatggctcgtgtacctgccaccagcagcccgcgagggaatgcgaggtgtga

SEQ ID NO: 90 - protein, 522 aa, Homo sapiens
MDFLLLGLCLYWLLRRPSGVVLCLLGACFQMLPAAPSGCPQLCRCEGRLLYCEALNLTEAPHNLSGLLGLSLRYNSLSELRAGQFTGLMQLTWLYLDHNHICSVQGDAFQKLRRVKELTLSSNQITQLPNTTFRPMPNLRSVDLSYNKLQALAPDLFHGLRKLTTLHMRANAIQFVPVRIFQDCRSLKFLDIGYNQLKSLARNSFAGLFKLTELHLEHNDLVKVNFAHFPRLISLHSLCLRRNKVAIVVSSLDWVWNLEKMDLSGNEIEYMEPHVFETVPHLQSLQLDSNRLTYIEPRILNSWKSLTSITLAGNLWDCGRNVCALASWLNNFQGRYDGNLQCASPEYAQGEDVLDAVYAFHLCEDGAEPTSGHLLSAVTNRSDLGPPASSATTLADGGEGQHDGTFEPATVALPGGEHAENAVQIHKVVTGTMALIFSFLIVVLVLYVSWKCFPASLRQLRQCFVTQRRKQKQKQTMHQMAAMSAQEYYVDYKPNHIEGALVIINEYGSCTCHQQPARECEV

SEQ ID NO: 91 - DNA, 870 bp, Homo sapiens
atgtttgtcttgctctatgttacaagttttgccatttgtgccagtggacaaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 92 - protein, 289 aa, Homo sapiens
MFVLLYVTSFAICASGQPRGNQLKGENYSPRYICSIPGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 93 - DNA, 1047 bp, Homo sapiens
atgtcccttgcttcaggccctggccctgggtggttactcttttcctttggaatggggctggtatcagggtcaaagtgtccaaataattgtctgtgtcaagcccaagaagtaatctgcacagggaagcagttaaccgaatacccccttgacatacccctgaacacccggaggctgttcctgaacgagaacagaatcactagtttgccagcaatgcatctaggactcctcagtgaccttgtttatttggactgtcagaacaaccggattcgagaggtgatggattataccttcatcggggtcttcaaactcatctaccttgacctcagctccaacaacctaacctcgatctccccattcactttctcggtgctcagcaacctggtgcagctgaacattgccaacaaccctcacctgttatcgcttcacaagttcacctttgccaacaccacctctttgaggtacctggacctcagaaataccggcttgcagaccctggacagtgctgccttataccacctcactactctggagaccctgtttctgagtggaaacccctggaagtgcaactgctctttcctggacttcgccatcttcttaatagtgttccatatggacccctcagatgatctaaatgccacatgtgtggagcccacagagctgacagggtggcccatcacccgggtggggaacccactccgatacatgtgcatcacgcacctggaccacaaagactacatcttcctgctgctcatcggcttctgcatcttcgccgcgggaactgtggctgcctggctcacaggtgtgtgtgctgtgctctaccagaacacccgccacaagtcgagtgaagaagatgaggacgaggccgggactagggtggaagtcagcaggcggatttttcaacccagacgagctcggtccaggagttccctcagcttatttagttgccagagaccactatcttatgtgcctcccccaggctccctgctttctctcttgccctccccatcccaccaccttggagctgtcatagagattgaaaccttctag

SEQ ID NO: 94 - protein, 348 aa, Homo sapiens
MSLASGPGPGWLLFSFGMGLVSGSKCPNNCLCQAQEVICTGKQLTEYPLDIPLNTRRLFLNENRITSLPAMHLGLLSDLVYLDCQNNRIREVMDYTFIGVFKLIYLDLSSNNLTSISPFTFSVLSNLVQLNIANNPHLLSLHKFTFANTTSLRYLDLRNTGLQTLDSAALYHLTTLETLFLSGNPWKCNCSFLDFAIFLIVFHMDPSDDLNATCVEPTELTGWPITRVGNPLRYMCITHLDHKDYIFLLLIGFCIFAAGTVAAWLTGVCAVLYQNTRHKSSEEDEDEAGTRVEVSRRIFQPRRARSRSSLSLFSCQRPLSYVPPPGSLLSLLPSPSHHLGAVIEIETF

SEQ ID NO: 95 - DNA, 1281 bp, Homo sapiens
atggcgcgcaggacagagccccccgacgggggctggggatgggtggtggtgctctcagcgttcttccagtcggcgcttgtgtttggggtgctccgctcctttggggtcttcttcgtggagtttgtggcggcgtttgaggagcaggcagcgcgcgtctcctggatcgcctccataggaatcgcggtgcagcagtttgggagcccggtaggcagtgccctgagcacgaagttcgggcccaggcccgtggtgatgactggaggcatcttggctgcgctggggatgctgctcgcctcttttgctacttccttgacccacctatacctgagtattgggttgctgtcaggctctggctgggctttgaccttcgctccgaccctggcctgcctgtcctgttatttctctcgccgacgatccctggccaccgggctggcactgacaggcgtgggcctctcctccttcacatttgccccctttttccagtggctgctcagccactacgcctggagggggtccctgctgctggtgtctgccctctccctccacctagtggcctgtggtgctctcctccgcccaccctccctggctgaggaccctgctgtgggtggtcccagggcccaactcacctctctcctccatcatggccccttcctccgttacactgttgccctcaccctgatcaacactggctacttcattccctacctccacctggtggcccatctccaggacctggattgggacccactacctgctgccttcctactctcagttgttgctatttctgacctcgtggggcgtgtggtctccggatggctgggagatgcagtcccagggcctgtgacacgactcctgatgctctggaccaccttgactggggtgtcactagccctgttccctgtagctcaggctcccacagccctggtggctctggctgtggcctacggcttcacatcaggggctctggccccactggccttctccgtgctgcctgaactaatagggactagaaggatttactgtggcctgggactgttgcagatgatagagagcatcggggggctgctggggcctcctctctcaggctacctccgggatgtgtcaggcaactacacggcttcttttgtggtggctggggccttccttctttcagggagtggcattctcctcaccctgccccacttcttctgcttctcaactactacctccgggcctcaggaccttgtaacagaagcactagatactaaagttcccctacccaaggaggggctggaagaggactga

SEQ ID NO: 96 - protein, 426 aa, Homo sapiens
MARRTEPPDGGWGWVVVLSAFFQSALVFGVLRSFGVFFVEFVAAFEEQAARVSWIASIGIAVQQFGSPVGSALSTKFGPRPVVMTGGILAALGMLLASFATSLTHLYLSIGLLSGSGWALTFAPTLACLSCYFSRRRSLATGLALTGVGLSSFTFAPFFQWLLSHYAWRGSLLLVSALSLHLVACGALLRPPSLAEDPAVGGPRAQLTSLLHHGPFLRYTVALTLINTGYFIPYLHLVAHLQDLDWDPLPAAFLLSVVAISDLVGRVVSGWLGDAVPGPVTRLLMLWTTLTGVSLALFPVAQAPTALVALAVAYGFTSGALAPLAFSVLPELIGTRRIYCGLGLLQMIESIGGLLGPPLSGYLRDVSGNYTASFVVAGAFLLSGSGILLTLPHFFCFSTTTSGPQDLVTEALDTKVPLPKEGLEED

(19) United States
(12) Patent Application Publication
(10) Pub. No.: US 2007/0238151 A1
(43) Pub. Date: Oct. 11, 2007

(54) Novel compounds

(75) Inventors: Pankaj Agarwal, King of Prussia, PA (US); John P. Cogswell, Research Triangle Park, NC (US); Ying-ta Lai, Upper Darby, PA (US); Shelby A. Martensen, Research Triangle Park, NC (US); Randall F. Smith, Lafayette Hill, PA (US); Jay C. Strum, Research Triangle park, NC (US); Qing Xie, Horsham, PA (US)

Correspondence Address: GLAXOSMITHKLINE;Corporate Intellectual Property - UW2220, P.O. Box 1539, King of Prussia, PA, 19406-0939, US

(73) Assignee: SmithKline Beecham Corp. plc & Glaxo Group Limited

(21) Appl. No.: 11/445,001

(22) Filed: Jun. 1, 2006

Related U.S. Application Data
(63) Continuation of application No. 10/380,561, filed on Mar. 13, 2003, now abandoned.
(60) Provisional application No. 60/232,463, filed on Sep. 13, 2000.
(60) Provisional application No. 60/232,455, filed on Sep. 13, 2000.
(60) Provisional application No. 60/237,293, filed on Oct. 2, 2000.
(60) Provisional application No. 60/246,269, filed on Nov. 7, 2000.
(60) Provisional application No. 60/252,049, filed on Nov. 20, 2000.

(51) Int. Cl. C12P 21/06; C07K 14/705; C07K 14/195; C12N 1/21; C07H 21/04

(52) U.S. Cl. 435/69.1; 435/320.1; 435/252.3; 435/325; 530/350; 536/23.5

(57) ABSTRACT

Polypeptides and polynucleotides of the genes set forth in Table I and methods for producing such polypeptides by recombinant techniques are disclosed. Also disclosed are methods for utilizing polypeptides and polynucleotides of the genes set forth in Table I in diagnostic assays.

### CROSS REFERENCE TO PRIOR APPLICATIONS

This is a continuation of application Ser. No. 10/380,561 filed Mar. 13, 2003, now pending, which is a 371 application of PCT/US01/28462 filed Sep. 13, 2001 which claims the benefit of U.S. Provisional 60/232,463 filed Sep. 13, 2000, now abandoned which claims the benefit of 60/232,455 filed Sep. 13, 2000, and claims the benefit of 60/237,293 filed Oct. 2, 2000, and claims the benefit of 60/246,269 filed Nov. 7, 2000, and claims the benefit of 60/252,049 filed Nov. 20, 2000. All of the above disclosures are hereby incorporated by reference in their entirety.

### FIELD OF INVENTION

This invention relates to newly identified polypeptides and polynucleotides encoding such polypeptides, to their use in diagnosis and in identifying compounds that may be agonists, antagonists that are potentially useful in therapy, and to production of such polypeptides and polynucleotides. The polynucleotides and polypeptides of the present invention also relate to proteins with signal sequences which allow them to be secreted extracellularly or membrane-associated (hereinafter often referred collectively as secreted proteins or secreted polypeptides).

### BACKGROUND OF THE INVENTION

The drug discovery process is currently undergoing a fundamental revolution as it embraces “functional genomics”, that is, high throughput genome- or gene-based biology. This approach as a means to identify genes and gene products as therapeutic targets is rapidly superseding earlier approaches based on “positional cloning”. A phenotype, that is a biological function or genetic disease, would be identified and this would then be tracked back to the responsible gene, based on its genetic map position.

Functional genomics relies heavily on high-throughput DNA sequencing technologies and the various tools of bioinformatics to identify gene sequences of potential interest from the many molecular biology databases now available. There is a continuing need to identify and characterise further genes and their related polypeptides/proteins, as targets for drug discovery.

Proteins and polypeptides that are naturally secreted into blood, lymph and other body fluids, or secreted into the cellular membrane are of primary interest for pharmaceutical research and development. The reason for this interest is the relative ease to target protein therapeutics into their place of action (body fluids or the cellular membrane). The natural pathway for protein secretion into extracellular space is the endoplasmic reticulum in eukaryotes and the inner membrane in prokaryotes (Palade, 1975, Science, 189, 347; Milstein, Brownlee, Harrison, and Mathews, 1972, Nature New Biol., 239, 117; Blobel, and Dobberstein, 1975, J. Cell. Biol., 67, 835). On the other hand, there is no known natural pathway for exporting a protein from the exterior of the cells into the cytosol (with the exception of pinocytosis, a mechanism of snake venom toxin intrusion into cells). Therefore targeting protein therapeutics into cells poses extreme difficulties.

The secreted and membrane-associated proteins include but are not limited to all peptide hormones and their receptors (including but not limited to insulin, growth hormones, chemokines, cytokines, neuropeptides, integrins, kallikreins, lamins, melanins, natriuretic hormones, neuropsin, neurotropins, pituitiary hormones, pleiotropins, prostaglandins, secretogranins, selectins, thromboglobulins, thymosins), the breast and colon cancer gene products, leptin, the obesity gene protein and its receptors, serum albumin, superoxide dismutase, spliceosome proteins, 7TM (transmembrane) proteins also called as G-protein coupled receptors, immunoglobulins, several families of serine proteinases (including but not limited to proteins of the blood coagulation cascade, digestive enzymes), deoxyribonuclease I, etc.

Therapeutics based on secreted or membrane-associated proteins approved by FDA or foreign agencies include but are not limited to insulin, glucagon, growth hormone, chorionic gonadotropin, follicle stimulating hormone, luteinizing hormone, calcitonin, adrenocorticotropic hormone (ACTH), vasopressin, interleukines, interferones, immunoglobulins, lactoferrin (diverse products marketed by several companies), tissue-type plasminogen activator (Alteplase by Genentech), hyaulorindase (Wydase by Wyeth-Ayerst), dornase alpha (Pulmozyme\by Genentech), Chymodiactin (chymopapain by Knoll), alglucerase (Ceredase by Genzyme), streptokinase (Kabikinase by Pharmacia) (Streptase by Astra), etc. This indicates that secreted and membrane-associated proteins have an established, proven history as therapeutic targets. Clearly, there is a need for identification and characterization of further secreted and membrane-associated proteins which can play a role in preventing, ameliorating or correcting dysfunction or disease, including but not limited to diabetes, breast-, prostate-, colon cancer and other malignant tumors, hyper- and hypotension, obesity, bulimia, anorexia, growth abnormalities, asthma, manic depression, dementia, delirium, mental retardation, Huntington's disease, Tourette's syndrome, schizophrenia, growth, mental or sexual development disorders, and dysfunctions of the blood cascade system including those leading to stroke. The proteins of the present invention which include the signal sequences are also useful to further elucidate the mechanism of protein transport which at present is not entirely understood, and thus can be used as research tools.

### SUMMARY OF THE INVENTION

The present invention relates to particular polypeptides and polynucleotides of the genes set forth in Table I, including recombinant materials and methods for their production. Such polypeptides and polynucleotides are of interest in relation to methods of treatment of certain diseases, including, but not limited to, the diseases set forth in Tables III and V, hereinafter referred to as “diseases of the invention”. In a further aspect, the invention relates to methods for identifying agonists and antagonists (e.g., inhibitors) using the materials provided by the invention, and treating conditions associated with imbalance of polypeptides and/or polynucleotides of the genes set forth in Table I with the identified compounds. In still a further aspect, the invention relates to diagnostic assays for detecting diseases associated with inappropriate activity or levels the genes set forth in Table I. Another aspect of the invention concerns a polynucleotide comprising any of the nucleotide sequences set forth in the Sequence Listing and a polypeptide comprising a polypeptide encoded by the nucleotide sequence. In another aspect, the invention relates to a polypeptide comprising any of the polypeptide sequences set forth in the Sequence Listing and recombinant materials and methods for their production. Another aspect of the invention relates to methods for using such polypeptides and polynucleotides. Such uses include the treatment of diseases, abnormalities and disorders (hereinafter simply referred to as diseases) caused by abnormal expression, production, function and or metabolism of the genes of this invention, and such diseases are readily apparent by those skilled in the art from the homology to other proteins disclosed for each attached sequence. In still another aspect, the invention relates to methods to identify agonists and antagonists using the materials provided by the invention, and treating conditions associated with the imbalance with the identified compounds. Yet another aspect of the invention relates to diagnostic assays for detecting diseases associated with inappropriate activity or levels of the secreted proteins of the present invention.

### DESCRIPTION OF THE INVENTION

In a first aspect, the present invention relates to polypeptides the genes set forth in Table I. Such polypeptides include:

(a) an isolated polypeptide encoded by a polynucleotide comprising a sequence set forth in the Sequence Listing, herein when referring to polynucleotides or polypeptides of the Sequence Listing, a reference is also made to the Sequence Listing referred to in the Sequence Listing;

(b) an isolated polypeptide comprising a polypeptide sequence having at least 95%, 96%, 97%, 98%, or 99% identity to a polypeptide sequence set forth in the Sequence Listing;

(c) an isolated polypeptide comprising a polypeptide sequence set forth in the Sequence Listing;

(d) an isolated polypeptide having at least 95%, 96%, 97%, 98%, or 99% identity to a polypeptide sequence set forth in the Sequence Listing;

(e) a polypeptide sequence set forth in the Sequence Listing; and

(f) an isolated polypeptide having or comprising a polypeptide sequence that has an Identity Index of 0.95, 0.96, 0.97, 0.98, or 0.99 compared to a polypeptide sequence set forth in the Sequence Listing;

(g) fragments and variants of such polypeptides in (a) to (f).

Polypeptides of the present invention are believed to be members of the gene families set forth in Table II. They are therefore of therapeutic and diagnostic interest for the reasons set forth in Tables III and V. The biological properties of the polypeptides and polynucleotides of the genes set forth in Table I are hereinafter referred to as “the biological activity” of polypeptides and polynucleotides of the genes set forth in Table I. Preferably, a polypeptide of the present invention exhibits at least one biological activity of the genes set forth in Table I.

Polypeptides of the present invention also include variants of the aforementioned polypeptides, including all allelic forms and splice variants. Such polypeptides vary from the reference polypeptide by insertions, deletions, and substitutions that may be conservative or non-conservative, or any combination thereof. Particularly preferred variants are those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination.

Preferred fragments of polypeptides of the present invention include an isolated polypeptide comprising an amino acid sequence having at least 30, 50 or 100 contiguous amino acids from an amino acid sequence set forth in the Sequence Listing, or an isolated polypeptide comprising an amino acid sequence having at least 30, 50 or 100 contiguous amino acids truncated or deleted from an amino acid sequence set forth in the Sequence Listing. Preferred fragments are biologically active fragments that mediate the biological activity of polypeptides and polynucleotides of the genes set forth in Table I, including those with a similar activity or an improved activity, or with a decreased undesirable activity. Also preferred are those fragments that are antigenic or immunogenic in an animal, especially in a human.

Fragments of a polypeptide of the invention may be employed for producing the corresponding full-length polypeptide by peptide synthesis; therefore, these variants may be employed as intermediates for producing the full-length polypeptides of the invention. A polypeptide of the present invention may be in the form of the “mature” protein or may be a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Polypeptides of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. Means for preparing such polypeptides are well understood in the art.

In a further aspect, the present invention relates to polynucleotides of the genes set forth in Table I. Such polynucleotides include:

(a) an isolated polynucleotide comprising a polynucleotide sequence having at least 95%, 96%, 97%, 98%, or 99% identity to a polynucleotide sequence set forth in the Sequence Listing;

(b) an isolated polynucleotide comprising a polynucleotide set forth in the Sequence Listing;

(c) an isolated polynucleotide having at least 95%, 96%, 97%, 98%, or 99% identity to a polynucleotide set forth in the Sequence Listing;

(d) an isolated polynucleotide set forth in the Sequence Listing;

(e) an isolated polynucleotide comprising a polynucleotide sequence encoding a polypeptide sequence having at least 95%, 96%, 97%, 98%, or 99% identity to a polypeptide sequence set forth in the Sequence Listing;

(f) an isolated polynucleotide comprising a polynucleotide sequence encoding a polypeptide set forth in the Sequence Listing;

(g) an isolated polynucleotide having a polynucleotide sequence encoding a polypeptide sequence having at least 95%, 96%, 97%, 98%, or 99% identity to a polypeptide sequence set forth in the Sequence Listing;

(h) an isolated polynucleotide encoding a polypeptide set forth in the Sequence Listing;

(i) an isolated polynucleotide having or comprising a polynucleotide sequence that has an Identity Index of 0.95, 0.96, 0.97, 0.98, or 0.99 compared to a polynucleotide sequence set forth in the Sequence Listing;

j) an isolated polynucleotide having or comprising a polynucleotide sequence encoding a polypeptide sequence that has an Identity Index of 0.95, 0.96, 0.97, 0.98, or 0.99 compared to a polypeptide sequence set forth in the Sequence Listing; and

polynucleotides that are fragments and variants of the above mentioned polynucleotides or that are complementary to above mentioned polynucleotides, over the entire length thereof.

Preferred fragments of polynucleotides of the present invention include an isolated polynucleotide comprising an nucleotide sequence having at least 15, 30, 50 or 100 contiguous nucleotides from a sequence set forth in the Sequence Listing, or an isolated polynucleotide comprising a sequence having at least 30, 50 or 100 contiguous nucleotides truncated or deleted from a sequence set forth in the Sequence Listing.

Preferred variants of polynucleotides of the present invention include splice variants, allelic variants, and polymorphisms, including polynucleotides having one or more single nucleotide polymorphisms (SNPs).

Polynucleotides of the present invention also include polynucleotides encoding polypeptide variants that comprise an amino acid sequence set forth in the Sequence Listing and in which several, for instance from 50 to 30, from 30 to 20, from 20 to 10, from 10 to 5, from 5 to 3, from 3 to 2, from 2 to 1 or 1 amino acid residues are substituted, deleted or added, in any combination.

In a further aspect, the present invention provides polynucleotides that are RNA transcripts of the DNA sequences of the present invention. Accordingly, there is provided an RNA polynucleotide that:

(a) comprises an RNA transcript of the DNA sequence encoding a polypeptide set forth in the Sequence Listing;

(b) is a RNA transcript of a DNA sequence encoding a polypeptide set forth in the Sequence Listing;

(c) comprises an RNA transcript of a DNA sequence set forth in the Sequence Listing; or

(d) is a RNA transcript of a DNA sequence set forth in the Sequence Listing; and RNA polynucleotides that are complementary thereto.

The polynucleotide sequences set forth in the Sequence Listing show homology with the polynucleotide sequences set forth in Table II. A polynucleotide sequence set forth in the Sequence Listing is a cDNA sequence that encodes a polypeptide set forth in the Sequence Listing. A polynucleotide sequence encoding a polypeptide set forth in the Sequence Listing may be identical to a polypeptide encoding a sequence set forth in the Sequence Listing or it may be a sequence other than a sequence set forth in the Sequence Listing, which, as a result of the redundancy (degeneracy) of the genetic code, also encodes a polypeptide set forth in the Sequence Listing. A polypeptide of a sequence set forth in the Sequence Listing is related to other proteins of the gene families set forth in Table II, having homology and/or structural similarity with the polypeptides set forth in Table II. Preferred polypeptides and polynucleotides of the present invention are expected to have, inter alia, similar biological functions/properties to their homologous polypeptides and polynucleotides. Furthermore, preferred polypeptides and polynucleotides of the present invention have at least one activity of the genes set forth in Table I.

Polynucleotides of the present invention may be obtained using standard cloning and screening techniques from a cDNA library derived from mRNA from the tissues set forth in Table IV (see for instance, Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro- protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc Natl Acad Sci USA (1989) 86:821-824, or is an HA tag. A polynucleotide may also contain non-coding 5′ and 3′ sequences, such as transcribed, non-translated sequences, splicing and polyadenylation signals, ribosome binding sites and sequences that stabilize mRNA.

Polynucleotides that are identical, or have sufficient identity to a polynucleotide sequence set forth in the Sequence Listing, may be used as hybridization probes for cDNA and genomic DNA or as primers for a nucleic acid amplification reaction (for instance, PCR). Such probes and primers may be used to isolate full-length cDNAs and genomic clones encoding polypeptides of the present invention and to isolate cDNA and genomic clones of other genes (including genes encoding paralogs from human sources and orthologs and paralogs from other species) that have a high sequence similarity to sequences set forth in the Sequence Listing, typically at least 95% identity. Preferred probes and primers will generally comprise at least 15 nucleotides, preferably, at least 30 nucleotides and may have at least 50, if not at least 100 nucleotides. Particularly preferred probes will have between 30 and 50 nucleotides. Particularly preferred primers will have between 20 and 25 nucleotides.

A polynucleotide encoding a polypeptide of the present invention, including homologs from other species, may be obtained by a process comprising the steps of screening a library under stringent hybridization conditions with a labeled probe having a sequence set forth in the Sequence Listing or a fragment thereof, preferably of at least 15 nucleotides; and isolating full-length cDNA and genomic clones containing the polynucleotide sequence set forth in the Sequence Listing. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 microgram/ml denatured, sheared salmon sperm DNA; followed by washing the filters in 0.1×SSC at about 65° C. Thus the present invention also includes isolated polynucleotides, preferably with a nucleotide sequence of at least 100, obtained by screening a library under stringent hybridization conditions with a labeled probe having the sequence set forth in the Sequence Listing or a fragment thereof, preferably of at least 15 nucleotides.

The skilled artisan will appreciate that, in many cases, an isolated cDNA sequence will be incomplete, in that the region coding for the polypeptide does not extend all the way through to the 5′ terminus. This is a consequence of reverse transcriptase, an enzyme with inherently low “processivity” (a measure of the ability of the enzyme to remain attached to the template during the polymerisation reaction), failing to complete a DNA copy of the mRNA template during first strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al. (ibid). Preferred methods of introducing polynucleotides into host cells include, for instance, calcium phosphate transfection, DEAE-dextran mediated transfection, transvection, micro-injection, cationic lipid-mediated transfection, electroporation, transduction, scrape loading, ballistic introduction or infection.

Representative examples of appropriate hosts include bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al., (ibid). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation and/or purification.

Polynucleotides of the present invention may be used as diagnostic reagents, through detecting mutations in the associated gene. Detection of a mutated form of a gene is characterized by the polynucleotides set forth in the Sequence Listing in the cDNA or genomic sequence and which is associated with a dysfunction. Will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques well known in the art.

Nucleic acids for diagnosis may be obtained from a subject's cells, such as from blood, urine, saliva, tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences of the genes set forth in Table I. Perfectly matched sequences can be distinguished from mismatched duplexes by RNase digestion or by differences in melting temperatures. DNA sequence difference may also be detected by alterations in the electrophoretic mobility of DNA fragments in gels, with or without denaturing agents, or by direct DNA sequencing (see, for instance, Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85: 4397-4401).

An array of oligonucleotides probes comprising polynucleotide sequences or fragments thereof of the genes set forth in Table I can be constructed to conduct efficient screening of e.g., genetic mutations. Such arrays are preferably high density arrays or grids. Array technology methods are well known and have general applicability and can be used to address a variety of questions in molecular genetics including gene expression, genetic linkage, and genetic variability, see, for example, M. Chee et al., Science, 274, 610-613 (1996) and other references cited therein.

Detection of abnormally decreased or increased levels of polypeptide or mRNA expression may also be used for diagnosing or determining susceptibility of a subject to a disease of the invention. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radio-immunoassays, competitive-binding assays, Western Blot analysis and ELISA assays.

Thus in another aspect, the present invention relates to a diagnostic kit comprising:

(a) a polynucleotide of the present invention, preferably the nucleotide sequence set forth in the Sequence Listing, or a fragment or an RNA transcript thereof;

(b) a nucleotide sequence complementary to that of (a);

(c) a polypeptide of the present invention, preferably the polypeptide set forth in the Sequence Listing or a fragment thereof; or

(d) an antibody to a polypeptide of the present invention, preferably to the polypeptide set forth in the Sequence Listing.

It will be appreciated that in any such kit, (a), (b), (c) or (d) may comprise a substantial component. Such a kit will be of use in diagnosing a disease or susceptibility to a disease, particularly diseases of the invention, amongst others.

The polynucleotide sequences of the present invention are valuable for chromosome localisation studies. The sequences set forth in the Sequence Listing are specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (co-inheritance of physically adjacent genes). Precise human chromosomal localisations for a genomic sequence (gene fragment etc.) can be determined using Radiation Hybrid (RH) Mapping (Walter, M. Spillett, D., Thomas, P., Weissenbach, J., and Goodfellow, P., (1994) A method for constructing radiation hybrid maps of whole genomes, Nature Genetics 7, 22-28). A number of RH panels are available from Research Genetics (Huntsville, Ala., USA) e.g. the GeneBridge4 RH panel (Hum Mol Genet 1996 March; 5(3):33946 A radiation hybrid map of the human genome. Gyapay G, Schmitt K, Fizames C, Jones H, Vega-Czarny N, Spillett D, Muselet D, Prud'Homme J F, Dib C, Auffray C, Morissette J, Weissenbach J, Goodfellow P N). To determine the chromosomal location of a gene using this panel, 93 PCRs are performed using primers designed from the gene of interest on RH DNAs. Each of these DNAs contains random human genomic fragments maintained in a hamster background (human/hamster hybrid cell lines). These PCRs result in 93 scores indicating the presence or absence of the PCR product of the gene of interest. These scores are compared with scores created using PCR products from genomic sequences of known location. This comparison is conducted at http://www.genome.wi.mit.edu/.

The polynucleotide sequences of the present invention are also valuable tools for tissue expression studies. Such studies allow the determination of expression patterns of polynucleotides of the present invention which may give an indication as to the expression patterns of the encoded polypeptides in tissues, by detecting the mRNAs that encode them. The techniques used are well known in the art and include in situ hydridization techniques to clones arrayed on a grid, such as cDNA microarray hybridization (Schena et al, Science, 270, 467-470, 1995 and Shalon et al, Genome Res, 6, 639-645, 1996) and nucleotide amplification techniques such as PCR. A preferred method uses the TAQMAN (Trade mark) technology available from Perkin Elmer. Results from these studies can provide an indication of the normal function of the polypeptide in the organism. In addition, comparative studies of the normal expression pattern of mRNAs with that of mRNAs encoded by an alternative form of the same gene (for example, one having an alteration in polypeptide coding potential or a regulatory mutation) can provide valuable insights into the role of the polypeptides of the present invention, or that of inappropriate expression thereof in disease. Such inappropriate expression may be of a temporal, spatial or simply quantitative nature.

A further aspect of the present invention relates to antibodies. The polypeptides of the invention or their fragments, or cells expressing them, can be used as immunogens to produce antibodies that are immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography. Antibodies against polypeptides of the present invention may also be employed to treat diseases of the invention, amongst others.

Polypeptides and polynucleotides of the present invention may also be used as vaccines. Accordingly, in a further aspect, the present invention relates to a method for inducing an immunological response in a mammal that comprises inoculating the mammal with a polypeptide of the present invention, adequate to produce antibody and/or T cell immune response, including, for example, cytokine-producing T cells or cytotoxic T cells, to protect said animal from disease, whether that disease is already established within the individual or not. An immunological response in a mammal may also be induced by a method comprises delivering a polypeptide of the present invention via a vector directing expression of the polynucleotide and coding for the polypeptide in vivo in order to induce such an immunological response to produce antibody to protect said animal from diseases of the invention. One way of administering the vector is by accelerating it into the desired cells as a coating on particles or otherwise. Such nucleic acid vector may comprise DNA, RNA, a modified nucleic acid, or a DNA/RNA hybrid. For use a vaccine, a polypeptide or a nucleic acid vector will be normally provided as a vaccine formulation (composition). The formulation may further comprise a suitable carrier. Since a polypeptide may be broken down in the stomach, it is preferably administered parenterally (for instance, subcutaneous, intramuscular, intravenous, or intra-dermal injection). Formulations suitable for parenteral administration include aqueous and non-aqueous sterile injection solutions that may contain anti-oxidants, buffers, bacteriostats and solutes that render the formulation instonic with the blood of the recipient; and aqueous and non-aqueous sterile suspensions that may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention have one or more biological functions that are of relevance in one or more disease states, in particular the diseases of the invention hereinbefore mentioned. It is therefore useful to identify compounds that stimulate or inhibit the function or level of the polypeptide. Accordingly, in a further aspect, the present invention provides for a method of screening compounds to identify those that stimulate or inhibit the function or level of the polypeptide. Such methods identify agonists or antagonists that may be employed for therapeutic and prophylactic purposes for such diseases of the invention as hereinbefore mentioned. Compounds may be identified from a variety of sources, for example, cells, cell-free preparations, chemical libraries, collections of chemical compounds, and natural product mixtures. Such agonists or antagonists so-identified may be natural or modified substrates, ligands, receptors, enzymes, etc., as the case may be, of the polypeptide; a structural or functional mimetic thereof (see Coligan et al., Current Protocols in Immunology 1(2):Chapter 5 (1991)) or a small molecule. Such small molecules preferably have a molecular weight below 2,000 daltons, more preferably between 300 and 1,000 daltons, and most preferably between 400 and 700 daltons. It is preferred that these small molecules are organic molecules.

The screening method may simply measure the binding of a candidate compound to the polypeptide, or to cells or membranes bearing the polypeptide, or a fusion protein thereof, by means of a label directly or indirectly associated with the candidate compound. Alternatively, the screening method may involve measuring or detecting (qualitatively or quantitatively) the competitive binding of a candidate compound to the polypeptide against a labeled competitor (e.g. agonist or antagonist). Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Further, the screening methods may simply comprise the steps of mixing a candidate compound with a solution containing a polypeptide of the present invention, to form a mixture, measuring an activity of the genes set forth in Table I in the mixture, and comparing activity of the mixture of the genes set forth in Table I to a control mixture which contains no candidate compound.

Polypeptides of the present invention may be employed in conventional low capacity screening methods and also in high-throughput screening (HTS) formats. Such HTS formats include not only the well-established use of 96- and, more recently, 384-well micotiter plates but also emerging methods such as the nanowell method described by Schullek et al, Anal Biochem., 246, 20-29, (1997).

Fusion proteins, such as those made from Fc portion and polypeptide of the genes set forth in Table I, as hereinbefore described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

A polypeptide of the present invention may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide that compete with the binding of the polypeptide to its receptors, if any. Standard methods for conducting such assays are well understood in the art.

Examples of antagonists of polypeptides of the present invention include antibodies or, in some cases, oligonucleotides or proteins that are closely related to the ligands, substrates, receptors, enzymes, etc., as the case may be, of the polypeptide, e.g., a fragment of the ligands, substrates, receptors, enzymes, etc.; or a small molecule that bind to the polypeptide of the present invention but do not elicit a response, so that the activity of the polypeptide is prevented.

Screening methods may also involve the use of transgenic technology and the genes set forth in Table I. The art of constructing transgenic animals is well established. For example, the genes set forth in Table I may be introduced through microinjection into the male pronucleus of fertilized oocytes, retroviral transfer into pre- or post-implantation embryos, or injection of genetically modified, such as by electroporation, embryonic stem cells into host blastocysts. Particularly useful transgenic animals are so-called “knock-in” animals in which an animal gene is replaced by the human equivalent within the genome of that animal. Knock-in transgenic animals are useful in the drug discovery process, for target validation, where the compound is specific for the human target. Other useful transgenic animals are so-called “knock-out” animals in which the expression of the animal ortholog of a polypeptide of the present invention and encoded by an endogenous DNA sequence in a cell is partially or completely annulled. The gene knock-out may be targeted to specific cells or tissues, may occur only in certain cells or tissues as a consequence of the limitations of the technology, or may occur in all, or substantially all, cells in the animal. Transgenic animal technology also offers a whole animal expression-cloning system in which introduced genes are expressed to give large amounts of polypeptides of the present invention

Screening kits for use in the above described methods form a further aspect of the present invention. Such screening kits comprise:

(a) a polypeptide of the present invention;

(b) a recombinant cell expressing a polypeptide of the present invention;

(c) a cell membrane expressing a polypeptide of the present invention; or

(d) an antibody to a polypeptide of the present invention;

which polypeptide is preferably that set forth in the Sequence Listing.

It will be appreciated that in any such kit, (a), (b), (c) or (d) may comprise a substantial component.

Glossary

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an

Fab or other immunoglobulin expression library.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Secreted protein activity or secreted polypeptide activity” or “biological activity of the secreted protein or secreted polypeptide” refers to the metabolic or physiologic function of said secreted protein including similar activities or improved activities or these activities with decreased undesirable side-effects. Also included are antigenic and immunogenic activities of said secreted protein.

“Secreted protein gene” refers to a polynucleotide comprising any of the attached nucleotide sequences or allelic variants thereof and/or their complements.

“Polynucleotide” generally refers to any polyribonucleotide (RNA) or polydeoxyribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et al., “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucleotide sequence that is shorter than the reference sequence set forth in the Sequence Listing.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448, 1988, available as part of the Wisconsin Sequence Analysis Package).

Preferably, the BLOSUM62 amino acid substitution matrix (Henikoff S and Henikoff J G, Proc. Nat. Acad. Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5 in every 100 of the nucleotides of the in the reference sequence may be deleted, substituted or inserted, or any combination thereof, as hereinbefore described. The same applies mutatis mutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutandis for other values of the Identity Index, for instance 0.96, 0.97, 0.98 and 0.99.

The relationship between the number of nucleotide or amino acid differences and the Identity Index may be expressed in the following equation:
nₐ≦xₐ−(xₐ·I)
in which:

nₐ is the number of nucleotide or amino acid differences,

xₐ is the total number of nucleotides or amino acids in a sequence set forth in the Sequence Listing,

I is the Identity Index,

· is the symbol for the multiplication operator, and

in which any non-integer product of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotideor polypeptide that within the same species which is functionally similar.

“Fusion protein” refers to a protein encoded by two, often unrelated, fused genes or fragments thereof. In one example, EP-A-0 464 533-A discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

TABLE I

| | | | |
|---|---|---|---|
| | GSK | Nucleic Acid | Corresponding Protein |
| Gene Name | Gene ID | SEQ ID NO’s | SEQ ID NO’s |

| | | | |
|---|---|---|---|
| sbg960509cbrecpt | 960509 | SEQ ID NO:1 | SEQ ID NO:45 |
| sbg614126complfH | 614126 | SEQ ID NO:2 | SEQ ID NO:46 |
| | | SEQ ID NO:3 | SEQ ID NO:47 |
| sbg120703RNase | 120703 | SEQ ID NO:4 | SEQ ID NO:48 |
| sbg98530TS | 98530 | SEQ ID NO:5 | SEQ ID NO:49 |
| | | SEQ ID NO:6 | SEQ ID NO:50 |
| sbg563917RDP | 63917 | SEQ ID NO:7 | SEQ ID NO:51 |
| | | SEQ ID NO:8 | SEQ ID NO:52 |
| sbg618069LRR | 618069 | SEQ ID NO:9 | SEQ ID NO:53 |
| | | SEQ ID NO:10 | SEQ ID NO:54 |
| sbg934114Relaxin | 934114 | SEQ ID NO:11 | SEQ ID NO:55 |
| sbg99174LOX-like | 99174 | SEQ ID NO:12 | SEQ ID NO:56 |
| sbg995002PIGR | 995002 | SEQ ID NO:13 | SEQ ID NO:57 |
| sbg1033026C1q | 1033026 | SEQ ID NO:14 | SEQ ID NO:58 |
| | | SEQ ID NO:15 | SEQ ID NO:59 |
| sbg1003675RNase | 1003675 | SEQ ID NO:16 | SEQ ID NO:60 |
| sbg1015258PLM | 1015258 | SEQ ID NO:17 | SEQ ID NO:61 |
| sbg1003328IG | 1003328 | SEQ ID NO:18 | SEQ ID NO:62 |
| | | SEQ ID NO:19 | SEQ ID NO:63 |
| sbg1020829SGLT | 1020829 | SEQ ID NO:20 | SEQ ID NO:64 |
| sbg1005450UDPGT | 1005450 | SEQ ID NO:21 | SEQ ID NO:65 |
| | | SEQ ID NO:22 | SEQ ID NO:66 |
| sbg1002620TIa | 1002620 | SEQ ID NO:23 | SEQ ID NO:67 |
| | | SEQ ID NO:24 | SEQ ID NO:68 |
| sbg1002620TIb | 1002620 | SEQ ID NO:25 | SEQ ID NO:69 |
| sbg102200MCTa | 102200 | SEQ ID NO:26 | SEQ ID NO:70 |
| | | SEQ ID NO:27 | SEQ ID NO:71 |
| sbg102200MCTb | 102200 | SEQ ID NO:28 | SEQ ID NO:72 |
| sbg1020380LYG | 1020380 | SEQ ID NO:29 | SEQ ID NO:73 |
| | | SEQ ID NO:30 | SEQ ID NO:74 |
| sbg1007026SGLT | 1007026 | SEQ ID NO:31 | SEQ ID NO:75 |
| sbg1012732GLUT | 1012732 | SEQ ID NO:32 | SEQ ID NO:76 |
| | | SEQ ID NO:33 | SEQ ID NO:77 |
| sbg1012732GLUTb | 1012732 | SEQ ID NO:34 | SEQ ID NO:78 |
| sbg1018172CSP | 1018172 | SEQ ID NO:35 | SEQ ID NO:79 |
| | | SEQ ID NO:36 | SEQ ID NO:80 |
| sbg1004570ERGIC | 1004570 | SEQ ID NO:37 | SEQ ID NO:81 |
| | | SEQ ID NO:38 | SEQ ID NO:82 |
| sbg1016995IGBrecpt | 1016995 | SEQ ID NO:39 | SEQ ID NO:83 |
| | | SEQ ID NO:40 | SEQ ID NO:84 |
| sbg1151bSREC | 1151 | SEQ ID NO:41 | SEQ ID NO:85 |
| | | SEQ ID NO:42 | SEQ ID NO:86 |
| sbg1399854ANK | 1399854 | SEQ ID NO:43 | SEQ ID NO:87 |
| | | SEQ ID NO:44 | SEQ ID NO:88 |

TABLE II

| | | | | Cell |
|---|---|---|---|---|
| | | | | Localization |
| | Gene | Closest Polynucleotide | Closest Polypeptide by | (by |
| Gene Name | Family | by homology | homology | homology) |
| sbg960509cbrecpt | Carbohydrate- | GB: AC007395 | Mouse Kupffer cell c- | Membrane- |
| | binding | Direct submitted (25- | type lectin receptor, gi: | bound |
| | receptor | APR-1999) Genome | 7949066 Submitted | |
| | | Sequencing Center, | (25-OCT-1996) to the | |
| | | Washington University | DDBJ/EMBL/GenBank | |
| | | School of Medicine, | databases. | |
| | | 4444 Forest Park | | |
| | | Parkway, St. Louis, MO | | |
| | | 63108, USA | | |
| sbg614126complfH | Complement | SC: AL353809 | Human H-factor like 1, | Secreted |
| | factor H | Submitted (20-JAN- | gi: 11321587 | |
| | | 2001) by Sanger Centre, | Estaller, C., | |
| | | Hinxton, | Koistinen, V., | |
| | | Cambridgeshire, CB10 | Schwaeble, W., | |
| | | 1SA, UK. | Dierich, M. P., and | |
| | | | Weiss, E. H. J. Immunol. | |
| | | | 146, 3190-3196 (1991) | |
| sbg120703RNase | RNase | GB: AL157687 | Human keratinocyte- | Secreted |
| | | Direct Submitted (24- | derived RNase-like | |
| | | MAY-2000) to the | protein, geneseqp: | |
| | | EMBL/GenBank/DDBJ | Y44192 Submitted by | |
| | | databases by Genoscope. | INNOGENETICS NV | |
| | | | Application number and | |
| | | | publication date: EP- | |
| | | | 943679-A1, 22-SEP-99 | |
| sbg98530TS | Thrombospondin | GB: AC027307 | Mouse RIKEN cDNA | Secreted |
| | type I | Submitted (30-MAR- | 2010109H09 gene, | |
| | | 2000) Production | gi: 13385092 | |
| | | Sequencing Facility, | The RIKEN Genome | |
| | | DOE Joint Genome | Exploration Research | |
| | | Institute, 2800 Mitchell | Group Phase II Team | |
| | | Drive, Walnut Creek, | and FANTOM | |
| | | CA 94598, USA | Consortium. Nature | |
| | | | 409, 685-690 (2001) | |
| sbg563917RDP | Renal | GB: AC009077 | Human putative | Secreted |
| | dipeptidase | Directly submitted (03- | metallopeptidase | |
| | | AUG-1999) by | (family M19) | |
| | | Production Sequencing | gi: 11641273 Chen, J. M., | |
| | | Facility, DOE Joint | Fortunato, M. and | |
| | | Genome Institute, 2800 | Barrett, A. J. | |
| | | Mitchell Drive, Walnut | Submitted (02-NOV- | |
| | | Creek, CA 94598, USA | 2000) Chen J. M., MRC | |
| | | | Molecular Enzymology | |
| | | | Laboratory, The | |
| | | | Babraham Institute, | |
| | | | Babraham, Cambridge, | |
| | | | CB2 4AT, UK | |
| sbg618069LRR | Leucine- | GB: AL589765 | Macaca fascicularis | Membrane- |
| | rich repeat | Submitted (16-MAR- | brain protein, gi: | bound |
| | | 2001) by Sanger Centre, | 9651088 Submitted | |
| | | Hinxton, Cambridgeshire, | (28-JUL-2000) to the | |
| | | CB10 1SA, UK. | DDBJ/EMBL/GenBank | |
| | | | databases. Katsuyuki | |
| | | | Hashimoto, National | |
| | | | Institute of Infectious | |
| | | | Diseases, Division of | |
| | | | Genetic Resources; 23-1, | |
| | | | Toyama 1-chome, | |
| | | | Shinjuku-ku, Tokyo | |
| | | | 162-8640, Japan | |
| sbg934114Relaxin | Insulin | JGI: CIT978SKB_55O6 | Mouse insulin-like | Secreted |
| | | Found at Joint Genome | peptide (relaxin/insulin- | |
| | | Institute | like protein), gi: 7387805 | |
| | | DoE/LLNL/LBNL/LANL. | Conklin D, Lofton-Day CE, | |
| | | | Haldeman BA, | |
| | | | Ching A, Whitmore TE, | |
| | | | Lok S, Jaspers S. 1999. | |
| | | | Genomics 60: 50-56. | |
| sbg99174LOX- | C-type | GB: AL137062 | Mouse putative protien, | Membrane- |
| like | lectin | Direct submitted (09- | gi: 12855891 The | bound |
| | | AUG-2000) Sanger | RIKEN Genome | |
| | | Centre, Hinxton, | Exploration Research | |
| | | Cambridgeshire, CB10 | Group Phase II Team | |
| | | 1SA, UK. | and FANTOM | |
| | | | Consortium. Nature | |
| | | | 409, 685-690 (2001) | |
| sbg995002PIGR | Polymeric - | GB: AC027192 | Human TANGO 354 | Membrane- |
| | immublobulin | Direct submitted (28- | protein, geneseqp: | bound |
| | receptor | MAR-2000) Whitehead | B66271 Submitted by | |
| | (PIGR) | Institute/MIT Center for | (MILL-) | |
| | | Genome Research, 320 | MILLENNIUM | |
| | | Charles Street, | PHARM INC | |
| | | Cambridge, MA 02141, | Application number and | |
| | | USA. | publication date: | |
| | | | WO200100673-A1, | |
| | | | 04-JAN-01 | |
| sbg1033026C1q | C1q | GB: AL359736 | Human adipocyte- | Secreted |
| | | Direct submitted (22- | specific secretory | |
| | | AUG-2000) Sanger | protein, gi: 4757760 | |
| | | Centre, Hinxton, | Maeda, K., Okubo, K., | |
| | | Cambridgeshire, CB10 | Shimomura, I., | |
| | | 1SA, UK. | Funahashi, T., | |
| | | | Matsuzawa, Y. and | |
| | | | Matsubara, K. | |
| | | | Biochem. Biophys. Res. | |
| | | | Commun. 221 (2), 286-289 | |
| | | | (1996) | |
| sbg1003675RNase | RNase | EMBL: CNS01RIH | Chinchilla brevicaudata | Secreted |
| | | Found at European | pancreatic ribonuclease, | |
| | | Molecular Biology | gi: 133205 Van Den | |
| | | Laboratory. | Berg A, Van Den | |
| | | | Hende-Timmer L, | |
| | | | Beintema JJ. 1976. | |
| | | | Biochim Biophys Acta | |
| | | | 453: 400-9. | |
| sbg1015258PLM | Phospholemman | GB: AL022345 | Human | Membrane- |
| | (PLM) | Direct submitted (10-DEC- | phospholemman-like | bound |
| | | 1999) by Sanger Centre, | protein, | |
| | | Hinxton, Cambridgeshire, | geneseqp: W51104 | |
| | | CB10 1SA, UK. | Submitted by (HUMA-) | |
| | | | HUMAN GENOME | |
| | | | SCI INC | |
| | | | Application number and | |
| | | | publication date: | |
| | | | WO9839448-A2, | |
| | | | 11-SEP-98 | |
| sbg1003328IG | Immunoglobulin | EMBL: HSBA536C5 | Human immune system | Membrane- |
| | | Found at European | molecule, | bound |
| | | Molecular Biology | geneseqp: B15536 | |
| | | Laboratory. | Submitted by (INCY-) | |
| | | | INCYTE PHARM INC | |
| | | | Application number and | |
| | | | publication date: | |
| | | | WO200060080-A2, | |
| | | | 12-OCT-00 | |
| sbg1020829SGLT | Na+/glucose | GB: AJ009617 | Oryctolagus cuniculus | Membrane- |
| | cotransporter | Directly submitted (17- | Na/glucose | bound |
| | | JUL-1998) by MPIMG, | cotransporters, | |
| | | Abt.Lehrach, Max Planck | gi: 520469 Pajor, A. M. | |
| | | Institut fuer Molekulare | 1994 Biochim. | |
| | | Genetik, Ihnestrasse 73, | Biophys. Acta 1194 (2), | |
| | | Berlin, 14195, Germany. | 349-51. | |
| sbg1005450UDPGT | UDP- | GB: AC016612 | Human PRO1780 | Membrane- |
| | glucuronosyltransferase | Submitted (04-DEC- | protein, geneseqp: | bound |
| | (UDPGT) | 1999) Production | B24025 | |
| | | Sequencing Facility, DOE | Submitted by | |
| | | Joint Genome Institute, | GENENTECH INC | |
| | | 2800 Mitchell Drive, | Application number and | |
| | | Walnut Creek, CA 94598, | publication date: | |
| | | USA | WO200053750-A1, | |
| | | | 14-SEP-00 | |
| sbg1002620TIa | Cysteine- | GB: AC025280 | Human hypothetical | Secreted |
| | rich | Submitted (08-MAR- | protein | |
| | secretory | 2000) by Production | DKFZp434B044, gi: | |
| | protein | Sequencing Facility, DOE | 13899332 Wiemann, S., | |
| | (CRISP) | Joint Genome Institute, | Weil, B. et al. Genome | |
| | trypsin | 2800 Mitchell Drive, | Res. 11 (3), 422-435 | |
| | inhibitor | Walnut Creek, CA 94598, | (2001) | |
| | | USA | | |
| sbg1002620TIb | Cysteine- | GB: AC025280 | Rat late gestation lung | Secreted |
| | rich | Submitted (08-MAR- | protein 1, gi: 4324682 | |
| | secretory | 2000) by Production | Kaplan, F., Ledoux, P., | |
| | protein | Sequencing Facility, DOE | Kassamali, F. Q., | |
| | (CRISP) | Joint Genome Institute, | Gagnon, S., Post, M., | |
| | trypsin | 2800 Mitchell Drive, | Koehler, D., Deimling, J. | |
| | inhibitor | Walnut Creek, CA 94598, | and Sweezey, N. B. | |
| | | USA | Am. J. Physiol. 276 (6), | |
| | | | L1027-L1036 (1999) | |
| sbg102200MCTa | Monocarboxylate | GB: AC015918 | Mouse unnamed | Membrane- |
| | cotransporter | Directly submitted (17- | protein product, | bound |
| | (MCT1) | NOV-1999) by | gi: 7670446 Submitted | |
| | | Whitehead Institute/MIT | (12-APR-2000) to the | |
| | | Center for Genome | DDBJ/EMBL/GenBank | |
| | | Research, 320 Charles | databases by Katsuyuki | |
| | | Street, Cambridge, MA | Hashimoto, National | |
| | | 02141, USA. | Institute of Infectious | |
| | | | Diseases, Division of | |
| | | | Genetic Resources; 23-1, | |
| | | | Toyama 1-chome, | |
| | | | Shinjuku-ku, Tokyo | |
| | | | 162-8640, Japan | |
| sbg102200MCTb | Monocarboxylate | GB: AC015918 | Human solute carrier 16 | Membrane- |
| | cotransporter | Directly submitted (17- | (monocarboxylic acid | bound |
| | (MCT1) | NOV-1999) by | transporters), member | |
| | | Whitehead Institute/MIT | 8, gi: 13655082 | |
| | | Center for Genome | Submitted (17-APR- | |
| | | Research, 320 Charles | 2001) by National | |
| | | Street, Cambridge, MA | Center for | |
| | | 02141, USA. | Biotechnology | |
| | | | Information, NIH, | |
| | | | Bethesda, MD 20894, | |
| | | | USA | |
| sbg1020380LYG | Goose- | GB: AC023965 | Lysozyme G (1,4-beta- | Secreted |
| | type | Directly submitted (20- | N-acetylmuramidas E) | |
| | lysozyme G | FEB-2000) by Whitehead | (Goose-type lysozyme). | |
| | | Institute/MIT Center for | gi: 126634 Schoentgen, F., | |
| | | Genome Research, 320 | Jolles, J. and Jolles, P. | |
| | | Charles Street, | Eur. J. Biochem. 123 | |
| | | Cambridge, MA 02141, | (3), 489-497 (1982) | |
| | | USA | | |
| sbg1007026SGLT | Sodium- | GB: AC046167 | Human transport | Membrane- |
| | glucose | Direct submitted (13- | protein TPPT-13, | bound |
| | cotransporter | APR-2000) Whitehead | geneseqp: B60093 | |
| | | Institute/MIT Center for | Submitted by INCYTE | |
| | | Genome Research, 320 | GENOMICS INC | |
| | | Charles Street, | Application number and | |
| | | Cambridge, MA 02141. | publication date: | |
| | | | WO200078953-A2, | |
| | | | 28-DEC-00 | |
| sbg1012732GLUT | Glucose | GB: AP000350 | Human glucose | Membrane- |
| | transporter | Direct submitted (10- | transporter GLUT10, | bound |
| | | JUN-1999) to the | gi: 13540598 | |
| | | DDBJ/EMBL/GenBank | Submitted (10-FEB- | |
| | | databases. Nobuyoshi | 2000) Joost H. G., | |
| | | Shimizu, Keio university, | Institute of | |
| | | school of medicine, | Pharmacology and | |
| | | Molecular Biology; 35 | Toxicology, Medical | |
| | | Shinanomachi, Shinjukuku, | Faculty, Technical | |
| | | Tokyo 160-0016, | University of Aachen, | |
| | | Japan. | Wendlingweg 2, | |
| | | | Aachen, D-52057, | |
| | | | GERMANY | |
| sbg1012732GLUTb | Glucose | GB: AP000350 | Human glucose | Membrane- |
| | transporter | Direct submitted (10- | transporter GLUT10, | bound |
| | | JUN-1999) to the | gi: 13540598 | |
| | | DDBJ/EMBL/GenBank | Submitted (10-FEB- | |
| | | databases. Nobuyoshi | 2000) Joost H. G., | |
| | | Shimizu, Keio university, | Institute of | |
| | | school of medicine, | Pharmacology and | |
| | | Molecular Biology; 35 | Toxicology, Medical | |
| | | Shinanomachi, Shinjukuku, | Faculty, Technical | |
| | | Tokyo 160-0016, | University of Aachen, | |
| | | Japan. | Wendlingweg 2, | |
| | | | Aachen, D-52057, | |
| | | | GERMANY | |
| sbg1018172CSP | Chondroitin | EMBL: AL354819, | Lytechinus variegatus | Secreted |
| | sulfate | and SC: AL590007. | embryonic blastocoelar | |
| | proteoglycan | Submitted (30-APR-2001 | extracellular matrix | |
| | | and 04-MAY-2001) by | protein, gi: 9837426 | |
| | | Sanger Centre, Hinxton, | Submitted (14-JUL- | |
| | | Cambridgeshire, CB10 | 2000) Biological | |
| | | 1SA, UK. | Sciences, Carnegie | |
| | | EMBL: AC017111, | Mellon University, | |
| | | Submitted (09-DEC- | 4400 Fifth Ave, | |
| | | 1999) Genome | Pittsburgh, PA 15213, | |
| | | Sequencing Center, | USA | |
| | | Washington University | | |
| | | School of Medicine, 4444 | | |
| | | Forest Park Parkway, St. | | |
| | | Louis, MO 63108, USA | | |
| sbg1004570ERGIC | ER-Golgi | GB: AC020705 | Human ERGL protein, | Membrane- |
| | intermediate | Submitted (08-JAN-2000) | gi: 11141891 | bound |
| | compartment | Genome Sequencing | Submitted (06-SEP- | |
| | protein | Center, Washington | 2000) Laboratory of | |
| | | University School of | Molecular Biology, | |
| | | Medicine, 4444 Forest | NCI, NIH, 37 Convent | |
| | | Park Parkway, St. Louis, | Dr., Bldg. 37, Rm. | |
| | | MO 63108, USA | 4B20, Bethesda, MD | |
| | | | 20892, USA | |
| sbg1016995IGBrecpt | Immunoglobulin | GB: AL353721 | Human | Membrane- |
| | receptor | Submitted (07-JUL-2001) | immunoglobulin | bound |
| | | Sanger Centre, Hinxton, | superfamily receptor | |
| | | Cambridgeshire, CB10 | translocation associated | |
| | | 1SA, UK. | 1, gi: 14550416 | |
| | | | Hatzivassiliou, G., | |
| | | | Miller, I. J., Takizawa, J., | |
| | | | et al. Immunity 14 (3), | |
| | | | 277-289 (2001) | |
| sbg1151bSREC | EGF-like | GB: AC005500 | Human nurse cell | Membrane- |
| | LDL | Chen, F., D, L., Do, T., | receptor B6TNC#10b, | bound |
| | receltor | Dumanski, J. P. and | geneseqp: B60395 | |
| | protein | Roe, B. A. Direct | Submitted by (SHIO) | |
| | | submission (31-MAY-01) | SHIONOGI & CO LTD | |
| | | Department Of Chemistry | Application number and | |
| | | and Biochemistry, The | publication date: | |
| | | University Of Oklahoma, | JP2000308492-A, | |
| | | 620 Parrington Oval, | 07-NOV-00 | |
| | | Room 208, Norman, OK | | |
| | | 73019, USA | | |
| sbg1399854ANK | The | GB: AC020658 | Human KIAA1223 | Cytosolic |
| | ankyrin | Direct submitted (08- | protein, gi: 6330617 | |
| | repeat | JAN-2000) | Nagase T, Ishikawa K, | |
| | family | Multimegabase | Kikuno R, Hirosawa M, | |
| | | Sequencing Center, | Nomura N, and Ohara O; | |
| | | University of | 1999 DNA Res | |
| | | Washington, PO BOX | 6: 337-45. | |
| | | 357730, Seattle, WA | | |
| | | 98195, USA | | |

TABLE III

| | | Associated |
|---|---|---|
| Gene Name | Uses | Diseases |
| sbg960509cbrecpt | An embodiment of the invention is the use of | Autoimmune |
| | sbg960509cbrecpt in the treatment or diagnosis of cancer. | disorder and |
| | A close homologue of sbg960509cbrecpt is Langerin. | cancer |
| | Langerin was a type II Ca2+-dependent lectin, an endocytic | |
| | receptor and expressed by Langerhans cells (LC). | |
| | Transfection of Langerin cDNA into fibroblasts created a | |
| | compact network of membrane structures with typical features | |
| | of Birbeck granules(BG). It was proposed that induction of | |
| | BG was a consequence of the antigen-capture function of | |
| | Langerin, allowing routing into these organelles and | |
| | providing access to a nonclassical antigen-processing pathway | |
| | (Valladeau J, Ravel O, Dezutter-Dambuyant C, Moore K, | |
| | Kleijmeer M, Liu Y, Duvert-Frances V, Vincent C, Schmitt D, | |
| | Davoust J, Caux C, Lebecque S, Saeland S. 2000. | |
| | Immunity Jan; 12(1): 71-81). A striking incongruity was found | |
| | between variably spliced transcripts for the second | |
| | asialoglycoprotein receptor polypeptide, H2, in normal and | |
| | transformed human liver cells (Paietta E, Stockert RJ, | |
| | Racevskis J. 1992. Hepatology Mar; 15(3): 395-402). Human | |
| | macrophage cell surface C-type lectin was demonstrated to | |
| | recognize Tn Ag, a well-known human carcinoma-associated | |
| | epitope (Suzuki N, Yamamoto K, Toyoshima S, Osawa T, | |
| | Irimura T. 1996. J Immunol Jan 1; 156(1): 128-35). | |
| sbg614126complfH | An embodiment of the invention is the use of | Alzheimer's |
| | sbg614126complfH in the diagnosis or treatment of cancer, | disease, cancer, |
| | Alzheimer disease, and/or tumor cell evasion. | tumor metastasis |
| | A close homologue of sbg614126complfH is Human | and autosomal |
| | complement factor H. Human complement factor H was | recessive atypical |
| | detected by the AM34 antibody in the cerebrospinal fluid | hemolytic uremic |
| | from an Alzheimer's disease patient. It was recently found | syndrome |
| | that AM34 was capable of staining senile plaques positively | |
| | and factor H was associated with senile plaques in the human | |
| | brain (Honda S, Itoh F, Yoshimoto M, Ohno S, Hinoda Y, | |
| | Imai K. 2000. J Gerontol A Biol Sci Med Sci. | |
| | May; 55(5): M265-9). It was also suggested that exceptional | |
| | resistance of human H2 glioblastoma cells to complement- | |
| | mediated killing was due to the production and binding of | |
| | factor H and factor H-like protein 1 (Junnikkala S, Jokiranta TS, | |
| | Friese MA, Jarva H, Zipfel PF, Meri S. 2000. J Immunol. | |
| | Jun 1; 164(11): 6075-81). Moreover, factor H was shown to | |
| | bind to bone sialoprotein and osteopontin and enable tumor | |
| | cell evasion of complement-mediated attack (Fedarko NS, | |
| | Fohr B, Robey PG, Young MF, Fisher LW. 2000. J Biol | |
| | Chem. Jun 2; 275(22): 16666-72). Finally, complement factor | |
| | H gene mutation was associated with autosomal recessive | |
| | atypical hemolytic uremic syndrome (Ying L, Katz Y, | |
| | Schlesinger M, Carmi R, Shalev H, Haider N, Beck G, | |
| | Sheffield VC, Landau D. 1999. Am J Hum Genet | |
| | Dec; 65(6): 1538-46). | |
| sbg120703RNase | An embodiment of the invention is the use of | Cancer and |
| | sbg120703Rnase as a tool for anticancer therapy, and treating | infection |
| | apoptosis-related disorders. It has been shown that a genetic- | |
| | engineered pancreatic RNase has cytotoxic action on mouse | |
| | and human tumor cells, but lacks any appreciable toxicity on | |
| | human and mouse normal cells. This variant of human | |
| | pancreatic RNase selectively sensitized cells derived from a | |
| | human thyroid tumor to apoptotic death. Because of its | |
| | selectivity for tumor cells, and because of its human origin, | |
| | this protein is thought to represent a promising tool for | |
| | anticancer therapy (Piccoli R, Di Gaetano S, De Lorenzo C, | |
| | Grauso M, Monaco C, Spalletti-Cernia D, Laccetti P, Cinatl J, | |
| | Matousek J, D′Alessio G. 1999. Proc Natl Acad Sci USA | |
| | 96: 7768-73). In addition, RNase itself can be used to treat an | |
| | RNA viral infection, and its antagonist may be useful in | |
| | treating apoptosis-related disorders. | |
| sbg98530TS | An embodiment of the invention is the use of sbg98530TS in | Cancer, wound |
| | the wound healing processes, development of the nervous | healing disorders |
| | system, and affecting cell migration, survival, or | |
| | angiogenesis. Close homologues of sbg98530TS are | |
| | thrombospondins. The thrombospondins are a family of | |
| | proteins found widely in the embryonic extracellular matrix, | |
| | and the expression patterns and in vitro properties of many | |
| | thrombospondins suggest potential roles in the guidance of | |
| | cell and growth cone migration, especially during the | |
| | development of the nervous system (Adams JC, 2000. Tucker | |
| | RP Dev Dyn 218: 280-99). Cell interactions with extracellular | |
| | matrices are important to pathological changes that occur | |
| | during cell transformation and tumorigenesis. The | |
| | thrombospondin-1 has been suggested to modulate tumor | |
| | phenotype by affecting cell migration, survival, or | |
| | angiogenesis (Liaw L, Crawford HC. 1999. Braz J Med Biol | |
| | Res 32: 805-12). In addition, thrombospondin-1 is also a | |
| | transient component of extracellular matrix in developing and | |
| | repairing tissues (Adams JC. 1997. Int J Biochem Cell Biol | |
| | 29: 861-5). | |
| sbg563917RDP | An embodiment of the invention is the use of sbg563917RDP | Renal diseases, |
| | in treatment or diagnosis of chronic renal failure and aged eye | aging, cataract, |
| | lenses and cataracts. Close homologues of sbg563917RDP | cancer, and |
| | are renal and lens dipeptidases. It has been reported that the | Alzheimer |
| | renal dipeptidase activity was significantly lower in the | disease |
| | chronic renal failure group (Fukumura Y, Kera Y, Oshitani S, | |
| | Ushijima Y, Kobayashi I, LiuZ, Watanabe T, Yamada R, | |
| | Kikuchi H, Kawazu S and Yabuuchi M. 1999 Ann Clin | |
| | Biochem Mar; 36 (Pt 2): 221-5). In contrast, increased lens | |
| | dipeptidase activity was detected in aging and cataracts | |
| | (Sulochana KN, Ramakrishnan S and Punitham R. 1999 Br J Ophthalmol | |
| | Jul; 83(7): 885). | |
| sbg618069LRR | An embodiment of the invention is the use of sbg618069LRR | Tango-associated |
| | in treatment or diagnosis of neural development and the adult | diseases, |
| | nervous system disorders. Close homologues of | disorders |
| | sbg618069LRR are Leucine-rich repeat proteins. Leucine-rich | associated with |
| | repeat protein, the spineless-aristapedia, has been shown to | the preservation |
| | interact with tango bHLH-PAS proteins for controlling | and maintenance |
| | antennal and tarsal development in Drosophila (Emmons RB, | of gastrointestinal |
| | Duncan D, Estes PA, Kiefel P, Mosher JT, Sonnenfeld M, | mucosa and the |
| | Ward MP, Duncan I and Crews ST. 1999. Development | repair of acute |
| | Sep; 126(17): 3937-45). In mouse, neuronal Leucine-Rich | and chronic |
| | Repeat NLRR-1 and NLRR-2 mRNAs were expressed | mucosal lesions, |
| | primarily in the central nervous system and may play | Parkinson's |
| | significant but distinct roles in neural development and in the | disease, |
| | adult nervous system (Taguchi A, Wanaka A, Mori T, | Alzheimer's |
| | Matsumoto K, Imai Y, Tagaki T and Tohyama M. 1996. | disease, ALS, |
| | Brain Res Mol Brain Res Jan; 35(1-2): 31-40). Furthermore, a | neuropathies, |
| | new member of the leucine-rich repeat superfamily GAC1 | cancer, wound |
| | was amplified and overexpressed in malignant gliomas | healing and tissue |
| | (Almeida A, Zhu XX, Vogt N, Tyagi R, Muleris M, | repair |
| | Dutrillaux AM, Dutrillaux B, Ross D, Malfoy B and Hanash S. | |
| | 1998. Oncogene Jun 11; 16(23): 2997-3002). | |
| sbg934114Relaxin | An embodiment of the invention is the use of | Cancer, |
| | sbg934114Relaxin in treatment or diagnosis of collagen | rheumatic |
| | remodeling, breast cancer, and uterine contractile disorders. A | diseases, heart |
| | close homologue of sbg934114Relaxin is Relaxin. Relaxin | diseases, |
| | has various biologic activities, including the induction of | systemic |
| | collagen remodeling and consequent softening of the tissues | sclerosis |
| | of the birth canal during delivery, the inhibition of uterine | (scleroderma), |
| | contractile activity, and the stimulation of growth and | and preterm birth |
| | differentiation of the mammary gland (Bani D. 1997. Gen | |
| | Pharmacol 28: 13-22). Relaxin belongs to the insulin | |
| | superfamily, and is produced primarily by the corpus luteum | |
| | in both pregnant and nonpregnant females. In males, relaxin is | |
| | synthesized in the prostate and released in the seminal fluid | |
| | (Goldsmith LT, Weiss G, Steinetz BG. 1995. Endocrinol | |
| | Metab Clin North Am 24: 171-86). It has been further | |
| | demonstrated that relaxin regulates growth and differentiation | |
| | of breast cancer cells in culture, promotes dilation of blood | |
| | vessels in several organs, including the uterus, the mammary | |
| | gland, the lung and the heart, has a chronotropic action on the | |
| | heart, inhibits the release of histamine by mast cells, | |
| | depresses aggregation of platelets and their release by | |
| | megakaryocytes, and influences the secretion of hormones by | |
| | the pituitary gland (Bani D. 1997. Gen Pharmacol 28: 13-22). | |
| | In addition, some reports have shown that relaxin is effective | |
| | in decreasing skin involvement in systemic sclerosis (Furst DE. | |
| | 1998. Curr Opin Rheumatol 10: 123-8). | |
| sbg99174LOX- | An embodiment of the invention is the use of | Cardiovascular |
| like | sbg99174LOX-like in treatment or diagnosis of endothelial | disorders (e.g. |
| | function or atherosclerosis. A close homologue of | atherosclerosis, |
| | sbg99174LOX-like is oxidized low-density lipoprotein | hypertension, |
| | receptor 1. sbg99174LOX-like as well as oxidized low- | stroke), |
| | density lipoprotein receptor 1, contain a C-type lectin domain | |
| | (CTL) (Colonna M, Samaridis J, Angman L. 2000. Eur J | |
| | Immunol 30: 697-704). Evidence suggests that oxidized low- | |
| | density lipoprotein (OxLDL) plays a critical role in the | |
| | changes in endothelial function. Lectin-like OxLDL receptor- | |
| | 1 (LOX-1) is the major endothelial OxLDL receptor. | |
| | Functional changes of endothelial cells are implicated in the | |
| | earliest stage of the pathogenesis of atherosclerosis (Aoyama T, | |
| | Sawamura T, Furutani Y, Matsuoka R, Yoshida MC, | |
| | Fujiwara H, Masaki T. Biochem J. 1999 339 (Pt 1): 177-84). | |
| sbg995002PIGR | An embodiment of the invention is the use of | Infection and |
| | sbg995002PIGR to actively transport IgA and IgM to the | inflammation |
| | apical surface of epithelia. A close homologue of | such as |
| | sbg995002PIGR is polymeric-immunoglobulin receptor. The | inflammatory |
| | polymeric-immunoglobulin receptor binds polymeric IgA and | bowel disease, |
| | IgM at the basolateral surface of epithelial cells. PIGR | gluten-sensitive |
| | knockout mice completely lack active external IgA and IgM | enterropathy, and |
| | translocation, but remain normal and fertile (Johansen FE, | urinary tract |
| | Pekna M, Norderhaug IN, Haneberg B, Hietala MA, Krajci P, | infection) |
| | Betsholtz C, Brandtzaeg P. 1999. J Exp Med 190: 915-22). In | |
| | addition, it has been reported that PIGR can be upregulated by | |
| | tumor necrosis factor (TNF)-alpha (Takenouchi-Ohkubo N, | |
| | Takahashi T, Tsuchiya M, Mestecky J, Moldoveanu Z, Moro I; | |
| | 2000. Immunogenetics 51: 289-95). | |
| sbg1033026C1q | An embodiment of the invention is the use of sbg1033026C1q | Central nervous |
| | to regulate central nervous system functions. A close | system disorder |
| | homologue of sbg1033026C1q is C1q-related factor. | |
| | C1q is a subunit of the C1 enzyme complex that activates the | |
| | serum complement system. It has been shown that human | |
| | C1q-related factor (CRF) transcript is expressed at highest | |
| | levels in the brain, particularly in the brainstem. Similarly, in | |
| | mouse brain CRF transcripts are most abundant in areas of the | |
| | nervous system involved in motor function (Berube NG, | |
| | Swanson XH, Bertram MJ, Kittle JD, Didenko V. Baskin DS, | |
| | Smith JR. and Pereira-Smith OM., 1999, Brain Res. Mol. | |
| | Brain Res. 63: 233-240). Moreover, ACRP30 is structurally | |
| | similar to complement factor C1q, and it forms large homo- | |
| | oligomers that undergo a series of post-translational | |
| | modifications. ACRP30 proteins may be a factor that | |
| | participates in the complex balanced system of energy | |
| | homeostasis involving food intake, carbohydrate catabolism, | |
| | and lipid catabolism (Scherer PE, Williams S, Fogliano M, | |
| | Baldini G, Lodish HF; 1995; J Biol Chem 270: 26746-9). | |
| sbg1003675RNase | An embodiment of the invention is the use of | Viral infection, |
| | sbg1003675RNase as a promising tool for anticancer therapy, | and tumor |
| | and apoptosis-related disorders. A close homologue of | |
| | sbg1003675RNase is RNase. It has been shown that a | |
| | genetic-engineered pancreatic RNase has cytotoxic action on | |
| | mouse and human tumor cells, but lacks any appreciable | |
| | toxicity on human and mouse normal cells. This variant of | |
| | human pancreatic RNase selectively sensitized cells derived | |
| | from a human thyroid tumor to apoptotic death. Because of its | |
| | selectivity for tumor cells, and because of its human origin, | |
| | this protein was thought to represent a promising tool for | |
| | anticancer therapy (Piccoli R, Di Gaetano S, De Lorenzo C, | |
| | Grauso M, Monaco C, Spalletti-Cernia D, Laccetti P, Cinatl J, | |
| | Matousek J, D'Alessio G. 1999. Proc Natl Acad Sci USA | |
| | 96: 7768-73). Moreover, RNase itself can be used to treat an | |
| | RNA viral infection, and its antagonist of this RNase may be | |
| | useful in treating apoptosis-related disorders. | |
| sbg1015258PLM | An embodiment of the invention is the use of | Myotonic |
| | sbg1015258PLM to regulate skeletal and cardiac muscle | muscular |
| | disorders. A close homologue of sbg1015258PLM is | dystrophy |
| | phospholemman. The phospholemman (PLM) is enriched in | |
| | skeletal muscle and the heart, and is a major substrate | |
| | phosphorylated in response to insulin and adrenergic | |
| | stimulation. All phospholemman proteins are small and have | |
| | a single transmembrane domain (Chen LS, Lo CF, Numann R, | |
| | Cuddy M. 1997. Genomics 41: 435-4). Phospholemman | |
| | can be phosphorylated by protein kinases A and C to induce a | |
| | hyperpolarization-activated chloride current, and therefore | |
| | may play a role in muscle contraction. Recently | |
| | phospholemman was shown to be a substrate for myotonic | |
| | dystrophy protein kinase, and therefore is associated with the | |
| | disease, an autosomal dominant-inherited disorder with | |
| | prominent effects on skeletal and cardiac muscle (Mounsey JP, | |
| | John JE 3rd, Helmke SM, Bush EW, Gilbert J, Roses AD, | |
| | Perryman MB, Jones LR, Moorman JR. 2000. J Biol Chem; | |
| | 275: 23362-7). | |
| sbg1003328IG | An embodiment of the invention is the use of | Cancer, infection, |
| | sbg1003328IG to generate immunosuppressants to suppress | autoimmune |
| | immune responses. A close homologue of sbg1003328IG is | disorder, |
| | V7, a human leukocyte surface protein (Stockinger H, Gadd SJ, | hematopoietic |
| | Eher R, Majdic O, Schreiber W, Kasinrerk W, Strass B, | disorder, wound |
| | Schnabl E, Knapp W. 1990. J Immunol 145: 3889-97). | healing disorders, |
| | sbg1003328IG is an immunoglobulin (Ig)-like membrane | and inflammation |
| | protein containing three potential Ig domains, and it has an | |
| | overall strong sequence similarity to V7. | |
| sbg1020829SGLT | An embodiment of the invention is the use of | Cancer, infection, |
| | sbg1020829SGLT to regulate Na(+)-dependent glucose | autoimmune |
| | transport. A close homologue of sbg1020829SGLT is | disorder, |
| | Na+/glucose cotransporters. The human intestinal Na+/glucose | hematopoietic |
| | cotransporter (SGLT1) was cloned and sequenced. Close | disorder, wound |
| | homology was observed between the human and rabbit | healing disorders, |
| | intestinal Na+/glucose cotransporters, and a significant | inflammation and |
| | homology was found between these and the Escherichia coli | glucose/galactose |
| | Na+/proline cotransporter (putP) indicating that the mammalian | malabsorption |
| | Na+/glucose and prokaryote Na+/proline cotransporters sharing | |
| | a common ancestral gene (Hediger MA, Turk E, Wright EM. | |
| | 1989 Proc Natl Acad Sci USA Aug; 86(15): 5748-52). In | |
| | addition, study of intestinal biopsies of glucose/galactose | |
| | malabsorption (GCM) patients has revealed a specific defect in | |
| | Na(+)-dependent absorption of glucose in the brush border. A | |
| | single missense mutation was found in SGLT1 amplified from | |
| | the genomic DNA derived from members of a family affected | |
| | with GGM. This mutated SGLT1 cosegregated with the GGM | |
| | phenotype and resulted in a complete loss of Na(+)-dependent | |
| | glucose transport in Xenopus oocytes injected with this | |
| | complementary RNA (Turk E, Zabel B, Mundlos S, Dyer J, | |
| | Wright EM. 1991 Nature Mar 28; 350(6316): 354-6). | |
| sbg1005450UDPGT | An embodiment of the invention is the use of | Cancer, infection, |
| | sbg1005450UDPGT to regulate estrogen and androgen | autoimmune |
| | catabolism in peripheral steroid target tissues. A close | disorder, |
| | homologue of sbg1005450UDPGT is UDP- | hematopoietic |
| | glucuronosyltransferase (UDPGT) gene. Mutations had been | disorder, wound |
| | found in the promoter and coding regions of UDP- | healing disorders, |
| | glucuronosyltransferase (UDPGT) gene in seven patients with | inflammation |
| | Crigler-Najjar syndrome type II caused by reduction in hepatic | Gilbert |
| | bilirubin UDPGT activity (Yamamoto K, Soeda Y, Kamisako T, | syndrome, |
| | Hosaka H, Fukano M, Sato H, Fujiyama Y, Adachi Y, Satoh Y, | Crigler-Najjar |
| | Bamba T. 1998. J Hum Genet 43(2): 111-4). A case of Gilbert | syndrome (CN) |
| | syndrome caused by a homozygous missense mutation of the | type II, and |
| | bilirubin UDPGT gene was also reported (Maruo Y, Sato H, | steroid hormone |
| | Yamano T, Doida Y, Shimada M. 1998. J Pediatr | catabolism |
| | Jun; 132(6): 1045-7). In addition, monkey UDPGT UGT1A9 | malfunction |
| | had been cloned and the mRNA was expressed in extrahepatic | |
| | estrogen-responsive tissues indicating its potential role in | |
| | estrogen metabolism (Albert C, Vallee M, Beaudry G, Belanger A, | |
| | Hum DW. 1999. Endocrinology Jul; 140(7): 3292-302). | |
| | Human UDPGT UGT2B23 transcript was also expressed in | |
| | extrahepatic tissues including prostate, mammary gland, | |
| | epididymis, testis, and ovary. The activity of UGT2B23 was | |
| | tested with 62 potential endogenous substrates and was | |
| | demonstrated to be active on 6 steroids and the bile acid, | |
| | hyodeoxycholic acid suggesting that UGT2B23 might play an | |
| | important role in estrogen and androgen catabolism in peripheral | |
| | steroid target tissues (Barbier O, Levesque E, Belanger A, Hum DW. | |
| | 1999. Endocrinology Dec; 140(12): 5538-48). | |
| sbg1002620TIa | An embodiment of the invention is the use of sbg1002620TIa | Cancer, infection, |
| | to regulate human tumor cells. A close homologue of | autoimmune |
| | sbg1002620TIa is human hypothetical protein | disorder, |
| | DKFZp434B044. This gene is also similar to trypsin inhibitor | hematopoietic |
| | which contains Sc7 family of extracellular domains at its N- | disorder, wound |
| | ternimal region (Genome Res. 11 (3), 422-435 (2001)). | healing disorders, |
| | Trypsin inhibitor P25TI sequence had similarity to CRISP | inflammation, |
| | family proteins including insect venom allergens, mammalian | blood coagulation |
| | testis-specific proteins and plant pathogenesis-related proteins. | disorders, cellular |
| | mRNA encoding P25TI and another two glioma pathogenesis- | adhesion |
| | related protein GliPR and RTVP-1, which were also shown to | disorders, |
| | be structurally similar to CRISP family proteins was frequently | pancreatitis, |
| | expressed in human tumor tissues but not detected in normal | shock, multi- |
| | human tissue cell lines (Yamakawa T, Miyata S, Ogawa N, | organ failure, and |
| | Koshikawa N, Yasumitsu H, Kanamori T, Miyazaki K 1998. | gastrointestinal |
| | Biochim Biophys Acta Jan 21; 1395(2): 202-8., Murphy EV, | ulceration |
| | Zhang Y, Zhu W, Biggs J. 1995. Gene Jun 14; 159(1): 131-5., | |
| | Rich T, Chen P, Furman F, Huynh N, Israel MA. 1996. Gene | |
| | Nov 21; 180(1-2): 125-30). | |
| sbg1002620TIb | An embodiment of the invention is the use of | Cancer, infection, |
| | sbg1002620TIb as a marker for some nervous system tumors, | autoimmune |
| | and to regulate expression of human neuroblastoma and | disorder, |
| | glioblastoma. A close homologue of sbg1002620TIb is late- | hematopoietic |
| | gestation lung 1 (LGL1) protein. Late-gestation lung 1 | disorder, wound |
| | (LGL1) protein showed 81% homology to P25TI, the trypsin | healing disorders, |
| | inhibitor purified from the culture medium of human | inflammation, |
| | glioblastoma cells (Kaplan F, Ledoux P, Kassamali FQ, | blood coagulation |
| | Gagnon S, Post M, Koehler D, Deimling J, Sweezey NB. | disorders, cellular |
| | 1999. Am J Physiol Jun; 276(6 Pt 1): L1027-36; Koshikawa N, | adhesion |
| | Nakamura T, Tsuchiya N, Isaji M, Yasumitsu H, Umeda M, | disorders, |
| | Miyazaki K. 1996. J Biochem (Tokyo) Feb; 119(2): 334-9). | pancreatitis, |
| | The cDNA encoding P25TI was isolated and the sequence | shock, multi- |
| | had similarity to CRISP family proteins including insect venom | organ failure, and |
| | allergens, mammalian testis-specific proteins and plant | gastrointestinal |
| | pathogenesis-related proteins. P25TI mRNA was frequently | ulceration |
| | expressed in human neuroblastoma and glioblastoma but not | |
| | detected in normal human tissues cell lines (Yamakawa T, | |
| | Miyata S, Ogawa N, Koshikawa N, Yasumitsu H, Kanamori T, | |
| | Miyazaki K 1998. Biochim Biophys Acta Jan 21; 1395(2): 202-8). | |
| | Another two glioma pathogenesis-related protein GliPR and | |
| | RTVP-1 were also shown to be structurally similar to CRISP | |
| | family proteins. The GLIPR gene was highly expressed in the | |
| | human brain tumor, glioblastoma multiforme/astrocytoma, but | |
| | neither in normal fetal or adult brain tissue, nor in other nervous | |
| | system tumors (Murphy EV, Zhang Y, Zhu W, Biggs J. 1995. | |
| | Gene Jun 14; 159(1): 131-5). Multiple RTVP-1 mRNA species | |
| | were highly expressed in a panel of cell lines from nervous | |
| | system tumors arising from glia, in contrast, the expression of | |
| | these RNAs was low or absent in nonglial-derived nervous | |
| | system tumor cell lines (Rich T, Chen P, Furman F, Huynh N, | |
| | Israel MA. 1996. Gene Nov 21; 180(1-2): 125-30). | |
| sbg102200MCTa | An embodiment of the invention is the use of | Cancer, infection, |
| | sbg102200MCTa in regulating cancer cells, including the | autoimmune |
| | hematopoietic lineages, Burkitt's lymphoma, and solid tumor | disorder, |
| | cells. A close homologue of sbg102200MCTa is MCT1 from | hematopoietic |
| | Chinese hamster and mouse. Mouse H+-monocarboxylate | disorder, wound |
| | cotransporter (MCT1) was cloned and sequenced from | healing disorders, |
| | Ehrlich Lettre tumour cells, the sequence of MCT1 is 93% | and inflammation |
| | and 87% homologous to MCT1 from Chinese hamster and | |
| | human, respectively. N-glycanase-F treatment and an in vitro | |
| | translation experiments demonstrated that glycosylation was | |
| | not required for MCT1 function (Carpenter L, Poole RC, | |
| | Halestrap AP. 1996. Biochim Biophys Acta Mar | |
| | 13; 1279(2): 157-63). Chick monocarboxylate transporter | |
| | MCT3 cloned from retinal pigment epithelium (RPE) cDNA | |
| | library was found only expressed in RPE cells. A rat thyroid | |
| | epithelial cell line FRTL transfected with pCl-neo/MCT3 | |
| | showed enhanced pyruvate uptake suggesting that MCT3 may | |
| | regulate lactate levels in the interphotoreceptor space (Yoon H, | |
| | Fanelli A, Grollman EF, Philp NJ. 1997. Biochem Biophys | |
| | Res Commun May 8; 234(1): 90-4). In human, MCT2 had | |
| | been implicated as a primary pyruvate transporter in man. | |
| | The mRNAs of MCT1 and MCT2 were found co-expressed in | |
| | various human cancer cell lines, including the hematopoietic | |
| | lineages HL60, K562, MOLT-4, Burkitt's lymphoma Raji, | |
| | and solid tumor cells such as SW480, A549, and G361. | |
| | These findings suggested that human MCT1 and MCT2 may | |
| | have distinct biological roles (Lin RY, Vera JC, Chaganti RS, | |
| | Golde DW. 1998. J Biol Chem Oct 30; 273(44): 28959-65). | |
| sbg102200MCTb | An embodiment of the invention is the use of | Cancer, infection, |
| | sbg102200MCTb in regulating cancer cells, including the | autoimmune |
| | hematopoietic lineages, Burkitt's lymphoma, and solid tumor | disorder, |
| | cells. A close homologue of sbg102200MCTb is MCT1 from | hematopoietic |
| | Chinese hamster and mouse. Mouse H+-monocarboxylate | disorder, wound |
| | cotransporter (MCT1) was cloned and sequenced from | healing disorders, |
| | Ehrlich Lettre tumour cells, the sequence of MCT1 is 93% | and inflammation |
| | and 87% homologous to MCT1 from Chinese hamster and | |
| | human, respectively. N-glycanase-F treatment and an in vitro | |
| | translation experiments demonstrated that glycosylation was | |
| | not required for MCT1 function (Carpenter L, Poole RC, | |
| | Halestrap AP. 1996. Biochim Biophys Acta Mar | |
| | 13; 1279(2): 157-63). Chick monocarboxylate transporter | |
| | MCT3 cloned from retinal pigment epithelium (RPE) cDNA | |
| | library was found only expressed in RPE cells. A rat thyroid | |
| | epithelial cell line FRTL transfected with pCl-neo/MCT3 | |
| | showed enhanced pyruvate uptake suggesting that MCT3 may | |
| | regulate lactate levels in the interphotoreceptor space (Yoon H, | |
| | Fanelli A, Grollman EF, Philp NJ. 1997. Biochem Biophys | |
| | Res Commun May 8; 234(1): 90-4). In human, MCT2 had | |
| | been implicated as a primary pyruvate transporter in man. | |
| | The mRNAs of MCT1 and MCT2 were found co-expressed in | |
| | various human cancer cell lines, including the hematopoietic | |
| | lineages HL60, K562, MOLT-4, Burkitt's lymphoma Raji, | |
| | and solid tumor cells such as SW480, A549, and G361. | |
| | These findings suggested that human MCT1 and MCT2 may | |
| | have distinct biological roles (Lin RY, Vera JC, Chaganti RS, | |
| | Golde DW. 1998. J Biol Chem Oct 30; 273(44): 28959-65). | |
| sbg1020380LYG | An embodiment of the invention is the use of | Cancer, infection, |
| | sbg1020380LYG in the immune system and enhance the | autoimmune |
| | activity of immunoagents and may serve as biomarkers of | disorder, |
| | periodontal disease activity. Close homologues of | hematopoietic |
| | sbg1020380LYG are lysozymes. Lysozymes are bacteriolytic | disorder, wound |
| | defensive agents and have been adapted to serve a digestive | healing disorders, |
| | function (Qasba PK, Kumar S, 1997, Crit Rev Biochem Mol | and inflammation |
| | Biol 32: 255-306). Those in tissue and body fluids are | |
| | involved in the immune system and enhance the activity of | |
| | immunoagents. Llysozymes may serve as biomarkers of | |
| | periodontal disease activity from inflammatory cell origin | |
| | (Eley BM, and Cox SW, 1998, Br Dent J 184: 323-8). | |
| sbg1007026SGLT | An embodiment of the invention is the use of | Glucose/galactose |
| | sbg1007026SGLT, a human sodium-glucose cotransporter, in | malabsorption |
| | regulation of Glucose/galactose malabsorption (GGM), familial | (GGM), familial |
| | renal glycosuria, and diabetic renal disorders. Close | renal glycosuria, |
| | homologues of sbg1007026SGLT are other sodium-glucose | and diabetic renal |
| | cotransporters from humans and rabbits. Human sodium- | disorders |
| | glucose cotransporters are responsible for the active | |
| | accumulation of glucose in cells (Hediger MA, Turk E, | |
| | Wright EM. 1989. Proc Natl Acad Sci USA 86: 5748-52). | |
| | The renal sodium-glucose cotrnasporter may be related to the | |
| | pathophysiology of renal diseases such as familial renal | |
| | glycosuria and diabetic renal disorders (Kanai Y, Lee WS, | |
| | You G, Brown D, Hediger MA. 1994. J Clin Invest 93: 397-404). | |
| | In addition, study of glucose/galactose malabsorption | |
| | (GGM) patients has revealed a specific defect in sodium- | |
| | dependent absorption of glucose in the brush border, and the | |
| | consequent severe diarrhea and dehydration caused by | |
| | glucose/galactose malabsorption are usually lethal unless | |
| | these sugars are eliminated from the diet (Turk E, Zabel B, | |
| | Mundlos S, Dyer J, Wright EM. 1991 Nature 350: 354-6). | |
| sbg1012732GLUT | An embodiment of the invention is the use of | Tumor, diabetic |
| | sbg1012732GLUT, in the maintenance of cellular | nephropathy, and |
| | homeostasis and metabolism. Close homologues of | insulin-induced |
| | sbg1012732GLUT are transmembrane glucose transporters | hypoglycemia |
| | (gluts). Glucose uptake is achieved by transmembrane | |
| | glucose transporters (gluts), and the transport of glucose | |
| | across plasma membranes is important for the maintenance of | |
| | cellular homeostasis and metabolism. Glucose is taken up by | |
| | cells and then phosphorylated to glucose-6-phosphate, and | |
| | lucose utilization by cancer cells is greatly enhanced when | |
| | compared with that by normal tissue. Tumor tissue is | |
| | frequently associated with the abnormal and/or over- | |
| | expression of glucose transporters, especially glut1 (Smith TA. | |
| | 1999. Br J Biomed Sci 56: 285-92). Increased utilization | |
| | of glucose in glomerular cells cause the increased expression | |
| | and activity of aldose reductase, protein kinase C and TGF- | |
| | beta, which have been implicated in excessive extracellular | |
| | matrix accumulation in diabetic nephropathy (Z Katedry i | |
| | Zakladu Patofizjologii, Akaemii Medycznej w Poznaniu. | |
| | 1999. Przegl Lek 56: 793-9). Changes in endothelial glucose | |
| | transport and GLUT1 abundance in the barriers of the brain | |
| | and retina may severely affect glucose delivery to these | |
| | tissues and major implications in the development of two | |
| | major diabetic complications, insulin-induced hypoglycemia | |
| | and diabetic retinopathy (Kumagai AK. 1999. Diabetes Metab | |
| | Res Rev 15: 261-73). | |
| sbg1012732GLUTb | An embodiment of the invention is the use of | Tumor, diabetic |
| | sbg1012732GLUTb, in the maintenance of cellular | nephropathy, and |
| | homeostasis and metabolism. Close homologues of | insulin-induced |
| | sbg1012732GLUTb are transmembrane glucose transporters | hypoglycemia |
| | (gluts). Glucose uptake is achieved by transmembrane glucose | |
| | transporters (gluts), and the transport of glucose across | |
| | plasma membranes is important for the maintenance of | |
| | cellular homeostasis and metabolism. Glucose is taken up by | |
| | cells and then phosphorylated to glucose-6-phosphate, and | |
| | lucose utilization by cancer cells is greatly enhanced when | |
| | compared with that by normal tissue. Tumor tissue is | |
| | frequently associated with the abnormal and/or over- | |
| | expression of glucose transporters, especially glut1 (Smith TA. | |
| | 1999. Br J Biomed Sci 56: 285-92). Increased utilization | |
| | of glucose in glomerular cells cause the increased expression | |
| | and activity of aldose reductase, protein kinase C and TGF- | |
| | beta, which have been implicated in excessive extracellular | |
| | matrix accumulation in diabetic nephropathy (Z Katedry i | |
| | Zakladu Patofizjologii, Akaemii Medycznej w Poznaniu. | |
| | 1999. Przegl Lek 56: 793-9). Changes in endothelial glucose | |
| | transport and GLUT1 abundance in the barriers of the brain | |
| | and retina may severely affect glucose delivery to these | |
| | tissues and major implications in the development of two | |
| | major diabetic complications, insulin-induced hypoglycemia | |
| | and diabetic retinopathy (Kumagai AK. 1999. Diabetes Metab | |
| | Res Rev 15: 261-73). | |
| sbg1018172CSP | An embodiment of the invention is the use of | Melanoma, |
| | sbg1018172CSP in regulation of melanoma, autoimmune | infection, |
| | disorders, hematopoietic disorder, wound healing, and | autoimmune |
| | inflammation. A close homologue of sbg1018172CSP is | disorder, |
| | melanoma-associated chondroitin sulfate proteoglycan | hematopoietic |
| | (MCSP) core protein NG2. The MCSP core protein NG2 can | disorder, wound |
| | act as a coreceptor for spreading and focal contact formation | healing, and |
| | in association with alpha 4 beta 1 integrin in melanoma cells | inflammation |
| | (Iida J, Meijne AM, Spiro RC, Roos E, Furcht LT, McCarthy JB. | |
| | 1995. Cancer Res Mar 15; 55(10): 2177-85). Cloning of | |
| | MCSP recognized by mAb 9.2.27 showed that the core | |
| | protein contained an open reading frame of 2322 AAs, | |
| | encompassing a large extracellular domain, a hydrophobic | |
| | transmembrane region, and a relatively short cytoplasmic tail. | |
| | MCSP RNA was detected in human melanoma cell lines and | |
| | in biopsies prepared from melanoma skin metastases but not | |
| | in other human cancer cells or a variety of human fetal and | |
| | adult tissues (Pluschke G, Vanek M, Evans A, Dittmar T, | |
| | Schmid P, Itin P, Filardo EJ, Reisfeld RA. 1996. Proc Natl | |
| | Acad Sci USA Sep 3; 93(18): 9710-5). | |
| sbg1004570ERGIC | An embodiment of the invention is the use of | Cancer, infection, |
| | sbg1004570ERGIC as a probe for studying protein trafficking | autoimmune |
| | in the secretory pathway which is crucial for the elucidation | disorder, |
| | and treatment of many inherited and acquired diseases, such | hematopoietic |
| | as cystic fibrosis, Alzheimer's disease and viral infectionsin | disorder, wound |
| | regulation of melanoma, autoimmune disorders, hematopoietic | healing disorders, |
| | disorder, wound healing, and inflammation. A close | inflammation, |
| | homologue of sbg1004570ERGIC is ERGIC-53, an ER-Golgi | and Alzheimer's |
| | intermediate compartment (ERGIC) protein. A ERGIC | disease |
| | protein was elevated more than two fold in HT-29 colon | |
| | adenocarcinoma cells resistant to the the antitumor drug | |
| | KRN5500. Together with other information, the cellular | |
| | secretory pathway was suggested a primary determinant of | |
| | sensitivity to KRN550 (Kamishohara M, Kenney S, | |
| | Domergue R, Vistica DT, Sausville EA. 2000 Exp Cell Res | |
| | May 1; 256(2): 468-79). Mutations in ERGIC-53 was shown to | |
| | cause combined deficiency of coagulation factors V and VIII | |
| | and it was suggested that ERGIC-53 might function as a | |
| | molecular chaperone for the transport from ER to Golgi of a | |
| | specific subset of secreted proteins, including coagulation | |
| | factors V and VII (Nichols WC, Seligsohn U, Zivelin A, | |
| | Terry VH, Hertel CE, Wheatley MA, Moussalli MJ, Hauri HP, | |
| | Ciavarella N, Kaufman RJ, Ginsburg D. 1998. Cell Apr | |
| | 3; 93(1): 61-70). In addition, ERGIC-53 was reviewed as an | |
| | attractive probe for studying numerous aspects of protein | |
| | trafficking in the secretory pathway which is crucial for the | |
| | elucidation and treatment of many inherited and acquired | |
| | diseases, such as cystic fibrosis, Alzheimer's disease and viral | |
| | infections (Hauri HP, Kappeler F, Andersson H, Appenzeller C. | |
| | 2000 J Cell Sci Feb; 113 (Pt 4): 587-96). | |
| sbg1016995IGBrecpt | An embodiment of the invention is the use of | Auto-immune |
| | sbg1016995IGBrecpt in the clearance of circulating | diseases, allergy, |
| | autoantibodies and immune complexes. A close homologue | and guillain- |
| | of sbg1016995IGBrecpt is guinea pig Fc receptor for | Barre syndrom |
| | immunoglobulin (Tominaga M, Sakata A, Ohmura T, | |
| | Yamashita T, Koyama J, Onoue K, 1990. Biochem Biophys | |
| | Res Commun Apr 30; 168(2): 683-9). IgG Fc-receptor | |
| | polymorphisms have been reported recently in patients with | |
| | guillain-Barre syndrome indicating the role of IgG Fc- | |
| | receptor in the clearance of circulating autoantibodies and | |
| | immune complexes (Vedeler CA, Raknes G, Myhr KM, | |
| | Nyland H. 2000 Neurology Sep 12; 55(5): 705-7). | |
| sbg1151bSREC | An embodiment of the invention is the use of sbg1151bSREC, | Atherosclerosis |
| | a scavenger receptor, in the regulation of pathogenesis in | disease |
| | atherosclerosis and the formation of foam cells in | |
| | atherosclerotic lesions. A close homologue of sbg1151bSREC | |
| | is scavenger receptor class A type I and type II. Most of the | |
| | scavenger receptors interacted with several structurally | |
| | different ligands such as oxidized low density lipoprotein | |
| | (Ox-LDL) and acetyl LDL. Several studies showed Ox-LDL | |
| | was involved in the pathogenesis of atherosclerosis | |
| | (Steinbrecher UP. 1999 Biochim Biophys Acta Jan | |
| | 4; 1436(3): 279-98). In macrophages scattered in aortic walls | |
| | without atherosclerotic lesions, scavenger receptor class A | |
| | type I and type II (SRA) was detected weakly but | |
| | consistently. In contrast, in atherosclerotic lesions, | |
| | macrophages around the core region showed a strong | |
| | immunoreactivity to SRA indicating the involvement of SRA | |
| | in the formation of foam cells in atherosclerotic lesions | |
| | (Nakata A, Nakagawa Y, Nishida M, Nozaki S, Miyagawa J, | |
| | Nakagawa T, Tamura R, Matsumoto K, Kameda-Takemura K, | |
| | Yamashita S and Matsuzawa Y. Arterioscler Thromb Vasc | |
| | Biol 1999 May; 19(5): 1333-9). | |
| sbg1399854ANK | An embodiment of the invention is the use of | Cancer, infection, |
| | sbg1399854ANK in protein-protein interactions and it may | autoimmune |
| | act by inhibiting protein of cyclin dependent kinase. The | disorder, |
| | present invention contains both death domain and ankyrin | hematopoietic |
| | repeat region. The death domain is involved in cell death | disorder, wound |
| | signaling (Cleveland J. and Ihle J. N. 1995. Cell 81: 479-482). | healing disorders, |
| | Ankyrin repeats (ANK) are tandem repeat modules of about | and inflammation |
| | 33 amino acids. Many ankyrin repeat regions are known to be | |
| | involved in protein-protein interaction (Svetlana Gorina and | |
| | Nikola P. Pavletich; 1996 Science 274: 1001-1005). | |

Table IV. Quantitative, Tissue-specific mRNA Expression Detected Using SybrMan

Quantitative, tissue-specific, mRNA expression patterns of the genes were measured using SYBR-Green Quantitative PCR (Applied Biosystems, Foster City, Calif.; see Schmittgen T. D. et al., Analytical Biochemistry 285:194-204, 2000) and human cDNAs prepared from various human tissues. Gene-specific PCR primers were designed using the first nucleic acid sequence listed in the Sequence List for each gene. The threshold cycle (Cₜ) is defined as the fractional cycle number at which the reporter fluorescence generated by cleavage of the probe reaches a threshold defined as 10 times the background. In cases sequence detection system software predicted more than one PCR product, Taqman was used for the specific PCR amplification as indicated under the specific genes.

In each gene's first subset table, two replicate measurements of gene of identification (GOI) mRNA were measured from various human tissues (column 3 and 4). The average GOI mRNA copies of the two replicates were made from each tissue RNA (column 5). The average amount of 18 S rRNA from each tissue RNA was measured (column 6) and used for normalization. To make each tissue with the same amount of 50 ng of 18 S rRNA, the normalization factor (column 7) was calculated by dividing 50 ng with the amount of 18 S rRNA measured from each tissue (column 6). The mRNA copies per 50 ng of total RNA were obtained by multipling each GOI normalization factor and the average mRNA copies (column 8).

Fold changes shown in each gene's second subset table were only calculated for disease tissues which have a normal counterpart. There are blanks in the fold change column for all samples that do not have counterparts. In addition, the fold change calculations are the fold change in the disease sample as compared to the normal sample. Accordingly, there will not be a fold change calculation next to any of the normal samples. For patient matched cancer pairs (colon, lung, and breast), each tumor is compared to its specific normal counterpart. When patient-matched normal/disease pairs do not exist, each disease sample was compared back to the average of all the normal samples of that same tissue type. For example, normal brain from the same patient that provided Alzheimer's brain is not applicable. Three normal brain samples and 4 Alzheimer's brain samples are used in the fold change. Three normal samples were averaged, and each of the Alzheimer's samples was compared back to that average.

Abbreviations

ALZ Alzheimer's Disease

CT CLONTECH (1020 East Meadow Circle Palo Alto, Calif. 94303-4230, USA)

KC Sample prepared by GSK investigator

COPD chronic obstructive pulmonary disease

endo endothelial

VEGF vascular endothelial growth factor

bFGF basic fibroblast growth factor

BM bone marrow

osteo osteoblast

OA osteoarthritis

RA rheumatoid arthritis

PBL peripheral blood lymphocytes

PBMNC peripheral blood mononuclear cells

HIV human immunodeficiency virus

HSV Herpes simplex virus

HPV human papilloma virus

Gene Name sbg960509cbrecpt

Lowest overall expression in normal and disease samples. Highest normal expression in the whole brain, fetal liver, and uterus. Highest disease expression in 2 of the lung tumor samples, one of the breast tumor samples, and one of the normal breast samples. Downregulation in 1 of 4 colon tumors implies an involvement in cancer of the colon. Downregulation in 2 of 4 AD brain samples as well as high expression in whole brain suggests an involvement in Alzheimer's disease. Downregulation in 3 of 3 COPD lung samples and downregulation in 4 of 4 asthmatic lung samples implicates this gene in COPD and asthma. Upregulation in 2 of 3 heart samples proposes roles in non-obstructive and obstructive DCM. Patterns difficult to interpret due to Cts>35. Moderate to low expression in immune cells. Moderate expression in OA and RA synovium.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | Ct | copies | copies | Average | 18S | 18S | 50 ng |
| Sample | (sample 1 | (sample | (sample | GOI | rRNA | rRNA | total |
| sbg960509cbrecpt | and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous | 40, 40 | 0 | 0 | 0.00 | 0.96 | 52.36 | 0.00 |
| Adipose Zenbio | | | | | | | |
| Adrenal Gland | 40, 40 | 0 | 0 | 0.00 | 0.61 | 81.97 | 0.00 |
| Clontech | | | | | | | |
| Whole Brain | 33.26, 32.07 | 24.63 | 48.4 | 36.52 | 7.24 | 6.91 | 252.18 |
| Clontech | | | | | | | |
| Fetal Brain Clontech | 40, 40 | 0 | 0 | 0.00 | 0.48 | 103.95 | 0.00 |
| Cerebellum Clontech | 40, 40 | 0 | 0 | 0.00 | 2.17 | 23.04 | 0.00 |
| Cervix | 40, 40 | 0 | 0 | 0.00 | 2.42 | 20.66 | 0.00 |
| Colon | 40, 40 | 0 | 0 | 0.00 | 2.71 | 18.45 | 0.00 |
| Endometrium | 40, 40 | 0.81 | 0 | 0.41 | 0.73 | 68.21 | 27.63 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 40, 40 | 0 | 0 | 0.00 | 2.58 | 19.38 | 0.00 |
| Jejunum | 35.56, 34.42 | 6.66 | 12.71 | 9.69 | 6.60 | 7.58 | 73.37 |
| Kidney | 40, 40 | 0 | 0 | 0.00 | 2.12 | 23.58 | 0.00 |
| Liver | 40, 40 | 0 | 0 | 0.00 | 1.50 | 33.33 | 0.00 |
| Fetal Liver Clontech | 33.46, 34.83 | 14.95 | 27.51 | 21.23 | 10.40 | 4.81 | 102.07 |
| Lung | 40, 40 | 0 | 0 | 0.00 | 2.57 | 19.46 | 0.00 |
| Mammary Gland | 40, 40 | 21.94 | 10.06 | 16.00 | 13.00 | 3.85 | 61.54 |
| Clontech | | | | | | | |
| Myometrium | 40, 40 | 0 | 0 | 0.00 | 2.34 | 21.37 | 0.00 |
| Omentum | 40, 40 | 0 | 0 | 0.00 | 3.94 | 12.69 | 0.00 |
| Ovary | 40, 40 | 0 | 0 | 0.00 | 4.34 | 11.52 | 0.00 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 40, 40 | 0 | 0 | 0.00 | 5.48 | 9.12 | 0.00 |
| Placenta Clontech | 40, 40 | 0.39 | 0 | 0.20 | 5.26 | 9.51 | 1.85 |
| Prostate | 40, 40 | 0 | 0 | 0.00 | 3.00 | 16.67 | 0.00 |
| Rectum | 40, 40 | 0 | 0 | 0.00 | 1.23 | 40.65 | 0.00 |
| Salivary Gland | 34.79, 40 | 10.31 | 0 | 5.16 | 7.31 | 6.84 | 35.26 |
| Clontech | | | | | | | |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine | 40, 40 | 0 | 0 | 0.00 | 0.98 | 51.07 | 0.00 |
| Clontech | | | | | | | |
| Spleen | 40, 40 | 0 | 0 | 0.00 | 4.92 | 10.16 | 0.00 |
| Stomach | 35.8, 38.29 | 5.82 | 1.41 | 3.62 | 2.73 | 18.32 | 66.21 |
| Testis Clontech | 40, 40 | 0 | 0 | 0.00 | 0.57 | 87.87 | 0.00 |
| Thymus Clontech | 35.13, 35.08 | 8.48 | 8.75 | 8.62 | 9.89 | 5.06 | 43.55 |
| Thyroid | 40, 40 | 0 | 0 | 0.00 | 2.77 | 18.05 | 0.00 |
| Trachea Clontech | 35.26, ND | 7.9 | ND | 7.90 | 9.71 | 5.15 | 40.68 |
| Urinary Bladder | 40, ND | 0 | ND | 0.00 | 5.47 | 9.14 | 0.00 |
| Uterus | 35.09, 33.87 | 8.67 | 17.4 | 13.04 | 5.34 | 9.36 | 122.05 |
| genomic | 26.62 | 1067.33 | | | | | |
| b-actin | 27.44 | 670.43 | | | | | |
| 1.00E+05 | 19.22 | 100000 | | | | | |
| 1.00E+05 | 19.38 | 100000 | | | | | |
| 1.00E+04 | 22.78 | 10000 | | | | | |
| 1.00E+04 | 20.52 | 10000 | | | | | |
| 1.00E+03 | 26.45 | 1000 | | | | | |
| 1.00E+03 | 27.03 | 1000 | | | | | |
| 1.00E+02 | 30.99 | 100 | | | | | |
| 1.00E+02 | 31.26 | 100 | | | | | |
| 1.00E+01 | 40 | 0 | | | | | |
| 1.00E+01 | 40 | 0 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | Fold |
| | Reg | | | detected/ | | Change |
| | number | | Mean | 50 ng | | in |
| Sample | (GSK | | GOI | total | | Disease |
| sbg960509cbrecpt | identifier) | Ct | copies | RNA | Sample | Population |
| colon normal GW98-167 | 21941 | 29.42 | | 332.32 | colon normal | |
| colon tumor GW98-166 | 21940 | 30.95 | 66.31 | 132.62 | colon tumor | −2.51 |
| colon normal GW98-178 | 22080 | 31.32 | 53.01 | 106.02 | colon normal | |
| colon tumor GW98-177 | 22060 | 30.57 | 83.1 | 166.20 | colon tumor | 1.57 |
| colon normal GW98-561 | 23514 | 31.44 | 49.16 | 98.32 | colon normal | |
| colon tumor GW98-560 | 23513 | 31.81 | 39.47 | 78.94 | colon tumor | −1.25 |
| colon normal GW98-894 | 24691 | 29.44 | 164.69 | 329.38 | colon normal | |
| colon tumor GW98-893 | 24690 | 34.42 | 8.18 | 16.36 | colon tumor | −20.13 |
| lung normal GW98-3 | 20742 | 28.04 | 383.11 | 766.22 | lung normal | |
| lung tumor GW98-2 | 20741 | 34.22 | 9.19 | 18.38 | lung tumor | −41.69 |
| lung normal GW97-179 | 20677 | 30.93 | 66.74 | 133.48 | lung normal | |
| lung tumor GW97-178 | 20676 | 27.11 | 667.61 | 1335.22 | lung tumor | 10.00 |
| lung normal GW98-165 | 21922 | 28.31 | 323.99 | 647.98 | lung normal | |
| lung tumor GW98-164 | 21921 | 30.92 | 67.22 | 134.44 | lung tumor | −4.82 |
| lung normal GW98-282 | 22584 | 31.76 | 40.67 | 81.34 | lung normal | |
| lung tumor GW98-281 | 22583 | 29.61 | 148.67 | 297.34 | lung tumor | 3.66 |
| breast normal GW00-392 | 28750 | 27.64 | 487.44 | 487.44 | breast normal | |
| breast tumor GW00-391 | 28746 | 27.47 | 539.99 | 1079.98 | breast tumor | 2.22 |
| breast normal GW00-413 | 28798 | 33.36 | 15.44 | 15.44 | breast normal | |
| breast tumor GW00-412 | 28797 | 30.88 | 68.84 | 137.68 | breast tumor | 8.92 |
| breast normal GW00- | 27592-95 | 34.74 | 6.73 | 6.73 | breast normal | |
| 235:238 | | | | | | |
| breast tumor GW00- | 27588-91 | 33.73 | 12.41 | 12.41 | breast tumor | 1.84 |
| 231:234 | | | | | | |
| breast normal GW98-621 | 23656 | 27.7 | 469.27 | 938.54 | breast normal | |
| breast tumor GW98-620 | 23655 | 33.1 | 18.13 | 36.26 | breast tumor | −25.88 |
| brain normal BB99-542 | 25507 | 31.46 | 48.61 | 97.22 | brain normal | |
| brain normal BB99-406 | 25509 | 34.17 | 9.52 | 19.04 | brain normal | |
| brain normal BB99-904 | 25546 | 35.69 | 3.79 | 7.58 | brain normal | |
| brain stage 5 ALZ BB99- | 25502 | 40 | 0 | 0.00 | brain stage 5 ALZ | −41.28 |
| 874 | | | | | | |
| brain stages 5 ALZ BB99- | 25503 | 34.96 | 5.91 | 11.82 | brain stage 5 ALZ | −3.49 |
| 887 | | | | | | |
| brain stage 5 ALZ BB99- | 25504 | 33.13 | 17.82 | 35.64 | brain stage 5 ALZ | −1.16 |
| 862 | | | | | | |
| brain stage 5 ALZ BB99- | 25542 | 40 | 0 | 0.00 | brain stage 5 ALZ | −41.28 |
| 927 | | | | | | |
| CT lung KC | normal | 29.53 | 155.88 | 311.76 | CT lung | |
| lung 26 KC | normal | | | | lung 26 | |
| lung 27 KC | normal | 39.2 | 0.46 | 0.46 | lung 27 | |
| lung 24 KC | COPD | 40 | 0 | 0.00 | lung 24 | −104.07 |
| lung 28 KC | COPD | 40 | 0 | 0.00 | lung 28 | −104.07 |
| lung 23 KC | COPD | 34.81 | 6.44 | 6.44 | lung 23 | −16.16 |
| lung 25 KC | normal | 40 | 0 | 0.00 | lung 25 | |
| asthmatic lung | 29321 | 38.99 | 0.52 | 0.52 | asthmatic lung | −200.14 |
| ODO3112 | | | | | | |
| asthmatic lung | 29323 | 33.69 | 12.65 | 25.30 | asthmatic lung | −4.11 |
| ODO3433 | | | | | | |
| asthmatic lung | 29322 | 33.53 | 13.98 | 27.96 | asthmatic lung | −3.72 |
| ODO3397 | | | | | | |
| asthmatic lung | 29325 | 34.27 | 8.96 | 17.92 | asthmatic lung | −5.81 |
| ODO4928 | | | | | | |
| endo cells KC | control | 40 | 0 | 0.00 | endo cells | |
| endo VEGF KC | | 40 | 0 | 0.00 | endo VEGF | 0.00 |
| endo bFGF KC | | 40 | 0 | 0.00 | endo bFGF | 0.00 |
| heart Clontech | normal | 35.53 | 4.19 | 8.38 | heart | |
| heart (T-1) ischemic | 29417 | 34 | 10.5 | 21.00 | heart T-1 | 2.51 |
| heart (T-14) non- | 29422 | 31.16 | 58.24 | 116.48 | heart T-14 | 13.90 |
| obstructive DCM | | | | | | |
| heart (T-3399) DCM | 29426 | 28.35 | 317.67 | 635.34 | heart T-3399 | 75.82 |
| adenoid GW99-269 | 26162 | 31.52 | 46.93 | 93.86 | adenoid | |
| tonsil GW98-280 | 22582 | 30.82 | 71.35 | 142.70 | tonsil | |
| T cells PC00314 | 28453 | 34.36 | 8.47 | 16.94 | T cells | |
| PBMNC | | 40 | 0 | 0.00 | PBMNC | |
| monocyte | | 40 | 0 | 0.00 | monocyte | |
| B cells PC00665 | 28455 | 40 | 0 | 0.00 | B cells | |
| dendritic cells 28441 | | 31.52 | 47.02 | 94.04 | dendritic cells | |
| neutrophils | 28440 | 36.13 | 2.91 | 2.91 | neutrophils | |
| eosinophils | 28446 | 40 | 0 | 0.00 | eosinophils | |
| BM unstim | | 40 | 0 | 0.00 | BM unstim | |
| BM stim | | 40 | 0 | 0.00 | BM stim | 0.00 |
| osteo dif | | 40 | 0 | 0.00 | osteo dif | 0.00 |
| osteo undif | | 40 | 0 | 0.00 | osteo undif | |
| chondrocytes | | 40 | 0 | 0.00 | chondrocytes | |
| OA Synovium IP12/01 | 29462 | 32.08 | 33.47 | 33.47 | OA Synovium | |
| OA Synovium NP10/01 | 29461 | 31.43 | 49.5 | 99.00 | OA Synovium | |
| OA Synovium NP57/00 | 28464 | 30.42 | 91.04 | 182.08 | OA Synovium | |
| RA Synovium NP03/01 | 28466 | 32.11 | 32.84 | 65.68 | RA Synovium | |
| RA Synovium NP71/00 | 28467 | 31.07 | 61.51 | 123.02 | RA Synovium | |
| RA Synovium NP45/00 | 28475 | 36.21 | 2.78 | 5.56 | RA Synovium | |
| OA bone (biobank) | 29217 | 31.49 | 47.85 | 47.85 | OA bone (biobank) | |
| OA bone Sample 1 | J. Emory | 30.11 | 109.44 | 218.88 | OA bone | |
| OA bone Sample 2 | J. Emory | 32.6 | 24.52 | 49.04 | OA bone | |
| Cartilage (pool) | Normal | 32.09 | 33.26 | 66.52 | Cartilage (pool) | |
| Cartilage (pool) | OA | 33.1 | 18.07 | 36.14 | Cartilage (pool) | −1.84 |
| PBL unifected | 28441 | 27.68 | 474.91 | 949.82 | PBL unifected | |
| PBL HIV IIIB | 28442 | 31.76 | 40.5 | 81.00 | PBL HIV IIIB | −11.73 |
| MRC5 uninfected | 29158 | 40 | 0 | 0.00 | MRC5 uninfected | |
| (100%) | | | | | (100%) | |
| MRC5 HSV strain F | 29178 | 34.15 | 9.61 | 19.22 | MRC5 HSV strain F | 19.22 |
| W12 cells | 29179 | 40 | 0 | 0.00 | W12 cells | |
| Keratinocytes | 29180 | 38.16 | 0.85 | 1.70 | Keratinocytes | |
| B-actin control | | 27.02 | 707.5 | | | |
| genomic | | 26.1 | 1232.73 | | | |
| 1.00E+05 | | 18.64 | 100000 | | | |
| 1.00E+05 | | 18.95 | 100000 | | | |
| 1.00E+04 | | 22.4 | 10000 | | | |
| 1.00E+04 | | 22.17 | 10000 | | | |
| 1.00E+03 | | 26.34 | 1000 | | | |
| 1.00E+03 | | 25.94 | 1000 | | | |
| 1.00E+02 | | 31.03 | 100 | | | |
| 1.00E+02 | | 32.83 | 100 | | | |
| 1.00E+01 | | 33.21 | 10 | | | |
| 1.00E+01 | | 32.93 | 10 | | | |
| 1.00E−00 | | 40 | 0 | | | |
| 1.00E−00 | | 40 | 0 | | | |
| NTC | | 40 | 0 | | | |

Gene Name sbg960509cbrecpt

| | Fold Change in Disease |
|---|---|
| | Population Relative to |
| Disease tissues | Normal |
| colon tumor | −2.51 |
| colon tumor | 1.57 |
| colon tumor | −1.25 |
| colon tumor | −20.13 |
| lung tumor | −41.69 |
| lung tumor | 10.00 |
| lung tumor | −4.82 |
| lung tumor | 3.66 |
| breast tumor | 2.22 |
| breast tumor | 8.92 |
| breast tumor | 1.84 |
| breast tumor | −25.88 |
| brain stage 5 ALZ | −41.28 |
| brain stage 5 ALZ | −3.49 |
| brain stage 5 ALZ | −1.16 |
| brain stage 5 ALZ | −41.28 |
| lung 24 | −104.07 |
| lung 28 | −104.07 |
| lung 23 | −16.16 |
| asthmatic lung | −200.14 |
| asthmatic lung | −4.11 |
| asthmatic lung | −3.72 |
| asthmatic lung | −5.81 |
| endo VEGF | 0.00 |
| endo bFGF | 0.00 |
| heart T-1 | 2.51 |
| heart T-14 | 13.90 |
| heart T-3399 | 75.82 |
| BM stim | 0.00 |
| osteo undif | 0.00 |
| Cartilage (pool) | −1.84 |
| PBL HIV IIIB | −11.73 |
| MRC5 HSV strain F | 19.22 |

Gene Name sbg614126complfH

Moderate to low overall expression in normal and disease samples. Highest normal expression in liver and fetal liver. Lower (but still significant expression) is seen in the whole brain, ovary, and uterus. Highest disease expression in 2 of the breast tumor samples, one of the normal brain samples, one of the normal lungs, one of the OA synovium samples, and the HSV-infected MRC5 cells. Upregulation in 1 of 4 colon tumors suggests a role in cancer of the colon. Downregulation in 2 of 4 lung tumors and upregulation in 1 of 4 breast tumors suggest roles in cancers of the lung and breast. Downregulation in 3 of 3 COPD lung samples as well as downregulation in 4 of 4 asthmatic lungs implies an involvement in chronic obstructive pulmonary disease and asthma. Upregulation in 1 of 3 heart samples suggests a role in DCM. Upregulation in HSV implicates involvement in herpes simplex virus as a potential host factor. Moderate to low expression in immune cells, RA and OA synovium bone, and chondrocytes.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | Ct | copies | copies | Average | 18S | 18S | 50 ng |
| Sample | (sample 1 | (sample | (sample | GOI | rRNA | rRNA | total |
| sbg614126complfH | and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous | 40, 40 | 0 | 0 | 0.00 | 0.96 | 52.36 | 0.00 |
| Adipose Zenbio | | | | | | | |
| Adrenal Gland | 40, 40 | 0 | 0 | 0.00 | 0.61 | 81.97 | 0.00 |
| Clontech | | | | | | | |
| Whole Brain | 32.34, 31.88 | 46.5 | 61.71 | 54.11 | 7.24 | 6.91 | 373.65 |
| Clontech | | | | | | | |
| Fetal Brain Clontech | 40, 40 | 0 | 0 | 0.00 | 0.48 | 103.95 | 0.00 |
| Cerebellum Clontech | 40, 40 | 0 | 0 | 0.00 | 2.17 | 23.04 | 0.00 |
| Cervix | 40, 35.04 | 0 | 8.88 | 4.44 | 2.42 | 20.66 | 91.74 |
| Colon | 40, 40 | 0 | 0 | 0.00 | 2.71 | 18.45 | 0.00 |
| Endometrium | 40, 40 | 0 | 0 | 0.00 | 0.73 | 68.21 | 0.00 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 39.79 | 0 | 0.48 | 0.24 | 0.32 | 155.28 | 37.27 |
| Ileum | 40, 36.32 | 0 | 4.04 | 2.02 | 2.58 | 19.38 | 39.15 |
| Jejunum | 33.25, 34.19 | 26.6 | 14.98 | 20.79 | 6.60 | 7.58 | 157.50 |
| Kidney | 40, 40 | 0 | 0 | 0.00 | 2.12 | 23.58 | 0.00 |
| Liver | 28.77, 28.81 | 417.4 | 407.38 | 412.39 | 1.50 | 33.33 | 13746.33 |
| Fetal Liver Clontech | 29.63, 29.5 | 246.38 | 266.67 | 256.53 | 10.40 | 4.81 | 1233.29 |
| Lung | 40, 40 | 0 | 0 | 0.00 | 2.57 | 19.46 | 0.00 |
| Mammary Gland | 34.19, 40 | 14.9 | 0 | 7.45 | 13.00 | 3.85 | 28.65 |
| Clontech | | | | | | | |
| Myometrium | 35.76, 40 | 5.7 | 0 | 2.85 | 2.34 | 21.37 | 60.90 |
| Omentum | 36.04, 33.62 | 4.81 | 21.16 | 12.99 | 3.94 | 12.69 | 164.78 |
| Ovary | 34.29, 32.95 | 14.02 | 31.93 | 22.98 | 4.34 | 11.52 | 264.69 |
| Pancreas | 40, 40 | 0 | 0 | 0.00 | 0.81 | 61.80 | 0.00 |
| Head of Pancreas | 38.98, 35.35 | 0.79 | 7.32 | 4.06 | 1.57 | 31.85 | 129.14 |
| Parotid Gland | 34.58, 33.83 | 11.74 | 18.68 | 15.21 | 5.48 | 9.12 | 138.78 |
| Placenta Clontech | 35.73, 35.66 | 5.82 | 6.06 | 5.94 | 5.26 | 9.51 | 56.46 |
| Prostate | 40, 40 | 0 | 0 | 0.00 | 3.00 | 16.67 | 0.00 |
| Rectum | 40, 40 | 0.38 | 0 | 0.19 | 1.23 | 40.65 | 7.72 |
| Salivary Gland | 40, 40 | 0.3 | 0 | 0.15 | 7.31 | 6.84 | 1.03 |
| Clontech | | | | | | | |
| Skeletal Muscle | 40, 40 | 0 | 0.28 | 0.14 | 1.26 | 39.68 | 5.56 |
| Clontech | | | | | | | |
| Skin | 40, 40 | 0 | 0.33 | 0.17 | 1.21 | 41.32 | 6.82 |
| Small Intestine | 40, 40 | 0 | 0 | 0.00 | 0.98 | 51.07 | 0.00 |
| Clontech | | | | | | | |
| Spleen | 40, 40 | 0 | 0 | 0.00 | 4.92 | 10.16 | 0.00 |
| Stomach | 40, 36 | 0 | 4.92 | 2.46 | 2.73 | 18.32 | 45.05 |
| Testis Clontech | 40, 40 | 0 | 0 | 0.00 | 0.57 | 87.87 | 0.00 |
| Thymus Clontech | 40, 37.06 | 0 | 2.56 | 1.28 | 9.89 | 5.06 | 6.47 |
| Thyroid | 40, 40 | 0 | 0.31 | 0.16 | 2.77 | 18.05 | 2.80 |
| Trachea Clontech | 40, 40 | 0.28 | 0 | 0.14 | 9.71 | 5.15 | 0.72 |
| Urinary Bladder | 40, 34.13 | 0 | 15.53 | 7.77 | 5.47 | 9.14 | 70.98 |
| Uterus | 33.21, 32.79 | 27.27 | 35.32 | 31.30 | 5.34 | 9.36 | 293.02 |
| genomic | 26.93 | 1288.98 | | | | | |
| b-actin | 27.55 | 878.74 | | | | | |
| 1.00E+05 | 20.07 | 100000 | | | | | |
| 1.00E+05 | 20.14 | 100000 | | | | | |
| 1.00E+04 | 23.43 | 10000 | | | | | |
| 1.00E+04 | 23.34 | 10000 | | | | | |
| 1.00E+03 | 26.84 | 1000 | | | | | |
| 1.00E+03 | 27.02 | 1000 | | | | | |
| 1.00E+02 | 31.72 | 100 | | | | | |
| 1.00E+02 | 31.32 | 100 | | | | | |
| 1.00E+01 | 33.78 | 10 | | | | | |
| 1.00E+01 | 35.79 | 10 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | |
| | Reg | | | detected/ | | Fold |
| | number | | Mean | 50 ng | | Change in |
| Sample | (GSK | | GOI | total | | Disease |
| sbg614126complfH | identifier) | Ct | copies | RNA | Sample | Population |
| colon normal GW98- | 21941 | 34.6 | 13.63 | 27.26 | colon normal | |
| 167 | | | | | | |
| colon tumor GW98-166 | 21940 | 35.71 | 7.35 | 14.70 | colon tumor | −1.85 |
| colon normal GW98- | 22080 | 40 | 0 | 0.00 | colon normal | |
| 178 | | | | | | |
| colon tumor GW98-177 | 22060 | 39.81 | 0.75 | 1.50 | colon tumor | 1.50 |
| colon normal GW98- | 23514 | 38.61 | 1.45 | 2.90 | colon normal | |
| 561 | | | | | | |
| colon tumor GW98-560 | 23513 | 34.84 | 11.95 | 23.90 | colon tumor | 8.24 |
| colon normal GW98- | 24691 | 39.05 | 1.14 | 2.28 | colon normal | |
| 894 | | | | | | |
| colon tumor GW98-893 | 24690 | 40 | 0 | 0.00 | colon tumor | −2.28 |
| lung normal GW98-3 | 20742 | 35.78 | 7.04 | 14.08 | lung normal | |
| lung tumor GW98-2 | 20741 | 40 | 0 | 0.00 | lung tumor | −14.08 |
| lung normal GW97-179 | 20677 | 33.99 | 19.21 | 38.42 | lung normal | |
| lung tumor GW97-178 | 20676 | 40 | 0.49 | 0.98 | lung tumor | −39.20 |
| lung normal GW98-165 | 21922 | 39.63 | 0.82 | 1.64 | lung normal | |
| lung tumor GW98-164 | 21921 | 38.89 | 1.24 | 2.48 | lung tumor | 1.51 |
| lung normal GW98-282 | 22584 | 40 | 0 | 0.00 | lung normal | |
| lung tumor GW98-281 | 22583 | 40 | 0 | 0.00 | lung tumor | 0.00 |
| breast normal GW00- | 28750 | 32.71 | 39.28 | 39.28 | breast normal | |
| 392 | | | | | | |
| breast tumor GW00-391 | 28746 | 31.65 | 70.89 | 141.78 | breast tumor | 3.61 |
| breast normal GW00- | 28798 | 35.83 | 6.88 | 6.88 | breast normal | |
| 413 | | | | | | |
| breast tumor GW00-412 | 28797 | 33.17 | 30.3 | 60.60 | breast tumor | 8.81 |
| breast normal GW00- | 27592-95 | 36.73 | 4.16 | 4.16 | breast normal | |
| 235:238 | | | | | | |
| breast tumor GW00- | 27588-91 | 35.98 | 6.33 | 6.33 | breast tumor | 1.52 |
| 231:234 | | | | | | |
| breast normal GW98- | 23656 | 37.38 | 2.89 | 5.78 | breast normal | |
| 621 | | | | | | |
| breast tumor GW98-620 | 23655 | 34.95 | 11.23 | 22.46 | breast tumor | 3.89 |
| brain normal BB99-542 | 25507 | 32.26 | 50.34 | 100.68 | brain normal | |
| brain normal BB99-406 | 25509 | 40 | 0.57 | 1.14 | brain normal | |
| brain normal BB99-904 | 25546 | 34.68 | 13.04 | 26.08 | brain normal | |
| brain stage 5 ALZ | 25502 | 40 | 0 | 0.00 | brain stage 5 | −42.63 |
| BB99-874 | | | | | ALZ | |
| brain stage 5 ALZ | 25503 | 35.87 | 6.73 | 13.46 | brain stage 5 | −3.17 |
| BB99-887 | | | | | ALZ | |
| brain stage 5 ALZ | 25504 | 39.2 | 1.05 | 2.10 | brain stage 5 | −20.30 |
| BB99-862 | | | | | ALZ | |
| brain stage 5 ALZ | 25542 | 40 | 0 | 0.00 | brain stage 5 | −42.63 |
| BB99-927 | | | | | ALZ | |
| CT lung KC | normal | 39.4 | 0.93 | 1.86 | CT lung | |
| lung 26 KC | normal | | | | lung 26 | |
| lung 27 KC | normal | 40 | 0 | 0.00 | lung 27 | |
| lung 24 KC | COPD | 40 | 0 | 0.00 | lung 24 | −0.62 |
| lung 28 KC | COPD | 40 | 0 | 0.00 | lung 28 | −0.62 |
| lung 23 KC | COPD | 40 | 0 | 0.00 | lung 23 | −0.62 |
| lung 25 KC | normal | 40 | 0 | 0.00 | lung 25 | |
| asthmatic lung | 29321 | 36.52 | 4.68 | 4.68 | asthmatic lung | 7.55 |
| ODO3112 | | | | | | |
| asthmatic lung | 29323 | 40 | 0 | 0.00 | asthmatic lung | −0.62 |
| ODO3433 | | | | | | |
| asthmatic lung | 29322 | 40 | 0 | 0.00 | asthmatic lung | −0.62 |
| ODO3397 | | | | | | |
| asthmatic lung | 29325 | 38.18 | 1.85 | 3.70 | asthmatic lung | 5.97 |
| ODO4928 | | | | | | |
| endo cells KC | control | 40 | 0 | 0.00 | endo cells | |
| endo VEGF KC | | 40 | 0 | 0.00 | endo VEGF | 0.00 |
| endo bFGF KC | | 40 | 0 | 0.00 | endo bFGF | 0.00 |
| heart Clontech | normal | 40 | 0 | 0.00 | heart | |
| heart (T-1) ischemic | 29417 | 40 | 0 | 0.00 | heart T-1 | 0.00 |
| heart (T-14) non- | 29422 | 40 | 0 | 0.00 | heart T-14 | 0.00 |
| obstructive DCM | | | | | | |
| heart (T-3399) DCM | 29426 | 36.03 | 6.13 | 12.26 | heart T-3399 | 12.26 |
| adenoid GW99-269 | 26162 | 34.08 | 18.19 | 36.38 | adenoid | |
| tonsil GW98-280 | 22582 | 37.46 | 2.77 | 5.54 | tonsil | |
| T cells PC00314 | 28453 | 40 | 0 | 0.00 | T cells | |
| PBMNC | | 40 | 0 | 0.00 | PBMNC | |
| monocyte | | 40 | 0 | 0.00 | monocyte | |
| B cells PC00665 | 28455 | 34.56 | 13.99 | 27.98 | B cells | |
| dendritic cells 28441 | | 40 | 0 | 0.00 | dendritic cells | |
| neutrophils | 28440 | 33.76 | 21.85 | 21.85 | neutrophils | |
| eosinophils | 28446 | 40 | 0 | 0.00 | eosinophils | |
| BM unstim | | 40 | 0 | 0.00 | BM unstim | |
| BM stim | | 40 | 0 | 0.00 | BM stim | 0.00 |
| osteo dif | | 40 | 0 | 0.00 | osteo dif | 0.00 |
| osteo undif | | 40 | 0 | 0.00 | osteo undif | |
| chondrocytes | | 34.25 | 16.55 | 41.38 | chondrocytes | |
| OA Synovium IP12/01 | 29462 | 40 | 0 | 0.00 | OA Synovium | |
| OA Synovium NP10/01 | 29461 | 40 | 0 | 0.00 | OA Synovium | |
| OA Synovium NP57/00 | 28464 | 33.1 | 31.54 | 63.08 | OA Synovium | |
| RA Synovium NP03/01 | 28466 | 40 | 0 | 0.00 | RA Synovium | |
| RA Synovium NP71/00 | 28467 | 40 | 0 | 0.00 | RA Synovium | |
| RA Synovium NP45/00 | 28475 | 40 | 0 | 0.00 | RA Synovium | |
| OA bone (biobank) | 29217 | 40 | 0 | 0.00 | OA bone | |
| | | | | | (biobank) | |
| OA bone Sample 1 | J. Emory | 40 | 0 | 0.00 | OA bone | |
| OA bone Sample 2 | J. Emory | 40 | 0 | 0.00 | OA bone | |
| Cartilage (pool) | Normal | 40 | 0 | 0.00 | Cartilage (pool) | |
| Cartilage (pool) | OA | 40 | 0 | 0.00 | Cartilage (pool) | 0.00 |
| PBL unifected | 28441 | 36.12 | 5.84 | 11.68 | PBL unifected | |
| PBL HIV IIIB | 28442 | 36.1 | 5.9 | 11.80 | PBL HIV IIIB | 1.01 |
| MRC5 uninfected | 29158 | 40 | 0 | 0.00 | MRC5 | |
| (100%) | | | | | uninfected | |
| | | | | | (100%) | |
| MRC5 HSV strain F | 29178 | 31.83 | 64.08 | 128.16 | MRC5 HSV | 128.16 |
| | | | | | strain F | |
| W12 cells | 29179 | 40 | 0 | 0.00 | W12 cells | |
| Keratinocytes | 29180 | 40 | 0 | 0.00 | Keratinocytes | |
| B-actin control | | 27.26 | 820.77 | | | |
| genomic | | 26.18 | 1496.25 | | | |
| 1.00E+05 | | 19.13 | 100000 | | | |
| 1.00E+05 | | 19.38 | 100000 | | | |
| 1.00E+04 | | 22.56 | 10000 | | | |
| 1.00E+04 | | 22.67 | 10000 | | | |
| 1.00E+03 | | 26.01 | 1000 | | | |
| 1.00E+03 | | 26.44 | 1000 | | | |
| 1.00E+02 | | 30.93 | 100 | | | |
| 1.00E+02 | | 30.1 | 100 | | | |
| 1.00E+01 | | 38.59 | 10 | | | |
| 1.00E+01 | | 33.26 | 10 | | | |
| 1.00E−00 | | 40 | 0 | | | |
| 1.00E−00 | | 40 | 0 | | | |
| NTC | | 40 | 0 | | | |

Gene Name sbg6141262complfH

| | Fold Change in Disease |
|---|---|
| | Population Relative to |
| Disease tissues | Normal |
| colon tumor | −1.85 |
| colon tumor | 1.50 |
| colon tumor | 8.24 |
| colon tumor | −2.28 |
| lung tumor | −14.08 |
| lung tumor | −39.20 |
| lung tumor | 1.51 |
| lung tumor | 0.00 |
| breast tumor | 3.61 |
| breast tumor | 8.81 |
| breast tumor | 1.52 |
| breast tumor | 3.89 |
| brain stage 5 ALZ | −42.63 |
| brain stage 5 ALZ | −3.17 |
| brain stage 5 ALZ | −20.30 |
| brain stage 5 ALZ | −42.63 |
| lung 24 | −0.62 |
| lung 28 | −0.62 |
| lung 23 | −0.62 |
| asthmatic lung | 7.55 |
| asthmatic lung | −0.62 |
| asthmatic lung | −0.62 |
| asthmatic lung | 5.97 |
| endo VEGF | 0.00 |
| endo bFGF | 0.00 |
| heart T-1 | 0.00 |
| heart T-14 | 0.00 |
| heart T-3399 | 12.26 |
| BM stim | 0.00 |
| osteo undif | 0.00 |
| Cartilage (pool) | 0.00 |
| PBL HIV IIIB | 1.01 |
| MRC5 HSV strain F | 128.16 |

Gene Name sbg120703RNase

Moderate to low overall expression in normal and disease samples. Highest normal expression in whole brain and salivary gland. Moderate expression in the fetal liver and the thymus. Highest disease expression in 2 of the normal lung samples, one of the lung tumor samples, the normal cartilage pool, and the HSV-infected MRC5 cells. Upregulation in 1 of 4 colon tumors suggests a role in cancer of the colon. Downregulation in 2 of 4 lung tumor samples suggests possible implication in lung cancer. Upregulation in 2 of 4 breast tumors implies an involvement in cancers of the breast. Downregulation in 3 of 3 COPD lung samples implies an involvement in COPD. Upregulation in 3 of 3 heart samples implicates this gene in diseases of the heart such as DCM and ischemia. High expression in the OA and RA synovium and the OA bone samples suggests a possible involvement in osteoarthritis and rheumatoid arthritis. Upregulation in HSV implicates this gene in herpes simplex virus as a potential host factor. Moderate to low expression in immune cells.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg120703RNase | 1 and 2) | 1) | 2) | Copies | (ng) | (ng) | RNA |
| Subcutaneous | 40, 36.24 | 0 | 2.54 | 1.27 | 3.06 | 16.34 | 20.75 |
| Adipocytes Zenbio | | | | | | | |
| Subcutaneous | 36.58, 40 | 2.07 | 0 | 1.04 | 0.96 | 52.36 | 54.19 |
| Adipose Zenbio | | | | | | | |
| Adrenal Gland | 40, 40 | 0.22 | 0 | 0.11 | 0.61 | 81.97 | 9.02 |
| Clontech | | | | | | | |
| Whole Brain | 28.62, 28.6 | 245.21 | 247.41 | 246.31 | 7.24 | 6.91 | 1701.04 |
| Clontech | | | | | | | |
| Fetal Brain Clontech | 40, 40 | 0.3 | 0 | 0.15 | 0.48 | 103.95 | 15.59 |
| Cerebellum | 40, 40 | 0.29 | 0 | 0.15 | 2.17 | 23.04 | 3.34 |
| Clontech | | | | | | | |
| Cervix | 35.3, 40 | 4.45 | 0 | 2.23 | 2.42 | 20.66 | 45.97 |
| Colon | 40, 40 | 0.26 | 0 | 0.13 | 2.71 | 18.45 | 2.40 |
| Endometrium | 40, 38.38 | 0 | 0.7 | 0.35 | 0.73 | 68.21 | 23.87 |
| Esophagus | 36.11, 37.01 | 2.74 | 1.6 | 2.17 | 1.37 | 36.50 | 79.20 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 39.31, 36.07 | 0.4 | 2.8 | 1.60 | 2.58 | 19.38 | 31.01 |
| Jejunum | 34.13, 39.51 | 8.98 | 0.36 | 4.67 | 6.60 | 7.58 | 35.38 |
| Kidney | 40, 40 | 0.48 | 0 | 0.24 | 2.12 | 23.58 | 5.66 |
| Liver | 34.4, 36.04 | 7.64 | 2.86 | 5.25 | 1.50 | 33.33 | 175.00 |
| Fetal Liver Clontech | 31.46, 31.39 | 44.65 | 46.4 | 45.53 | 10.40 | 4.81 | 218.87 |
| Lung | 34.21, 35.61 | 8.59 | 3.71 | 6.15 | 2.57 | 19.46 | 119.65 |
| Mammary Gland | 34.9, 35.65 | 5.67 | 3.6 | 4.64 | 13.00 | 3.85 | 17.83 |
| Clontech | | | | | | | |
| Myometrium | 40, 38.99 | 0 | 0.49 | 0.25 | 2.34 | 21.37 | 5.24 |
| Omentum | 38.39, 34.35 | 0.7 | 7.89 | 4.30 | 3.94 | 12.69 | 54.51 |
| Ovary | 35, 33.21 | 5.34 | 15.64 | 10.49 | 4.34 | 11.52 | 120.85 |
| Pancreas | 40 | 0 | 0 | 0.00 | 0.81 | 61.80 | 0.00 |
| Head of Pancreas | 40 | 0 | 0 | 0.00 | 1.57 | 31.85 | 0.00 |
| Parotid Gland | 32.22, 33.49 | 28.28 | 13.18 | 20.73 | 5.48 | 9.12 | 189.14 |
| Placenta Clontech | 37, 39.59 | 1.6 | 0.34 | 0.97 | 5.26 | 9.51 | 9.22 |
| Prostate | 35.03, 35.75 | 5.23 | 3.4 | 4.32 | 3.00 | 16.67 | 71.92 |
| Rectum | 38.25, 40 | 0.76 | 0.21 | 0.49 | 1.23 | 40.65 | 19.72 |
| Salivary Gland | 30.01, 29.73 | 106.25 | 125.78 | 116.02 | 7.31 | 6.84 | 793.54 |
| Clontech | | | | | | | |
| Skeletal Muscle | 40, 39.16 | 0.41 | 0.44 | 0.43 | 1.26 | 39.68 | 16.87 |
| Clontech | | | | | | | |
| Skin | 37.21, 35.01 | 1.42 | 5.31 | 3.37 | 1.21 | 41.32 | 139.05 |
| Small Intestine | 40, 40 | 0 | 0.19 | 0.10 | 0.98 | 51.07 | 4.85 |
| Clontech | | | | | | | |
| Spleen | 35.4, 35.9 | 4.2 | 3.11 | 3.66 | 4.92 | 10.16 | 37.14 |
| Stomach | 36.12, 40 | 2.73 | 0.21 | 1.47 | 2.73 | 18.32 | 26.92 |
| Testis Clontech | 40, 40 | 0 | 0 | 0.00 | 0.57 | 87.87 | 0.00 |
| Thymus Clontech | 31.88, 31.42 | 34.61 | 45.62 | 40.12 | 9.89 | 5.06 | 202.81 |
| Thyroid | 40, 35.22 | 0 | 4.67 | 2.34 | 2.77 | 18.05 | 42.15 |
| Trachea Clontech | 35.38, 37.52 | 4.26 | 1.17 | 2.72 | 9.71 | 5.15 | 13.98 |
| Urinary Bladder | 38.77, 40 | 0.56 | 0.31 | 0.44 | 5.47 | 9.14 | 3.98 |
| Uterus | 33.66, 37.55 | 11.93 | 1.16 | 6.55 | 5.34 | 9.36 | 61.28 |
| genomic | 25.78 | 1342.66 | | | | | |
| b-actin | 27.27 | 551.42 | | | | | |
| 1.00E+05 | 19.03 | 100000 | | | | | |
| 1.00E+05 | 19.08 | 100000 | | | | | |
| 1.00E+04 | 22.28 | 10000 | | | | | |
| 1.00E+04 | 22.27 | 10000 | | | | | |
| 1.00E+03 | 25.85 | 1000 | | | | | |
| 1.00E+03 | 25.6 | 1000 | | | | | |
| 1.00E+02 | 30.44 | 100 | | | | | |
| 1.00E+02 | 29.33 | 100 | | | | | |
| 1.00E+01 | 34.4 | 10 | | | | | |
| 1.00E+01 | 34.48 | 10 | | | | | |
| 1.00E−00 | | | | | | | |
| 1.00E−00 | | | | | | | |
| NTC | 40 | −1 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| | (GSK | | GOI | total | | Disease |
| Sample sbg120703RNase | identifier) | Ct | copies | RNA | Sample | Population |
| colon normal GW98-167 | 21941 | 29.03 | 142.85 | 285.70 | colon normal | |
| colon tumor GW98-166 | 21940 | 28.31 | 226.87 | 453.74 | colon tumor | 1.59 |
| colon normal GW98-178 | 22080 | 33.08 | 10.78 | 21.56 | colon normal | |
| colon tumor GW98-177 | 22060 | 29.33 | 118.09 | 236.18 | colon tumor | 10.95 |
| colon normal GW98-561 | 23514 | 30.02 | 76.09 | 152.18 | colon normal | |
| colon tumor GW98-560 | 23513 | 30.42 | 58.89 | 117.78 | colon tumor | −1.29 |
| colon normal GW98-894 | 24691 | 29.07 | 139.29 | 278.58 | colon normal | |
| colon tumor GW98-893 | 24690 | 30.3 | 63.5 | 127.00 | colon tumor | −2.19 |
| lung normal GW98-3 | 20742 | 26.86 | 574.4 | 1148.80 | lung normal | |
| lung tumor GW98-2 | 20741 | 30.07 | 73.89 | 147.78 | lung tumor | −7.77 |
| lung normal GW97-179 | 20677 | 29.74 | 90.79 | 181.58 | lung normal | |
| lung tumor GW97-178 | 20676 | 27.63 | 351.24 | 702.48 | lung tumor | 3.87 |
| lung normal GW98-165 | 21922 | 26.63 | 663.94 | 1327.88 | lung normal | |
| lung tumor GW98-164 | 21921 | 29.38 | 114.52 | 229.04 | lung tumor | −5.80 |
| lung normal GW98-282 | 22584 | 30 | 77.02 | 154.04 | lung normal | |
| lung tumor GW98-281 | 22583 | 29.64 | 97.04 | 194.08 | lung tumor | 1.26 |
| breast normal GW00-392 | 28750 | 29.08 | 138.57 | 138.57 | breast normal | |
| breast tumor GW00-391 | 28746 | 28.77 | 169.53 | 339.06 | breast tumor | 2.45 |
| breast normal GW00-413 | 28798 | 32.72 | 13.55 | 13.55 | breast normal | |
| breast tumor GW00-412 | 28797 | 31.01 | 40.4 | 80.80 | breast tumor | 5.96 |
| breast normal GW00- | 27592-95 | 34.39 | 4.68 | 4.68 | breast normal | |
| 235:238 | | | | | | |
| breast tumor GW00- | 27588-91 | 31.4 | 31.48 | 31.48 | breast tumor | 6.73 |
| 231:234 | | | | | | |
| breast normal GW98-621 | 23656 | 28.54 | 195.6 | 391.20 | breast normal | |
| breast tumor GW98-620 | 23655 | 30.37 | 60.84 | 121.68 | breast tumor | −3.21 |
| brain normal BB99-542 | 25507 | 32.94 | 11.79 | 23.58 | brain normal | |
| brain normal BB99-406 | 25509 | 32.22 | 18.66 | 37.32 | brain normal | |
| brain normal BB99-904 | 25546 | 32.3 | 17.71 | 35.42 | brain normal | |
| brain stage 5 ALZ BB99- | 25502 | 32.82 | 12.76 | 25.52 | brain stage 5 | −1.26 |
| 874 | | | | | ALZ | |
| brain stage 5 ALZ BB99- | 25503 | 30.31 | 63.18 | 126.36 | brain stage 5 | 3.94 |
| 887 | | | | | ALZ | |
| brain stage 5 ALZ BB99- | 25504 | 31.42 | 31.08 | 62.16 | brain stage 5 | 1.94 |
| 862 | | | | | ALZ | |
| brain stage 5 ALZ BB99- | 25542 | 33.35 | 9.08 | 18.16 | brain stage 5 | −1.77 |
| 927 | | | | | ALZ | |
| CT lung KC | normal | 30.41 | 59.49 | 118.98 | CT lung | |
| lung 26 KC | normal | | | | lung 26 | |
| lung 27 KC | normal | 37.69 | 0.57 | 0.57 | lung 27 | |
| lung 24 KC | COPD | 40 | 0 | 0.00 | lung 24 | −40.17 |
| lung 28 KC | COPD | 40 | 0 | 0.00 | lung 28 | −40.17 |
| lung 23 KC | COPD | 40 | 0 | 0.00 | lung 23 | −40.17 |
| lung 25 KC | normal | 36.86 | 0.97 | 0.97 | lung 25 | |
| asthmatic lung ODO3112 | 29321 | 33.08 | 10.79 | 10.79 | asthmatic lung | −3.72 |
| asthmatic lung ODO3433 | 29323 | 29.94 | 80.31 | 160.62 | asthmatic lung | 4.00 |
| asthmatic lung ODO3397 | 29322 | 29.79 | 87.94 | 175.88 | asthmatic lung | 4.38 |
| asthmatic lung ODO4928 | 29325 | 30.08 | 73.39 | 146.78 | asthmatic lung | 3.65 |
| endo cells KC | control | 40 | 0.13 | 0.13 | endo cells | |
| endo VEGF KC | | 40 | 0 | 0.00 | endo VEGF | −0.13 |
| endo bFGF KC | | 40 | 0.12 | 0.12 | endo bFGF | −1.08 |
| heart Clontech | normal | 34.66 | 3.95 | 7.90 | heart | |
| heart (T-1) ischemic | 29417 | 30.43 | 58.48 | 116.96 | heart T-1 | 14.81 |
| heart (T-14) non- | 29422 | 30.3 | 63.76 | 127.52 | heart T-14 | 16.14 |
| obstructive DCM | | | | | | |
| heart (T-3399) DCM | 29426 | 31.14 | 37.27 | 74.54 | heart T-3399 | 9.44 |
| adenoid GW99-269 | 26162 | 33.15 | 10.31 | 20.62 | adenoid | |
| tonsil GW98-280 | 22582 | 30.26 | 65.22 | 130.44 | tonsil | |
| T cells PC00314 | 28453 | 33.29 | 9.45 | 18.90 | T cells | |
| PBMNC | | 40 | 0 | 0.00 | PBMNC | |
| monocyte | | 40 | 0 | 0.00 | monocyte | |
| B cells PC00665 | 28455 | 32.25 | 18.35 | 36.70 | B cells | |
| dendritic cells 28441 | | 30.52 | 55.34 | 110.68 | dendritic cells | |
| neutrophils | 28440 | 31.61 | 27.61 | 27.61 | neutrophils | |
| eosinophils | 28446 | 33.2 | 10.01 | 20.02 | eosinophils | |
| BM unstim | | 40 | 0 | 0.00 | BM unstim | |
| BM stim | | 40 | 0 | 0.00 | BM stim | 0.00 |
| osteo dif | | 40 | 0 | 0.00 | osteo dif | 0.00 |
| osteo undif | | 40 | 0 | 0.00 | osteo undif | |
| chondrocytes | | 29.65 | 96.25 | 240.63 | chondrocytes | |
| OA Synovium IP12/01 | 29462 | 28.59 | 190.09 | 190.09 | OA Synovium | |
| OA Synovium NP10/01 | 29461 | 29.06 | 140.66 | 281.32 | OA Synovium | |
| OA Synovium NP57/00 | 28464 | 28.38 | 216.61 | 433.22 | OA Synovium | |
| RA Synovium NP03/01 | 28466 | 29.43 | 111 | 222.00 | RA Synovium | |
| RA Synovium NP71/00 | 28467 | 28.35 | 220.48 | 440.96 | RA Synovium | |
| RA Synovium NP45/00 | 28475 | 28.7 | 176.79 | 353.58 | RA Synovium | |
| OA bone (biobank) | 29217 | 30.6 | 52.6 | 52.60 | OA bone | |
| | | | | | (biobank) | |
| OA bone Sample 1 | J. Emory | 29.64 | 97.1 | 194.20 | OA bone | |
| OA bone Sample 2 | J. Emory | 30.85 | 44.71 | 89.42 | OA bone | |
| Cartilage (pool) | Normal | 28.07 | 264.86 | 529.72 | Cartilage | |
| | | | | | (pool) | |
| Cartilage (pool) | OA | 30.47 | 56.97 | 113.94 | Cartilage | −4.65 |
| | | | | | (pool) | |
| PBL unifected | 28441 | 33.41 | 8.73 | 17.46 | PBL unifected | |
| PBL HIV IIIB | 28442 | 32.1 | 20.17 | 40.34 | PBL HIV IIIB | 2.31 |
| MRC5 uninfected (100%) | 29158 | 31.09 | 38.5 | 77.00 | MRC5 | |
| | | | | | uninfected | |
| | | | | | (100%) | |
| MRC5 HSV strain F | 29178 | 28.24 | 237.46 | 474.92 | MRC5 HSV | 6.17 |
| | | | | | strain F | |
| W12 cells | 29179 | 28.83 | 162.45 | 324.90 | W12 cells | |
| Keratinocytes | 29180 | 29.21 | 127.89 | 255.78 | Keratinocytes | |
| B-actin control | | 26.99 | 528.52 | | | |
| genomic | | 25.66 | 1229.15 | | | |
| 1.00E+05 | | 18.76 | 100000 | | | |
| 1.00E+05 | | 19.03 | 100000 | | | |
| 1.00E+04 | | 22.01 | 10000 | | | |
| 1.00E+04 | | 22.05 | 10000 | | | |
| 1.00E+03 | | 26.01 | 1000 | | | |
| 1.00E+03 | | 25.68 | 1000 | | | |
| 1.00E+02 | | 30.57 | 100 | | | |
| 1.00E+02 | | 30.32 | 100 | | | |
| 1.00E+01 | | 32.24 | 10 | | | |
| 1.00E+01 | | 40 | 0 | | | |
| 1.00E−00 | | 40 | 0 | | | |
| 1.00E−00 | | 40 | 0 | | | |
| NTC | | 40 | 0 | | | |

Gene Name sbg120703RNase

| | Fold Change in Disease |
|---|---|
| | Population Relative to |
| Disease tissues | Normal |
| colon tumor | 1.59 |
| colon tumor | 10.95 |
| colon tumor | −1.29 |
| colon tumor | −2.19 |
| lung tumor | −7.77 |
| lung tumor | 3.87 |
| lung tumor | −5.80 |
| lung tumor | 1.26 |
| breast tumor | 2.45 |
| breast tumor | 5.96 |
| breast tumor | 6.73 |
| breast tumor | −3.21 |
| brain stage 5 ALZ | −1.26 |
| brain stage 5 ALZ | 3.94 |
| brain stage 5 ALZ | 1.94 |
| brain stage 5 ALZ | −1.77 |
| lung 24 | −40.17 |
| lung 28 | −40.17 |
| lung 23 | −40.17 |
| asthmatic lung | −3.72 |
| asthmatic lung | 4.00 |
| asthmatic lung | 4.38 |
| asthmatic lung | 3.65 |
| endo VEGF | −0.13 |
| endo bFGF | −1.08 |
| heart T-1 | 14.81 |
| heart T-14 | 16.14 |
| heart T-3399 | 9.44 |
| BM stim | 0.00 |
| osteo undif | 0.00 |
| Cartilage (pool) | −4.65 |
| PBL HIV IIIB | 2.31 |
| MRC5 HSV strain F | 6.17 |

Gene Name sbg98530TS

Moderate overall expression in normal and disease samples. Highest normal expression in whole brain, endometrium, and testis. Moderate expression in normal heart, skeletal muscle, and esophagus. Shows expression in most of the GI tract samples as well as the female reproductive tract samples. Highest disease expression in one of the colon tumor samples, all 3 of the heart samples, and the chondrocytes. Data predominantly shows a muscle-specific pattern of expression. Upregulation in 1 of 4 colon tumors and upregulation in 2 of 4 breast tumors implies an involvement in cancers of the colon and breast. Downregulation in 3 of 3 COPD samples implies a role in chronic obstructive pulmonary disease. Downregulation in HSV implicates involvement in herpes simplex virus as a potential host factor. Moderate to low overall expression in immune cells. High expression in chondrocytes and OA and RA synovium suggests possible involvement in osteoarthritis and rheumatoid arthritis.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg98530TS | 1 and 2) | 1) | 2) | Copies | (ng) | (ng) | RNA |
| Subcutaneous | 40, 36.35 | 0 | 4.22 | 2.11 | 3.06 | 16.34 | 34.48 |
| Adipocytes Zenbio | | | | | | | |
| Subcutaneous Adipose | 35.78, 40 | 5.89 | 0 | 2.95 | 0.96 | 52.36 | 154.19 |
| Zenbio | | | | | | | |
| Adrenal Gland | 40, 40 | 0 | 0 | 0.00 | 0.61 | 81.97 | 0.00 |
| Clontech | | | | | | | |
| Whole Brain Clontech | 26.63, 26.42 | 1253.02 | 1414.2 | 1333.61 | 7.24 | 6.91 | 9210.01 |
| Fetal Brain Clontech | 40, 37.33 | 0 | 2.38 | 1.19 | 0.48 | 103.95 | 123.70 |
| Cerebellum Clontech | 35.9, 40 | 5.5 | 0 | 2.75 | 2.17 | 23.04 | 63.36 |
| Cervix | 33.47, 34.27 | 22.86 | 14.26 | 18.56 | 2.42 | 20.66 | 383.47 |
| Colon | 34.49, 34.05 | 12.58 | 16.28 | 14.43 | 2.71 | 18.45 | 266.24 |
| Endometrium | 33.28, 32.94 | 25.41 | 31.15 | 28.28 | 0.73 | 68.21 | 1929.06 |
| Esophagus | 33.61, 32.9 | 21.02 | 31.85 | 26.44 | 1.37 | 36.50 | 964.78 |
| Heart Clontech | 33.32, 33.03 | 24.91 | 29.42 | 27.17 | 1.32 | 37.88 | 1028.98 |
| Hypothalamus | 40, 40 | 0 | 0 | 0.00 | 0.32 | 155.28 | 0.00 |
| Ileum | 35.89, 33.37 | 5.53 | 24.14 | 14.84 | 2.58 | 19.38 | 287.50 |
| Jejunum | 31.55, 31.42 | 70.2 | 75.71 | 72.96 | 6.60 | 7.58 | 552.69 |
| Kidney | 40, 35.5 | 0 | 6.96 | 3.48 | 2.12 | 23.58 | 82.08 |
| Liver | 34.29, 33.63 | 14.07 | 20.78 | 17.43 | 1.50 | 33.33 | 580.83 |
| Fetal Liver Clontech | 32.16, 32.92 | 49.02 | 31.52 | 40.27 | 10.40 | 4.81 | 193.61 |
| Lung | 40, 35.78 | 0 | | 2.95 | 2.57 | 19.46 | 57.30 |
| Mammary Gland | 31.42, 32.08 | 75.62 | 51.54 | 63.58 | 13.00 | 3.85 | 244.54 |
| Clontech | | | | | | | |
| Myometrium | 32.93, 32.03 | 31.21 | 52.84 | 42.03 | 2.34 | 21.37 | 897.97 |
| Omentum | 35.21, 40 | 8.23 | 0 | 4.12 | 3.94 | 12.69 | 52.22 |
| Ovary | 35.36, 35.51 | 7.53 | 6.89 | 7.21 | 4.34 | 11.52 | 83.06 |
| Pancreas | 40, 40 | 0 | 0 | 0.00 | 0.81 | 61.80 | 0.00 |
| Head of Pancreas | 40, 37.54 | 0 | 2.11 | 1.06 | 1.57 | 31.85 | 33.60 |
| Parotid Gland | 31.67, 31.01 | 65.33 | 96.46 | 80.90 | 5.48 | 9.12 | 738.09 |
| Placenta Clontech | 33.13, 32.05 | 27.88 | 52.3 | 40.09 | 5.26 | 9.51 | 381.08 |
| Prostate | 35.03, 40 | 9.13 | 5.22 | 7.18 | 3.00 | 16.67 | 119.58 |
| Rectum | 40, 35.19 | 0 | 8.32 | 4.16 | 1.23 | 40.65 | 169.11 |
| Salivary Gland | 32.41, 34.06 | 42.32 | 16.15 | 29.24 | 7.31 | 6.84 | 199.97 |
| Clontech | | | | | | | |
| Skeletal Muscle | 33.93, 33.76 | 17.41 | 19.28 | 18.35 | 1.26 | 39.68 | 727.98 |
| Clontech | | | | | | | |
| Skin | 40, 40 | 0 | 0 | 0.00 | 1.21 | 41.32 | 0.00 |
| Small Intestine | 34.11 | 11.73 | 15.7 | 13.72 | 0.98 | 51.07 | 700.46 |
| Clontech | | | | | | | |
| Spleen | 36.08, 40 | 4.94 | 0.37 | 2.66 | 4.92 | 10.16 | 26.98 |
| Stomach | 40, 40 | 0 | 0 | 0.00 | 2.73 | 18.32 | 0.00 |
| Testis Clontech | 35.54, 33.26 | 6.79 | 25.83 | 16.31 | 0.57 | 87.87 | 1433.22 |
| Thymus Clontech | 33.66, 34.12 | 20.35 | 15.62 | 17.99 | 9.89 | 5.06 | 90.93 |
| Thyroid | 40, 35.46 | 0 | 7.12 | 3.56 | 2.77 | 18.05 | 64.26 |
| Trachea Clontech | 32.08, 31.84 | 51.54 | 59.21 | 55.38 | 9.71 | 5.15 | 285.14 |
| Urinary Bladder | 34.75, 36.99 | 10.8 | 2.91 | 6.86 | 5.47 | 9.14 | 62.66 |
| Uterus | 31.79, 32.2 | 60.97 | 47.95 | 54.46 | 5.34 | 9.36 | 509.93 |
| genomic | 26.8 | 1133.17 | | | | | |
| b-actin | 27.6 | 706.62 | | | | | |
| 1.00E+05 | 19.53 | 100000 | | | | | |
| 1.00E+05 | 19.54 | 100000 | | | | | |
| 1.00E+04 | 22.8 | 10000 | | | | | |
| 1.00E+04 | 23.02 | 10000 | | | | | |
| 1.00E+03 | 26.14 | 1000 | | | | | |
| 1.00E+03 | 26.59 | 1000 | | | | | |
| 1.00E+02 | 31.41 | 100 | | | | | |
| 1.00E+02 | 30.97 | 100 | | | | | |
| 1.00E+01 | 40 | 0 | | | | | |
| 1.00E+01 | 35.24 | 10 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | Fold |
| | Reg | | | detected/ | | Change |
| | number | | Mean | 50 ng | | in |
| Sample | (GSK | | GOI | total | | Disease |
| sbg98530TS | identifier) | Ct | copies | RNA | Sample | Population |
| colon normal GW98-167 | 21941 | 26.26 | 1792.89 | 3585.78 | colon normal | |
| colon tumor GW98-166 | 21940 | 26.2 | 1856.22 | 3712.44 | colon tumor | 1.04 |
| colon normal GW98-178 | 22080 | 27.25 | 986.8 | 1973.60 | colon normal | |
| colon tumor GW98-177 | 22060 | 26.7 | 1369.12 | 2738.24 | colon tumor | 1.39 |
| colon normal GW98-561 | 23514 | 27.55 | 821.35 | 1642.70 | colon normal | |
| colon tumor GW98-560 | 23513 | 24.64 | 4748.96 | 9497.92 | colon tumor | 5.78 |
| colon normal GW98-894 | 24691 | 27.27 | 971.87 | 1943.74 | colon normal | |
| colon tumor GW98-893 | 24690 | 25.35 | 3093.47 | 6186.94 | colon tumor | 3.18 |
| lung normal GW98-3 | 20742 | 27.02 | 1133.68 | 2267.36 | lung normal | |
| lung tumor GW98-2 | 20741 | 27.26 | 981.94 | 1963.88 | lung tumor | −1.15 |
| lung normal GW97-179 | 20677 | 29.14 | 315.07 | 630.14 | lung normal | |
| lung tumor GW97-178 | 20676 | 28.15 | 571.76 | 1143.52 | lung tumor | 1.81 |
| lung normal GW98-165 | 21922 | 27.86 | 682.2 | 1364.40 | lung normal | |
| lung tumor GW98-164 | 21921 | 27.45 | 871.19 | 1742.38 | lung tumor | 1.28 |
| lung normal GW98-282 | 22584 | 28.12 | 581.74 | 1163.48 | lung normal | |
| lung tumor GW98-281 | 22583 | 29.32 | 283.71 | 567.42 | lung tumor | −2.05 |
| breast normal GW00-392 | 28750 | 27.85 | 687.38 | 687.38 | breast normal | |
| breast tumor GW00-391 | 28746 | 26.61 | 1444.19 | 2888.38 | breast tumor | 4.20 |
| breast normal GW00-413 | 28798 | 28.43 | 483.03 | 483.03 | breast normal | |
| breast tumor GW00-412 | 28797 | 25.49 | 2836.66 | 5673.32 | breast tumor | 11.75 |
| breast normal GW00- | 27592-95 | 32.26 | 48.29 | 48.29 | breast normal | |
| 235:238 | | | | | | |
| breast tumor GW00- | 27588-91 | 29.07 | 328.46 | 328.46 | breast tumor | 6.80 |
| 231:234 | | | | | | |
| breast normal GW98-621 | 23656 | 26.82 | 1279.07 | 2558.14 | breast normal | |
| breast tumor GW98-620 | 23655 | 26.8 | 1289.27 | 2578.54 | breast tumor | 1.01 |
| brain normal BB99-542 | 25507 | 29.03 | 337.63 | 675.26 | brain normal | |
| brain normal BB99-406 | 25509 | 29.19 | 305.6 | 611.20 | brain normal | |
| brain normal BB99-904 | 25546 | 30.44 | 144.55 | 289.10 | brain normal | |
| brain stage 5 ALZ BB99- | 25502 | 28.47 | 471.8 | 943.60 | brain stage 5 ALZ | 1.80 |
| 874 | | | | | | |
| brain stage 5 ALZ BB99- | 25503 | 27.3 | 955.52 | 1911.04 | brain stage 5 ALZ | 3.64 |
| 887 | | | | | | |
| brain stage 5 ALZ BB99- | 25504 | 27.42 | 891.77 | 1783.54 | brain stage 5 ALZ | 3.40 |
| 862 | | | | | | |
| brain stage 5 ALZ BB99- | 25542 | 29.31 | 285.16 | 570.32 | brain stage 5 ALZ | 1.09 |
| 927 | | | | | | |
| CT lung KC | normal | 27.96 | 643.88 | 1287.76 | CT lung | |
| lung 26 KC | normal | 35.82 | 5.66 | 5.66 | lung 26 | |
| lung 27 KC | normal | 40 | 0 | 0.00 | lung 27 | |
| lung 24 KC | COPD | 40 | 0 | 0.00 | lung 24 | −325.52 |
| lung 28 KC | COPD | 35.3 | 7.73 | 7.73 | lung 28 | −42.11 |
| lung 23 KC | COPD | 36.67 | 3.39 | 3.39 | lung 23 | −96.02 |
| lung 25 KC | normal | 35.11 | 8.67 | 8.67 | lung 25 | |
| asthmatic lung ODO3112 | 29321 | 31.01 | 102.08 | 102.08 | asthmatic lung | −3.19 |
| asthmatic lung ODO3433 | 29323 | 29.76 | 216.81 | 433.62 | asthmatic lung | 1.33 |
| asthmatic lung ODO3397 | 29322 | 29.83 | 208.08 | 416.16 | asthmatic lung | 1.28 |
| asthmatic lung ODO4928 | 29325 | 30.37 | 150.17 | 300.34 | asthmatic lung | −1.08 |
| endo cells KC | control | 37.54 | 2 | 2.00 | endo cells | |
| endo VEGF KC | | 35.77 | 5.83 | 5.83 | endo VEGF | 2.92 |
| endo bFGF KC | | 40 | 0 | 0.00 | endo bFGF | −2.00 |
| heart Clontech | normal | 26.09 | 1982.44 | 3964.88 | heart | |
| heart (T-1) ischemic | 29417 | 24 | 6956.27 | 13912.54 | heart T-1 | 3.51 |
| heart (T-14) non- | 29422 | 24.55 | 5010.03 | 10020.06 | heart T-14 | 2.53 |
| obstructive DCM | | | | | | |
| heart (T-3399) DCM | 29426 | 24.05 | 6766.57 | 13533.14 | heart T-3399 | 3.41 |
| adenoid GW99-269 | 26162 | 30.56 | 134.11 | 268.22 | adenoid | |
| tonsil GW98-280 | 22582 | 27.94 | 651.01 | 1302.02 | tonsil | |
| T cells PC00314 | 28453 | 29.8 | 212.45 | 424.90 | T cells | |
| PBMNC | | 33.01 | 30.69 | 30.69 | PBMNC | |
| monocyte | | 33.42 | 23.9 | 47.80 | monocyte | |
| B cells PC00665 | 28455 | 33.52 | 22.59 | 45.18 | B cells | |
| dendritic cells 28441 | | 29.07 | 329.58 | 659.16 | dendritic cells | |
| neutrophils | 28440 | 30.39 | 149 | 149.00 | neutrophils | |
| eosinophils | 28446 | 35.41 | 7.25 | 14.50 | eosinophils | |
| BM unstim | | 34.24 | 14.65 | 14.65 | BM unstim | |
| BM stim | | 36.61 | 3.51 | 3.51 | BM stim | −4.17 |
| osteo dif | | 30.55 | 135.33 | 135.33 | osteo dif | 3.02 |
| osteo undif | | 32.38 | 44.88 | 44.88 | osteo undif | |
| chondrocytes | | 25.35 | 3089.54 | 7723.85 | chondrocytes | |
| OA Synovium IP12/01 | 29462 | 28.75 | 398.53 | 398.53 | OA Synovium | |
| OA Synovium NP10/01 | 29461 | 27.04 | 1119.77 | 2239.54 | OA Synovium | |
| OA Synovium NP57/00 | 28464 | 28.85 | 375.92 | 751.84 | OA Synovium | |
| RA Synovium NP03/01 | 28466 | 28.14 | 574.66 | 1149.32 | RA Synovium | |
| RA Synovium NP71/00 | 28467 | 27.58 | 806.11 | 1612.22 | RA Synovium | |
| RA Synovium NP45/00 | 28475 | 28.04 | 611.1 | 1222.20 | RA Synovium | |
| OA bone (biobank) | 29217 | 30.25 | 161.3 | 161.30 | OA bone | |
| | | | | | (biobank) | |
| OA bone Sample 1 | J. Emory | 31.24 | 89.29 | 178.58 | OA bone | |
| OA bone Sample 2 | J. Emory | 30.98 | 104.34 | 208.68 | OA bone | |
| Cartilage (pool) | Normal | 29.86 | 204.47 | 408.94 | Cartilage (pool) | |
| Cartilage (pool) | OA | 29.37 | 275.09 | 550.18 | Cartilage (pool) | 1.35 |
| PBL unifected | 28441 | 26.45 | 1598.39 | 3196.78 | PBL unifected | |
| PBL HIV IIIB | 28442 | 27.57 | 814.58 | 1629.16 | PBL HIV IIIB | −1.96 |
| MRC5 uninfected | 29158 | 25.13 | 3539.95 | 7079.90 | MRC5 uninfected | |
| (100%) | | | | | (100%) | |
| MRC5 HSV strain F | 29178 | 30.49 | 139.89 | 279.78 | MRC5 HSV strain F | −25.31 |
| W12 cells | 29179 | 26.72 | 1359.04 | 2718.08 | W12 cells | |
| Keratinocytes | 29180 | 26.41 | 1633.77 | 3267.54 | Keratinocytes | |
| B-actin control | | 27.87 | 678.8 | | | |
| genomic | | 26.9 | 1214.71 | | | |
| 1.00E+05 | | 19.86 | 100000 | | | |
| 1.00E+05 | | 19.82 | 100000 | | | |
| 1.00E+04 | | 23.15 | 10000 | | | |
| 1.00E+04 | | 23.21 | 10000 | | | |
| 1.00E+03 | | 26.62 | 1000 | | | |
| 1.00E+03 | | 26.79 | 1000 | | | |
| 1.00E+02 | | 31.2 | 100 | | | |
| 1.00E+02 | | 32.2 | 100 | | | |
| 1.00E+01 | | 40 | 0 | | | |
| 1.00E+01 | | 34.53 | 10 | | | |
| 1.00E−00 | | 40 | 0 | | | |
| 1.00E−00 | | 40 | 0 | | | |
| NTC | | 40 | 0 | | | |

Gene Name sbg98530TS

| | Fold Change in Disease |
|---|---|
| | Population Relative to |
| Disease tissues | Normal |
| colon tumor | 1.04 |
| colon tumor | 1.39 |
| colon tumor | 5.78 |
| colon tumor | 3.18 |
| lung tumor | −1.15 |
| lung tumor | 1.81 |
| lung tumor | 1.28 |
| lung tumor | −2.05 |
| breast tumor | 4.20 |
| breast tumor | 11.75 |
| breast tumor | 6.80 |
| breast tumor | 1.01 |
| brain stage 5 ALZ | 1.80 |
| brain stage 5 ALZ | 3.64 |
| brain stage 5 ALZ | 3.40 |
| brain stage 5 ALZ | 1.09 |
| lung 24 | −325.52 |
| lung 28 | −42.11 |
| lung 23 | −96.02 |
| asthmatic lung | −3.19 |
| asthmatic lung | 1.33 |
| asthmatic lung | 1.28 |
| asthmatic lung | −1.08 |
| endo VEGF | 2.92 |
| endo bFGF | −2.00 |
| heart T-1 | 3.51 |
| heart T-14 | 2.53 |
| heart T-3399 | 3.41 |
| BM stim | −4.17 |
| osteo undif | 3.02 |
| Cartilage (pool) | 1.35 |
| PBL HIV IIIB | −1.96 |
| MRC5 HSV strain F | −25.31 |

Gene Name sbg563917RDP

Moderate to low overall expression in normal and disease samples. Highest normal expression in testis liver, trachea, and whole brain. Shows good expression in most of the GI tract samples. Highest disease expression in T cells, B cells, neutrophils, and eosinophils. Upregulation in 1 of 4 breast tumors implies involvement in cancer of the breast. Downregulation in 3 of 3 COPD lungs suggests an involvement in chronic obstructive pulmonary disease. Downregulation in the ischemic heart sample implicates this gene in ischemic heart disease. Downregulation in the VEGF and bFGF-treated endothelial cells suggests a role in angiogenesis. Upregulation in HSV implicates involvement in herpes simplex virus as a potential host factor.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg563917RDP | 1 and 2) | 1) | 2) | Copies | (ng) | (ng) | RNA |
| Subcutaneous | 35.34, 40 | 3.66 | 0 | 1.83 | 3.06 | 16.34 | 29.90 |
| Adipocytes Zenbio | | | | | | | |
| Subcutaneous Adipose | 40, 40 | 0 | | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 30.09, 30.01 | 91.85 | 96.8 | 94.33 | 7.24 | 6.91 | 651.42 |
| Fetal Brain Clontech | 40, 40 | 0 | 0 | 0.00 | 0.48 | 103.95 | 0.00 |
| Cerebellum Clontech | 35.19, 40 | 4.03 | 0 | 2.02 | 2.17 | 23.04 | 46.43 |
| Cervix | 36.08, 40 | 2.33 | 0 | 1.17 | 2.42 | 20.66 | 24.07 |
| Colon | 36.07, 35.1 | 2.35 | 4.24 | 3.30 | 2.71 | 18.45 | 60.79 |
| Endometrium | 35.01, 40 | 4.49 | 0 | 2.25 | 0.73 | 68.21 | 153.14 |
| Esophagus | 34.94, 40 | 4.68 | 0 | 2.34 | 1.37 | 36.50 | 85.40 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 35.24, 40 | 3.89 | 0 | 1.95 | 2.58 | 19.38 | 37.69 |
| Jejunum | 35.37, 33.35 | 3.6 | 12.44 | 8.02 | 6.60 | 7.58 | 60.76 |
| Kidney | 40, 34.97 | 0 | 4.6 | 2.30 | 2.12 | 23.58 | 54.25 |
| Liver | 33.51, 34.6 | 11.25 | 5.78 | 8.52 | 1.50 | 33.33 | 283.83 |
| Fetal Liver Clontech | 33.19, 35.54 | 13.75 | 3.25 | 8.50 | 10.40 | 4.81 | 40.87 |
| Lung | 34.32 | 6.84 | 2.28 | 4.56 | 2.57 | 19.46 | 88.72 |
| Mammary Gland | 40, 35.14 | 0 | 4.15 | 2.08 | 13.00 | 3.85 | 7.98 |
| Clontech | | | | | | | |
| Myometrium | 40, 40 | 0 | 0.09 | 0.05 | 2.34 | 21.37 | 0.96 |
| Omentum | 36.17, 33.01 | 2.2 | 15.32 | 8.76 | 3.94 | 12.69 | 111.17 |
| Ovary | 40, 40 | 0 | 0 | 0.00 | 4.34 | 11.52 | 0.00 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 35.43, 39.81 | 3.46 | 0.24 | 1.85 | 5.48 | 9.12 | 16.88 |
| Placenta Clontech | 33.7, 35.45 | 10.02 | 3.42 | 6.72 | 5.26 | 9.51 | 63.88 |
| Prostate | 40, 40 | 0 | 0 | 0.00 | 3.00 | 16.67 | 0.00 |
| Rectum | 40, 40 | 0 | 0 | 0.00 | 1.23 | 40.65 | 0.00 |
| Salivary Gland | 34.89, 40 | 4.83 | 0 | 2.42 | 7.31 | 6.84 | 16.52 |
| Clontech | | | | | | | |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine | 40, 40 | 0 | 0 | 0.00 | 0.98 | 51.07 | 0.00 |
| Clontech | | | | | | | |
| Spleen | 35.01, 33.36 | 4.48 | 12.38 | 8.43 | 4.92 | 10.16 | 85.67 |
| Stomach | 33.38, 34.18 | 12.18 | 7.48 | 9.83 | 2.73 | 18.32 | 180.04 |
| Testis Clontech | 34.25, 32.86 | 7.17 | 16.84 | 12.01 | 0.57 | 87.87 | 1054.92 |
| Thymus Clontech | 32.14, 33.25 | 26.17 | 13.24 | 19.71 | 9.89 | 5.06 | 99.62 |
| Thyroid | 40, 40 | 0 | 0.09 | 0.05 | 2.77 | 18.05 | 0.81 |
| Trachea Clontech | 31.41, 31 | 41.04 | 52.65 | 46.85 | 9.71 | 5.15 | 241.22 |
| Urinary Bladder | 40, 35.05 | 0 | 4.38 | 2.19 | 5.47 | 9.14 | 20.02 |
| Uterus | 33.77, 33.41 | 9.62 | 12 | 10.81 | 5.34 | 9.36 | 101.22 |
| genomic | 26.54 | 813.56 | | | | | |
| b-actin | 27.39 | 481.34 | | | | | |
| 1.00E+05 | 18.71 | 100000 | | | | | |
| 1.00E+05 | 18.92 | 100000 | | | | | |
| 1.00E+04 | 22.44 | 10000 | | | | | |
| 1.00E+04 | 22.11 | 10000 | | | | | |
| 1.00E+03 | 26.05 | 1000 | | | | | |
| 1.00E+03 | 26.11 | 1000 | | | | | |
| 1.00E+02 | 30.4 | 100 | | | | | |
| 1.00E+02 | 30.17 | 100 | | | | | |
| 1.00E+01 | 33.87 | 10 | | | | | |
| 1.00E+01 | 33.26 | 10 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | |
| | Reg | | | detected/ | | Fold |
| | number | | Mean | 50 ng | | Change in |
| Sample | (GSK | | GOI | total | | Disease |
| sbg563917RDP | identifier) | Ct | copies | RNA | Sample | Population |
| colon normal GW98-167 | 21941 | 30.39 | 148.35 | 296.70 | colon normal | |
| colon tumor GW98-166 | 21940 | 30.93 | 110.14 | 220.28 | colon tumor | −1.35 |
| colon normal GW98-178 | 22080 | 32.93 | 36.71 | 73.42 | colon normal | |
| colon tumor GW98-177 | 22060 | 34.8 | 13.09 | 26.18 | colon tumor | −2.80 |
| colon normal GW98-561 | 23514 | 31.41 | 84.68 | 169.36 | colon normal | |
| colon tumor GW98-560 | 23513 | 32.09 | 58.33 | 116.66 | colon tumor | −1.45 |
| colon normal GW98-894 | 24691 | 30.02 | 182.15 | 364.30 | colon normal | |
| colon tumor GW98-893 | 24690 | 31.12 | 99.26 | 198.52 | colon tumor | −1.84 |
| lung normal GW98-3 | 20742 | 28.4 | 443.99 | 887.98 | lung normal | |
| lung tumor GW98-2 | 20741 | 33.59 | 25.44 | 50.88 | lung tumor | −17.45 |
| lung normal GW97-179 | 20677 | 28.63 | 391.85 | 783.70 | lung normal | |
| lung tumor GW97-178 | 20676 | 29.16 | 292.08 | 584.16 | lung tumor | −1.34 |
| lung normal GW98-165 | 21922 | 29.13 | 296.8 | 593.60 | lung normal | |
| lung tumor GW98-164 | 21921 | 30.22 | 163.43 | 326.86 | lung tumor | −1.82 |
| lung normal GW98-282 | 22584 | 31.71 | 71.72 | 143.44 | lung normal | |
| lung tumor GW98-281 | 22583 | 31.74 | 70.77 | 141.54 | lung tumor | −1.01 |
| breast normal GW00-392 | 28750 | 31.49 | 81.02 | 81.02 | breast normal | |
| breast tumor GW00-391 | 28746 | 33.58 | 25.62 | 51.24 | breast tumor | −1.58 |
| breast normal GW00-413 | 28798 | 35.07 | 11.31 | 11.31 | breast normal | |
| breast tumor GW00-412 | 28797 | 31.99 | 61.52 | 123.04 | breast tumor | 10.88 |
| breast normal GW00- | 27592-95 | 35.63 | 8.3 | 8.30 | breast normal | |
| 235:238 | | | | | | |
| breast tumor GW00- | 27588-91 | 34.33 | 16.97 | 16.97 | breast tumor | 2.04 |
| 231:234 | | | | | | |
| breast normal GW98-621 | 23656 | 32.07 | 58.95 | 117.90 | breast normal | |
| breast tumor GW98-620 | 23655 | 32.71 | 41.33 | 82.66 | breast tumor | −1.43 |
| brain normal BB99-542 | 25507 | 30.16 | 168.86 | 337.72 | brain normal | |
| brain normal BB99-406 | 25509 | 31.12 | 99.35 | 198.70 | brain normal | |
| brain normal BB99-904 | 25546 | 31.14 | 98.44 | 196.88 | brain normal | |
| brain stage 5 ALZ BB99- | 25502 | 33.16 | 32.39 | 64.78 | brain stage 5 ALZ | −3.77 |
| 874 | | | | | | |
| brain stage 5 ALZ BB99- | 25503 | 29.32 | 267.28 | 534.56 | brain stage 5 ALZ | 2.19 |
| 887 | | | | | | |
| brain stage 5 ALZ BB99- | 25504 | 30.36 | 150.72 | 301.44 | brain stage 5 ALZ | 1.23 |
| 862 | | | | | | |
| brain stage 5 ALZ BB99- | 25542 | 30.1 | 174.01 | 348.02 | brain stage 5 ALZ | 1.42 |
| 927 | | | | | | |
| CT lung KC | normal | 31.06 | 102.88 | 205.76 | CT lung | |
| lung 26 KC | normal | 32.15 | 56.21 | 56.21 | lung 26 | |
| lung 27 KC | normal | 35.96 | 6.92 | 6.92 | lung 27 | |
| lung 24 KC | COPD | 40 | 0 | 0.00 | lung 24 | −70.34 |
| lung 28 KC | COPD | 36.21 | 6.05 | 6.05 | lung 28 | −11.63 |
| lung 23 KC | COPD | 34.83 | 12.87 | 12.87 | lung 23 | −5.47 |
| lung 25 KC | normal | 34.89 | 12.45 | 12.45 | lung 25 | |
| asthmatic lung ODO3112 | 29321 | 32.57 | 44.76 | 44.76 | asthmatic lung | −1.57 |
| asthmatic lung ODO3433 | 29323 | 32.4 | 49.04 | 98.08 | asthmatic lung | 1.39 |
| asthmatic lung ODO3397 | 29322 | 31.79 | 68.64 | 137.28 | asthmatic lung | 1.95 |
| asthmatic lung ODO4928 | 29325 | 31.34 | 88.11 | 176.22 | asthmatic lung | 2.51 |
| endo cells KC | control | 35.77 | 7.68 | 7.68 | endo cells | |
| endo VEGF KC | | 40 | 0 | 0.00 | endo VEGF | −7.68 |
| endo bFGF KC | | 40 | 0 | 0.00 | endo bFGF | −7.68 |
| heart Clontech | normal | 31.09 | 100.75 | 201.50 | heart | |
| heart (T-1) ischemic | 29417 | 34.75 | 13.46 | 26.92 | heart T-1 | −7.49 |
| heart (T-14) non- | 29422 | 33.69 | 24.17 | 48.34 | heart T-14 | −4.17 |
| obstructive DCM | | | | | | |
| heart (T-3399) DCM | 29426 | 33.48 | 27.16 | 54.32 | heart T-3399 | −3.71 |
| adenoid GW99-269 | 26162 | 30.49 | 140.7 | 281.40 | adenoid | |
| tonsil GW98-280 | 22582 | 30.07 | 177.32 | 354.64 | tonsil | |
| T cells PC00314 | 28453 | 27.79 | 622.1 | 1244.20 | T cells | |
| PBMNC | | 36.19 | 6.11 | 6.11 | PBMNC | |
| monocyte | | 33.24 | 30.91 | 61.82 | monocyte | |
| B cells PC00665 | 28455 | 26.37 | 1355.2 | 2710.40 | B cells | |
| dendritic cells 28441 | | 28.69 | 378.62 | 757.24 | dendritic cells | |
| neutrophils | 28440 | 23.28 | 7420.47 | 7420.47 | neutrophils | |
| eosinophils | 28446 | 25.33 | 2408.94 | 4817.88 | eosinophils | |
| BM unstim | | 32.24 | 53.57 | 53.57 | BM unstim | |
| BM stim | | 31.92 | 64.05 | 64.05 | BM stim | 1.20 |
| osteo dif | | 40 | 0 | 0.00 | osteo dif | 0.00 |
| osteo undif | | 40 | 0 | 0.00 | osteo undif | |
| chondrocytes | | 37.59 | 2.82 | 7.05 | chondrocytes | |
| OA Synovium IP12/01 | 29462 | 31.77 | 69.48 | 69.48 | OA Synovium | |
| OA Synovium NP10/01 | 29461 | 31.16 | 96.93 | 193.86 | OA Synovium | |
| OA Synovium NP57/00 | 28464 | 30.84 | 115.69 | 231.38 | OA Synovium | |
| RA Synovium NP03/01 | 28466 | 30.96 | 108.37 | 216.74 | RA Synovium | |
| RA Synovium NP71/00 | 28467 | 29.2 | 285.91 | 571.82 | RA Synovium | |
| RA Synovium NP45/00 | 28475 | 29.87 | 198.12 | 396.24 | RA Synovium | |
| OA bone (biobank) | 29217 | 29.67 | 220.64 | 220.64 | OA bone | |
| | | | | | (biobank) | |
| OA bone Sample 1 | J. Emory | 30.54 | 136.41 | 272.82 | OA bone | |
| OA bone Sample 2 | J. Emory | 29.38 | 259.07 | 518.14 | OA bone | |
| Cartilage (pool) | Normal | 31.34 | 87.88 | 175.76 | Cartilage (pool) | |
| Cartilage (pool) | OA | 32.9 | 37.23 | 74.46 | Cartilage (pool) | −2.36 |
| PBL unifected | 28441 | 30.55 | 135.85 | 271.70 | PBL unifected | |
| PBL HIV IIIB | 28442 | 31.02 | 104.8 | 209.60 | PBL HIV IIIB | −1.30 |
| MRC5 uninfected | 29158 | 35.11 | 11.06 | 22.12 | MRC5 uninfected | |
| (100%) | | | | | (100%) | |
| MRC5 HSV strain F | 29178 | 29.63 | 226.14 | 452.28 | MRC5 HSV strain F | 20.45 |
| W12 cells | 29179 | 37.87 | 2.42 | 4.84 | W12 cells | |
| Keratinocytes | 29180 | 36.14 | 6.26 | 12.52 | Keratinocytes | |
| B-actin control | | 27.14 | 887.42 | | | |
| genomic | | 26.16 | 1520.17 | | | |
| 1.00E+05 | | 19.22 | 100000 | | | |
| 1.00E+05 | | 19.2 | 100000 | | | |
| 1.00E+04 | | 22.49 | 10000 | | | |
| 1.00E+04 | | 22.62 | 10000 | | | |
| 1.00E+03 | | 26.23 | 1000 | | | |
| 1.00E+03 | | 26.05 | 1000 | | | |
| 1.00E+02 | | 30.26 | 100 | | | |
| 1.00E+02 | | 31.03 | 100 | | | |
| 1.00E+01 | | 38.68 | 10 | | | |
| 1.00E+01 | | 33.47 | 10 | | | |
| 1.00E−00 | | 40 | 0 | | | |
| 1.00E−00 | | 40 | 0 | | | |
| NTC | | 40 | 0 | | | |

Gene Name sbg563917RDP

| | Fold Change in Disease |
|---|---|
| | Population Relative to |
| Disease tissues | Normal |
| colon tumor | −1.35 |
| colon tumor | −2.80 |
| colon tumor | −1.45 |
| colon tumor | −1.84 |
| lung tumor | −17.45 |
| lung tumor | −1.34 |
| lung tumor | −1.82 |
| lung tumor | −1.01 |
| breast tumor | −1.58 |
| breast tumor | 10.88 |
| breast tumor | 2.04 |
| breast tumor | −1.43 |
| brain stage 5 ALZ | −3.77 |
| brain stage 5 ALZ | 2.19 |
| brain stage 5 ALZ | 1.23 |
| brain stage 5 ALZ | 1.42 |
| lung 24 | −70.34 |
| lung 28 | −11.63 |
| lung 23 | −5.47 |
| asthmatic lung | −1.57 |
| asthmatic lung | 1.39 |
| asthmatic lung | 1.95 |
| asthmatic lung | 2.51 |
| endo VEGF | −7.68 |
| endo bFGF | −7.68 |
| heart T-1 | −7.49 |
| heart T-14 | −4.17 |
| heart T-3399 | −3.71 |
| BM stim | 1.20 |
| osteo undif | 0.00 |
| Cartilage (pool) | −2.36 |
| PBL HIV IIIB | −1.30 |
| MRC5 HSV strain F | 20.45 |

Gene Name sbg618069LRR

Low overall expression in normal and disease samples. Highest normal expression in whole brain, fetal brain, cerebellum, and thymus. Highest disease expression in one of the colon tumor samples, one of the lung tumor samples, and the uninfected PBL cells. Downregulation in 2 of 4 colon tumors suggests a role in cancer of the colon. Upregulation in 1 of 4 lung tumors and upregulation in 2 of 4 breast tumors suggest roles in cancers of the lung and breast. Downregulation in 3 of 3 COPD lung samples implicates a role for this gene in COPD. Upregulation in the stimulated bone marrow. Downregulation in an HIV-infected cell line as well as moderate expression in immune cells suggests an involvement in HIV. Upregulation in HSV implicates involvement in herpes simplex virus as a potential host factor.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg618069LRR | 1 and 2) | 1) | 2) | Copies | (ng) | (ng) | RNA |
| Subcutaneous | 35.24, 34.33 | 4.38 | 7.65 | 6.02 | 3.06 | 16.34 | 98.28 |
| Adipocytes Zenbio | | | | | | | |
| Subcutaneous Adipose | 40, 40 | 0 | 0.08 | 0.04 | 0.96 | 52.36 | 2.09 |
| Zenbio | | | | | | | |
| Adrenal Gland Clontech | 39.9, 38.74 | 0.13 | 0.51 | 0.32 | 0.61 | 81.97 | 26.23 |
| Whole Brain Clontech | 27.02, 26.51 | 673.36 | 916.93 | 795.15 | 7.24 | 6.91 | 5491.33 |
| Fetal Brain Clontech | 40, 40 | 0.13 | 6.01 | 3.07 | 0.48 | 103.95 | 319.13 |
| Cerebellum Clontech | 32.15, 32.13 | 28.98 | 29.44 | 29.21 | 2.17 | 23.04 | 673.04 |
| Cervix | 40, 40 | 0 | 0 | 0.00 | 2.42 | 20.66 | 0.00 |
| Colon | 36.53, 39.88 | 1.98 | 0.25 | 1.12 | 2.71 | 18.45 | 20.57 |
| Endometrium | 37.98, 40 | 0.82 | 0.19 | 0.51 | 0.73 | 68.21 | 34.45 |
| Esophagus | 38.86, 40 | 0.48 | 0.15 | 0.32 | 1.37 | 36.50 | 11.50 |
| Heart Clontech | 34.17, 34.72 | 8.41 | 6.03 | 7.22 | 1.32 | 37.88 | 273.48 |
| Hypothalamus | 40, 40 | 0 | 0.07 | 0.04 | 0.32 | 155.28 | 5.43 |
| Ileum | 40, 40 | 0.17 | 0.11 | 0.14 | 2.58 | 19.38 | 2.71 |
| Jejunum | 33.07, 34.34 | 16.52 | 7.58 | 12.05 | 6.60 | 7.58 | 91.29 |
| Kidney | 36.05, 40 | 2.67 | 0.21 | 1.44 | 2.12 | 23.58 | 33.96 |
| Liver | 38.72, 40 | 0.52 | 0.61 | 0.57 | 1.50 | 33.33 | 18.83 |
| Fetal Liver Clontech | 33.28, 36.35 | 14.52 | 2.22 | 8.37 | 10.40 | 4.81 | 40.24 |
| Lung | 40, 40 | 0.13 | 0.08 | 0.11 | 2.57 | 19.46 | 2.04 |
| Mammary Gland | 40, 34.19 | 0 | 8.33 | 4.17 | 13.00 | 3.85 | 16.02 |
| Clontech | | | | | | | |
| Myometrium | 40, 40 | 0.28 | 0 | 0.14 | 2.34 | 21.37 | 2.99 |
| Omentum | 35.01, 35.36 | 5.04 | 4.07 | 4.56 | 3.94 | 12.69 | 57.80 |
| Ovary | 34.24, 40 | 8.07 | 0.26 | 4.17 | 4.34 | 11.52 | 47.98 |
| Pancreas | 40, 40 | 0.11 | 0.13 | 0.12 | 0.81 | 61.80 | 7.42 |
| Head of Pancreas | 40, 40 | 0 | 0 | 0.00 | 1.57 | 31.85 | 0.00 |
| Parotid Gland | 34.01, 34.28 | 9.31 | 7.9 | 8.61 | 5.48 | 9.12 | 78.51 |
| Placenta Clontech | 40, 40 | 0 | 0.07 | 0.04 | 5.26 | 9.51 | 0.33 |
| Prostate | 40, 39.13 | 0.15 | 0.4 | 0.28 | 3.00 | 16.67 | 4.58 |
| Rectum | 40, 39.55 | 0.21 | 0.31 | 0.26 | 1.23 | 40.65 | 10.57 |
| Salivary Gland | 32.35, 33.08 | 25.74 | 16.45 | 21.10 | 7.31 | 6.84 | 144.29 |
| Clontech | | | | | | | |
| Skeletal Muscle | 34.78, 40 | 5.81 | 0.27 | 3.04 | 1.26 | 39.68 | 120.63 |
| Clontech | | | | | | | |
| Skin | 40, 40 | 0 | 0 | 0.00 | 1.21 | 41.32 | 0.00 |
| Small Intestine | 40, 40 | 0.16 | 0 | 0.08 | 0.98 | 51.07 | 4.09 |
| Clontech | | | | | | | |
| Spleen | 35.59, 40 | 3.53 | 0.59 | 2.06 | 4.92 | 10.16 | 20.93 |
| Stomach | 36.73, 40 | 1.76 | 0.11 | 0.94 | 2.73 | 18.32 | 17.12 |
| Testis Clontech | 37.91, 40 | 0.86 | 0.1 | 0.48 | 0.57 | 87.87 | 42.18 |
| Thymus Clontech | 30.22, 29.94 | 94.88 | 112.23 | 103.56 | 9.89 | 5.06 | 523.53 |
| Thyroid | 35.15, 40 | 4.62 | 0 | 2.31 | 2.77 | 18.05 | 41.70 |
| Trachea Clontech | 33.49, 34.21 | 12.75 | 8.22 | 10.49 | 9.71 | 5.15 | 53.99 |
| Urinary Bladder | 40, 40 | 0.09 | 0.08 | 0.09 | 5.47 | 9.14 | 0.78 |
| Uterus | 35.26, 33.03 | 4.31 | 16.97 | 10.64 | 5.34 | 9.36 | 99.63 |
| genomic | 26.04 | 1229.54 | | | | | |
| b-actin | 27.25 | 584.19 | | | | | |
| 1.00E+05 | 19.09 | 100000 | | | | | |
| 1.00E+05 | 19.04 | 100000 | | | | | |
| 1.00E+04 | 22.35 | 10000 | | | | | |
| 1.00E+04 | 22.35 | 10000 | | | | | |
| 1.00E+03 | 26.07 | 1000 | | | | | |
| 1.00E+03 | 26.26 | 1000 | | | | | |
| 1.00E+02 | 30.64 | 100 | | | | | |
| 1.00E+02 | 30.38 | 100 | | | | | |
| 1.00E+01 | 34.04 | 10 | | | | | |
| 1.00E+01 | 33.52 | 10 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | Fold |
| | Reg | | | detected/ | | Change |
| | number | | Mean | 50 ng | | in |
| Sample | (GSK | | GOI | total | | Disease |
| sbg618069LRR | identifier) | Ct | copies | RNA | Sample | Population |
| colon normal GW98-167 | 21941 | 29.52 | 176.51 | 353.02 | colon normal | |
| colon tumor GW98-166 | 21940 | 33.45 | 18.5 | 37.00 | colon tumor | −9.54 |
| colon normal GW98-178 | 22080 | 31.82 | 47.14 | 94.28 | colon normal | |
| colon tumor GW98-177 | 22060 | 29.68 | 160.67 | 321.34 | colon tumor | 3.41 |
| colon normal GW98-561 | 23514 | 30.33 | 110.78 | 221.56 | colon normal | |
| colon tumor GW98-560 | 23513 | 33.11 | 22.39 | 44.78 | colon tumor | −4.95 |
| colon normal GW98-894 | 24691 | 28.1 | 396.95 | 793.90 | colon normal | |
| colon tumor GW98-893 | 24690 | 26.93 | 779.99 | 1559.98 | colon tumor | 1.96 |
| lung normal GW98-3 | 20742 | 30.41 | 105.78 | 211.56 | lung normal | |
| lung tumor GW98-2 | 20741 | 26.28 | 1128.28 | 2256.56 | lung tumor | 10.67 |
| lung normal GW97-179 | 20677 | 29.28 | 201.91 | 403.82 | lung normal | |
| lung tumor GW97-178 | 20676 | 28.35 | 345.36 | 690.72 | lung tumor | 1.71 |
| lung normal GW98-165 | 21922 | 28.42 | 331.95 | 663.90 | lung normal | |
| lung tumor GW98-164 | 21921 | 30.98 | 76.05 | 152.10 | lung tumor | −4.36 |
| lung normal GW98-282 | 22584 | 34.15 | 12.36 | 24.72 | lung normal | |
| lung tumor GW98-281 | 22583 | 32.08 | 40.6 | 81.20 | lung tumor | 3.28 |
| breast normal GW00-392 | 28750 | 29.67 | 161.68 | 161.68 | breast normal | |
| breast tumor GW00-391 | 28746 | 28.98 | 239.65 | 479.30 | breast tumor | 2.96 |
| breast normal GW00-413 | 28798 | 31.78 | 48.04 | 48.04 | breast normal | |
| breast tumor GW00-412 | 28797 | 29.69 | 159.55 | 319.10 | breast tumor | 6.64 |
| breast normal GW00- | 27592-95 | 34.18 | 12.14 | 12.14 | breast normal | |
| 235:238 | | | | | | |
| breast tumor GW00- | 27588-91 | 29.2 | 211.28 | 211.28 | breast tumor | 17.40 |
| 231:234 | | | | | | |
| breast normal GW98-621 | 23656 | 29.72 | 157.4 | 314.80 | breast normal | |
| breast tumor GW98-620 | 23655 | 31.12 | 70.17 | 140.34 | breast tumor | −2.24 |
| brain normal BB99-542 | 25507 | 30.81 | 83.89 | 167.78 | brain normal | |
| brain normal BB99-406 | 25509 | 31.02 | 74.28 | 148.56 | brain normal | |
| brain normal BB99-904 | 25546 | 31.39 | 60.08 | 120.16 | brain normal | |
| brain stage 5 ALZ BB99- | 25502 | 32.54 | 31.07 | 62.14 | brain stage 5 ALZ | −2.34 |
| 874 | | | | | | |
| brain stage 5 ALZ BB99- | 25503 | 30.65 | 92.1 | 184.20 | brain stage 5 ALZ | 1.27 |
| 887 | | | | | | |
| brain stage 5 ALZ BB99- | 25504 | 31.68 | 50.92 | 101.84 | brain stage 5 ALZ | −1.43 |
| 862 | | | | | | |
| brain stage 5 ALZ BB99- | 25542 | 31.39 | 60.36 | 120.72 | brain stage 5 ALZ | −1.21 |
| 927 | | | | | | |
| CT lung KC | normal | 30.47 | 101.87 | 203.74 | CT lung | |
| lung 26 KC | normal | | | | lung 26 | |
| lung 27 KC | normal | 39.27 | 0.65 | 0.65 | lung 27 | |
| lung 24 KC | COPD | 40 | 0 | 0.00 | lung 24 | −68.13 |
| lung 28 KC | COPD | 37.38 | 1.93 | 1.93 | lung 28 | −35.30 |
| lung 23 KC | COPD | 34.28 | 11.47 | 11.47 | lung 23 | −5.94 |
| lung 25 KC | normal | 40 | 0 | 0.00 | lung 25 | |
| asthmatic lung ODO3112 | 29321 | 33.17 | 21.73 | 21.73 | asthmatic lung | −3.14 |
| asthmatic lung ODO3433 | 29323 | 32.35 | 34.64 | 69.28 | asthmatic lung | 1.02 |
| asthmatic lung ODO3397 | 29322 | 30.83 | 83.1 | 166.20 | asthmatic lung | 2.44 |
| asthmatic lung ODO4928 | 29325 | 30.94 | 77.99 | 155.98 | asthmatic lung | 2.29 |
| endo cells KC | control | 40 | 0 | 0.00 | endo cells | |
| endo VEGF KC | | 40 | 0 | 0.00 | endo VEGF | 0.00 |
| endo bFGF KC | | 40 | 0 | 0.00 | endo bFGF | 0.00 |
| heart Clontech | normal | 30.52 | 99.45 | 198.90 | heart | |
| heart (T-1) ischemic | 29417 | 28.78 | 270.18 | 540.36 | heart T-1 | 2.72 |
| heart (T-14) non- | 29422 | 29.62 | 166.76 | 333.52 | heart T-14 | 1.68 |
| obstructive DCM | | | | | | |
| heart (T-3399) DCM | 29426 | 30.05 | 129.76 | 259.52 | heart T-3399 | 1.30 |
| adenoid GW99-269 | 26162 | 29.05 | 230.93 | 461.86 | adenoid | |
| tonsil GW98-280 | 22582 | 29.77 | 152.22 | 304.44 | tonsil | |
| T cells PC00314 | 28453 | 31.6 | 53.48 | 106.96 | T cells | |
| PBMNC | | 39.8 | 0.48 | 0.48 | PBMNC | |
| monocyte | | 40 | 0 | 0.00 | monocyte | |
| B cells PC00665 | 28455 | 31.56 | 54.77 | 109.54 | B cells | |
| dendritic cells 28441 | | 34.09 | 12.8 | 25.60 | dendritic cells | |
| neutrophils | 28440 | 34.03 | 13.21 | 13.21 | neutrophils | |
| eosinophils | 28446 | 40 | 0 | 0.00 | eosinophils | |
| BM unstim | | 40 | 0 | 0.00 | BM unstim | |
| BM stim | | 35.71 | 5.04 | 5.04 | BM stim | 5.04 |
| osteo dif | | 40 | 0 | 0.00 | osteo dif | 0.00 |
| osteo undif | | 40 | 0 | 0.00 | osteo undif | |
| chondrocytes | | 33.5 | 17.89 | 44.73 | chondrocytes | |
| OA Synovium IP12/01 | 29462 | 32.24 | 37.02 | 37.02 | OA Synovium | |
| OA Synovium NP10/01 | 29461 | 27.95 | 434.95 | 869.90 | OA Synovium | |
| OA Synovium NP57/00 | 28464 | 30.9 | 79.82 | 159.64 | OA Synovium | |
| RA Synovium NP03/01 | 28466 | 31.79 | 47.9 | 95.80 | RA Synovium | |
| RA Synovium NP71/00 | 28467 | 33.06 | 23.05 | 46.10 | RA Synovium | |
| RA Synovium NP45/00 | 28475 | 32.76 | 27.41 | 54.82 | RA Synovium | |
| OA bone (biobank) | 29217 | 32.02 | 42.02 | 42.02 | OA bone | |
| | | | | | (biobank) | |
| OA bone Sample 1 | J. Emory | 31.32 | 62.63 | 125.26 | OA bone | |
| OA bone Sample 2 | J. Emory | 33.13 | 22.24 | 44.48 | OA bone | |
| Cartilage (pool) | Normal | 30.76 | 86.51 | 173.02 | Cartilage (pool) | |
| Cartilage (pool) | OA | 33.13 | 22.15 | 44.30 | Cartilage (pool) | −3.91 |
| PBL unifected | 28441 | 26.78 | 847.01 | 1694.02 | PBL unifected | |
| PBL HIV IIIB | 28442 | 28.21 | 374.26 | 748.52 | PBL HIV IIIB | −2.26 |
| MRC5 uninfected | 29158 | 40 | 0 | 0.00 | MRC5 uninfected | |
| (100%) | | | | | (100%) | |
| MRC5 HSV strain F | 29178 | 31.26 | 64.96 | 129.92 | MRC5 HSV strain F | 129.92 |
| W12 cells | 29179 | 40 | 0 | 0.00 | W12 cells | |
| Keratinocytes | 29180 | 39.77 | 0.49 | 0.98 | Keratinocytes | |
| B-actin control | | 27.18 | 675.17 | | | |
| genomic | | 25.96 | 1358.6 | | | |
| 1.00E+05 | | 18.58 | 100000 | | | |
| 1.00E+05 | | 18.53 | 100000 | | | |
| 1.00E+04 | | 22.04 | 10000 | | | |
| 1.00E+04 | | 22.1 | 10000 | | | |
| 1.00E+03 | | 25.85 | 1000 | | | |
| 1.00E+03 | | 26.11 | 1000 | | | |
| 1.00E+02 | | 34.02 | 100 | | | |
| 1.00E+02 | | 30.53 | 100 | | | |
| 1.00E+01 | | 32.98 | 10 | | | |
| 1.00E+01 | | 40 | 0 | | | |
| 1.00E−00 | | 40 | 0 | | | |
| 1.00E−00 | | 38.21 | 1 | | | |
| NTC | | 40 | 0 | | | |

Gene Name sbg618069LRR

| | Fold Change in Disease |
|---|---|
| | Population Relative to |
| Disease tissues | Normal |
| colon tumor | −9.54 |
| colon tumor | 3.41 |
| colon tumor | −4.95 |
| colon tumor | 1.96 |
| lung tumor | 10.67 |
| lung tumor | 1.71 |
| lung tumor | −4.36 |
| lung tumor | 3.28 |
| breast tumor | 2.96 |
| breast tumor | 6.64 |
| breast tumor | 17.40 |
| breast tumor | −2.24 |
| brain stage 5 ALZ | −2.34 |
| brain stage 5 ALZ | 1.27 |
| brain stage 5 ALZ | −1.43 |
| brain stage 5 ALZ | −1.21 |
| lung 24 | −68.13 |
| lung 28 | −35.30 |
| lung 23 | −5.94 |
| asthmatic lung | −3.14 |
| asthmatic lung | 1.02 |
| asthmatic lung | 2.44 |
| asthmatic lung | 2.29 |
| endo VEGF | 0.00 |
| endo bFGF | 0.00 |
| heart T-1 | 2.72 |
| heart T-14 | 1.68 |
| heart T-3399 | 1.30 |
| BM stim | 5.04 |
| osteo dif | 0.00 |
| Cartilage (pool) | −3.91 |
| PBL HIV IIIB | −2.26 |
| MRC5 HSV strain F | 129.92 |

Gene Name sbg934114Relaxin

Low overall expression in normal and disease samples. Highest normal expression in testis, liver, and whole brain. Highest disease expression in 3 of the normal lung samples, one of the normal tumor samples, the HSV-infected MRC5 cells, the adenoid, and the T cells. Highest disease expression in 2 of the normal lung samples, one of the lung tumor samples, 1 of the normal breast samples, 1 of the breast tumor samples, and the uninfected PBL samples. Downregulation in 1 of 4 colon tumors and downregulation in 2 of 4 lung tumors imply roles in cancers of the colon and lung. Downregulation in 3 of 3 COPD lung samples and upregulation in 3 of 4 asthmatic lung samples implicates this gene in COPD and asthma. Upregulation in 2 of 3 heart samples proposes roles in non-obstructive and obstructive DCM. Downregulation in the OA cartilage pool and low expression in RA and OA synovium, OA bone, and chondrocytes suggests an involvement in osteoarthritis and rheumatoid arthritis. Downregulation in an HIV-infected primary cell line suggests an involvement in HIV. Upregulation in HSV implicates involvement in herpes simplex virus as a potential host factor.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg934114Relaxin | 1 and 2) | 1) | 2) | Copies | (ng) | (ng) | RNA |
| Subcutaneous | 37.4, 40 | 5.41 | 0 | 2.71 | 3.06 | 16.34 | 44.20 |
| Adipocytes Zenbio | | | | | | | |
| Subcutaneous Adipose | 40, 40 | 0.73 | 0 | 0.37 | 0.96 | 52.36 | 19.11 |
| Zenbio | | | | | | | |
| Adrenal Gland Clontech | 40, 40 | 0.73 | 0.69 | 0.71 | 0.61 | 81.97 | 58.20 |
| Whole Brain Clontech | 33.52, 34.72 | 47.76 | 24.26 | 36.01 | 7.24 | 6.91 | 248.69 |
| Fetal Brain Clontech | 39.51, 40 | 1.65 | 0.86 | 1.26 | 0.48 | 103.95 | 130.46 |
| Cerebellum Clontech | 40, 39.84 | 0.99 | 1.37 | 1.18 | 2.17 | 23.04 | 27.19 |
| Cervix | 40, 40 | 1.05 | 0 | 0.53 | 2.42 | 20.66 | 10.85 |
| Colon | 40, 37.31 | 0 | 5.7 | 2.85 | 2.71 | 18.45 | 52.58 |
| Endometrium | 40, 40 | 0 | 0 | 0.00 | 0.73 | 68.21 | 0.00 |
| Esophagus | 40, 40 | 0 | 0 | 0.00 | 1.37 | 36.50 | 0.00 |
| Heart Clontech | 38.11, 40 | 3.63 | 1.31 | 2.47 | 1.32 | 37.88 | 93.56 |
| Hypothalamus | 40, 40 | 0 | 1.06 | 0.53 | 0.32 | 155.28 | 82.30 |
| Ileum | 40, 36.79 | 0 | 7.62 | 3.81 | 2.58 | 19.38 | 73.84 |
| Jejunum | 35.14, 35.9 | 19.25 | 12.54 | 15.90 | 6.60 | 7.58 | 120.42 |
| Kidney | 40, 40 | 0 | 0 | 0.00 | 2.12 | 23.58 | 0.00 |
| Liver | 37.25, 35.17 | 5.88 | 18.92 | 12.40 | 1.50 | 33.33 | 413.33 |
| Fetal Liver Clontech | 40, 37.38 | 0.73 | 5.47 | 3.10 | 10.40 | 4.81 | 14.90 |
| Lung | 37.07, 40 | 6.52 | 0 | 3.26 | 2.57 | 19.46 | 63.42 |
| Mammary Gland | 40, 40 | 0 | 0 | 0.00 | 13.00 | 3.85 | 0.00 |
| Clontech | | | | | | | |
| Myometrium | 37.95, 40 | 3.98 | 0 | 1.99 | 2.34 | 21.37 | 42.52 |
| Omentum | 36.24, 37.04 | 10.39 | 6.63 | 8.51 | 3.94 | 12.69 | 107.99 |
| Ovary | 35.29, 36.26 | 17.66 | 10.28 | 13.97 | 4.34 | 11.52 | 160.94 |
| Pancreas | 39.48, 40 | 1.69 | 0 | 0.85 | 0.81 | 61.80 | 52.22 |
| Head of Pancreas | 36.17, 39.82 | 10.79 | 1.39 | 6.09 | 1.57 | 31.85 | 193.95 |
| Parotid Gland | 40, 38.66 | 0 | 2.67 | 1.34 | 5.48 | 9.12 | 12.18 |
| Placenta Clontech | 40, 36.57 | 0 | 8.63 | 4.32 | 5.26 | 9.51 | 41.02 |
| Prostate | 36.91, 40 | 7.14 | 0 | 3.57 | 3.00 | 16.67 | 59.50 |
| Rectum | 40, 37.34 | 0 | 5.61 | 2.81 | 1.23 | 40.65 | 114.02 |
| Salivary Gland | 40, 40 | 0 | 0 | 0.00 | 7.31 | 6.84 | 0.00 |
| Clontech | | | | | | | |
| Skeletal Muscle | 40, 39.45 | 0 | 1.71 | 0.86 | 1.26 | 39.68 | 33.93 |
| Clontech | | | | | | | |
| Skin | 39.2, 40 | 1.98 | 0 | 0.99 | 1.21 | 41.32 | 40.91 |
| Small Intestine | 40, 40 | 0 | 0 | 0.00 | 0.98 | 51.07 | 0.00 |
| Clontech | | | | | | | |
| Spleen | 36, 40 | 11.84 | 0 | 5.92 | 4.92 | 10.16 | 60.16 |
| Stomach | 35.48, 39.19 | 15.9 | 1.98 | 8.94 | 2.73 | 18.32 | 163.74 |
| Testis Clontech | 40, 36.09 | 0 | 11.27 | 5.64 | 0.57 | 87.87 | 495.17 |
| Thymus Clontech | 34.34, 36.66 | 30.03 | 8.2 | 19.12 | 9.89 | 5.06 | 96.64 |
| Thyroid | 40, 40 | 0 | 0 | 0.00 | 2.77 | 18.05 | 0.00 |
| Trachea Clontech | 40, 37.8 | 0 | 4.33 | 2.17 | 9.71 | 5.15 | 11.15 |
| Urinary Bladder | 40, 40 | 0 | 0 | 0.00 | 5.47 | 9.14 | 0.00 |
| Uterus | 37.11, 35.16 | 6.36 | 18.96 | 12.66 | 5.34 | 9.36 | 118.54 |
| genomic | 27.61 | 1307.98 | | | | | |
| b-actin | 27.15 | 1697.68 | | | | | |
| 1.00E+05 | 19.88 | 100000 | | | | | |
| 1.00E+05 | 20.05 | 100000 | | | | | |
| 1.00E+04 | 24.01 | 10000 | | | | | |
| 1.00E+04 | 23.92 | 10000 | | | | | |
| 1.00E+03 | 27.98 | 1000 | | | | | |
| 1.00E+03 | 27.6 | 1000 | | | | | |
| 1.00E+02 | 32.89 | 100 | | | | | |
| 1.00E+02 | 32.2 | 100 | | | | | |
| 1.00E+01 | 36.1 | 10 | | | | | |
| 1.00E+01 | | | | | | | |
| 1.00E−00 | | | | | | | |
| 1.00E−00 | | | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | Fold |
| | Reg | | | detected/ | | Change |
| | number | | Mean | 50 ng | | in |
| Sample | (GSK | | GOI | total | | Disease |
| sbg934114Relaxin | identifier) | Ct | copies | RNA | Sample | Population |
| colon normal GW98-167 | 21941 | 36.1 | 22.33 | 44.66 | colon normal | |
| colon tumor GW98-166 | 21940 | 36.47 | 18.37 | 36.74 | colon tumor | −1.22 |
| colon normal GW98-178 | 22080 | 35.7 | 27.58 | 55.16 | colon normal | |
| colon tumor GW98-177 | 22060 | 38.05 | 7.98 | 15.96 | colon tumor | −3.46 |
| colon normal GW98-561 | 23514 | 33.57 | 84.85 | 169.70 | colon normal | |
| colon tumor GW98-560 | 23513 | 37.66 | 9.8 | 19.60 | colon tumor | −8.66 |
| colon normal GW98-894 | 24691 | 36.39 | 19.09 | 38.18 | colon normal | |
| colon tumor GW98-893 | 24690 | 36.43 | 18.74 | 37.48 | colon tumor | −1.02 |
| lung normal GW98-3 | 20742 | 32.48 | 150.6 | 301.20 | lung normal | |
| lung tumor GW98-2 | 20741 | 36.65 | 16.64 | 33.28 | lung tumor | −9.05 |
| lung normal GW97-179 | 20677 | 33.22 | 102.07 | 204.14 | lung normal | |
| lung tumor GW97-178 | 20676 | 32.31 | 164.63 | 329.26 | lung tumor | 1.61 |
| lung normal GW98-165 | 21922 | 32.08 | 185.96 | 371.92 | lung normal | |
| lung tumor GW98-164 | 21921 | 35.14 | 37.09 | 74.18 | lung tumor | −5.01 |
| lung normal GW98-282 | 22584 | 36.41 | 18.93 | 37.86 | lung normal | |
| lung tumor GW98-281 | 22583 | 35.24 | 35.13 | 70.26 | lung tumor | 1.86 |
| breast normal GW00-392 | 28750 | 34.04 | 66.25 | 66.25 | breast normal | |
| breast tumor GW00-391 | 28746 | 37.9 | 8.61 | 17.22 | breast tumor | −3.85 |
| breast normal GW00-413 | 28798 | 36.36 | 19.44 | 19.44 | breast normal | |
| breast tumor GW00-412 | 28797 | 36.79 | 15.49 | 30.98 | breast tumor | 1.59 |
| breast normal GW00- | 27592-95 | 36.91 | 14.52 | 14.52 | breast normal | |
| 235:238 | | | | | | |
| breast tumor GW00- | 27588-91 | 35.43 | 31.71 | 31.71 | breast tumor | 2.18 |
| 231:234 | | | | | | |
| breast normal GW98-621 | 23656 | 36.26 | 20.51 | 41.02 | breast normal | |
| breast tumor GW98-620 | 23655 | 35.76 | 26.68 | 53.36 | breast tumor | 1.30 |
| brain normal BB99-542 | 25507 | 37.99 | 8.21 | 16.42 | brain normal | |
| brain normal BB99-406 | 25509 | 40 | 1.41 | 2.82 | brain normal | |
| brain normal BB99-904 | 25546 | 40 | 0 | 0.00 | brain normal | |
| brain stage 5 ALZ BB99- | 25502 | 38.65 | 5.82 | 11.64 | brain stage 5 ALZ | 1.81 |
| 874 | | | | | | |
| brain stage 5 ALZ BB99- | 25503 | 37 | 13.9 | 27.80 | brain stage 5 ALZ | 4.33 |
| 887 | | | | | | |
| brain stage 5 ALZ BB99- | 25504 | 37.4 | 11.24 | 22.48 | brain stage 5 ALZ | 3.51 |
| 862 | | | | | | |
| brain stage 5 ALZ BB99- | 25542 | 38 | 8.19 | 16.38 | brain stage 5 ALZ | 2.55 |
| 927 | | | | | | |
| CT lung KC | normal | 35.32 | 33.59 | 67.18 | CT lung | |
| lung 26 KC | normal | 37.02 | 13.72 | 13.72 | lung 26 | |
| lung 27 KC | normal | 40 | 0 | 0.00 | lung 27 | |
| lung 24 KC | COPD | 39.97 | 2.78 | 2.78 | lung 24 | −7.81 |
| lung 28 KC | COPD | 40 | 0 | 0.00 | lung 28 | −21.71 |
| lung 23 KC | COPD | 39.06 | 4.67 | 4.67 | lung 23 | −4.65 |
| lung 25 KC | normal | 38.61 | 5.92 | 5.92 | lung 25 | |
| asthmatic lung ODO3112 | 29321 | 36.02 | 23.31 | 23.31 | asthmatic lung | 1.07 |
| asthmatic lung ODO3433 | 29323 | 34.12 | 63.36 | 126.72 | asthmatic lung | 5.84 |
| asthmatic lung ODO3397 | 29322 | 33.99 | 68.06 | 136.12 | asthmatic lung | 6.27 |
| asthmatic lung ODO4928 | 29325 | 33.77 | 76.08 | 152.16 | asthmatic lung | 7.01 |
| endo cells KC | control | 40 | 0 | 0.00 | endo cells | |
| endo VEGF KC | | 40 | 0 | 0.00 | endo VEGF | 0.00 |
| endo bFGF KC | | 38.16 | 7.54 | 7.54 | endo bFGF | 7.54 |
| heart Clontech | normal | 40 | 0 | 0.00 | heart | |
| heart (T-1) ischemic | 29417 | 40 | 1.36 | 2.72 | heart T-1 | 2.72 |
| heart (T-14) non- | 29422 | 36.31 | 20.01 | 40.02 | heart T-14 | 40.02 |
| obstructive DCM | | | | | | |
| heart (T-3399) DCM | 29426 | 37.17 | 12.7 | 25.40 | heart T-3399 | 25.40 |
| adenoid GW99-269 | 26162 | 33.33 | 96.07 | 192.14 | adenoid | |
| tonsil GW98-280 | 22582 | 34.86 | 42.85 | 85.70 | tonsil | |
| T cells PC00314 | 28453 | 33.48 | 88.65 | 177.30 | T cells | |
| PBMNC | | 40 | 0 | 0.00 | PBMNC | |
| monocyte | | 40 | 0 | 0.00 | monocyte | |
| B cells PC00665 | 28455 | 32.44 | 153.68 | 307.36 | B cells | |
| dendritic cells 28441 | | 35.78 | 26.47 | 52.94 | dendritic cells | |
| neutrophils | 28440 | 36.18 | 21.43 | 21.43 | neutrophils | |
| eosinophils | 28446 | 39.2 | 4.34 | 8.68 | eosinophils | |
| BM unstim | | 39.56 | 3.6 | 3.60 | BM unstim | |
| BM stim | | 40 | 1.34 | 1.34 | BM stim | −2.69 |
| osteo dif | | 40 | 0 | 0.00 | osteo dif | 0.00 |
| osteo undif | | 40 | 0 | 0.00 | osteo undif | |
| chondrocytes | | 36.64 | 16.79 | 41.98 | chondrocytes | |
| OA Synovium IP12/01 | 29462 | 34.75 | 45.45 | 45.45 | OA Synovium | |
| OA Synovium NP10/01 | 29461 | 36.02 | 23.28 | 46.56 | OA Synovium | |
| OA Synovium NP57/00 | 28464 | 34.24 | 59.37 | 118.74 | OA Synovium | |
| RA Synovium NP03/01 | 28466 | 37.88 | 8.71 | 17.42 | RA Synovium | |
| RA Synovium NP71/00 | 28467 | 36.02 | 23.22 | 46.44 | RA Synovium | |
| RA Synovium NP45/00 | 28475 | 34.9 | 41.9 | 83.80 | RA Synovium | |
| OA bone (biobank) | 29217 | 33.59 | 83.75 | 83.75 | OA bone | |
| | | | | | (biobank) | |
| OA bone Sample 1 | J. Emory | 37.31 | 11.8 | 23.60 | OA bone | |
| OA bone Sample 2 | J. Emory | 37.47 | 10.81 | 21.62 | OA bone | |
| Cartilage (pool) | Normal | 34.61 | 49.07 | 98.14 | Cartilage (pool) | |
| Cartilage (pool) | OA | 40 | 0 | 0.00 | Cartilage (pool) | −98.14 |
| PBL unifected | 28441 | 33.86 | 72.8 | 145.60 | PBL unifected | |
| PBL HIV IIIB | 28442 | 37.08 | 13.28 | 26.56 | PBL HIV IIIB | −5.48 |
| MRC5 uninfected | 29158 | 39.01 | 4.81 | 9.62 | MRC5 uninfected | |
| (100%) | | | | | (100%) | |
| MRC5 HSV strain F | 29178 | 31.5 | 252.68 | 505.36 | MRC5 HSV strain F | 52.53 |
| W12 cells | 29179 | 39.84 | 3.1 | 6.20 | W12 cells | |
| Keratinocytes | 29180 | 39.07 | 4.64 | 9.28 | Keratinocytes | |
| B-actin control | | 27.9 | 1683.49 | | | |
| genomic | | 28.13 | 1492.71 | | | |
| 1.00E+05 | | 20.5 | 100000 | | | |
| 1.00E+05 | | 20.61 | 100000 | | | |
| 1.00E+04 | | 24.56 | 10000 | | | |
| 1.00E+04 | | 24.17 | 10000 | | | |
| 1.00E+03 | | 28.14 | 1000 | | | |
| 1.00E+03 | | 28.32 | 1000 | | | |
| 1.00E+02 | | 34.26 | 100 | | | |
| 1.00E+02 | | 32.76 | 100 | | | |
| 1.00E+01 | | 38.07 | 10 | | | |
| 1.00E+01 | | 37.53 | 10 | | | |
| 1.00E−00 | | 40 | 0 | | | |
| 1.00E−00 | | 40 | 1 | | | |
| NTC | | 38.42 | −1 | | | |

Gene Name sbg934114Relaxin

| | Fold Change in Disease |
|---|---|
| | Population Relative to |
| Disease tissues | Normal |
| colon tumor | −1.22 |
| colon tumor | −3.46 |
| colon tumor | −8.66 |
| colon tumor | −1.02 |
| lung tumor | −9.05 |
| lung tumor | 1.61 |
| lung tumor | −5.01 |
| lung tumor | 1.86 |
| breast tumor | −3.85 |
| breast tumor | 1.59 |
| breast tumor | 2.18 |
| breast tumor | 1.30 |
| brain stage 5 ALZ | 1.81 |
| brain stage 5 ALZ | 4.33 |
| brain stage 5 ALZ | 3.51 |
| brain stage 5 ALZ | 2.55 |
| lung 24 | −7.81 |
| lung 28 | −21.71 |
| lung 23 | −4.65 |
| asthmatic lung | 1.07 |
| asthmatic lung | 5.84 |
| asthmatic lung | 6.27 |
| asthmatic lung | 7.01 |
| endo VEGF | 0.00 |
| endo bFGF | 7.54 |
| heart T-1 | 2.72 |
| heart T-14 | 40.02 |
| heart T-3399 | 25.40 |
| BM stim | −2.69 |
| osteo dif | 0.00 |
| Cartilage (pool) | −98.14 |
| PBL HIV IIIB | −5.48 |
| MRC5 HSV strain F | 52.53 |

Gene Name sbg99174LOX-like

Moderate overall expression in normal and disease samples. Highest normal expression in whole brain, liver, skin, spleen, testis. Shows relatively good expression in the female reproductive samples as well as the GI tract samples. Highest disease expression in one of the normal lung samples, one of the asthmatic lung samples, neutrophils, eosinophils, 2 of the RA synovium samples, and one of the OA bone samples. Downregulation in 1 of 4 lung tumor samples suggests possible implication in lung cancer. Upregulation in 2 of 4 breast tumors implies an involvement in cancers of the breast. Downregulation in 1 of 4 AD brains along with the high expression seen in the brain suggests an involvement in Alzheimer's disease. Downregulation in 2 of 3 COPD lung samples implies an involvement in chronic obstructive pulmonary disease. Upregulation in 1 of 4 asthmatic lung samples implies a role in asthma. Downregulation in OA cartilage and high expression in OA and RA synovium suggests possible involvement in osteoarthritis and rheumatoid arthritis. Corroborating high expression in the T cells provides additional evidence for a role in RA/OA. Moderate expression in other immune cells.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg99174LOX-like | 1 and 2) | 1) | 2) | Copies | (ng) | (ng) | RNA |
| Subcutaneous | 39.67, 34.48 | 0.53 | 11.91 | 6.22 | 3.06 | 16.34 | 101.63 |
| Adipocytes Zenbio | | | | | | | |
| Subcutaneous Adipose | 40, 39.75 | 0.39 | 0.51 | 0.45 | 0.96 | 52.36 | 23.56 |
| Zenbio | | | | | | | |
| Adrenal Gland | 38.61, 37.69 | 1 | 1.75 | 1.38 | 0.61 | 81.97 | 112.70 |
| Clontech | | | | | | | |
| Whole Brain Clontech | 30.59, 31.07 | 122.95 | 92.57 | 107.76 | 7.24 | 6.91 | 744.20 |
| Fetal Brain Clontech | 40, 40 | 0 | 0.29 | 0.15 | 0.48 | 103.95 | 15.07 |
| Cerebellum Clontech | 40, 40 | 0.4 | 0.4 | 0.40 | 2.17 | 23.04 | 9.22 |
| Cervix | 35.77, 40 | 5.52 | 0.62 | 3.07 | 2.42 | 20.66 | 63.43 |
| Colon | 40, 39.41 | 0.34 | 0.62 | 0.48 | 2.71 | 18.45 | 8.86 |
| Endometrium | 40, 38.65 | 0 | 0.98 | 0.49 | 0.73 | 68.21 | 33.42 |
| Esophagus | 40, 40 | 0.35 | 0.41 | 0.38 | 1.37 | 36.50 | 13.87 |
| Heart Clontech | 39.33, 40 | 0.65 | 0 | 0.33 | 1.32 | 37.88 | 12.31 |
| Hypothalamus | 40, 39.54 | 0 | 0.58 | 0.29 | 0.32 | 155.28 | 45.03 |
| Ileum | 35.42, 35.76 | 6.79 | 5.55 | 6.17 | 2.58 | 19.38 | 119.57 |
| Jejunum | 34.13, 33.4 | 14.77 | 22.9 | 18.84 | 6.60 | 7.58 | 142.69 |
| Kidney | 40, 37.22 | 0.68 | 2.32 | 1.50 | 2.12 | 23.58 | 35.38 |
| Liver | 33.5, 33.86 | 21.47 | 17.3 | 19.39 | 1.50 | 33.33 | 646.17 |
| Fetal Liver Clontech | 32.78, 33.23 | 33.08 | 25.33 | 29.21 | 10.40 | 4.81 | 140.41 |
| Lung | 33.96, 34.44 | 16.33 | 12.25 | 14.29 | 2.57 | 19.46 | 278.02 |
| Mammary Gland | 35.05, 36.02 | 8.51 | 4.76 | 6.64 | 13.00 | 3.85 | 25.52 |
| Clontech | | | | | | | |
| Myometrium | 35.08, 35.49 | 8.34 | 6.52 | 7.43 | 2.34 | 21.37 | 158.76 |
| Omentum | 37.6, 35.05 | 1.83 | 8.49 | 5.16 | 3.94 | 12.69 | 65.48 |
| Ovary | 35.57, 32.76 | 6.21 | 33.46 | 19.84 | 4.34 | 11.52 | 228.51 |
| Pancreas | 40, 40 | 0 | 0.42 | 0.21 | 0.81 | 61.80 | 12.98 |
| Head of Pancreas | 40, 38.65 | 0.56 | 0.98 | 0.77 | 1.57 | 31.85 | 24.52 |
| Parotid Gland | 35.28, 40 | 7.4 | 0.64 | 4.02 | 5.48 | 9.12 | 36.68 |
| Placenta Clontech | 40, 39.17 | 0.32 | 0.72 | 0.52 | 5.26 | 9.51 | 4.94 |
| Prostate | 40, 35.34 | 0 | 7.15 | 3.58 | 3.00 | 16.67 | 59.58 |
| Rectum | 37.29, 39.02 | 2.22 | 0.78 | 1.50 | 1.23 | 40.65 | 60.98 |
| Salivary Gland | 40, 38.15 | 0.27 | 1.32 | 0.80 | 7.31 | 6.84 | 5.44 |
| Clontech | | | | | | | |
| Skeletal Muscle | 40, 38.56 | 0.56 | 1.03 | 0.80 | 1.26 | 39.68 | 31.55 |
| Clontech | | | | | | | |
| Skin | 35.1, 34.19 | 8.22 | 14.22 | 11.22 | 1.21 | 41.32 | 463.64 |
| Small Intestine | 40, 40 | 0 | 0.85 | 0.43 | 0.98 | 51.07 | 21.71 |
| Clontech | | | | | | | |
| Spleen | 32.2, 32.08 | 46.76 | 50.49 | 48.63 | 4.92 | 10.16 | 494.16 |
| Stomach | 34.28, 34.07 | 13.5 | 15.3 | 14.40 | 2.73 | 18.32 | 263.74 |
| Testis Clontech | 34.05, 32.7 | 15.46 | 34.82 | 25.14 | 0.57 | 87.87 | 2209.14 |
| Thymus Clontech | 33.85, 32.4 | 17.44 | 41.71 | 29.58 | 9.89 | 5.06 | 149.52 |
| Thyroid | 34.22, 34.29 | 13.93 | 13.37 | 13.65 | 2.77 | 18.05 | 246.39 |
| Trachea Clontech | 32.51, 32.4 | 38.89 | 41.53 | 40.21 | 9.71 | 5.15 | 207.05 |
| Urinary Bladder | 35.47, 40 | 6.6 | 0 | 3.30 | 5.47 | 9.14 | 30.16 |
| Uterus | 33.8, 33.6 | 18.01 | 20.22 | 19.12 | 5.34 | 9.36 | 178.98 |
| genomic | 26.31 | 1603.29 | | | | | |
| b-actin | 27.35 | 860.96 | | | | | |
| 1.00E+05 | 19.71 | 100000 | | | | | |
| 1.00E+05 | 19.88 | 100000 | | | | | |
| 1.00E+04 | 22.98 | 10000 | | | | | |
| 1.00E+04 | 23.01 | 10000 | | | | | |
| 1.00E+03 | 26.44 | 1000 | | | | | |
| 1.00E+03 | 26.54 | 1000 | | | | | |
| 1.00E+02 | 31.28 | 100 | | | | | |
| 1.00E+02 | 31.29 | 100 | | | | | |
| 1.00E+01 | 35.79 | 10 | | | | | |
| 1.00E+01 | 34.36 | 10 | | | | | |
| 1.00E−00 | 38.32 | 1 | | | | | |
| 1.00E−00 | 40 | 1 | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | Fold |
| | Reg | | | detected/ | | Change |
| | number | | Mean | 50 ng | | in |
| Sample | (GSK | | GOI | total | | Disease |
| sbg99174LOX-like | identifier) | Ct | copies | RNA | Sample | Population |
| colon normal GW98-167 | 21941 | 29.81 | 184.31 | 368.62 | colon normal | |
| colon tumor GW98-166 | 21940 | 29.25 | 254.48 | 508.96 | colon tumor | 1.38 |
| colon normal GW98-178 | 22080 | 32.06 | 50.31 | 100.62 | colon normal | |
| colon tumor GW98-177 | 22060 | 31.77 | 59.28 | 118.56 | colon tumor | 1.18 |
| colon normal GW98-561 | 23514 | 33.21 | 25.83 | 51.66 | colon normal | |
| colon tumor GW98-560 | 23513 | 31.74 | 60.51 | 121.02 | colon tumor | 2.34 |
| colon normal GW98-894 | 24691 | 30.17 | 149.84 | 299.68 | colon normal | |
| colon tumor GW98-893 | 24690 | 29.41 | 232.23 | 464.46 | colon tumor | 1.55 |
| lung normal GW98-3 | 20742 | 25.76 | 1914.72 | 3829.44 | lung normal | |
| lung tumor GW98-2 | 20741 | 30.12 | 154.63 | 309.26 | lung tumor | −12.38 |
| lung normal GW97-179 | 20677 | 29.59 | 209.5 | 419.00 | lung normal | |
| lung tumor GW97-178 | 20676 | 27.2 | 835.04 | 1670.08 | lung tumor | 3.99 |
| lung normal GW98-165 | 21922 | 28.22 | 462.22 | 924.44 | lung normal | |
| lung tumor GW98-164 | 21921 | 29.27 | 251.87 | 503.74 | lung tumor | −1.84 |
| lung normal GW98-282 | 22584 | 30.18 | 149.17 | 298.34 | lung normal | |
| lung tumor GW98-281 | 22583 | 28.47 | 399.41 | 798.82 | lung tumor | 2.68 |
| breast normal GW00-392 | 28750 | 30.24 | 143.58 | 143.58 | breast normal | |
| breast tumor GW00-391 | 28746 | 30.16 | 151.08 | 302.16 | breast tumor | 2.10 |
| breast normal GW00-413 | 28798 | 31.51 | 68.87 | 68.87 | breast normal | |
| breast tumor GW00-412 | 28797 | 28.91 | 310.66 | 621.32 | breast tumor | 9.02 |
| breast normal GW00- | 27592-95 | 40 | 0 | 0.00 | breast normal | |
| 235:238 | | | | | | |
| breast tumor GW00- | 27588-91 | 33.55 | 21.28 | 21.28 | breast tumor | 21.28 |
| 231:234 | | | | | | |
| breast normal GW98-621 | 23656 | 31.57 | 66.56 | 133.12 | breast normal | |
| breast tumor GW98-620 | 23655 | 31.15 | 84.8 | 169.60 | breast tumor | 1.27 |
| brain normal BB99-542 | 25507 | 29.55 | 214.38 | 428.76 | brain normal | |
| brain normal BB99-406 | 25509 | 29.15 | 270.89 | 541.78 | brain normal | |
| brain normal BB99-904 | 25546 | 30.48 | 124.98 | 249.96 | brain normal | |
| brain stage 5 ALZ BB99- | 25502 | 33.57 | 20.99 | 41.98 | brain stage 5 ALZ | −9.69 |
| 874 | | | | | | |
| brain stage 5 ALZ BB99- | 25503 | 29.89 | 176.42 | 352.84 | brain stage 5 ALZ | −1.15 |
| 887 | | | | | | |
| brain stage 5 ALZ BB99- | 25504 | 31.65 | 63.83 | 127.66 | brain stage 5 ALZ | −3.19 |
| 862 | | | | | | |
| brain stage 5 ALZ BB99- | 25542 | 31.04 | 90.73 | 181.46 | brain stage 5 ALZ | −2.24 |
| 927 | | | | | | |
| CT lung KC | normal | 29.94 | 170.8 | 341.60 | CT lung | |
| lung 26 KC | normal | 30.63 | 115.04 | 115.04 | lung 26 | |
| lung 27 KC | normal | 32.22 | 45.83 | 45.83 | lung 27 | |
| lung 24 KC | COPD | 34.16 | 14.91 | 14.91 | lung 24 | −9.21 |
| lung 28 KC | COPD | 33.51 | 21.7 | 21.70 | lung 28 | −6.33 |
| lung 23 KC | COPD | 32.51 | 38.67 | 38.67 | lung 23 | −3.55 |
| lung 25 KC | normal | 32.18 | 46.79 | 46.79 | lung 25 | |
| asthmatic lung ODO3112 | 29321 | 31.84 | 56.95 | 56.95 | asthmatic lung | −2.41 |
| asthmatic lung ODO3433 | 29323 | 31.12 | 86.42 | 172.84 | asthmatic lung | 1.26 |
| asthmatic lung ODO3397 | 29322 | 27.13 | 867.31 | 1734.62 | asthmatic lung | 12.63 |
| asthmatic lung ODO4928 | 29325 | 30.25 | 142.92 | 285.84 | asthmatic lung | 2.08 |
| endo cells KC | control | 31.23 | 81.05 | 81.05 | endo cells | |
| endo VEGF KC | | 31.87 | 56.15 | 56.15 | endo VEGF | −1.44 |
| endo bFGF KC | | 32.64 | 35.97 | 35.97 | endo bFGF | −2.25 |
| heart Clontech | normal | 35.46 | 7.06 | 14.12 | heart | |
| heart (T-1) ischemic | 29417 | 35.73 | 6.03 | 12.06 | heart T-1 | −1.17 |
| heart (T-14) non- | 29422 | 34.34 | 13.49 | 26.98 | heart T-14 | 1.91 |
| obstructive DCM | | | | | | |
| heart (T-3399) DCM | 29426 | 33.65 | 20.01 | 40.02 | heart T-3399 | 2.83 |
| adenoid GW99-269 | 26162 | 31.2 | 82.69 | 165.38 | adenoid | |
| tonsil GW98-280 | 22582 | 30.8 | 103.96 | 207.92 | tonsil | |
| T cells PC00314 | 28453 | 28.16 | 480.4 | 960.80 | T cells | |
| PBMNC | | 30.25 | 143.11 | 143.11 | PBMNC | |
| monocyte | | 30.05 | 160.68 | 321.36 | monocyte | |
| B cells PC00665 | 28455 | 30.75 | 107.02 | 214.04 | B cells | |
| dendritic cells 28441 | | 30.32 | 137.17 | 274.34 | dendritic cells | |
| neutrophils | 28440 | 26.32 | 1390.87 | 1390.87 | neutrophils | |
| eosinophils | 28446 | 25.07 | 2854.44 | 5708.88 | eosinophils | |
| BM unstim | | 30.72 | 109.05 | 109.05 | BM unstim | |
| BM stim | | 28.61 | 369.2 | 369.20 | BM stim | 3.39 |
| osteo dif | | 40 | 0 | 0.00 | osteo dif | −0.28 |
| osteo undif | | 40 | 0.28 | 0.28 | osteo undif | |
| chondrocytes | | 34.3 | 13.76 | 34.40 | chondrocytes | |
| OA Synovium IP12/01 | 29462 | 27.56 | 676.63 | 676.63 | OA Synovium | |
| OA Synovium NP10/01 | 29461 | 31.41 | 73.19 | 146.38 | OA Synovium | |
| OA Synovium NP57/00 | 28464 | 28.02 | 518.05 | 1036.10 | OA Synovium | |
| RA Synovium NP03/01 | 28466 | 27.03 | 921.88 | 1843.76 | RA Synovium | |
| RA Synovium NP71/00 | 28467 | 27.04 | 914.02 | 1828.04 | RA Synovium | |
| RA Synovium NP45/00 | 28475 | 29.06 | 285.08 | 570.16 | RA Synovium | |
| OA bone (biobank) | 29217 | 26.78 | 1065.84 | 1065.84 | OA bone | |
| | | | | | (biobank) | |
| OA bone Sample 1 | J. Emory | 30.27 | 141.07 | 282.14 | OA bone | |
| OA bone Sample 2 | J. Emory | 27.04 | 917.04 | 1834.08 | OA bone | |
| Cartilage (pool) | Normal | 28.23 | 461.21 | 922.42 | Cartilage (pool) | |
| Cartilage (pool) | OA | 33.16 | 26.65 | 53.30 | Cartilage (pool) | −17.31 |
| PBL unifected | 28441 | 28.72 | 346.97 | 693.94 | PBL unifected | |
| PBL HIV IIIB | 28442 | 28.54 | 384.02 | 768.04 | PBL HIV IIIB | 1.11 |
| MRC5 uninfected | 29158 | 31.64 | 64.07 | 128.14 | MRC5 uninfected | |
| (100%) | | | | | (100%) | |
| MRC5 HSV strain F | 29178 | 31.53 | 68.31 | 136.62 | MRC5 HSV strain F | 1.07 |
| W12 cells | 29179 | 34.44 | 12.72 | 25.44 | W12 cells | |
| Keratinocytes | 29180 | 36.02 | 5.1 | 10.20 | Keratinocytes | |
| B-actin control | | 27.2 | 835.98 | | | |
| genomic | | 26.76 | 1073.72 | | | |
| 1.00E+05 | 19.13 | 100000 | | | | |
| 1.00E+05 | 19.61 | 100000 | | | | |
| 1.00E+04 | 22.79 | 10000 | | | | |
| 1.00E+04 | 22.5 | 10000 | | | | |
| 1.00E+03 | 26.36 | 1000 | | | | |
| 1.00E+03 | 26.23 | 1000 | | | | |
| 1.00E+02 | 31.25 | 100 | | | | |
| 1.00E+02 | 30.82 | 100 | | | | |
| 1.00E+01 | 35.02 | 10 | | | | |
| 1.00E+01 | 35.15 | 10 | | | | |
| 1.00E−00 | 40 | 0 | | | | |
| 1.00E−00 | 40 | 0 | | | | |
| NTC | 40 | 0 | | | | |

Gene Name sbg99174LOX-like

| | Fold Change in Disease |
|---|---|
| | Population Relative to |
| Disease tissues | Normal |
| colon tumor | 1.38 |
| colon tumor | 1.18 |
| colon tumor | 2.34 |
| colon tumor | 1.55 |
| lung tumor | −12.38 |
| lung tumor | 3.99 |
| lung tumor | −1.84 |
| lung tumor | 2.68 |
| breast tumor | 2.10 |
| breast tumor | 9.02 |
| breast tumor | 21.28 |
| breast tumor | 1.27 |
| brain stage 5 ALZ | −9.69 |
| brain stage 5 ALZ | −1.15 |
| brain stage 5 ALZ | −3.19 |
| brain stage 5 ALZ | −2.24 |
| lung 24 | −9.21 |
| lung 28 | −6.33 |
| lung 23 | −3.55 |
| asthmatic lung | −2.41 |
| asthmatic lung | 1.26 |
| asthmatic lung | 12.63 |
| asthmatic lung | 2.08 |
| endo VEGF | −1.44 |
| endo bFGF | −2.25 |
| heart T-1 | −1.17 |
| heart T-14 | 1.91 |
| heart T-3399 | 2.83 |
| BM stim | 3.39 |
| osteo dif | −0.28 |
| Cartilage (pool) | −17.31 |
| PBL HIV IIIB | 1.11 |
| MRC5 HSV strain F | 1.07 |

Gene Name sbg995002PIGR (Taqman)

Extremely low overall expression in normal and disease samples. Highest normal expression in the colon and the parotid gland. Highest disease expression in the one of the lung tumors and one of the colon tumors. Upregulation in 1 of 4 colon tumors and 1 of 4 lung tumors imply roles in cancers of the colon and lung. Downregulation in 3 of 4 AD brain samples as well as high expression in whole brain suggests an involvement in Alzheimer's disease. Downregulation in 3 of 3 COPD lung samples implicates this gene in COPD. Downregulation in the ischemic and non-obstructive DCM heart samples suggests a role for this gene in cardiovascular disease. Upregulation in the stimulated bone marrow sample. Upregulation in HSV implicates involvement in herpes simplex virus as a potential host factor. High expression in neutrophils and eosinophils.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg995002PIGR | 1 and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 36.78, 40 | 11.16 | 0 | 5.58 | 7.24 | 6.91 | 38.54 |
| Fetal Brain Clontech | 40, 40 | 0 | 0 | 0.00 | 0.48 | 103.95 | 0.00 |
| Cerebellum Clontech | 40, 40 | 0 | 0 | 0.00 | 2.17 | 23.04 | 0.00 |
| Cervix | 40, 40 | 0 | 0 | 0.00 | 2.42 | 20.66 | 0.00 |
| Colon | 37.16, 37.12 | 8.81 | 9.05 | 8.93 | 2.71 | 18.45 | 164.76 |
| Endometrium | 40, 40 | 0 | 0 | 0.00 | 0.73 | 68.21 | 0.00 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 40, 40 | 0 | 0 | 0.00 | 2.58 | 19.38 | 0.00 |
| Jejunum | 40, 40 | 0 | 0 | 0.00 | 6.60 | 7.58 | 0.00 |
| Kidney | 40, 40 | 0 | 0 | 0.00 | 2.12 | 23.58 | 0.00 |
| Liver | 40, 39.16 | 0 | 2.58 | 1.29 | 1.50 | 33.33 | 43.00 |
| Fetal Liver Clontech | 40, 40 | 0 | 0 | 0.00 | 10.40 | 4.81 | 0.00 |
| Lung | 40, 40 | 0 | 0 | 0.00 | 2.57 | 19.46 | 0.00 |
| Mammary Gland | 40, 39.18 | 0 | 2.55 | 1.28 | 13.00 | 3.85 | 4.90 |
| Clontech | | | | | | | |
| Myometrium | 40, 40 | 0 | 0 | 0.00 | 2.34 | 21.37 | 0.00 |
| Omentum | 40, 38.04 | 0 | 5.15 | 2.58 | 3.94 | 12.69 | 32.68 |
| Ovary | 39.29, 40 | 2.37 | 0 | 1.19 | 4.34 | 11.52 | 13.65 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 36.7, 37.94 | 11.72 | 5.46 | 8.59 | 5.48 | 9.12 | 78.38 |
| Placenta Clontech | 40, 40 | 0 | 0 | 0.00 | 5.26 | 9.51 | 0.00 |
| Prostate | 40, 40 | 0 | 0 | 0.00 | 3.00 | 16.67 | 0.00 |
| Rectum | 40, 40 | 0 | 0 | 0.00 | 1.23 | 40.65 | 0.00 |
| Salivary Gland | 37.23, 39.21 | 8.45 | 2.5 | 5.48 | 7.31 | 6.84 | 37.45 |
| Clontech | | | | | | | |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine | 40, 40 | 0 | 0 | 0.00 | 0.98 | 51.07 | 0.00 |
| Clontech | | | | | | | |
| Spleen | 40, 38.76 | 1.25 | 3.3 | 2.28 | 4.92 | 10.16 | 23.12 |
| Stomach | 40, 39.25 | 1.25 | 2.43 | 1.84 | 2.73 | 18.32 | 33.70 |
| Testis Clontech | 40, 40 | 0 | 0 | 0.00 | 0.57 | 87.87 | 0.00 |
| Thymus Clontech | 37.82, 40 | 5.88 | 0 | 2.94 | 9.89 | 5.06 | 14.86 |
| Thyroid | 40, 40 | 0 | 0 | 0.00 | 2.77 | 18.05 | 0.00 |
| Trachea Clontech | 40, 40 | 0 | 0 | 0.00 | 9.71 | 5.15 | 0.00 |
| Urinary Bladder | 40, 40 | 0 | 0 | 0.00 | 5.47 | 9.14 | 0.00 |
| Uterus | 38.34, 40 | 4.27 | 0 | 2.14 | 5.34 | 9.36 | 19.99 |
| genomic | 29.66 | 888.99 | | | | | |
| b-actin | 30.72 | 462.87 | | | | | |
| 1.00E+05 | 22.2 | 100000 | | | | | |
| 1.00E+05 | 22.14 | 100000 | | | | | |
| 1.00E+04 | 25.72 | 10000 | | | | | |
| 1.00E+04 | 25.66 | 10000 | | | | | |
| 1.00E+03 | 29.16 | 1000 | | | | | |
| 1.00E+03 | 29.07 | 1000 | | | | | |
| 1.00E+02 | 32.37 | 100 | | | | | |
| 1.00E+02 | 34.12 | 100 | | | | | |
| 1.00E+01 | 37.12 | 10 | | | | | |
| 1.00E+01 | 37.11 | 10 | | | | | |
| 1.00E−00 | 40 | 0 | | | | | |
| 1.00E−00 | 40 | 0 | | | | | |
| NTC | 40 | 0 | | | | | |
| NTC | 38.03 | −1 | | | | | |

| | | | | copies | | |
|---|---|---|---|---|---|---|
| | | | | of | | |
| | | | | mRNA | | Fold |
| | Reg | | | detected/ | | Change |
| | number | | Mean | 50 ng | | in |
| Sample | (GSK | | GOI | total | | Disease |
| sbg995002PIGR | identifier) | Ct | copies | RNA | Sample | Population |
| colon normal GW98-167 | 21941 | 33.43 | 149.84 | 299.68 | colon normal | |
| colon tumor GW98-166 | 21940 | 32.49 | 274.64 | 549.28 | colon tumor | 1.83 |
| colon normal GW98-178 | 22080 | 40 | 0 | 0.00 | colon normal | |
| colon tumor GW98-177 | 22060 | 31.11 | 675.8 | 1351.60 | colon tumor | 1351.60 |
| colon normal GW98-561 | 23514 | 37.74 | 9.09 | 18.18 | colon normal | |
| colon tumor GW98-560 | 23513 | 35.81 | 31.94 | 63.88 | colon tumor | 3.51 |
| colon normal GW98-894 | 24691 | 33.17 | 177.11 | 354.22 | colon normal | |
| colon tumor GW98-893 | 24690 | 31.61 | 488.49 | 976.98 | colon tumor | 2.76 |
| lung normal GW98-3 | 20742 | 35.48 | 39.55 | 79.10 | lung normal | |
| lung tumor GW98-2 | 20741 | 28.32 | 4121.56 | 8243.12 | lung tumor | 104.21 |
| lung normal GW97-179 | 20677 | 36.47 | 20.69 | 41.38 | lung normal | |
| lung tumor GW97-178 | 20676 | 37.24 | 12.59 | 25.18 | lung tumor | −1.64 |
| lung normal GW98-165 | 21922 | 37.32 | 11.96 | 23.92 | lung normal | |
| lung tumor GW98-164 | 21921 | 36.07 | 26.9 | 53.80 | lung tumor | 2.25 |
| lung normal GW98-282 | 22584 | 38.49 | 5.58 | 11.16 | lung normal | |
| lung tumor GW98-281 | 22583 | 37.89 | 8.24 | 16.48 | lung tumor | 1.48 |
| breast normal GW00-392 | 28750 | 39.03 | 3.93 | 3.93 | breast normal | |
| breast tumor GW00-391 | 28746 | 38.01 | 7.65 | 15.30 | breast tumor | 3.89 |
| breast normal GW00-413 | 28798 | 40 | 0 | 0.00 | breast normal | |
| breast tumor GW00-412 | 28797 | 38.67 | 4.98 | 9.96 | breast tumor | 9.96 |
| breast normal GW00- | 27592-95 | 40 | 0 | 0.00 | breast normal | |
| 235:238 | | | | | | |
| breast tumor GW00- | 27588-91 | 40 | 0 | 0.00 | breast tumor | 0.00 |
| 231:234 | | | | | | |
| breast normal GW98-621 | 23656 | 36.12 | 26.13 | 52.26 | breast normal | |
| breast tumor GW98-620 | 23655 | 40 | 0 | 0.00 | breast tumor | −52.26 |
| brain normal BB99-542 | 25507 | 37.14 | 13.43 | 26.86 | brain normal | |
| brain normal BB99-406 | 25509 | 40 | 0 | 0.00 | brain normal | |
| brain normal BB99-904 | 25546 | 40 | 0 | 0.00 | brain normal | |
| brain stage 5 ALZ BB99- | 25502 | 40 | 0 | 0.00 | brain stage 5 ALZ | −8.95 |
| 874 | | | | | | |
| brain stage 5 ALZ BB99- | 25503 | 40 | 0.51 | 1.02 | brain stage 5 ALZ | −8.78 |
| 887 | | | | | | |
| brain stage 5 ALZ BB99- | 25504 | 40 | 0.88 | 1.76 | brain stage 5 ALZ | −5.09 |
| 862 | | | | | | |
| brain stage 5 ALZ BB99- | 25542 | 40 | 1.9 | 3.80 | brain stage 5 ALZ | −2.36 |
| 927 | | | | | | |
| CT lung KC | normal | 36.59 | 19.23 | 38.46 | CT lung | |
| lung 26 KC | normal | 40 | 0 | 0.00 | lung 26 | |
| lung 27 KC | normal | 40 | 0 | 0.00 | lung 27 | |
| lung 24 KC | COPD | 40 | 0 | 0.00 | lung 24 | −9.62 |
| lung 28 KC | COPD | 40 | 0 | 0.00 | lung 28 | −9.62 |
| lung 23 KC | COPD | 40 | 0 | 0.00 | lung 23 | −9.62 |
| lung 25 KC | normal | 40 | 0 | 0.00 | lung 25 | |
| asthmatic lung ODO3112 | 29321 | 40 | 0 | 0.00 | asthmatic lung | −9.62 |
| asthmatic lung ODO3433 | 29323 | 38.36 | 6.1 | 12.20 | asthmatic lung | 1.27 |
| asthmatic lung ODO3397 | 29322 | 37.29 | 12.16 | 24.32 | asthmatic lung | 2.53 |
| asthmatic lung ODO4928 | 29325 | 38.01 | 7.64 | 15.28 | asthmatic lung | 1.59 |
| endo cells KC | control | 40 | 0 | 0.00 | endo cells | |
| endo VEGF KC | | 40 | 0 | 0.00 | endo VEGF | 0.00 |
| endo bFGF KC | | 40 | 0 | 0.00 | endo bFGF | 0.00 |
| heart Clontech | normal | 37.85 | 8.49 | 16.98 | heart | |
| heart (T-1) ischemic | 29417 | 40 | 0 | 0.00 | heart T-1 | −16.98 |
| heart (T-14) non- | 29422 | 40 | 0 | 0.00 | heart T-14 | −16.98 |
| obstructive DCM | | | | | | |
| heart (T-3399) DCM | 29426 | 39.5 | 2.9 | 5.80 | heart T-3399 | −2.93 |
| adenoid GW99-269 | 26162 | 40 | 0 | 0.00 | adenoid | |
| tonsil GW98-280 | 22582 | 36.35 | 22.46 | 44.92 | tonsil | |
| T cells PC00314 | 28453 | 36.17 | 25.23 | 50.46 | T cells | |
| PBMNC | | 40 | 0 | 0.00 | PBMNC | |
| monocyte | | 40 | 0.45 | 0.90 | monocyte | |
| B cells PC00665 | 28455 | 40 | 0.6 | 1.20 | B cells | |
| dendritic cells 28441 | | 38.07 | 7.37 | 14.74 | dendritic cells | |
| neutrophils | 28440 | 32.73 | 236.09 | 236.09 | neutrophils | |
| eosinophils | 28446 | 33.68 | 126.65 | 253.30 | eosinophils | |
| BM unstim | | 40 | 0 | 0.00 | BM unstim | |
| BM stim | | 36.52 | 20.1 | 20.10 | BM stim | 20.10 |
| osteo dif | | 40 | 0 | 0.00 | osteo dif | 0.00 |
| osteo undif | | 40 | 0 | 0.00 | osteo undif | |
| chondrocytes | | 39.61 | 2.7 | 6.75 | chondrocytes | |
| OA Synovium IP12/01 | 29462 | 39.91 | 1.59 | 1.59 | OA Synovium | |
| OA Synovium NP10/01 | 29461 | 38.98 | 4.07 | 8.14 | OA Synovium | |
| OA Synovium NP57/00 | 28464 | 36.29 | 23.27 | 46.54 | OA Synovium | |
| RA Synovium NP03/01 | 28466 | 35.81 | 31.94 | 63.88 | RA Synovium | |
| RA Synovium NP71/00 | 28467 | 34.79 | 61.79 | 123.58 | RA Synovium | |
| RA Synovium NP45/00 | 28475 | 35.08 | 51.15 | 102.30 | RA Synovium | |
| OA bone (biobank) | 29217 | 40 | 1.12 | 1.12 | OA bone | |
| | | | | | (biobank) | |
| OA bone Sample 1 | J. Emory | 36.8 | 16.77 | 33.54 | OA bone | |
| OA bone Sample 2 | J. Emory | 34.72 | 64.74 | 129.48 | OA bone | |
| Cartilage (pool) | Normal | 37.46 | 10.93 | 21.86 | Cartilage (pool) | |
| Cartilage (pool) | OA | 40 | 0 | 0.00 | Cartilage (pool) | −21.86 |
| PBL unifected | 28441 | 35.05 | 52.27 | 104.54 | PBL unifected | |
| PBL HIV IIIB | 28442 | 37.31 | 12.04 | 24.08 | PBL HIV IIIB | −4.34 |
| MRC5 uninfected | 29158 | 40 | 0 | 0.00 | MRC5 uninfected | |
| (100%) | | | | | (100%) | |
| MRC5 HSV strain F | 29178 | 38.19 | 6.8 | 13.60 | MRC5 HSV strain F | 13.60 |
| W12 cells | 29179 | 40 | 0 | 0.00 | W12 cells | |
| Keratinocytes | 29180 | 40 | 0 | 0.00 | Keratinocytes | |
| B-actin control | B-actin | 29.77 | 1604.25 | | | |
| | control | | | | | |
| genomic | genomic | 30.5 | 1001.61 | | | |
| 1.00E+05 | 1.00E+05 | 22.95 | 100000 | | | |
| 1.00E+05 | 1.00E+05 | 22.91 | 100000 | | | |
| 1.00E+04 | 1.00E+04 | 26.49 | 10000 | | | |
| 1.00E+04 | 1.00E+04 | 26.66 | 10000 | | | |
| 1.00E+03 | 1.00E+03 | 30.62 | 1000 | | | |
| 1.00E+03 | 1.00E+03 | 30.43 | 1000 | | | |
| 1.00E+02 | 1.00E+02 | 36.12 | 100 | | | |
| 1.00E+02 | 1.00E+02 | 34.73 | 100 | | | |
| 1.00E+01 | 1.00E+01 | 38.58 | 10 | | | |
| 1.00E+01 | 1.00E+01 | 38.82 | 10 | | | |
| 1.00E−00 | 1.00E−00 | 40 | 0 | | | |
| 1.00E−00 | 1.00E−00 | 37.86 | 1 | | | |
| NTC | NTC | 40 | 0 | | | |

Gene Name sbg995002PIGR

| | Fold Change in Disease |
|---|---|
| | Population Relative to |
| Disease tissues | Normal |
| colon tumor | 1.83 |
| colon tumor | 1351.60 |
| colon tumor | 3.51 |
| colon tumor | 2.76 |
| lung tumor | 104.21 |
| lung tumor | −1.64 |
| lung tumor | 2.25 |
| lung tumor | 1.48 |
| breast tumor | 3.89 |
| breast tumor | 9.96 |
| breast tumor | 0.00 |
| breast tumor | −52.26 |
| brain stage 5 ALZ | −8.95 |
| brain stage 5 ALZ | −8.78 |
| brain stage 5 ALZ | −5.09 |
| brain stage 5 ALZ | −2.36 |
| lung 24 | −9.62 |
| lung 28 | −9.62 |
| lung 23 | −9.62 |
| asthmatic lung | −9.62 |
| asthmatic lung | 1.27 |
| asthmatic lung | 2.53 |
| asthmatic lung | 1.59 |
| endo VEGF | 0.00 |
| endo bFGF | 0.00 |
| heart T-1 | −16.98 |
| heart T-14 | −16.98 |
| heart T-3399 | −2.93 |
| BM stim | 20.10 |
| osteo dif | 0.00 |
| Cartilage (pool) | −21.86 |
| PBL HIV IIIB | −4.34 |
| MRC5 HSV strain F | 13.60 |

Gene Name sbg1033026C1q

Low to moderate overall expression in normal and disease samples. Highest normal expression in the subcutaneous adipocytes, subcutaneous adipose, whole brain, and heart. Highest disease expression in the 3 heart samples. Downregulation in 1 of 4 lung tumor samples and upregulation in 2 of 4 breast tumor samples indicates a role for this gene in cancers of the lung and breast. Upregulation in 2 of 4 AD brain samples suggests an involvement in Alzheimer's disease. Upregulation in 3 of 3 heart samples implies an involvement in cardiovascular diseases such as non-obstructive and obstructive DCM as well as ischemia. Low expression in all of the immune cells. Low to moderate expression in the OA synovium and bone samples as well as in the RA synovium samples.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1033026C1q | 1 and 2) | 1) | 2) | Copies | (ng) | (ng) | RNA |
| Subcutaneous | 32.32, 31.18 | 11.99 | 24.91 | 18.45 | 3.06 | 16.34 | 301.47 |
| Adipocytes Zenbio | | | | | | | |
| Subcutaneous Adipose | 34.95, 33.78 | 2.2 | 4.69 | 3.45 | 0.96 | 52.36 | 180.37 |
| Zenbio | | | | | | | |
| Adrenal Gland | 40, 40 | 0 | 0 | 0.00 | 0.61 | 81.97 | 0.00 |
| Clontech | | | | | | | |
| Whole Brain Clontech | 28.5, 28.04 | 140.11 | 187.98 | 164.05 | 7.24 | 6.91 | 1132.91 |
| Fetal Brain Clontech | 40, 40 | 0 | 0 | 0.00 | 0.48 | 103.95 | 0.00 |
| Cerebellum Clontech | 40, 40 | 0 | 0 | 0.00 | 2.17 | 23.04 | 0.00 |
| Cervix | 40, 40 | 0 | 0 | 0.00 | 2.42 | 20.66 | 0.00 |
| Colon | 34.82, 38.44 | 2.39 | 0.23 | 1.31 | 2.71 | 18.45 | 24.17 |
| Endometrium | 40, 35.09 | 0 | 2.01 | 1.01 | 0.73 | 68.21 | 68.55 |
| Esophagus | 40, 40 | 0 | 0 | 0.00 | 1.37 | 36.50 | 0.00 |
| Heart Clontech | 32.53, 34.31 | 10.45 | 3.32 | 6.89 | 1.32 | 37.88 | 260.80 |
| Hypothalamus | 40, 40 | 0 | 0 | 0.00 | 0.32 | 155.28 | 0.00 |
| Ileum | 40, 34.55 | 0 | 2.85 | 1.43 | 2.58 | 19.38 | 27.62 |
| Jejunum | 33.04, 34.86 | 7.51 | 2.33 | 4.92 | 6.60 | 7.58 | 37.27 |
| Kidney | 40, 40 | 0 | 0 | 0.00 | 2.12 | 23.58 | 0.00 |
| Liver | 35.81, 33.92 | 1.26 | 4.28 | 2.77 | 1.50 | 33.33 | 92.33 |
| Fetal Liver Clontech | 32.05, 40 | 14.25 | 0 | 7.13 | 10.40 | 4.81 | 34.25 |
| Lung | 40, 33.51 | 0 | 5.58 | 2.79 | 2.57 | 19.46 | 54.28 |
| Mammary Gland | 31.05, 30.69 | 27.02 | 34.2 | 30.61 | 13.00 | 3.85 | 117.73 |
| Clontech | | | | | | | |
| Myometrium | 33.29, 35.1 | 6.42 | 2 | 4.21 | 2.34 | 21.37 | 89.96 |
| Omentum | 34.44, 40 | 3.07 | 0 | 1.54 | 3.94 | 12.69 | 19.48 |
| Ovary | 32.59, 35.04 | 10.03 | 2.07 | 6.05 | 4.34 | 11.52 | 69.70 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 34.63, 33.09 | 2.71 | 7.29 | 5.00 | 5.48 | 9.12 | 45.62 |
| Placenta Clontech | 32.77, 33.01 | 8.94 | 7.7 | 8.32 | 5.26 | 9.51 | 79.09 |
| Prostate | 40, 40 | 0 | 0 | 0.00 | 3.00 | 16.67 | 0.00 |
| Rectum | 40, 34.44 | 0 | 3.06 | 1.53 | 1.23 | 40.65 | 62.20 |
| Salivary Gland | 32.96, 40 | 7.94 | 0 | 3.97 | 7.31 | 6.84 | 27.15 |
| Clontech | | | | | | | |
| Skeletal Muscle | 40, 40 | 0 | 0 | 0.00 | 1.26 | 39.68 | 0.00 |
| Clontech | | | | | | | |
| Skin | 40, 33.46 | 0.6 | 5.75 | 3.18 | 1.21 | 41.32 | 131.20 |
| Small Intestine | 40, 40 | 0 | 0 | 0.00 | 0.98 | 51.07 | 0.00 |
| Clontech | | | | | | | |
| Spleen | 35.65, 35 | 1.4 | 2.13 | 1.77 | 4.92 | 10.16 | 17.94 |
| Stomach | 40, 34.73 | 0 | 2.54 | 1.27 | 2.73 | 18.32 | 23.26 |
| Testis Clontech | 40, 35.12 | 0 | 1.98 | 0.99 | 0.57 | 87.87 | 86.99 |
| Thymus Clontech | 32.44, 31.57 | 11.11 | 19.37 | 15.24 | 9.89 | 5.06 | 77.05 |
| Thyroid | 40, 40 | 0 | 0 | 0.00 | 2.77 | 18.05 | 0.00 |
| Trachea Clontech | 34.58, 33.56 | 2.79 | 5.4 | 4.10 | 9.71 | 5.15 | 21.09 |
| Urinary Bladder | 33.45, 33.34 | 5.8 | 6.21 | 6.01 | 5.47 | 9.14 | 54.89 |
| Uterus | 33.19, 32.41 | 6.82 | 11.32 | 9.07 | 5.34 | 9.36 | 84.93 |
| genomic | 25.47 | 981.57 | | | | | |
| b-actin | 26.87 | 398.61 | | | | | |
| 1.00E+05 | 18.24 | 100000 | | | | | |
| 1.00E+05 | 18.35 | 100000 | | | | | |
| 1.00E+04 | 21.53 | 10000 | | | | | |
| 1.00E+04 | 21.62 | 10000 | | | | | |
| 1.00E+03 | 25.17 | 1000 | | | | | |
| 1.00E+03 | 25.03 | 1000 | | | | | |
| 1.00E+02 | 30.53 | 100 | | | | | |
| 1.00E+02 | 30.49 | 100 | | | | | |
| 1.00E+01 | 30.85 | 10 | | | | | |
| 1.00E+01 | 40 | 10 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | Fold |
| | Reg | | | detected/ | | Change |
| | number | | Mean | 50 ng | | in |
| Sample | (GSK | | GOI | total | | Disease |
| sbg1033026C1q | identifier) | Ct | copies | RNA | Sample | Population |
| colon normal GW98-167 | 21941 | 27.03 | 397.15 | 794.30 | colon normal | |
| colon tumor GW98-166 | 21940 | 29.53 | 91.39 | 182.78 | colon tumor | −4.35 |
| colon normal GW98-178 | 22080 | 30.3 | 57.81 | 115.62 | colon normal | |
| colon tumor GW98-177 | 22060 | 29.31 | 103.84 | 207.68 | colon tumor | 1.80 |
| colon normal GW98-561 | 23514 | 28.79 | 140.64 | 281.28 | colon normal | |
| colon tumor GW98-560 | 23513 | 30.18 | 62.18 | 124.36 | colon tumor | −2.26 |
| colon normal GW98-894 | 24691 | 28.31 | 187.28 | 374.56 | colon normal | |
| colon tumor GW98-893 | 24690 | 28.75 | 143.93 | 287.86 | colon tumor | −1.30 |
| lung normal GW98-3 | 20742 | 28.18 | 201.78 | 403.56 | lung normal | |
| lung tumor GW98-2 | 20741 | 32.35 | 17.41 | 34.82 | lung tumor | −11.59 |
| lung normal GW97-179 | 20677 | 29.94 | 71.52 | 143.04 | lung normal | |
| lung tumor GW97-178 | 20676 | 28.76 | 143.36 | 286.72 | lung tumor | 2.00 |
| lung normal GW98-165 | 21922 | 28.69 | 149.49 | 298.98 | lung normal | |
| lung tumor GW98-164 | 21921 | 31.28 | 32.63 | 65.26 | lung tumor | −4.58 |
| lung normal GW98-282 | 22584 | 31.42 | 30.07 | 60.14 | lung normal | |
| lung tumor GW98-281 | 22583 | 30.33 | 56.83 | 113.66 | lung tumor | 1.89 |
| breast normal GW00-392 | 28750 | 28.72 | 146.7 | 146.70 | breast normal | |
| breast tumor GW00-391 | 28746 | 27.02 | 398.56 | 797.12 | breast tumor | 5.43 |
| breast normal GW00-413 | 28798 | 30.95 | 39.63 | 39.63 | breast normal | |
| breast tumor GW00-412 | 28797 | 30.58 | 49.03 | 98.06 | breast tumor | 2.47 |
| breast normal GW00- | 27592-95 | 32.53 | 15.6 | 15.60 | breast normal | |
| 235:238 | | | | | | |
| breast tumor GW00- | 27588-91 | 29.58 | 88.49 | 88.49 | breast tumor | 5.67 |
| 231:234 | | | | | | |
| breast normal GW98-621 | 23656 | 27.5 | 300.39 | 600.78 | breast normal | |
| breast tumor GW98-620 | 23655 | 29.28 | 105.43 | 210.86 | breast tumor | −2.85 |
| brain normal BB99-542 | 25507 | 30.67 | 46.59 | 93.18 | brain normal | |
| brain normal BB99-406 | 25509 | 29.54 | 90.66 | 181.32 | brain normal | |
| brain normal BB99-904 | 25546 | 31.13 | 35.58 | 71.16 | brain normal | |
| brain stage 5 ALZ BB99- | 25502 | 31.4 | 30.36 | 60.72 | brain stage 5 ALZ | −1.90 |
| 874 | | | | | | |
| brain stage 5 ALZ BB99- | 25503 | 28.39 | 177.94 | 355.88 | brain stage 5 ALZ | 3.09 |
| 887 | | | | | | |
| brain stage 5 ALZ BB99- | 25504 | 28.92 | 130.19 | 260.38 | brain stage 5 ALZ | 2.26 |
| 862 | | | | | | |
| brain stage 5 ALZ BB99- | 25542 | 28.2 | 198.98 | 397.96 | brain stage 5 ALZ | 3.45 |
| 927 | | | | | | |
| CT lung KC | normal | 31.24 | 33.37 | 66.74 | CT lung | |
| lung 26 KC | normal | 33.59 | 8.37 | 8.37 | lung 26 | |
| lung 27 KC | normal | 37.8 | 0.7 | 0.70 | lung 27 | |
| lung 24 KC | COPD | 34.56 | 4.73 | 4.73 | lung 24 | −4.39 |
| lung 28 KC | COPD | 35.39 | 2.91 | 2.91 | lung 28 | −7.13 |
| lung 23 KC | COPD | 34.74 | 4.26 | 4.26 | lung 23 | −4.87 |
| lung 25 KC | normal | 33.85 | 7.19 | 7.19 | lung 25 | |
| asthmatic lung ODO3112 | 29321 | 35.04 | 3.57 | 3.57 | asthmatic lung | −5.81 |
| asthmatic lung ODO3433 | 29323 | 32.44 | 16.48 | 32.96 | asthmatic lung | 1.59 |
| asthmatic lung ODO3397 | 29322 | 29.4 | 98.44 | 196.88 | asthmatic lung | 9.49 |
| asthmatic lung ODO4928 | 29325 | 31.1 | 36.23 | 72.46 | asthmatic lung | 3.49 |
| endo cells KC | control | 40 | 0 | 0.00 | endo cells | |
| endo VEGF KC | | 40 | 0 | 0.00 | endo VEGF | 0.00 |
| endo bFGF KC | | 35.4 | 2.89 | 2.89 | endo bFGF | 2.89 |
| heart Clontech | normal | 29.05 | 120.78 | 241.56 | heart | |
| heart (T-1) ischemic | 29417 | 26.23 | 633.79 | 1267.58 | heart T-1 | 5.25 |
| heart (T-14) non- | 29422 | 25.74 | 847.85 | 1695.70 | heart T-14 | 7.02 |
| obstructive DCM | | | | | | |
| heart (T-3399) DCM | 29426 | 25.03 | 1289.37 | 2578.74 | heart T-3399 | 10.68 |
| adenoid GW99-269 | 26162 | 35.24 | 3.17 | 6.34 | adenoid | |
| tonsil GW98-280 | 22582 | 31 | 38.35 | 76.70 | tonsil | |
| T cells PC00314 | 28453 | 31.75 | 24.69 | 49.38 | T cells | |
| PBMNC | | 40 | 0 | 0.00 | PBMNC | |
| monocyte | | 40 | 0 | 0.00 | monocyte | |
| B cells PC00665 | 28455 | 31.35 | 31.31 | 62.62 | B cells | |
| dendritic cells 28441 | | 32.81 | 13.25 | 26.50 | dendritic cells | |
| neutrophils | 28440 | 31.76 | 24.51 | 24.51 | neutrophils | |
| eosinophils | 28446 | 33.08 | 11.29 | 22.58 | eosinophils | |
| BM unstim | | 36.9 | 1.19 | 1.19 | BM unstim | |
| BM stim | | 38.86 | 0.38 | 0.38 | BM stim | −3.13 |
| osteo dif | | 37.77 | 0.72 | 0.72 | osteo dif | 0.72 |
| osteo undif | | 40 | 0 | 0.00 | osteo undif | |
| chondrocytes | | 33.14 | 10.88 | 27.20 | chondrocytes | |
| OA Synovium IP12/01 | 29462 | 30.61 | 48.28 | 48.28 | OA Synovium | |
| OA Synovium NP10/01 | 29461 | 27.5 | 300.97 | 601.94 | OA Synovium | |
| OA Synovium NP57/00 | 28464 | 29.28 | 105.55 | 211.10 | OA Synovium | |
| RA Synovium NP03/01 | 28466 | 30.18 | 62.3 | 124.60 | RA Synovium | |
| RA Synovium NP71/00 | 28467 | 29.22 | 109.5 | 219.00 | RA Synovium | |
| RA Synovium NP45/00 | 28475 | 30.06 | 66.71 | 133.42 | RA Synovium | |
| OA bone (biobank) | 29217 | 32.6 | 14.99 | 14.99 | OA bone (biobank) | |
| OA bone Sample 1 | J. Emory | 30.48 | 52.25 | 104.50 | OA bone | |
| OA bone Sample 2 | J. Emory | 32.31 | 17.76 | 35.52 | OA bone | |
| Cartilage (pool) | Normal | 30.45 | 53.05 | 106.10 | Cartilage (pool) | |
| Cartilage (pool) | OA | 30.81 | 43.01 | 86.02 | Cartilage (pool) | −1.23 |
| PBL unifected | 28441 | 30.19 | 61.92 | 123.84 | PBL unifected | |
| PBL HIV IIIB | 28442 | 31.19 | 34.35 | 68.70 | PBL HIV IIIB | −1.80 |
| MRC5 uninfected | 29158 | 30.19 | 62.02 | 124.04 | MRC5 uninfected | |
| (100%) | | | | | (100%) | |
| MRC5 HSV strain F | 29178 | 31.13 | 35.48 | 70.96 | MRC5 HSV strain F | −1.75 |
| W12 cells | 29179 | 32 | 21.32 | 42.64 | W12 cells | |
| Keratinocytes | 29180 | 33.3 | 9.92 | 19.84 | Keratinocytes | |
| B-actin control | | 26.66 | 492.23 | | | |
| genomic | | 24.83 | 1443.91 | | | |
| 1.00E+05 | | 18.12 | 100000 | | | |
| 1.00E+05 | | 18.12 | 100000 | | | |
| 1.00E+04 | | 21.28 | 10000 | | | |
| 1.00E+04 | | 21.31 | 10000 | | | |
| 1.00E+03 | | 24.92 | 1000 | | | |
| 1.00E+03 | | 24.9 | 1000 | | | |
| 1.00E+02 | | 29.22 | 100 | | | |
| 1.00E+02 | | 29.26 | 100 | | | |
| 1.00E+01 | | 33.13 | 10 | | | |
| 1.00E+01 | | 34.32 | 10 | | | |
| 1.00E−00 | | 40 | 0 | | | |
| 1.00E−00 | | 40 | 0 | | | |
| NTC | | 40 | 0 | | | |

Gene Name sbg1033026C1q

| | Fold Change in Disease |
|---|---|
| | Population Relative to |
| Disease tissues | Normal |
| colon tumor | −4.35 |
| colon tumor | 1.80 |
| colon tumor | −2.26 |
| colon tumor | −1.30 |
| lung tumor | −11.59 |
| lung tumor | 2.00 |
| lung tumor | −4.58 |
| lung tumor | 1.89 |
| breast tumor | 5.43 |
| breast tumor | 2.47 |
| breast tumor | 5.67 |
| breast tumor | −2.85 |
| brain stage 5 ALZ | −1.90 |
| brain stage 5 ALZ | 3.09 |
| brain stage 5 ALZ | 2.26 |
| brain stage 5 ALZ | 3.45 |
| lung 24 | −4.39 |
| lung 28 | −7.13 |
| lung 23 | −4.87 |
| asthmatic lung | −5.81 |
| asthmatic lung | 1.59 |
| asthmatic lung | 9.49 |
| asthmatic lung | 3.49 |
| endo VEGF | 0.00 |
| endo bFGF | 2.89 |
| heart T-1 | 5.25 |
| heart T-14 | 7.02 |
| heart T-3399 | 10.68 |
| BM stim | −3.13 |
| osteo dif | 0.72 |
| Cartilage (pool) | −1.23 |
| PBL HIV IIIB | −1.80 |
| MRC5 HSV strain F | −1.75 |

Gene Name sbg1003675Rnase
FAILED
Gene Name sbg1015258PLM

Low overall expression in normal and disease samples. Highest normal expression in the endometrium, hypothalamus, liver small intestine, and the testis. Highest disease expression in one of the breast normal/tumor pairs, one of the normal brain samples, two of the Alzheimer's disease brain samples, the B cells and the HSV-infected MRC5 cells. Downregulation in 1 of 4 lung tumor samples is sufficient to claim a role in cancer of the lung. Upregulation in 2 of 4 breast tumor samples indicates an involvement in cancer of the breast. Downregulation in 2 of 3 COPD samples and in 1 of 4 asthmatic lung samples implies a role in chronic obstructive pulmonary disease and asthma. Upregulation in the obstructive DCM heart sample suggests a potential role in cardiovascular disease. Downregulation in the stimulated bone marrow sample. Downregulation in the OA cartilage pool implicates this gene in osteoarthritis. Upregulation in the HSV-infected MRC5 cells suggests that this gene may be a host factor in HSV. Low expression in all immune cells except the B cells which show moderate expression.

| | | | | | | | copies of |
|---|---|---|---|---|---|---|---|
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1015258PLM | 1 and 2) | 1) | 2) | Copies | (ng) | (ng) | RNA |
| Subcutaneous | 39.96, 39.32 | 0.39 | 0.73 | 0.56 | 3.06 | 16.34 | 9.15 |
| Adipocytes Zenbio | | | | | | | |
| Subcutaneous Adipose | 39.61, 37.77 | 0.62 | 1.84 | 1.23 | 0.96 | 52.36 | 64.40 |
| Zenbio | | | | | | | |
| Adrenal Gland Clontech | 36.89, 37.65 | 3.12 | 1.98 | 2.55 | 0.61 | 81.97 | 209.02 |
| Whole Brain Clontech | 32.77, 33.06 | 36.18 | 30.37 | 33.28 | 7.24 | 6.91 | 229.80 |
| Fetal Brain Clontech | 38.01, 37.52 | 1.6 | 2.14 | 1.87 | 0.48 | 103.95 | 194.39 |
| Cerebellum Clontech | 38.34, 37.8 | 1.31 | 1.81 | 1.56 | 2.17 | 23.04 | 35.94 |
| Cervix | 36.9, 37.17 | 3.1 | 2.64 | 2.87 | 2.42 | 20.66 | 59.30 |
| Colon | 35.93, 37.95 | 5.51 | 1.66 | 3.59 | 2.71 | 18.45 | 66.14 |
| Endometrium | 34.62, 37.51 | 12.02 | 2.15 | 7.09 | 0.73 | 68.21 | 483.29 |
| Esophagus | 35.76, 37.39 | 6.1 | 2.31 | 4.21 | 1.37 | 36.50 | 153.47 |
| Heart Clontech | 36.78, 38.53 | 3.33 | 1.17 | 2.25 | 1.32 | 37.88 | 85.23 |
| Hypothalamus | 37.12, 36.52 | 2.71 | 3.87 | 3.29 | 0.32 | 155.28 | 510.87 |
| Ileum | 36.15, 36.19 | 4.84 | 4.72 | 4.78 | 2.58 | 19.38 | 92.64 |
| Jejunum | 34.6, 33.48 | 12.14 | 23.65 | 17.90 | 6.60 | 7.58 | 135.57 |
| Kidney | 37.16, 37.9 | 2.65 | 1.7 | 2.18 | 2.12 | 23.58 | 51.30 |
| Liver | 34.6, 36.06 | 12.13 | 5.11 | 8.62 | 1.50 | 33.33 | 287.33 |
| Fetal Liver Clontech | 34.32, 36.91 | 14.35 | 3.07 | 8.71 | 10.40 | 4.81 | 41.88 |
| Lung | 36.94, 35.31 | 3.02 | 7.98 | 5.50 | 2.57 | 19.46 | 107.00 |
| Mammary Gland | 38.03, 36.89 | 1.58 | 3.11 | 2.35 | 13.00 | 3.85 | 9.02 |
| Clontech | | | | | | | |
| Myometrium | 38.44, 38 | 1.24 | 1.6 | 1.42 | 2.34 | 21.37 | 30.34 |
| Omentum | 35.41, 35.38 | 7.51 | 7.64 | 7.58 | 3.94 | 12.69 | 96.13 |
| Omentum | | | | | | | |
| Ovary | 34.32, 34.36 | 14.4 | 14.07 | 14.24 | 4.34 | 11.52 | 164.00 |
| Pancreas | 36.44, 36.82 | 4.07 | 3.25 | 3.66 | 0.81 | 61.80 | 226.21 |
| Head of Pancreas | 37.54, 36.96 | 2.11 | 2.98 | 2.55 | 1.57 | 31.85 | 81.05 |
| Parotid Gland | 37.1, 35.46 | 2.75 | 7.29 | 5.02 | 5.48 | 9.12 | 45.80 |
| Placenta Clontech | 36.07, 35.36 | 5.08 | 7.74 | 6.41 | 5.26 | 9.51 | 60.93 |
| Prostate | 37.4, 37.82 | 2.3 | 1.79 | 2.05 | 3.00 | 16.67 | 34.08 |
| Rectum | 36.65, 37.39 | 3.59 | 2.32 | 2.96 | 1.23 | 40.65 | 120.12 |
| Salivary Gland | 38.55, 39.31 | 1.16 | 0.74 | 0.95 | 7.31 | 6.84 | 6.50 |
| Clontech | | | | | | | |
| Skeletal Muscle | 37.59, 36.87 | 2.06 | 3.15 | 2.61 | 1.26 | 39.68 | 103.37 |
| Clontech | | | | | | | |
| Skin | 38.36, 36.33 | 1.3 | 4.34 | 2.82 | 1.21 | 41.32 | 116.53 |
| Small Intestine | 36.05, 36.76 | 5.14 | 3.37 | 4.26 | 0.98 | 51.07 | 217.31 |
| Clontech | | | | | | | |
| Spleen | 37.62, 35.34 | 2.02 | 7.83 | 4.93 | 4.92 | 10.16 | 50.05 |
| Stomach | 35.8, 35.15 | 5.95 | 8.76 | 7.36 | 2.73 | 18.32 | 134.71 |
| Testis Clontech | 35.14, 37.08 | 8.82 | 2.77 | 5.80 | 0.57 | 87.87 | 509.23 |
| Thymus Clontech | 35.89, 35.06 | 5.65 | 9.22 | 7.44 | 9.89 | 5.06 | 37.59 |
| Thyroid | 37.59, 37.2 | 2.06 | 2.59 | 2.33 | 2.77 | 18.05 | 41.97 |
| Trachea Clontech | 37.52, 37.95 | 2.14 | 1.66 | 1.90 | 9.71 | 5.15 | 9.78 |
| Urinary Bladder | 37.47, 35.44 | 2.2 | 7.38 | 4.79 | 5.47 | 9.14 | 43.78 |
| Uterus | 34.17, 34.12 | 15.66 | 16.17 | 15.92 | 5.34 | 9.36 | 149.02 |
| genomic | 27.03 | 1097.52 | | | | | |
| b-actin | 27.23 | 974.77 | | | | | |
| 1.00E+05 | 19.25 | 100000 | | | | | |
| 1.00E+05 | 19.2 | 100000 | | | | | |
| 1.00E+04 | 22.99 | 10000 | | | | | |
| 1.00E+04 | 22.94 | 10000 | | | | | |
| 1.00E+03 | 27.09 | 1000 | | | | | |
| 1.00E+03 | 27.28 | 1000 | | | | | |
| 1.00E+02 | 31.49 | 100 | | | | | |
| 1.00E+02 | 31.46 | 100 | | | | | |
| 1.00E+01 | 37.86 | 10 | | | | | |
| 1.00E+01 | 35.45 | 10 | | | | | |
| 1.00E−00 | 37.08 | 1 | | | | | |
| 1.00E−00 | 37.4 | 1 | | | | | |
| NTC | 36.45 | −1 | | | | | |
| NTC | 36.15 | −1 | | | | | |

| | | | | copies of | | |
|---|---|---|---|---|---|---|
| | | | | mRNA | | |
| | Reg | | | detected/ | | Fold |
| | number | | Mean | 50 ng | | Change in |
| Sample | (GSK | | GOI | total | | Disease |
| sbg1015258PLM | identifier) | Ct | copies | RNA | Sample | Population |
| colon normal GW98-167 | 21941 | 35.4 | 9.91 | 19.82 | colon normal | |
| colon tumor GW98-166 | 21940 | 35.13 | 11.61 | 23.22 | colon tumor | 1.17 |
| colon normal GW98-178 | 22080 | 34.84 | 13.83 | 27.66 | colon normal | |
| colon tumor GW98-177 | 22060 | 35 | 12.59 | 25.18 | colon tumor | −1.10 |
| colon normal GW98-561 | 23514 | 35.48 | 9.4 | 18.80 | colon normal | |
| colon tumor GW98-560 | 23513 | 36.2 | 6.14 | 12.28 | colon tumor | −1.53 |
| colon normal GW98-894 | 24691 | 34.53 | 16.62 | 33.24 | colon normal | |
| colon tumor GW98-893 | 24690 | 34.29 | 19.25 | 38.50 | colon tumor | 1.16 |
| lung normal GW98-3 | 20742 | 34.53 | 16.69 | 33.38 | lung normal | |
| lung tumor GW98-2 | 20741 | 37.87 | 2.25 | 4.50 | lung tumor | −7.42 |
| lung normal GW97-179 | 20677 | 34.59 | 16.02 | 32.04 | lung normal | |
| lung tumor GW97-178 | 20676 | 34.45 | 17.43 | 34.86 | lung tumor | 1.09 |
| lung normal GW98-165 | 21922 | 33.89 | 24.39 | 48.78 | lung normal | |
| lung tumor GW98-164 | 21921 | 35.48 | 9.43 | 18.86 | lung tumor | −2.59 |
| lung normal GW98-282 | 22584 | 36.67 | 4.62 | 9.24 | lung normal | |
| lung tumor GW98-281 | 22583 | 37.87 | 2.26 | 4.52 | lung tumor | −2.04 |
| breast normal GW00- | 28750 | 34.17 | 20.64 | 20.64 | breast normal | |
| 392 | | | | | | |
| breast tumor GW00-391 | 28746 | 33.54 | 30.09 | 60.18 | breast tumor | 2.92 |
| breast normal GW00- | 28798 | 39.05 | 1.11 | 1.11 | breast normal | |
| 413 | | | | | | |
| breast tumor GW00-412 | 28797 | 35.01 | 12.46 | 24.92 | breast tumor | 22.45 |
| breast normal GW00- | 27592-95 | 40 | 0.55 | 0.55 | breast normal | |
| 235:238 | | | | | | |
| breast tumor GW00- | 27588-91 | 35.07 | 12.02 | 12.02 | breast tumor | 21.85 |
| 231:234 | | | | | | |
| breast normal GW98- | 23656 | 33 | 41.58 | 83.16 | breast normal | |
| 621 | | | | | | |
| breast tumor GW98-620 | 23655 | 33.05 | 40.47 | 80.94 | breast tumor | −1.03 |
| brain normal BB99-542 | 25507 | 32.73 | 48.93 | 97.86 | brain normal | |
| brain normal BB99-406 | 25509 | 33.97 | 23.34 | 46.68 | brain normal | |
| brain normal BB99-904 | 25546 | 37.88 | 2.24 | 4.48 | brain normal | |
| brain stage 5 ALZ | 25502 | 35.92 | 7.25 | 14.50 | brain stage 5 ALZ | −3.43 |
| BB99-874 | | | | | | |
| brain stage 5 ALZ | 25503 | 31.57 | 97.99 | 195.98 | brain stage 5 ALZ | 3.95 |
| BB99-887 | | | | | | |
| brain stage 5 ALZ | 25504 | 36.08 | 6.57 | 13.14 | brain stage 5 ALZ | −3.78 |
| BB99-862 | | | | | | |
| brain stage 5 ALZ | 25542 | 32.66 | 50.98 | 101.96 | brain stage 5 ALZ | 2.05 |
| BB99-927 | | | | | | |
| CT lung KC | normal | 37.37 | 3.04 | 6.08 | CT lung | |
| lung 26 KC | normal | | | | lung 26 | |
| lung 27 KC | normal | 38.52 | 1.52 | 1.52 | lung 27 | |
| lung 24 KC | COPD | 40 | 0 | 0.00 | lung 24 | −3.07 |
| lung 28 KC | COPD | 37.4 | 2.99 | 2.99 | lung 28 | −1.03 |
| lung 23 KC | COPD | 39.56 | 0.82 | 0.82 | lung 23 | −3.74 |
| lung 25 KC | normal | 38.43 | 1.61 | 1.61 | lung 25 | |
| asthmatic lung | 29321 | 39.45 | 0.88 | 0.88 | asthmatic lung | −3.49 |
| ODO3112 | | | | | | |
| asthmatic lung | 29323 | 36.48 | 5.19 | 10.38 | asthmatic lung | 3.38 |
| ODO3433 | | | | | | |
| asthmatic lung | 29322 | 35.56 | 8.99 | 17.98 | asthmatic lung | 5.86 |
| ODO3397 | | | | | | |
| asthmatic lung | 29325 | 37.06 | 3.66 | 7.32 | asthmatic lung | 2.38 |
| ODO4928 | | | | | | |
| endo cells KC | control | 39.29 | 0.96 | 0.96 | endo cells | |
| endo VEGF KC | | 37.65 | 2.57 | 2.57 | endo VEGF | 2.68 |
| endo bFGF KC | | 40 | 0.48 | 0.48 | endo bFGF | −2.00 |
| heart Clontech | normal | 40 | 0.68 | 1.36 | heart | |
| heart (T-1) ischemic | 29417 | 39.29 | 0.96 | 1.92 | heart T-1 | 1.41 |
| heart (T-14) non- | 29422 | 38.21 | 1.84 | 3.68 | heart T-14 | 2.71 |
| obstructive DCM | | | | | | |
| heart (T-3399) DCM | 29426 | 35.49 | 9.35 | 18.70 | heart T-3399 | 13.75 |
| adenoid GW99-269 | 26162 | 39.63 | 0.78 | 1.56 | adenoid | |
| tonsil GW98-280 | 22582 | 34.5 | 16.92 | 33.84 | tonsil | |
| T cells PC00314 | 28453 | 34.57 | 16.25 | 32.50 | T cells | |
| PBMNC | | 37.32 | 3.13 | 3.13 | PBMNC | |
| monocyte | | 37.74 | 2.44 | 4.88 | monocyte | |
| B cells PC00665 | 28455 | 33.32 | 34.36 | 68.72 | B cells | |
| dendritic cells 28441 | | 37.29 | 3.19 | 6.38 | dendritic cells | |
| neutrophils | 28440 | 36.01 | 6.85 | 6.85 | neutrophils | |
| eosinophils | 28446 | 35.38 | 9.98 | 19.96 | eosinophils | |
| BM unstim | | 37.44 | 2.91 | 2.91 | BM unstim | |
| BM stim | | 40 | 0.53 | 0.53 | BM stim | −5.49 |
| osteo dif | | 38.15 | 1.91 | 1.91 | osteo dif | −1.38 |
| osteo undif | | 37.6 | 2.64 | 2.64 | osteo undif | |
| chondrocytes | | 36.1 | 6.51 | 16.28 | chondrocytes | |
| OA Synovium IP12/01 | 29462 | 35.58 | 8.86 | 8.86 | OA Synovium | |
| OA Synovium NP10/01 | 29461 | 35.46 | 9.54 | 19.08 | OA Synovium | |
| OA Synovium NP57/00 | 28464 | 34.05 | 22.23 | 44.46 | OA Synovium | |
| RA Synovium NP03/01 | 28466 | 37.92 | 2.19 | 4.38 | RA Synovium | |
| RA Synovium NP71/00 | 28467 | 35.39 | 9.92 | 19.84 | RA Synovium | |
| RA Synovium NP45/00 | 28475 | 35.03 | 12.37 | 24.74 | RA Synovium | |
| OA bone (biobank) | 29217 | 39.98 | 0.67 | 0.67 | OA bone | |
| | | | | | (biobank) | |
| OA bone Sample 1 | J. Emory | 36.06 | 6.68 | 13.36 | OA bone | |
| OA bone Sample 2 | J. Emory | 39.89 | 0.67 | 1.34 | OA bone | |
| Cartilage (pool) | Normal | 35.57 | 8.92 | 17.84 | Cartilage (pool) | |
| Cartilage (pool) | OA | 39.5 | 0.85 | 1.70 | Cartilage (pool) | −10.49 |
| PBL unifected | 28441 | 36.65 | 4.67 | 9.34 | PBL unifected | |
| PBL HIV IIIB | 28442 | 36.94 | 3.92 | 7.84 | PBL HIV IIIB | −1.19 |
| MRC5 uninfected | 29158 | 37.54 | 2.74 | 5.48 | MRC5 uninfected | |
| (100%) | | | | | (100%) | |
| MRC5 HSV strain F | 29178 | 33.19 | 37.18 | 74.36 | MRC5 HSV strain F | 13.57 |
| W12 cells | 29179 | 37.23 | 3.3 | 6.60 | W12 cells | |
| Keratinocytes | 29180 | 37.18 | 3.4 | 6.80 | Keratinocytes | |
| B-actin control | | 27.26 | 1296.87 | | | |
| genomic | | 27.2 | 1345.34 | | | |
| 1.00E+05 | | 19.44 | 100000 | | | |
| 1.00E+05 | | 19.81 | 100000 | | | |
| 1.00E+04 | | 23.63 | 10000 | | | |
| 1.00E+04 | | 23.41 | 10000 | | | |
| 1.00E+03 | | 27.77 | 1000 | | | |
| 1.00E+03 | | 27.89 | 1000 | | | |
| 1.00E+02 | | 32.32 | 100 | | | |
| 1.00E+02 | | 32.3 | 100 | | | |
| 1.00E+01 | | 36.77 | 10 | | | |
| 1.00E+01 | | 36.39 | 10 | | | |
| 1.00E−00 | | 38.06 | 1 | | | |
| 1.00E−00 | | 37.56 | 1 | | | |
| NTC | | 38.18 | −1 | | | |

Gene Name sbg1015258PLM

| | Fold Change in Disease |
|---|---|
| | Population Relative to |
| Disease tissues | Normal |
| colon tumor | 1.17 |
| colon tumor | −1.10 |
| colon tumor | −1.53 |
| colon tumor | 1.16 |
| lung tumor | −7.42 |
| lung tumor | 1.09 |
| lung tumor | −2.59 |
| lung tumor | −2.04 |
| breast tumor | 2.92 |
| breast tumor | 22.45 |
| breast tumor | 21.85 |
| breast tumor | −1.03 |
| brain stage 5 ALZ | −3.43 |
| brain stage 5 ALZ | 3.95 |
| brain stage 5 ALZ | −3.78 |
| brain stage 5 ALZ | 2.05 |
| lung 24 | −3.07 |
| lung 28 | −1.03 |
| lung 23 | −3.74 |
| asthmatic lung | −3.49 |
| asthmatic lung | 3.38 |
| asthmatic lung | 5.86 |
| asthmatic lung | 2.38 |
| endo VEGF | 2.68 |
| endo bFGF | −2.00 |
| heart T-1 | 1.41 |
| heart T-14 | 2.71 |
| heart T-3399 | 13.75 |
| BM stim | −5.49 |
| osteo dif | −1.38 |
| Cartilage (pool) | −10.49 |
| PBL HIV IIIB | −1.19 |
| MRC5 HSV strain F | 13.57 |

Gene Name sbg1003328IG (Taqman)

Moderate overall expression. Highest normal expression in whole brain, fetal brain, and cerebellum with slightly lower levels of expression in the colon and mammary gland. Highest disease expression in the colon and lung tumor pairs as well as the normal and Alzheimer's brain. Significant upregulation in 2 of 4 breast tumor samples with slight upregulation in 1 of 4 breast tumor samples implicates this gene in breast cancer. Downregulation in 3 of 3 COPD samples may suggest an involvement in chronic obstructive pulmonary disease. Downregulation in 1 of 4 asthma samples suggests a potential role for this gene in asthma. Downregulation in the HSV-infected MRC5 cells suggests that this gene may play a role in HSV. High expression in 3 of 3 OA synovium samples, 3 of 3 RA synovium samples, 2 of 2 OA bone samples, and corroborating high expression in the T cells and B cells implicates this gene in osteoarthritis and rheumatoid arthritis.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1003328IG | 1 and 2) | 1) | 2) | Copies | (ng) | (ng) | RNA |
| Subcutaneous | 32.35, 32.1 | 109.15 | 127.12 | 118.14 | 3.06 | 16.34 | 1930.31 |
| Adipocytes Zenbio | | | | | | | |
| Subcutaneous Adipose | 37.92, 40 | 3.84 | 0 | 1.92 | 0.96 | 52.36 | 100.52 |
| Zenbio | | | | | | | |
| Adrenal Gland | 38.07, 35.73 | 3.5 | 14.32 | 8.91 | 0.61 | 81.97 | 730.33 |
| Clontech | | | | | | | |
| Whole Brain Clontech | 22.72, 23.09 | 35640.5 | 28576.52 | 32108.51 | 7.24 | 6.91 | 221743.85 |
| Fetal Brain Clontech | 33.39, 34.06 | 58.43 | 39.04 | 48.74 | 0.48 | 103.95 | 5066.01 |
| Cerebellum Clontech | 31.32, 31.02 | 202.49 | 242.95 | 222.72 | 2.17 | 23.04 | 5131.80 |
| Cervix | 36.36, 35.6 | 9.78 | 15.49 | 12.64 | 2.42 | 20.66 | 261.05 |
| Colon | 30.74, 32.11 | 286.9 | 125.78 | 206.34 | 2.71 | 18.45 | 3807.01 |
| Endometrium | 34.58, 36.01 | 28.47 | 12.11 | 20.29 | 0.73 | 68.21 | 1384.04 |
| Esophagus | 37.54, 36.05 | 4.82 | 11.78 | 8.30 | 1.37 | 36.50 | 302.92 |
| Heart Clontech | 36.23, 36.85 | 10.56 | 7.3 | 8.93 | 1.32 | 37.88 | 338.26 |
| Hypothalamus | 40, 36.96 | 0 | 6.81 | 3.41 | 0.32 | 155.28 | 528.73 |
| Ileum | 34.79, 34.48 | 25.09 | 30.4 | 27.75 | 2.58 | 19.38 | 537.69 |
| Jejunum | 30.14, 31.02 | 412.72 | 242.47 | 327.60 | 6.60 | 7.58 | 2481.78 |
| Kidney | 33.94, 34.61 | 41.89 | 28.12 | 35.01 | 2.12 | 23.58 | 825.59 |
| Liver | 36.11, 35.38 | 11.41 | 17.61 | 14.51 | 1.50 | 33.33 | 483.67 |
| Fetal Liver Clontech | 30.24, 30.27 | 388.12 | 381.28 | 384.70 | 10.40 | 4.81 | 1849.52 |
| Lung | 35.59, 35.09 | 15.59 | 20.98 | 18.29 | 2.57 | 19.46 | 355.74 |
| Mammary Gland | 29.21, 28.52 | 718.74 | 1090.18 | 904.46 | 13.00 | 3.85 | 3478.69 |
| Clontech | | | | | | | |
| Myometrium | 33.7, 34.11 | 48.42 | 37.85 | 43.14 | 2.34 | 21.37 | 921.69 |
| Omentum | 33.83, 33.57 | 44.75 | 52.27 | 48.51 | 3.94 | 12.69 | 615.61 |
| Ovary | 32.47, 32.34 | 101.42 | 109.96 | 105.69 | 4.34 | 11.52 | 1217.63 |
| Pancreas | 38.16, 40 | 3.31 | 0 | 1.66 | 0.81 | 61.80 | 102.29 |
| Head of Pancreas | 40, 36.68 | 0 | 8.07 | 4.04 | 1.57 | 31.85 | 128.50 |
| Parotid Gland | 31.68, 31.03 | 162.92 | 241.11 | 202.02 | 5.48 | 9.12 | 1843.20 |
| Placenta Clontech | 30.48, 30.82 | 335.37 | 274.53 | 304.95 | 5.26 | 9.51 | 2898.76 |
| Prostate | 33.39, 32.15 | 58.39 | 122.82 | 90.61 | 3.00 | 16.67 | 1510.08 |
| Rectum | 35.95, 35.32 | 12.5 | 18.32 | 15.41 | 1.23 | 40.65 | 626.42 |
| Salivary Gland | 31.25, 30.65 | 211.88 | 303.2 | 257.54 | 7.31 | 6.84 | 1761.56 |
| Clontech | | | | | | | |
| Skeletal Muscle | 36.61, 37.62 | 8.44 | 4.6 | 6.52 | 1.26 | 39.68 | 258.73 |
| Clontech | | | | | | | |
| Skin | 36.37, 36.08 | 9.71 | 11.56 | 10.64 | 1.21 | 41.32 | 439.46 |
| Small Intestine | 36.74, 34.51 | 7.79 | 29.79 | 18.79 | 0.98 | 51.07 | 959.65 |
| Clontech | | | | | | | |
| Spleen | 34.78, 35.63 | 25.25 | 15.18 | 20.22 | 4.92 | 10.16 | 205.44 |
| Stomach | 40, 35.13 | 0.88 | 20.56 | 10.72 | 2.73 | 18.32 | 196.34 |
| Testis Clontech | 35.01, 39.68 | 22.07 | 1.33 | 11.70 | 0.57 | 87.87 | 1028.12 |
| Thymus Clontech | 29.15, 29.2 | 749.23 | 724.61 | 736.92 | 9.89 | 5.06 | 3725.58 |
| Thyroid | 32.02, 32.14 | 133.23 | 123.77 | 128.50 | 2.77 | 18.05 | 2319.49 |
| Trachea Clontech | 31.28, 30.17 | 207.67 | 405.04 | 306.36 | 9.71 | 5.15 | 1577.52 |
| Urinary Bladder | 33.07, 32.91 | 70.6 | 78.07 | 74.34 | 5.47 | 9.14 | 679.48 |
| Uterus | 33.01, 33.15 | 73.56 | 67.48 | 70.52 | 5.34 | 9.36 | 660.30 |
| genomic | 29.15 | 746.77 | | | | | |
| b-actin | 30.2 | 397.52 | | | | | |
| 1.00E+05 | 21.06 | 100000 | | | | | |
| 1.00E+05 | 20.94 | 100000 | | | | | |
| 1.00E+04 | 25.06 | 10000 | | | | | |
| 1.00E+04 | 24.54 | 10000 | | | | | |
| 1.00E+03 | 28.32 | 1000 | | | | | |
| 1.00E+03 | 28.77 | 1000 | | | | | |
| 1.00E+02 | 33 | 100 | | | | | |
| 1.00E+02 | 32.74 | 100 | | | | | |
| 1.00E+01 | 35.9 | 10 | | | | | |
| 1.00E+01 | 40 | 0 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1003328IG | identifier) | Ct | copies | RNA | Sample | Population |
| colon normal GW98-167 | 21941 | 24.26 | 15509.01 | 31018.02 | colon normal | |
| colon tumor GW98-166 | 21940 | 24.5 | 13727.44 | 27454.88 | colon tumor | −1.13 |
| colon normal GW98-178 | 22080 | 28.01 | 2276.97 | 4553.94 | colon normal | |
| colon tumor GW98-177 | 22060 | 27.11 | 3618.2 | 7236.40 | colon tumor | 1.59 |
| colon normal GW98-561 | 23514 | 24.47 | 13993.67 | 27987.34 | colon normal | |
| colon tumor GW98-560 | 23513 | 25.32 | 9040.47 | 18080.94 | colon tumor | −1.55 |
| colon normal GW98-894 | 24691 | 24.17 | 16251.61 | 32503.22 | colon normal | |
| colon tumor GW98-893 | 24690 | 25.15 | 9872.46 | 19744.92 | colon tumor | −1.65 |
| lung normal GW98-3 | 20742 | 24.4 | 14498.52 | 28997.04 | lung normal | |
| lung tumor GW98-2 | 20741 | 24.66 | 12640.32 | 25280.64 | lung tumor | −1.15 |
| lung normal GW97-179 | 20677 | 24.12 | 16680.84 | 33361.68 | lung normal | |
| lung tumor GW97-178 | 20676 | 24.69 | 12468.91 | 24937.82 | lung tumor | −1.34 |
| lung normal GW98-165 | 21922 | 25.09 | 10168.18 | 20336.36 | lung normal | |
| lung tumor GW98-164 | 21921 | 25.49 | 8296.37 | 16592.74 | lung tumor | −1.23 |
| lung normal GW98-282 | 22584 | 26.85 | 4131.52 | 8263.04 | lung normal | |
| lung tumor GW98-281 | 22583 | 26.59 | 4702 | 9404.00 | lung tumor | 1.14 |
| breast normal GW00- | 28750 | 25.9 | 6719.98 | 6719.98 | breast normal | |
| 392 | | | | | | |
| breast tumor GW00-391 | 28746 | 25.04 | 10402.11 | 20804.22 | breast tumor | 3.10 |
| breast normal GW00- | 28798 | 32.51 | 226.94 | 226.94 | breast normal | |
| 413 | | | | | | |
| breast tumor GW00-412 | 28797 | 26.37 | 5261.73 | 10523.46 | breast tumor | 46.37 |
| breast normal GW00- | 27592-95 | 34.58 | 78.63 | 78.63 | breast normal | |
| 235:238 | | | | | | |
| breast tumor GW00- | 27588-91 | 28.45 | 1812.86 | 1812.86 | breast tumor | 23.06 |
| 231:234 | | | | | | |
| breast normal GW98- | 23656 | 25.26 | 9289.36 | 18578.72 | breast normal | |
| 621 | | | | | | |
| breast tumor GW98-620 | 23655 | 25.66 | 7579.13 | 15158.26 | breast tumor | −1.23 |
| brain normal BB99-542 | 25507 | 22.52 | 37845.47 | 75690.94 | brain normal | |
| brain normal BB99-406 | 25509 | 23.07 | 28574.8 | 57149.60 | brain normal | |
| brain normal BB99-904 | 25546 | 23.85 | 19214.49 | 38428.98 | brain normal | |
| brain stage 5 ALZ | 25502 | 25.98 | 6442.51 | 12885.02 | brain stage 5 ALZ | −4.43 |
| BB99-874 | | | | | | |
| brain stage 5 ALZ | 25503 | 23.19 | 26936.06 | 53872.12 | brain stage 5 ALZ | −1.06 |
| BB99-887 | | | | | | |
| brain stage 5 ALZ | 25504 | 23.42 | 23948.83 | 47897.66 | brain stage 5 ALZ | −1.19 |
| BB99-862 | | | | | | |
| brain stage 5 ALZ | 25542 | 24.15 | 16419.33 | 32838.66 | brain stage 5 ALZ | −1.74 |
| BB99-927 | | | | | | |
| CT lung KC | normal | 25.63 | 7714.35 | 15428.70 | CT lung | |
| lung 26 KC | normal | 32.34 | 247.99 | 247.99 | lung 26 | |
| lung 27 KC | normal | 33.71 | 122.77 | 122.77 | lung 27 | |
| lung 24 KC | COPD | 32.47 | 231.47 | 231.47 | lung 24 | −17.21 |
| lung 28 KC | COPD | 32.63 | 213.14 | 213.14 | lung 28 | −18.70 |
| lung 23 KC | COPD | 31.2 | 444.95 | 444.95 | lung 23 | −8.96 |
| lung 25 KC | normal | 33.46 | 139.4 | 139.40 | lung 25 | |
| asthmatic lung | 29321 | 31.6 | 360.95 | 360.95 | asthmatic lung | −11.04 |
| ODO3112 | | | | | | |
| asthmatic lung | 29323 | 28.66 | 1634.71 | 3269.42 | asthmatic lung | −1.22 |
| ODO3433 | | | | | | |
| asthmatic lung | 29322 | 26.36 | 5294.26 | 10588.52 | asthmatic lung | 2.66 |
| ODO3397 | | | | | | |
| asthmatic lung | 29325 | 28.23 | 2033.93 | 4067.86 | asthmatic lung | 1.02 |
| ODO4928 | | | | | | |
| endo cells KC | control | 30.68 | 580.47 | 580.47 | endo cells | |
| endo VEGF KC | | 31.08 | 471.26 | 471.26 | endo VEGF | −1.23 |
| endo bFGF KC | | 32.25 | 259.04 | 259.04 | endo bFGF | −2.24 |
| heart Clontech | normal | 27.28 | 3312.82 | 6625.64 | heart | |
| heart (T-1) ischemic | 29417 | 27.48 | 2979.22 | 5958.44 | heart T-1 | −1.11 |
| heart (T-14) non- | 29422 | 27.74 | 2613.8 | 5227.60 | heart T-14 | −1.27 |
| obstructive DCM | | | | | | |
| heart (T-3399) DCM | 29426 | 26.66 | 4541.38 | 9082.76 | heart T-3399 | 1.37 |
| adenoid GW99-269 | 26162 | 27.83 | 2493.31 | 4986.62 | adenoid | |
| tonsil GW98-280 | 22582 | 25.68 | 7506.04 | 15012.08 | tonsil | |
| T cells PC00314 | 28453 | 27.18 | 3487.61 | 6975.22 | T cells | |
| PBMNC | | 32.6 | 216.73 | 216.73 | PBMNC | |
| monocyte | | 32.27 | 256.89 | 513.78 | monocyte | |
| B cells PC00665 | 28455 | 27.83 | 2492.12 | 4984.24 | B cells | |
| dendritic cells 28441 | | 26.67 | 4528.97 | 9057.94 | dendritic cells | |
| neutrophils | 28440 | 28.4 | 1862.35 | 1862.35 | neutrophils | |
| eosinophils | 28446 | 31.69 | 344.59 | 689.18 | eosinophils | |
| BM unstim | | 32.04 | 289.03 | 289.03 | BM unstim | |
| BM stim | | 30.59 | 607.18 | 607.18 | BM stim | 2.10 |
| osteo dif | | 28.43 | 1831.57 | 1831.57 | osteo dif | 3.42 |
| osteo undif | | 30.83 | 536 | 536.00 | osteo undif | |
| chondrocytes | | 26.74 | 4368.46 | 10921.15 | chondrocytes | |
| OA Synovium IP12/01 | 29462 | 27.91 | 2391.03 | 2391.03 | OA Synovium | |
| OA Synovium NP10/01 | 29461 | 27.4 | 3109.93 | 6219.86 | OA Synovium | |
| OA Synovium NP57/00 | 28464 | 27.05 | 3729.47 | 7458.94 | OA Synovium | |
| RA Synovium NP03/01 | 28466 | 25.53 | 8116.96 | 16233.92 | RA Synovium | |
| RA Synovium NP71/00 | 28467 | 26.06 | 6167.42 | 12334.84 | RA Synovium | |
| RA Synovium NP45/00 | 28475 | 25.35 | 8888.66 | 17777.32 | RA Synovium | |
| OA bone (biobank) | 29217 | 30.23 | 729.87 | 729.87 | OA bone | |
| | | | | | (biobank) | |
| OA bone Sample 1 | J. Emory | 27.65 | 2743.38 | 5486.76 | OA bone | |
| OA bone Sample 2 | J. Emory | 28.02 | 2258.96 | 4517.92 | OA bone | |
| Cartilage (pool) | Normal | 25.82 | 7006.64 | 14013.28 | Cartilage (pool) | |
| Cartilage (pool) | OA | 27.22 | 3408.61 | 6817.22 | Cartilage (pool) | −2.06 |
| PBL unifected | 28441 | 24.24 | 15680.49 | 31360.98 | PBL unifected | |
| PBL HIV IIIB | 28442 | 25.43 | 8521.98 | 17043.96 | PBL HIV IIIB | −1.84 |
| MRC5 uninfected | 29158 | 25.58 | 7922.19 | 15844.38 | MRC5 uninfected | |
| (100%) | | | | | (100%) | |
| MRC5 HSV strain F | 29178 | 32.02 | 291.12 | 582.24 | MRC5 HSV strain F | −27.21 |
| W12 cells | 29179 | 26.03 | 6269.24 | 12538.48 | W12 cells | |
| Keratinocytes | 29180 | 25.43 | 8538.15 | 17076.30 | Keratinocytes | |
| B-actin control | | 29.74 | 938.61 | | | |
| genomic | | 28.79 | 1522.16 | | | |
| 1.00E+05 | | 20.84 | 100000 | | | |
| 1.00E+05 | | 21.4 | 100000 | | | |
| 1.00E+04 | | 24.5 | 10000 | | | |
| 1.00E+04 | | 25.2 | 10000 | | | |
| 1.00E+03 | | 28.45 | 1000 | | | |
| 1.00E+03 | | 29.25 | 1000 | | | |
| 1.00E+02 | | 35.34 | 100 | | | |
| 1.00E+02 | | 33.61 | 100 | | | |
| 1.00E+01 | | 38.95 | 10 | | | |
| 1.00E+01 | | 40 | 0 | | | |
| 1.00E−00 | | 40 | 0 | | | |
| 1.00E−00 | | 40 | 0 | | | |
| NTC | | 40 | 0 | | | |

Gene Name sbg1003328IG

| | Fold Change in Disease |
|---|---|
| | Population Relative to |
| Disease tissues | Normal |
| colon tumor | −1.13 |
| colon tumor | 1.59 |
| colon tumor | −1.55 |
| colon tumor | −1.65 |
| lung tumor | −1.15 |
| lung tumor | −1.34 |
| lung tumor | −1.23 |
| lung tumor | 1.14 |
| breast tumor | 3.10 |
| breast tumor | 46.37 |
| breast tumor | 23.06 |
| breast tumor | −1.23 |
| brain stage 5 ALZ | −4.43 |
| brain stage 5 ALZ | −1.06 |
| brain stage 5 ALZ | −1.19 |
| brain stage 5 ALZ | −1.74 |
| lung 24 | −17.21 |
| lung 28 | −18.70 |
| lung 23 | −8.96 |
| asthmatic lung | −11.04 |
| asthmatic lung | −1.22 |
| asthmatic lung | 2.66 |
| asthmatic lung | 1.02 |
| endo VEGF | −1.23 |
| endo bFGF | −2.24 |
| heart T-1 | −1.11 |
| heart T-14 | −1.27 |
| heart T-3399 | 1.37 |
| BM stim | 2.10 |
| osteo dif | 3.42 |
| Cartilage (pool) | −2.06 |
| PBL HIV IIIB | −1.84 |
| MRC5 HSV strain F | −27.21 |

Gene Name sbg1020829SGLT

Low overall expression in normal and disease samples. Highest normal expression in the whole brain, kidney, and thymus. Highest disease expression in the adenoid, tonsil, T cells, B cells, and eosinophils. Highly immune cell specific. Downregulation in 1 of 4 lung tumor samples and upregulation in 1 of 4 breast tumor samples indicates involvement in cancers of the lung and breast. Upregulation in 3 of 4 AD brain samples suggests an involvement in Alzheimer's disease. Downregulation in 3 of 3 COPD samples implies a role in chronic obstructive pulmonary disease. Downregulation in the stimulated bone marrow sample. Upregulation in the differentiated osteoblast sample. Upregulation in the HSV-infected MRC5 cells suggests that this gene may be a host factor in HSV. Moderate to high expression in the OA and RA samples indicates a potential role in osteoarthritis and rheumatoid arthritis.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1020829SGLT | 1 and 2) | 1) | 2) | Copies | (ng) | (ng) | RNA |
| Subcutaneous | 37.07, 40 | 7.33 | 0 | 3.67 | 3.06 | 16.34 | 59.89 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland Clontech | 40, 40 | 0 | 0 | 0.00 | 0.61 | 81.97 | 0.00 |
| Whole Brain Clontech | 32.13, 32.38 | 138.5 | 119.48 | 128.99 | 7.24 | 6.91 | 890.81 |
| Fetal Brain Clontech | 40, 40 | 0 | 0 | 0.00 | 0.48 | 103.95 | 0.00 |
| Cerebellum Clontech | 40, 40 | 0 | 0 | 0.00 | 2.17 | 23.04 | 0.00 |
| Cervix | 36.13, 40 | 12.82 | 0 | 6.41 | 2.42 | 20.66 | 132.44 |
| Colon | 36.93, 36.84 | 7.97 | 8.41 | 8.19 | 2.71 | 18.45 | 151.11 |
| Endometrium | 37.45, 37.41 | 5.84 | 5.98 | 5.91 | 0.73 | 68.21 | 403.14 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 36.53, 35.17 | 10.09 | 22.67 | 16.38 | 2.58 | 19.38 | 317.44 |
| Jejunum | 40, 35.73 | 0 | 16.2 | 8.10 | 6.60 | 7.58 | 61.36 |
| Kidney | 31.93, 31.01 | 155.27 | 269.39 | 212.33 | 2.12 | 23.58 | 5007.78 |
| Liver | 36.1, 35.88 | 13.05 | 14.82 | 13.94 | 1.50 | 33.33 | 464.50 |
| Fetal Liver Clontech | 35.06, 34.36 | 24.26 | 36.78 | 30.52 | 10.40 | 4.81 | 146.73 |
| Lung | 37.24, 40 | 6.63 | 0 | 3.32 | 2.57 | 19.46 | 64.49 |
| Mammary Gland | 34.25, 34.21 | 39.27 | 40.17 | 39.72 | 13.00 | 3.85 | 152.77 |
| Clontech | | | | | | | |
| Myometrium | 38.48, 35.31 | 3.16 | 20.87 | 12.02 | 2.34 | 21.37 | 256.73 |
| Omentum | 40, 38.91 | 0 | 2.45 | 1.23 | 3.94 | 12.69 | 15.55 |
| Ovary | 37.87, 37.06 | 4.56 | 7.37 | 5.97 | 4.34 | 11.52 | 68.72 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 36.06, 37.07 | 13.32 | 7.31 | 10.32 | 5.48 | 9.12 | 94.11 |
| Placenta Clontech | 35.98, 38.73 | 13.96 | 2.72 | 8.34 | 5.26 | 9.51 | 79.28 |
| Prostate | 40, 40 | 0 | 0 | 0.00 | 3.00 | 16.67 | 0.00 |
| Rectum | 40, 40 | 0 | 0 | 0.00 | 1.23 | 40.65 | 0.00 |
| Salivary Gland | 37.05, 37.06 | 7.4 | 7.36 | 7.38 | 7.31 | 6.84 | 50.48 |
| Clontech | | | | | | | |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine | 40, 40 | 0 | 0 | 0.00 | 0.98 | 51.07 | 0.00 |
| Clontech | | | | | | | |
| Spleen | 35.62, 35.72 | 17.34 | 16.35 | 16.85 | 4.92 | 10.16 | 171.19 |
| Stomach | 40, 40 | 0 | 1.61 | 0.81 | 2.73 | 18.32 | 14.74 |
| Testis Clontech | 40, 40 | 0 | 0 | 0.00 | 0.57 | 87.87 | 0.00 |
| Thymus Clontech | 31.3, 31.09 | 226.24 | 257.15 | 241.70 | 9.89 | 5.06 | 1221.92 |
| Thyroid | 40, 40 | 0 | 0 | 0.00 | 2.77 | 18.05 | 0.00 |
| Trachea Clontech | 36.6, 36.64 | 9.68 | 9.44 | 9.56 | 9.71 | 5.15 | 49.23 |
| Urinary Bladder | 40, 40 | 0 | 0 | 0.00 | 5.47 | 9.14 | 0.00 |
| Uterus | 34.64, 36.65 | 31.06 | 9.41 | 20.24 | 5.34 | 9.36 | 189.47 |
| genomic | 29.07 | 853.08 | | | | | |
| b-actin | 27.08 | 2793.5 | | | | | |
| 1.00E+05 | 20.85 | 100000 | | | | | |
| 1.00E+05 | 21.11 | 100000 | | | | | |
| 1.00E+04 | 24.81 | 10000 | | | | | |
| 1.00E+04 | 24.95 | 10000 | | | | | |
| 1.00E+03 | 28.39 | 1000 | | | | | |
| 1.00E+03 | 28.9 | 1000 | | | | | |
| 1.00E+02 | 34.1 | 100 | | | | | |
| 1.00E+02 | 32.86 | 100 | | | | | |
| 1.00E+01 | 35.52 | 10 | | | | | |
| 1.00E+01 | 40 | 0 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1020829SGLT | identifier) | Ct | copies | RNA | Sample | Population |
| colon normal GW98-167 | 21941 | 31.06 | 206.55 | 413.10 | colon normal | |
| colon tumor GW98-166 | 21940 | 31.18 | 193.59 | 387.18 | colon tumor | −1.07 |
| colon normal GW98-178 | 22080 | 30.74 | 244.37 | 488.74 | colon normal | |
| colon tumor GW98-177 | 22060 | 30.37 | 299.28 | 598.56 | colon tumor | 1.22 |
| colon normal GW98-561 | 23514 | 29.31 | 527.04 | 1054.08 | colon normal | |
| colon tumor GW98-560 | 23513 | 31.86 | 134.48 | 268.96 | colon tumor | −3.92 |
| colon normal GW98-894 | 24691 | 31.84 | 135.33 | 270.66 | colon normal | |
| colon tumor GW98-893 | 24690 | 31.9 | 131.57 | 263.14 | colon tumor | −1.03 |
| lung normal GW98-3 | 20742 | 28.81 | 689.14 | 1378.28 | lung normal | |
| lung tumor GW98-2 | 20741 | 31.91 | 130.71 | 261.42 | lung tumor | −5.27 |
| lung normal GW97-179 | 20677 | 30.04 | 356.26 | 712.52 | lung normal | |
| lung tumor GW97-178 | 20676 | 29.05 | 605.73 | 1211.46 | lung tumor | 1.70 |
| lung normal GW98-165 | 21922 | 28.42 | 852.41 | 1704.82 | lung normal | |
| lung tumor GW98-164 | 21921 | 30.51 | 277.13 | 554.26 | lung tumor | −3.08 |
| lung normal GW98-282 | 22584 | 31.23 | 188.34 | 376.68 | lung normal | |
| lung tumor GW98-281 | 22583 | 30.46 | 285 | 570.00 | lung tumor | 1.51 |
| breast normal GW00-392 | 28750 | 31.14 | 197.15 | 197.15 | breast normal | |
| breast tumor GW00-391 | 28746 | 32.15 | 114.65 | 229.30 | breast tumor | 1.16 |
| breast normal GW00-413 | 28798 | 34.87 | 26.64 | 26.64 | breast normal | |
| breast tumor GW00-412 | 28797 | 31.83 | 136.42 | 272.84 | breast tumor | 10.24 |
| breast normal GW00- | 27592-95 | 36.34 | 12.09 | 12.09 | breast normal | |
| 235:238 | | | | | | |
| breast tumor GW00- | 27588-91 | 33.48 | 56.11 | 56.11 | breast tumor | 4.64 |
| 231:234 | | | | | | |
| breast normal GW98-621 | 23656 | 32.39 | 100.78 | 201.56 | breast normal | |
| breast tumor GW98-620 | 23655 | 31.4 | 171.82 | 343.64 | breast tumor | 1.70 |
| brain normal BB99-542 | 25507 | 34.49 | 32.75 | 65.50 | brain normal | |
| brain normal BB99-406 | 25509 | 34.01 | 42.2 | 84.40 | brain normal | |
| brain normal BB99-904 | 25546 | 36.17 | 13.3 | 26.60 | brain normal | |
| brain stage 5 ALZ BB99- | 25502 | 31.16 | 195.23 | 390.46 | brain stage 5 ALZ | 6.64 |
| 874 | | | | | | |
| brain stage 5 ALZ BB99- | 25503 | 31.56 | 157.33 | 314.66 | brain stage 5 ALZ | 5.35 |
| 887 | | | | | | |
| brain stage 5 ALZ BB99- | 25504 | 32.62 | 89.2 | 178.40 | brain stage 5 ALZ | 3.03 |
| 862 | | | | | | |
| brain stage 5 ALZ BB99- | 25542 | 33.26 | 63.43 | 126.86 | brain stage 5 ALZ | 2.16 |
| 927 | | | | | | |
| CT lung KC | normal | 30.82 | 234.88 | 469.76 | CT lung | |
| lung 26 KC | normal | 30.21 | 325.42 | 325.42 | lung 26 | |
| lung 27 KC | normal | 36.89 | 9 | 9.00 | lung 27 | |
| lung 24 KC | COPD | 36.17 | 13.24 | 13.24 | lung 24 | −15.84 |
| lung 28 KC | COPD | 38.38 | 4.06 | 4.06 | lung 28 | −51.66 |
| lung 23 KC | COPD | 35.53 | 18.67 | 18.67 | lung 23 | −11.23 |
| lung 25 KC | normal | 34.37 | 34.83 | 34.83 | lung 25 | |
| asthmatic lung ODO3112 | 29321 | 33.65 | 51.41 | 51.41 | asthmatic lung | −4.08 |
| asthmatic lung ODO3433 | 29323 | 30.62 | 260.95 | 521.90 | asthmatic lung | 2.49 |
| asthmatic lung ODO3397 | 29322 | 31.31 | 180.14 | 360.28 | asthmatic lung | 1.72 |
| asthmatic lung ODO4928 | 29325 | 31.14 | 197.09 | 394.18 | asthmatic lung | 1.88 |
| endo cells KC | control | 32.56 | 92.23 | 92.23 | endo cells | |
| endo VEGF KC | | 33.29 | 62.39 | 62.39 | endo VEGF | −1.48 |
| endo bFGF KC | | 32.55 | 92.65 | 92.65 | endo bFGF | 1.00 |
| heart Clontech | normal | 33.17 | 66.25 | 132.50 | heart | |
| heart (T-1) ischemic | 29417 | 33.07 | 70.16 | 140.32 | heart T-1 | 1.06 |
| heart (T-14) non- | 29422 | 34.64 | 30.13 | 60.26 | heart T-14 | −2.20 |
| obstructive DCM | | | | | | |
| heart (T-3399) DCM | 29426 | 32.53 | 93.63 | 187.26 | heart T-3399 | 1.41 |
| adenoid GW99-269 | 26162 | 28.92 | 650.55 | 1301.10 | adenoid | |
| tonsil GW98-280 | 22582 | 27.11 | 1719.42 | 3438.84 | tonsil | |
| T cells PC00314 | 28453 | 28.05 | 1037.04 | 2074.08 | T cells | |
| PBMNC | | 36.57 | 10.71 | 10.71 | PBMNC | |
| monocyte | | 33.22 | 64.68 | 129.36 | monocyte | |
| B cells PC00665 | 28455 | 27.07 | 1757.79 | 3515.58 | B cells | |
| dendritic cells 28441 | | 33.77 | 48.05 | 96.10 | dendritic cells | |
| neutrophils | 28440 | 30.71 | 248.56 | 248.56 | neutrophils | |
| eosinophils | 28446 | 27.3 | 1549.7 | 3099.40 | eosinophils | |
| BM unstim | | 30.06 | 352.26 | 352.26 | BM unstim | |
| BM stim | | 34.14 | 39.39 | 39.39 | BM stim | −8.94 |
| osteo dif | | 36.29 | 12.42 | 12.42 | osteo dif | 12.42 |
| osteo undif | | 40 | 0 | 0.00 | osteo undif | |
| chondrocytes | | 32.11 | 117.39 | 293.48 | chondrocytes | |
| OA Synovium IP12/01 | 29462 | 30.17 | 331.7 | 331.70 | OA Synovium | |
| OA Synovium NP10/01 | 29461 | 32.05 | 120.98 | 241.96 | OA Synovium | |
| OA Synovium NP57/00 | 28464 | 30.13 | 339.03 | 678.06 | OA Synovium | |
| RA Synovium NP03/01 | 28466 | 31.28 | 182.96 | 365.92 | RA Synovium | |
| RA Synovium NP71/00 | 28467 | 29.81 | 402.34 | 804.68 | RA Synovium | |
| RA Synovium NP45/00 | 28475 | 30.22 | 324.14 | 648.28 | RA Synovium | |
| OA bone (biobank) | 29217 | 28.45 | 837.78 | 837.78 | OA bone (biobank) | |
| OA bone Sample 1 | J. Emory | 30.21 | 325.03 | 650.06 | OA bone | |
| OA bone Sample 2 | J. Emory | 29.8 | 406 | 812.00 | OA bone | |
| Cartilage (pool) | Normal | 31.09 | 203.28 | 406.56 | Cartilage (pool) | |
| Cartilage (pool) | OA | 32.18 | 112.77 | 225.54 | Cartilage (pool) | −1.80 |
| PBL unifected | 28441 | 29.17 | 567.22 | 1134.44 | PBL unifected | |
| PBL HIV IIIB | 28442 | 30.73 | 246.69 | 493.38 | PBL HIV IIIB | −2.30 |
| MRC5 uninfected | 29158 | 35.54 | 18.61 | 37.22 | MRC5 uninfected | |
| (100%) | | | | | (100%) | |
| MRC5 HSV strain F | 29178 | 30.54 | 272.3 | 544.60 | MRC5 HSV strain F | 14.63 |
| W12 cells | 29179 | 32.28 | 107.25 | 214.50 | W12 cells | |
| Keratinocytes | 29180 | 34.27 | 36.84 | 73.68 | Keratinocytes | |
| B-actin control | | 27.03 | 1793.92 | | | |
| genomic | | 27.77 | 1204.8 | | | |
| 1.00E+05 | | 19.84 | 100000 | | | |
| 1.00E+05 | | 19.86 | 100000 | | | |
| 1.00E+04 | | 23.46 | 10000 | | | |
| 1.00E+04 | | 23.8 | 10000 | | | |
| 1.00E+03 | | 27.45 | 1000 | | | |
| 1.00E+03 | | 27.94 | 1000 | | | |
| 1.00E+02 | | 33.86 | 100 | | | |
| 1.00E+02 | | 31.41 | 100 | | | |
| 1.00E+01 | | 40 | 0 | | | |
| 1.00E+01 | | 36.88 | 10 | | | |
| 1.00E−00 | | 40 | 0 | | | |
| 1.00E−00 | | 40 | 0 | | | |
| NTC | | 40 | 0 | | | |

Gene Name sbg1020829SGLT

| | Fold Change in Disease |
|---|---|
| | Population Relative to |
| Disease tissues | Normal |
| colon tumor | −1.07 |
| colon tumor | 1.22 |
| colon tumor | −3.92 |
| colon tumor | −1.03 |
| lung tumor | −5.27 |
| lung tumor | 1.70 |
| lung tumor | −3.08 |
| lung tumor | 1.51 |
| breast tumor | 1.16 |
| breast tumor | 10.24 |
| breast tumor | 4.64 |
| breast tumor | 1.70 |
| brain stage 5 ALZ | 6.64 |
| brain stage 5 ALZ | 5.35 |
| brain stage 5 ALZ | 3.03 |
| brain stage 5 ALZ | 2.16 |
| lung 24 | −15.84 |
| lung 28 | −51.66 |
| lung 23 | −11.23 |
| asthmatic lung | −4.08 |
| asthmatic lung | 2.49 |
| asthmatic lung | 1.72 |
| asthmatic lung | 1.88 |
| endo VEGF | −1.48 |
| endo bFGF | 1.00 |
| heart T-1 | 1.06 |
| heart T-14 | −2.20 |
| heart T-3399 | 1.41 |
| BM stim | −8.94 |
| osteo dif | 12.42 |
| Cartilage (pool) | −1.80 |
| PBL HIV IIIB | −2.30 |
| MRC5 HSV strain F | 14.63 |

Gene Name sbg1005450UDPGT

Low to moderate overall expression. Highest normal expression in endometrium, esophagus, and spleen with lower levels of expression in cerebellum, hypothalamus, rectum, and uterus. Highest disease expression in one of the OA synovium samples. Downregulation in 1 of 4 colon tumor samples is sufficient to make a disease claim in cancer of the colon. Upregulation in 1 of 4 lung tumor samples indicates a potential role for this gene in cancer of the lung. Downregulation in 2 of 4 AD brain samples suggests an involvement in Alzheimer's disease. Downregulation in 3 of 3 COPD lung samples and 4 of 4 asthmatic lung samples suggests involvement in chronic obstructive pulmonary disease and asthma. Upregulation in the HSV-infected MRC5 cells suggests that this gene may be a host factor in HSV. Moderate expression in the B cells and the dendritic cells.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | Ct | copies | copies | Average | 18S | 18S | 50 ng |
| Sample | (sample 1 | (sample | (sample | GOI | rRNA | rRNA | total |
| sbg1005450UDPGT | and 2) | 1) | 2) | Copies | (ng) | (ng) | RNA |
| Subcutaneous | 40, 40 | 0.15 | 0.17 | 0.16 | 3.06 | 16.34 | 2.61 |
| Adipocytes Zenbio | | | | | | | |
| Subcutaneous Adipose | 40, 40 | 0 | 0 | 0.00 | 0.96 | 52.36 | 0.00 |
| Zenbio | | | | | | | |
| Adrenal Gland | 40, 40 | 0 | 0.14 | 0.07 | 0.61 | 81.97 | 5.74 |
| Clontech | | | | | | | |
| Whole Brain Clontech | 33.74, 40 | 12.07 | 0 | 6.04 | 7.24 | 6.91 | 41.68 |
| Fetal Brain Clontech | 40, 40 | 0 | 0 | 0.00 | 0.48 | 103.95 | 0.00 |
| Cerebellum Clontech | 32.07, 33.2 | 32.85 | 16.64 | 24.75 | 2.17 | 23.04 | 570.16 |
| Cervix | 40, 40 | 0 | 0 | 0.00 | 2.42 | 20.66 | 0.00 |
| Colon | 40, 35.16 | 0 | 5.12 | 2.56 | 2.71 | 18.45 | 47.23 |
| Endometrium | 32.73, 31.85 | 22.19 | 37.5 | 29.85 | 0.73 | 68.21 | 2035.81 |
| Esophagus | 32.67, 29.39 | 22.91 | 165.34 | 94.13 | 1.37 | 36.50 | 3435.22 |
| Heart Clontech | 37.12, 35.03 | 1.58 | 5.55 | 3.57 | 1.32 | 37.88 | 135.04 |
| Hypothalamus | 34.08, 40 | 9.84 | 0 | 4.92 | 0.32 | 155.28 | 763.98 |
| Ileum | 34.35 | 8.33 | | 8.33 | 2.58 | 19.38 | 161.43 |
| Jejunum | 40, 40 | 0 | 0 | 0.00 | 6.60 | 7.58 | 0.00 |
| Kidney | 38.89, 40 | 0.54 | 0 | 0.27 | 2.12 | 23.58 | 6.37 |
| Liver | 36.32, 40 | 2.55 | 0 | 1.28 | 1.50 | 33.33 | 42.50 |
| Fetal Liver Clontech | 36.96 | 1.74 | | 1.74 | 10.40 | 4.81 | 8.37 |
| Lung | 40, 40 | 0 | 0 | 0.00 | 2.57 | 19.46 | 0.00 |
| Mammary Gland | 40, 40 | 0 | 0 | 0.00 | 13.00 | 3.85 | 0.00 |
| Clontech | | | | | | | |
| Myometrium | 40, 38.22 | 0 | 0.82 | 0.41 | 2.34 | 21.37 | 8.76 |
| Omentum | 36.17, 40 | 2.8 | 0 | 1.40 | 3.94 | 12.69 | 17.77 |
| Ovary | 40, 40 | 0 | 0 | 0.00 | 4.34 | 11.52 | 0.00 |
| Pancreas | 40, 40 | 0 | 0 | 0.00 | 0.81 | 61.80 | 0.00 |
| Head of Pancreas | 40, 38.35 | 0 | 0.75 | 0.38 | 1.57 | 31.85 | 11.94 |
| Parotid Gland | 40, 40 | 0 | 0 | 0.00 | 5.48 | 9.12 | 0.00 |
| Placenta Clontech | 39.06, 35.49 | 0.49 | 4.22 | 2.36 | 5.26 | 9.51 | 22.39 |
| Prostate | 38.81, 40 | 0.57 | 0 | 0.29 | 3.00 | 16.67 | 4.75 |
| Rectum | 35.22, 33.25 | 4.94 | 16.2 | 10.57 | 1.23 | 40.65 | 429.67 |
| Salivary Gland | 32.56, 34.56 | 24.55 | 7.36 | 15.96 | 7.31 | 6.84 | 109.13 |
| Clontech | | | | | | | |
| Skeletal Muscle | 40, 40 | 0 | 0 | 0.00 | 1.26 | 39.68 | 0.00 |
| Clontech | | | | | | | |
| Skin | 34.26, 40 | 8.8 | 0 | 4.40 | 1.21 | 41.32 | 181.82 |
| Small Intestine | 40 | 0 | 6.23 | 3.12 | 0.98 | 51.07 | 159.09 |
| Clontech | | | | | | | |
| Spleen | 40, 27.36 | 0 | 560.92 | 280.46 | 4.92 | 10.16 | 2850.20 |
| Stomach | 33.49, 39.12 | 13.98 | 0.47 | 7.23 | 2.73 | 18.32 | 132.33 |
| Testis Clontech | 40, 40 | 0 | 0 | 0.00 | 0.57 | 87.87 | 0.00 |
| Thymus Clontech | 40, 35.33 | 0 | 4.64 | 2.32 | 9.89 | 5.06 | 11.73 |
| Thyroid | 37.18, 35.52 | 1.53 | 4.13 | 2.83 | 2.77 | 18.05 | 51.08 |
| Trachea Clontech | 40, 40 | 0 | 0 | 0.00 | 9.71 | 5.15 | 0.00 |
| Urinary Bladder | 40, 40 | 0 | 0.16 | 0.08 | 5.47 | 9.14 | 0.73 |
| Uterus | 30.11 | 106.94 | 0 | 53.47 | 5.34 | 9.36 | 500.66 |
| genomic | 35.81 | 3.47 | | | | | |
| b-actin | 26.86 | 757.01 | | | | | |
| 1.00E+05 | 18.99 | 100000 | | | | | |
| 1.00E+05 | 19.13 | 100000 | | | | | |
| 1.00E+04 | 22.43 | 10000 | | | | | |
| 1.00E+04 | 22.31 | 10000 | | | | | |
| 1.00E+03 | 25.74 | 1000 | | | | | |
| 1.00E+03 | 25.99 | 1000 | | | | | |
| 1.00E+02 | 31.47 | 100 | | | | | |
| 1.00E+02 | 29.82 | 100 | | | | | |
| 1.00E+01 | 40 | 0 | | | | | |
| 1.00E+01 | 40 | 0 | | | | | |
| 1.00E−00 | 40 | 0 | | | | | |
| 1.00E−00 | 40 | 0 | | | | | |
| NTC | 26.02 | −1 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1005450UDPGT | identifier) | Ct | copies | RNA | Sample | Population |
| colon normal GW98-167 | 21941 | 40 | 0.17 | 0.34 | colon normal | |
| colon tumor GW98-166 | 21940 | 39.88 | 0.29 | 0.58 | colon tumor | 1.71 |
| colon normal GW98-178 | 22080 | 40 | 0 | 0.00 | colon normal | |
| colon tumor GW98-177 | 22060 | 40 | 0 | 0.00 | colon tumor | 0.00 |
| colon normal GW98-561 | 23514 | 40 | 0 | 0.00 | colon normal | |
| colon tumor GW98-560 | 23513 | 40 | 0 | 0.00 | colon tumor | 0.00 |
| colon normal GW98-894 | 24691 | 33.84 | 10.47 | 20.94 | colon normal | |
| colon tumor GW98-893 | 24690 | 40 | 0 | 0.00 | colon tumor | −20.94 |
| lung normal GW98-3 | 20742 | 40 | 0 | 0.00 | lung normal | |
| lung tumor GW98-2 | 20741 | 40 | 0 | 0.00 | lung tumor | 0.00 |
| lung normal GW97-179 | 20677 | 31.67 | 37.94 | 75.88 | lung normal | |
| lung tumor GW97-178 | 20676 | 33.08 | 16.47 | 32.94 | lung tumor | −2.30 |
| lung normal GW98-165 | 21922 | 40 | 0 | 0.00 | lung normal | |
| lung tumor GW98-164 | 21921 | 40 | 0 | 0.00 | lung tumor | 0.00 |
| lung normal GW98-282 | 22584 | 40 | 0 | 0.00 | lung normal | |
| lung tumor GW98-281 | 22583 | 35.03 | 5.16 | 10.32 | lung tumor | 10.32 |
| breast normal GW00-392 | 28750 | 32.64 | 21.38 | 21.38 | breast normal | |
| breast tumor GW00-391 | 28746 | 31.67 | 37.98 | 75.96 | breast tumor | 3.55 |
| breast normal GW00-413 | 28798 | 32.54 | 22.63 | 22.63 | breast normal | |
| breast tumor GW00-412 | 28797 | 29.23 | 161.71 | 323.42 | breast tumor | 14.29 |
| breast normal GW00- | 27592-95 | 37.05 | 1.55 | 1.55 | breast normal | |
| 235:238 | | | | | | |
| breast tumor GW00- | 27588-91 | 35.03 | 5.17 | 5.17 | breast tumor | 3.34 |
| 231:234 | | | | | | |
| breast normal GW98-621 | 23656 | 34.12 | 8.87 | 17.74 | breast normal | |
| breast tumor GW98-620 | 23655 | 40 | 0 | 0.00 | breast tumor | −17.74 |
| brain normal BB99-542 | 25507 | 34.28 | 8.05 | 16.10 | brain normal | |
| brain normal BB99-406 | 25509 | 40 | 0 | 0.00 | brain normal | |
| brain normal BB99-904 | 25546 | 40 | 0 | 0.00 | brain normal | |
| brain stage 5 ALZ BB99- | 25502 | 38.8 | 0.55 | 1.10 | brain stage 5 ALZ | −4.88 |
| 874 | | | | | | |
| brain stage 5 ALZ BB99- | 25503 | 40 | 0 | 0.00 | brain stage 5 ALZ | −5.37 |
| 887 | | | | | | |
| brain stage 5 ALZ BB99- | 25504 | 36.16 | 2.64 | 5.28 | brain stage 5 ALZ | −1.02 |
| 862 | | | | | | |
| brain stage 5 ALZ BB99- | 25542 | 40 | 0 | 0.00 | brain stage 5 ALZ | −5.37 |
| 927 | | | | | | |
| CT lung KC | normal | 36.61 | 2.02 | 4.04 | CT lung | |
| lung 26 KC | normal | | | | lung 26 | |
| lung 27 KC | normal | 40 | 0 | 0.00 | lung 27 | |
| lung 24 KC | COPD | 40 | 0 | 0.00 | lung 24 | −1.35 |
| lung 28 KC | COPD | 40 | 0 | 0.00 | lung 28 | −1.35 |
| lung 23 KC | COPD | 40 | 0 | 0.00 | lung 23 | −1.35 |
| lung 25 KC | normal | 40 | 0 | 0.00 | lung 25 | |
| asthmatic lung ODO3112 | 29321 | 38.19 | 0.79 | 0.79 | asthmatic lung | −1.70 |
| asthmatic lung ODO3433 | 29323 | 36.09 | 2.76 | 5.52 | asthmatic lung | 4.10 |
| asthmatic lung ODO3397 | 29322 | 40 | 0 | 0.00 | asthmatic lung | −1.35 |
| asthmatic lung ODO4928 | 29325 | 40 | 0 | 0.00 | asthmatic lung | −1.35 |
| endo cells KC | control | 40 | 0 | 0.00 | endo cells | |
| endo VEGF KC | | 40 | 0 | 0.00 | endo VEGF | 0.00 |
| endo bFGF KC | | 40 | 0 | 0.00 | endo bFGF | 0.00 |
| heart Clontech | normal | 40 | 0 | 0.00 | heart | |
| heart (T-1) ischemic | 29417 | 38.36 | 0.71 | 1.42 | heart T-1 | 1.42 |
| heart (T-14) non- | 29422 | 40 | 0 | 0.00 | heart T-14 | 0.00 |
| obstructive DCM | | | | | | |
| heart (T-3399) DCM | 29426 | 40 | 0 | 0.00 | heart T-3399 | 0.00 |
| adenoid GW99-269 | 26162 | 38.96 | 0.5 | 1.00 | adenoid | |
| tonsil GW98-280 | 22582 | 35.44 | 4.04 | 8.08 | tonsil | |
| T cells PC00314 | 28453 | 38.83 | 0.54 | 1.08 | T cells | |
| PBMNC | | 40 | 0 | 0.00 | PBMNC | |
| monocyte | | 35.1 | 4.94 | 9.88 | monocyte | |
| B cells PC00665 | 28455 | 33.32 | 14.31 | 28.62 | B cells | |
| dendritic cells 28441 | | 32.53 | 22.85 | 45.70 | dendritic cells | |
| neutrophils | 28440 | 34.43 | 7.39 | 7.39 | neutrophils | |
| eosinophils | 28446 | 40 | 0 | 0.00 | eosinophils | |
| BM unstim | | 40 | 0 | 0.00 | BM unstim | |
| BM stim | | 34.12 | 8.87 | 8.87 | BM stim | 8.87 |
| osteo dif | | 40 | 0 | 0.00 | osteo dif | 0.00 |
| osteo undif | | 40 | 0 | 0.00 | osteo undif | |
| chondrocytes | | 34.51 | 7.05 | 17.63 | chondrocytes | |
| OA Synovium IP12/01 | 29462 | 40 | 0 | 0.00 | OA Synovium | |
| OA Synovium NP10/01 | 29461 | 27.21 | 538.51 | 1077.02 | OA Synovium | |
| OA Synovium NP57/00 | 28464 | 33.5 | 12.85 | 25.70 | OA Synovium | |
| RA Synovium NP03/01 | 28466 | 39.09 | 0.46 | 0.92 | RA Synovium | |
| RA Synovium NP71/00 | 28467 | 40 | 0 | 0.00 | RA Synovium | |
| RA Synovium NP45/00 | 28475 | 40 | 0 | 0.00 | RA Synovium | |
| OA bone (biobank) | 29217 | 40 | 0 | 0.00 | OA bone | |
| | | | | | (biobank) | |
| OA bone Sample 1 | J. Emory | 35.23 | 4.59 | 9.18 | OA bone | |
| OA bone Sample 2 | J. Emory | 37.1 | 1.51 | 3.02 | OA bone | |
| Cartilage (pool) | Normal | 35.45 | 4.01 | 8.02 | Cartilage (pool) | |
| Cartilage (pool) | OA | 40 | 0 | 0.00 | Cartilage (pool) | −8.02 |
| PBL unifected | 28441 | 40 | 0 | 0.00 | PBL unifected | |
| PBL HIV IIIB | 28442 | 40 | 0 | 0.00 | PBL HIV IIIB | 0.00 |
| MRC5 uninfected (100%) | 29158 | 40 | 0.17 | 0.34 | MRC5 uninfected | |
| | | | | | (100%) | |
| MRC5 HSV strain F | 29178 | 30.15 | 93.76 | 187.52 | MRC5 HSV strain F | 551.53 |
| W12 cells | 29179 | 40 | 0 | 0.00 | W12 cells | |
| Keratinocytes | 29180 | 35.72 | 3.44 | 6.88 | Keratinocytes | |
| B-actin control | | 26.57 | 788.4 | | | |
| genomic | | 25.69 | 1326.94 | | | |
| 1.00E+05 | | 18.72 | 100000 | | | |
| 1.00E+05 | | 18.74 | 100000 | | | |
| 1.00E+04 | | 22.11 | 10000 | | | |
| 1.00E+04 | | 22.15 | 10000 | | | |
| 1.00E+03 | | 25.57 | 1000 | | | |
| 1.00E+03 | | 25.54 | 1000 | | | |
| 1.00E+02 | | 31.37 | 100 | | | |
| 1.00E+02 | | 29.65 | 100 | | | |
| 1.00E+01 | | 40 | 0 | | | |
| 1.00E+01 | | 40 | 0 | | | |
| 1.00E−00 | | 40 | 0 | | | |
| 1.00E−00 | | 40 | 0 | | | |
| NTC | | 40 | 0 | | | |

Gene Name sbg1005450UDPGT

| | Fold Change in Disease |
|---|---|
| | Population Relative to |
| Disease tissues | Normal |
| colon tumor | 1.71 |
| colon tumor | 0.00 |
| colon tumor | 0.00 |
| colon tumor | −20.94 |
| lung tumor | 0.00 |
| lung tumor | −2.30 |
| lung tumor | 0.00 |
| lung tumor | 10.32 |
| breast tumor | 3.55 |
| breast tumor | 14.29 |
| breast tumor | 3.34 |
| breast tumor | −17.74 |
| brain stage 5 ALZ | −4.88 |
| brain stage 5 ALZ | −5.37 |
| brain stage 5 ALZ | −1.02 |
| brain stage 5 ALZ | −5.37 |
| lung 24 | −1.35 |
| lung 28 | −1.35 |
| lung 23 | −1.35 |
| asthmatic lung | −1.70 |
| asthmatic lung | 4.10 |
| asthmatic lung | −1.35 |
| asthmatic lung | −1.35 |
| endo VEGF | 0.00 |
| endo bFGF | 0.00 |
| heart T-1 | 1.42 |
| heart T-14 | 0.00 |
| heart T-3399 | 0.00 |
| BM stim | 8.87 |
| osteo undif | 0.00 |
| Cartilage (pool) | −8.02 |
| PBL HIV IIIB | 0.00 |
| MRC5 HSV strain F | 551.53 |

Gene Name sbg1002620Tia

Moderate overall expression. Highest normal expression in the whole brain, endometrium, myometrium, placenta, and rectum. Highest disease expression in the one of the colon normal/tumor pairs, the normal lung samples, one of the asthmatic lung samples, the neutrophils, the eosinophils, and one of the RA synovium samples. Expressed at high levels in all of the samples representative of the GI tract indicating a potential role for this gene in IBS, IBD, and Crohn's disease. Downregulation in 1 of 3 COPD lung samples suggests involvement in chronic obstructive pulmonary disease. Upregulation in 1 of 4 asthmatic lung samples implies a role in asthma. High expression in the OA synovium and bone samples as well as in the RA synovium samples. Also high expression in the chondrocytes. Variable expression in the immune cells with highest expression in the neutrophils and eosinophils and lowest expression in the dendritic cells. Corroborating high expression in B and T cells as well as OA samples implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1002620TIa | 1 and 2) | 1) | 2) | Copies | (ng) | (ng) | RNA |
| Subcutaneous | 35.04, 34.4 | 201.66 | 274.43 | 238.05 | 3.06 | 16.34 | 3889.62 |
| Adipocytes Zenbio | | | | | | | |
| Subcutaneous Adipose | 38.03, 38.45 | 48.67 | 39.85 | 44.26 | 0.96 | 52.36 | 2317.28 |
| Zenbio | | | | | | | |
| Adrenal Gland Clontech | 38.71, 38.14 | 35.25 | 46.23 | 40.74 | 0.61 | 81.97 | 3339.34 |
| Whole Brain Clontech | 29.27, 29.32 | 3152.23 | 3071.49 | 3111.86 | 7.24 | 6.91 | 21490.75 |
| Fetal Brain Clontech | 40, 37.57 | 0 | 60.7 | 30.35 | 0.48 | 103.95 | 3154.89 |
| Cerebellum Clontech | 39.37, 39.14 | 25.78 | 28.75 | 27.27 | 2.17 | 23.04 | 628.23 |
| Cervix | 34.05, 34.32 | 323.57 | 285.02 | 304.30 | 2.42 | 20.66 | 6287.09 |
| Colon | 32.64, 32.98 | 633.12 | 537.54 | 585.33 | 2.71 | 18.45 | 10799.45 |
| Endometrium | 34.44, 33.84 | 269.09 | 357.16 | 313.13 | 0.73 | 68.21 | 21359.14 |
| Esophagus | 35.48, 35.21 | 163.52 | 186.15 | 174.84 | 1.37 | 36.50 | 6380.84 |
| Heart Clontech | 38.67, 39.08 | 35.94 | 29.52 | 32.73 | 1.32 | 37.88 | 1239.77 |
| Hypothalamus | 40, 40 | 0 | 0 | 0.00 | 0.32 | 155.28 | 0.00 |
| Ileum | 33.07, 32.9 | 516.19 | 559.94 | 538.07 | 2.58 | 19.38 | 10427.62 |
| Jejunum | 30.58, 30.66 | 1688.39 | 1625.61 | 1657.00 | 6.60 | 7.58 | 12553.03 |
| Kidney | 34.9, 33.68 | 216.07 | 385.19 | 300.63 | 2.12 | 23.58 | 7090.33 |
| Liver | 37.17, 36.49 | 73.4 | 101.31 | 87.36 | 1.50 | 33.33 | 2911.83 |
| Fetal Liver Clontech | 33.99, 34.79 | 332.15 | 227.82 | 279.99 | 10.40 | 4.81 | 1346.08 |
| Lung | 34.67, 34.06 | 240.47 | 321.54 | 281.01 | 2.57 | 19.46 | 5467.02 |
| Mammary Gland | 29.3, 29.19 | 3098.36 | 3272.39 | 3185.38 | 13.00 | 3.85 | 12251.44 |
| Clontech | | | | | | | |
| Myometrium | 32.45, 31.79 | 692.54 | 946.75 | 819.65 | 2.34 | 21.37 | 17513.78 |
| Omentum | 32.88, 33.43 | 563.23 | 434.44 | 498.84 | 3.94 | 12.69 | 6330.39 |
| Ovary | 33.02, 32.92 | 528.43 | 553.26 | 540.85 | 4.34 | 11.52 | 6230.93 |
| Pancreas | 37.31, 39.81 | 68.49 | 20.84 | 44.67 | 0.81 | 61.80 | 2760.51 |
| Head of Pancreas | 38.5, 39.16 | 38.99 | 28.45 | 33.72 | 1.57 | 31.85 | 1073.89 |
| Parotid Gland | 34.48, 34.22 | 263.15 | 298.49 | 280.82 | 5.48 | 9.12 | 2562.23 |
| Placenta Clontech | 31.16, 30.91 | 1280.82 | 1442.99 | 1361.91 | 5.26 | 9.51 | 12945.87 |
| Prostate | 33.5, 33.11 | 420.13 | 506.76 | 463.45 | 3.00 | 16.67 | 7724.08 |
| Rectum | 34.48, 33.88 | 263.61 | 350.22 | 306.92 | 1.23 | 40.65 | 12476.22 |
| Salivary Gland | 34.48, 34.32 | 263.4 | 284.18 | 273.79 | 7.31 | 6.84 | 1872.71 |
| Clontech | | | | | | | |
| Skeletal Muscle | 40, 39.37 | 0 | 25.73 | 12.87 | 1.26 | 39.68 | 510.52 |
| Clontech | | | | | | | |
| Skin | 35.52, 35.13 | 160.58 | 193.62 | 177.10 | 1.21 | 41.32 | 7318.18 |
| Small Intestine | 36.79, 36.59 | 87.74 | 96.5 | 92.12 | 0.98 | 51.07 | 4704.80 |
| Clontech | | | | | | | |
| Spleen | 34.45, 34.51 | 267.45 | 260 | 263.73 | 4.92 | 10.16 | 2680.13 |
| Stomach | 35.16, 33.89 | 191.03 | 348.48 | 269.76 | 2.73 | 18.32 | 4940.57 |
| Testis Clontech | 38.19, 37.07 | 45.22 | 76.91 | 61.07 | 0.57 | 87.87 | 5365.99 |
| Thymus Clontech | 33.74, 33.57 | 374.59 | 406.79 | 390.69 | 9.89 | 5.06 | 1975.18 |
| Thyroid | 34.18, 33.46 | 304.38 | 427.57 | 365.98 | 2.77 | 18.05 | 6606.05 |
| Trachea Clontech | 32.67, 31.27 | 623.94 | 1213.65 | 918.80 | 9.71 | 5.15 | 4731.18 |
| Urinary Bladder | 32.07, 31.34 | 830.04 | 1176.15 | 1003.10 | 5.47 | 9.14 | 9169.06 |
| Uterus | 31.75, 31.37 | 968.5 | 1157.09 | 1062.80 | 5.34 | 9.36 | 9951.26 |
| genomic | 31.33 | 1181.44 | | | | | |
| b-actin | 28.56 | 4411.32 | | | | | |
| 1.00E+05 | 22.12 | 100000 | | | | | |
| 1.00E+05 | 22.12 | 100000 | | | | | |
| 1.00E+04 | 26.72 | 10000 | | | | | |
| 1.00E+04 | 26.91 | 10000 | | | | | |
| 1.00E+03 | 31.28 | 1000 | | | | | |
| 1.00E+03 | 31.5 | 1000 | | | | | |
| 1.00E+02 | 36.35 | 100 | | | | | |
| 1.00E+02 | 37.09 | 100 | | | | | |
| 1.00E+01 | 40 | 10 | | | | | |
| 1.00E+01 | 40 | 10 | | | | | |
| 1.00E−00 | 40 | 1 | | | | | |
| 1.00E−00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1002620TIa | identifier) | Ct | copies | RNA | Sample | Population |
| colon normal GW98-167 | 21941 | 24.54 | 22693.01 | 45386.02 | colon normal | |
| colon tumor GW98-166 | 21940 | 24.18 | 26862.61 | 53725.22 | colon tumor | 1.18 |
| colon normal GW98-178 | 22080 | 27.08 | 6895.34 | 13790.68 | colon normal | |
| colon tumor GW98-177 | 22060 | 28.41 | 3692.19 | 7384.38 | colon tumor | −1.87 |
| colon normal GW98-561 | 23514 | 26.58 | 8698.68 | 17397.36 | colon normal | |
| colon tumor GW98-560 | 23513 | 27.85 | 4799.22 | 9598.44 | colon tumor | −1.81 |
| colon normal GW98-894 | 24691 | 25.9 | 11972.57 | 23945.14 | colon normal | |
| colon tumor GW98-893 | 24690 | 28.04 | 4396.4 | 8792.80 | colon tumor | −2.72 |
| lung normal GW98-3 | 20742 | 24.25 | 26016.9 | 52033.80 | lung normal | |
| lung tumor GW98-2 | 20741 | 27.64 | 5300.37 | 10600.74 | lung tumor | −4.91 |
| lung normal GW97-179 | 20677 | 25.1 | 17476.66 | 34953.32 | lung normal | |
| lung tumor GW97-178 | 20676 | 25.53 | 14274.54 | 28549.08 | lung tumor | −1.22 |
| lung normal GW98-165 | 21922 | 24.62 | 21917.37 | 43834.74 | lung normal | |
| lung tumor GW98-164 | 21921 | 25.64 | 13526.49 | 27052.98 | lung tumor | −1.62 |
| lung normal GW98-282 | 22584 | 27.08 | 6884.03 | 13768.06 | lung normal | |
| lung tumor GW98-281 | 22583 | 25.37 | 15385.8 | 30771.60 | lung tumor | 2.23 |
| breast normal GW00- | 28750 | 26.07 | 11065.25 | 11065.25 | breast normal | |
| 392 | | | | | | |
| breast tumor GW00-391 | 28746 | 26.87 | 7611.48 | 15222.96 | breast tumor | 1.38 |
| breast normal GW00- | 28798 | 28.65 | 3294.68 | 3294.68 | breast normal | |
| 413 | | | | | | |
| breast tumor GW00-412 | 28797 | 28.52 | 3496.94 | 6993.88 | breast tumor | 2.12 |
| breast normal GW00- | 27592-95 | 29.47 | 2243.69 | 2243.69 | breast normal | |
| 235:238 | | | | | | |
| breast tumor GW00- | 27588-91 | 25.83 | 12385.23 | 12385.23 | breast tumor | 5.52 |
| 231:234 | | | | | | |
| breast normal GW98- | 23656 | 26.05 | 11188.07 | 22376.14 | breast normal | |
| 621 | | | | | | |
| breast tumor GW98-620 | 23655 | 26.03 | 11303.95 | 22607.90 | breast tumor | 1.01 |
| brain normal BB99-542 | 25507 | 27.68 | 5198.79 | 10397.58 | brain normal | |
| brain normal BB99-406 | 25509 | 29.81 | 1909.01 | 3818.02 | brain normal | |
| brain normal BB99-904 | 25546 | 31.84 | 735.25 | 1470.50 | brain normal | |
| brain stage 5 ALZ | 25502 | 28.43 | 3650.66 | 7301.32 | brain stage 5 ALZ | 1.40 |
| BB99-874 | | | | | | |
| brain stage 5 ALZ | 25503 | 29.01 | 2785.56 | 5571.12 | brain stage 5 ALZ | 1.07 |
| BB99-887 | | | | | | |
| brain stage 5 ALZ | 25504 | 29.65 | 2059.62 | 4119.24 | brain stage 5 ALZ | −1.27 |
| BB99-862 | | | | | | |
| brain stage 5 ALZ | 25542 | 30.01 | 1742.3 | 3484.60 | brain stage 5 ALZ | −1.50 |
| BB99-927 | | | | | | |
| CT lung KC | normal | 25.49 | 14553.17 | 29106.34 | CT lung | |
| lung 26 KC | normal | 31.8 | 749.93 | 749.93 | lung 26 | |
| lung 27 KC | normal | 33.35 | 362.66 | 362.66 | lung 27 | |
| lung 24 KC | COPD | 30.67 | 1275.68 | 1275.68 | lung 24 | −6.03 |
| lung 28 KC | COPD | 29.25 | 2490.39 | 2490.39 | lung 28 | −3.09 |
| lung 23 KC | COPD | 30.11 | 1661.24 | 1661.24 | lung 23 | −4.63 |
| lung 25 KC | normal | 32.45 | 553.75 | 553.75 | lung 25 | |
| asthmatic lung | 29321 | 27.3 | 6215.19 | 6215.19 | asthmatic lung | −1.24 |
| ODO3112 | | | | | | |
| asthmatic lung | 29323 | 26.66 | 8407.3 | 16814.60 | asthmatic lung | 2.19 |
| ODO3433 | | | | | | |
| asthmatic lung | 29322 | 24.06 | 28466.73 | 56933.46 | asthmatic lung | 7.40 |
| ODO3397 | | | | | | |
| asthmatic lung | 29325 | 26.22 | 10313.71 | 20627.42 | asthmatic lung | 2.68 |
| ODO4928 | | | | | | |
| endo cells KC | control | 40 | 0 | 0.00 | endo cells | |
| endo VEGF KC | | 40 | 0 | 0.00 | endo VEGF | 0.00 |
| endo bFGF KC | | 40 | 0 | 0.00 | endo bFGF | 0.00 |
| heart Clontech | normal | 27.58 | 5449.78 | 10899.56 | heart | |
| heart (T-1) ischemic | 29417 | 28.42 | 3670.86 | 7341.72 | heart T-1 | −1.48 |
| heart (T-14) non- | 29422 | 27.18 | 6570.11 | 13140.22 | heart T-14 | 1.21 |
| obstructive DCM | | | | | | |
| heart (T-3399) DCM | 29426 | 26.23 | 10277.2 | 20554.40 | heart T-3399 | 1.89 |
| adenoid GW99-269 | 26162 | 31.98 | 688.86 | 1377.72 | adenoid | |
| tonsil GW98-280 | 22582 | 29.31 | 2421.67 | 4843.34 | tonsil | |
| T cells PC00314 | 28453 | 29.53 | 2178.21 | 4356.42 | T cells | |
| PBMNC | | 33.23 | 383.88 | 383.88 | PBMNC | |
| monocyte | | 31.07 | 1057.9 | 2115.80 | monocyte | |
| B cells PC00665 | 28455 | 35.97 | 106.01 | 212.02 | B cells | |
| dendritic cells 28441 | | 33.56 | 328.62 | 657.24 | dendritic cells | |
| neutrophils | 28440 | 22.32 | 64510.36 | 64510.36 | neutrophils | |
| eosinophils | 28446 | 24.18 | 26910.17 | 53820.34 | eosinophils | |
| BM unstim | | 30.35 | 1480.07 | 1480.07 | BM unstim | |
| BM stim | | 31.71 | 782.56 | 782.56 | BM stim | −1.89 |
| osteo dif | | 31.42 | 895.71 | 895.71 | osteo dif | 2.03 |
| osteo undif | | 32.93 | 440.66 | 440.66 | osteo undif | |
| chondrocytes | | 28.98 | 2820.8 | 7052.00 | chondrocytes | |
| OA Synovium IP12/01 | 29462 | 25.37 | 15383.84 | 15383.84 | OA Synovium | |
| OA Synovium NP10/01 | 29461 | 27.12 | 6763.44 | 13526.88 | OA Synovium | |
| OA Synovium NP57/00 | 28464 | 26.48 | 9130.81 | 18261.62 | OA Synovium | |
| RA Synovium NP03/01 | 28466 | 27.78 | 4967.23 | 9934.46 | RA Synovium | |
| RA Synovium NP71/00 | 28467 | 24.72 | 20923.66 | 41847.32 | RA Synovium | |
| RA Synovium NP45/00 | 28475 | 26.15 | 10658.82 | 21317.64 | RA Synovium | |
| OA bone (biobank) | 29217 | 28.68 | 3248.19 | 3248.19 | OA bone | |
| | | | | | (biobank) | |
| OA bone Sample 1 | J. Emory | 27.19 | 6545.82 | 13091.64 | OA bone | |
| OA bone Sample 2 | J. Emory | 27.24 | 6384.92 | 12769.84 | OA bone | |
| Cartilage (pool) | Normal | 26.28 | 10016.65 | 20033.30 | Cartilage (pool) | |
| Cartilage (pool) | OA | 26.67 | 8342.92 | 16685.84 | Cartilage (pool) | −1.20 |
| PBL unifected | 28441 | 31.05 | 1069.84 | 2139.68 | PBL unifected | |
| PBL HIV IIIB | 28442 | 31.7 | 788.06 | 1576.12 | PBL HIV IIIB | −1.36 |
| MRC5 uninfected | 29158 | 26.37 | 9631.13 | 19262.26 | MRC5 uninfected | |
| (100%) | | | | | (100%) | |
| MRC5 HSV strain F | 29178 | 28.38 | 3747.38 | 7494.76 | MRC5 HSV | −2.57 |
| | | | | | strain F | |
| W12 cells | 29179 | 35.15 | 155.25 | 310.50 | W12 cells | |
| Keratinocytes | 29180 | 34.93 | 172.87 | 345.74 | Keratinocytes | |
| B-actin control | | 28.06 | 4342.74 | | | |
| genomic | | 30.54 | 1356.79 | | | |
| 1.00E+05 | | 21.39 | 100000 | | | |
| 1.00E+05 | | 21.64 | 100000 | | | |
| 1.00E+04 | | 26.21 | 10000 | | | |
| 1.00E+04 | | 26.24 | 10000 | | | |
| 1.00E+03 | | 30.9 | 1000 | | | |
| 1.00E+03 | | 30.97 | 1000 | | | |
| 1.00E+02 | | 36.34 | 100 | | | |
| 1.00E+02 | | 36.22 | 100 | | | |
| 1.00E+01 | | 40 | 10 | | | |
| 1.00E+01 | | 40 | 0 | | | |
| 1.00E−00 | | 40 | 0 | | | |
| 1.00E−00 | | 40 | 0 | | | |
| NTC | | 40 | 0 | | | |

Gene Name sbg1002620TIa

| | Fold Change in Disease |
|---|---|
| | Population Relative to |
| Disease tissues | Normal |
| colon tumor | 1.18 |
| colon tumor | −1.87 |
| colon tumor | −1.81 |
| colon tumor | −2.72 |
| lung tumor | −4.91 |
| lung tumor | −1.22 |
| lung tumor | −1.62 |
| lung tumor | 2.23 |
| breast tumor | 1.38 |
| breast tumor | 2.12 |
| breast tumor | 5.52 |
| breast tumor | 1.01 |
| brain stage 5 ALZ | 1.40 |
| brain stage 5 ALZ | 1.07 |
| brain stage 5 ALZ | −1.27 |
| brain stage 5 ALZ | −1.50 |
| lung 24 | −6.03 |
| lung 28 | −3.09 |
| lung 23 | −4.63 |
| asthmatic lung | −1.24 |
| asthmatic lung | 2.19 |
| asthmatic lung | 7.40 |
| asthmatic lung | 2.68 |
| endo VEGF | 0.00 |
| endo bFGF | 0.00 |
| heart T-1 | −1.48 |
| heart T-14 | 1.21 |
| heart T-3399 | 1.89 |
| BM stim | −1.89 |
| osteo dif | 2.03 |
| Cartilage (pool) | −1.20 |
| PBL HIV IIIB | −1.36 |
| MRC5 HSV strain F | −2.57 |

Gene Name sbg1002620TIb

Moderate to high overall expression. Highest normal expression in whole brain, endometrium, jejunum, placenta, thymus, and urinary bladder. Highest disease expression in one of the colon normal/tumor pairs, one of the normal lung samples, one of the asthmatic lung samples, the neutrophils, and the eosinophils. Strong expression in all of the GI tract samples implicates this gene in IBS, IBD, and Crohn's disease. Downregulation in 1 of 4 lung tumor samples is sufficient to make a disease claim in cancer of the lung. Downregulation in 3 of 3 COPD lung samples suggests involvement in chronic obstructive pulmonary disease. Upregulation in 1 of 4 asthmatic lung samples implies a role in asthma. Upregulation in 2 of 3 heart samples suggests this gene may play a role in non-obstructive and obstructive dilated cardiac myopathy. High expression in the RA and OA synovium samples as well as high expression in the chondrocytes and T cells implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1002620TIb | 1 and 2) | 1) | 2) | Copies | (ng) | (ng) | RNA |
| Subcutaneous | 31.35, 31.44 | 58.54 | 55.11 | 56.83 | 3.06 | 16.34 | 928.51 |
| Adipocytes Zenbio | | | | | | | |
| Subcutaneous Adipose | 35.12, 34.21 | 5 | 9.05 | 7.03 | 0.96 | 52.36 | 367.80 |
| Zenbio | | | | | | | |
| Adrenal Gland Clontech | 40, 34.29 | 0 | 8.61 | 4.31 | 0.61 | 81.97 | 352.87 |
| Whole Brain Clontech | 26.02, 26.06 | 1897.9 | 1849.19 | 1873.55 | 7.24 | 6.91 | 12938.85 |
| Fetal Brain Clontech | 40, 36.43 | 0 | 2.13 | 1.07 | 0.48 | 103.95 | 110.71 |
| Cerebellum Clontech | 40, 36.05 | 0 | 2.74 | 1.37 | 2.17 | 23.04 | 31.57 |
| Cervix | 32.23, 33.04 | 33.11 | 19.47 | 26.29 | 2.42 | 20.66 | 543.18 |
| Colon | 30.44, 30.45 | 105.93 | 105.29 | 105.61 | 2.71 | 18.45 | 1948.52 |
| Endometrium | 30.86, 30.56 | 80.75 | 97.92 | 89.34 | 0.73 | 68.21 | 6093.79 |
| Esophagus | 33.03, 32.34 | 19.62 | 30.66 | 25.14 | 1.37 | 36.50 | 917.52 |
| Heart Clontech | 40, 35.05 | 0 | 5.26 | 2.63 | 1.32 | 37.88 | 99.62 |
| Hypothalamus | 40, 36.17 | 0 | 2.53 | 1.27 | 0.32 | 155.28 | 196.43 |
| Ileum | 31.25, 30.31 | 62.52 | 115.58 | 89.05 | 2.58 | 19.38 | 1725.78 |
| Jejunum | 27.75, 27.93 | 612.01 | 543.46 | 577.74 | 6.60 | 7.58 | 4376.78 |
| Kidney | 32.59, 31.86 | 26.03 | 42.14 | 34.09 | 2.12 | 23.58 | 803.89 |
| Liver | 34.66, 34.5 | 6.77 | 7.52 | 7.15 | 1.50 | 33.33 | 238.17 |
| Fetal Liver Clontech | 29.08, 28.58 | 256.83 | 356.67 | 306.75 | 10.40 | 4.81 | 1474.76 |
| Lung | 30.74, 30.68 | 87.44 | 90.91 | 89.18 | 2.57 | 19.46 | 1734.92 |
| Mammary Gland | 27.49, 26.81 | 725.02 | 1132.42 | 928.72 | 13.00 | 3.85 | 3572.00 |
| Clontech | | | | | | | |
| Myometrium | 31.2, 30.29 | 64.54 | 117.26 | 90.90 | 2.34 | 21.37 | 1942.31 |
| Omentum | 31.19, 30.12 | 65.05 | 130.86 | 97.96 | 3.94 | 12.69 | 1243.08 |
| Ovary | 30.24, 30.54 | 120.95 | 99.3 | 110.13 | 4.34 | 11.52 | 1268.72 |
| Pancreas | 36.01, 36.55 | 2.81 | 1.97 | 2.39 | 0.81 | 61.80 | 147.71 |
| Head of Pancreas | 33.95, 35.73 | 10.72 | 3.36 | 7.04 | 1.57 | 31.85 | 224.20 |
| Parotid Gland | 32.16, 33.16 | 34.51 | 18.05 | 26.28 | 5.48 | 9.12 | 239.78 |
| Placenta Clontech | 28.42, 28.02 | 395.01 | 512.77 | 453.89 | 5.26 | 9.51 | 4314.54 |
| Prostate | 30.61, 31.28 | 95.23 | 61.5 | 78.37 | 3.00 | 16.67 | 1306.08 |
| Rectum | 30.5, 30.93 | 101.73 | 76.9 | 89.32 | 1.23 | 40.65 | 3630.69 |
| Salivary Gland | 31.17, 31.07 | 65.95 | 70.23 | 68.09 | 7.31 | 6.84 | 465.73 |
| Clontech | | | | | | | |
| Skeletal Muscle | 40, 40 | 0 | 0 | 0.00 | 1.26 | 39.68 | 0.00 |
| Clontech | | | | | | | |
| Skin | 34.37, 33.12 | 8.18 | 18.53 | 13.36 | 1.21 | 41.32 | 551.86 |
| Small Intestine | 36.71, 34.96 | 1.78 | 5.55 | 3.67 | 0.98 | 51.07 | 187.18 |
| Clontech | | | | | | | |
| Spleen | 30.54, 31.29 | 99.47 | 60.88 | 80.18 | 4.92 | 10.16 | 814.79 |
| Stomach | 32.4, 31.53 | 29.49 | 52 | 40.75 | 2.73 | 18.32 | 746.25 |
| Testis Clontech | 34.4, 35.19 | 8.03 | 4.79 | 6.41 | 0.57 | 87.87 | 563.27 |
| Thymus Clontech | 27.18, 27.12 | 888.84 | 924.29 | 906.57 | 9.89 | 5.06 | 4583.24 |
| Thyroid | 32.17, 30.89 | 34.36 | 79.27 | 56.82 | 2.77 | 18.05 | 1025.54 |
| Trachea Clontech | 30.01, 29.25 | 140.31 | 230.28 | 185.30 | 9.71 | 5.15 | 954.15 |
| Urinary Bladder | 28.33, 27.87 | 420.47 | 565.71 | 493.09 | 5.47 | 9.14 | 4507.22 |
| Uterus | 29.09, 28.81 | 255.27 | 308 | 281.64 | 5.34 | 9.36 | 2637.03 |
| genomic | 27.16 | 900.78 | | | | | |
| b-actin | 27.4 | 769.87 | | | | | |
| 1.00E+05 | 19.87 | 100000 | | | | | |
| 1.00E+05 | 19.95 | 100000 | | | | | |
| 1.00E+04 | 23.4 | 10000 | | | | | |
| 1.00E+04 | 23.39 | 10000 | | | | | |
| 1.00E+03 | 26.94 | 1000 | | | | | |
| 1.00E+03 | 26.95 | 1000 | | | | | |
| 1.00E+02 | 31.02 | 100 | | | | | |
| 1.00E+02 | 30.96 | 100 | | | | | |
| 1.00E+01 | 33.46 | 10 | | | | | |
| 1.00E+01 | 40 | 0 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1002620TIb | identifier) | Ct | copies | RNA | Sample | Population |
| colon normal GW98-167 | 21941 | 22.85 | 18631.2 | 37262.40 | colon normal | |
| colon tumor GW98-166 | 21940 | 22.51 | 23090.96 | 46181.92 | colon tumor | 1.24 |
| colon normal GW98-178 | 22080 | 25.49 | 3620.45 | 7240.90 | colon normal | |
| colon tumor GW98-177 | 22060 | 26.88 | 1527.3 | 3054.60 | colon tumor | −2.37 |
| colon normal GW98-561 | 23514 | 25.37 | 3901.36 | 7802.72 | colon normal | |
| colon tumor GW98-560 | 23513 | 26.08 | 2512.21 | 5024.42 | colon tumor | −1.55 |
| colon normal GW98-894 | 24691 | 23.78 | 10441.49 | 20882.98 | colon normal | |
| colon tumor GW98-893 | 24690 | 25.54 | 3515.48 | 7030.96 | colon tumor | −2.97 |
| lung normal GW98-3 | 20742 | 22.6 | 21810.56 | 43621.12 | lung normal | |
| lung tumor GW98-2 | 20741 | 26.19 | 2349.32 | 4698.64 | lung tumor | −9.28 |
| lung normal GW97-179 | 20677 | 23.38 | 13423.28 | 26846.56 | lung normal | |
| lung tumor GW97-178 | 20676 | 24.51 | 6653.03 | 13306.06 | lung tumor | −2.02 |
| lung normal GW98-165 | 21922 | 23.68 | 11120.91 | 22241.82 | lung normal | |
| lung tumor GW98-164 | 21921 | 24.37 | 7242.32 | 14484.64 | lung tumor | −1.54 |
| lung normal GW98-282 | 22584 | 25.05 | 4745.83 | 9491.66 | lung normal | |
| lung tumor GW98-281 | 22583 | 23.57 | 11943.28 | 23886.56 | lung tumor | 2.52 |
| breast normal GW00- | 28750 | 24.84 | 5415.88 | 5415.88 | breast normal | |
| 392 | | | | | | |
| breast tumor GW00-391 | 28746 | 24.98 | 4973.9 | 9947.80 | breast tumor | 1.84 |
| breast normal GW00- | 28798 | 24.69 | 5954.77 | 5954.77 | breast normal | |
| 413 | | | | | | |
| breast tumor GW00-412 | 28797 | 26.38 | 2081.99 | 4163.98 | breast tumor | −1.43 |
| breast normal GW00- | 27592-95 | 25.04 | 4792.18 | 4792.18 | breast normal | |
| 235:238 | | | | | | |
| breast tumor GW00- | 27588-91 | 23.63 | 11520.86 | 11520.86 | breast tumor | 2.40 |
| 231:234 | | | | | | |
| breast normal GW98- | 23656 | 23.22 | 14836.24 | 29672.48 | breast normal | |
| 621 | | | | | | |
| breast tumor GW98-620 | 23655 | 24.24 | 7879 | 15758.00 | breast tumor | −1.88 |
| brain normal BB99-542 | 25507 | 25.16 | 4447.15 | 8894.30 | brain normal | |
| brain normal BB99-406 | 25509 | 27.05 | 1377.71 | 2755.42 | brain normal | |
| brain normal BB99-904 | 25546 | 29.35 | 330.53 | 661.06 | brain normal | |
| brain stage 5 ALZ | 25502 | 27.64 | 956.16 | 1912.32 | brain stage 5 | −2.15 |
| BB99-874 | | | | | ALZ | |
| brain stage 5 ALZ | 25503 | 27.02 | 1400.04 | 2800.08 | brain stage 5 | −1.47 |
| BB99-887 | | | | | ALZ | |
| brain stage 5 ALZ | 25504 | 27.4 | 1105.21 | 2210.42 | brain stage 5 | −1.86 |
| BB99-862 | | | | | ALZ | |
| brain stage 5 ALZ | 25542 | 27.1 | 1336.02 | 2672.04 | brain stage 5 | −1.54 |
| BB99-927 | | | | | ALZ | |
| CT lung KC | normal | 23.52 | 12295.29 | 24590.58 | CT lung | |
| lung 26 KC | normal | 31.42 | 91.19 | 91.19 | lung 26 | |
| lung 27 KC | normal | 32.34 | 51.71 | 51.71 | lung 27 | |
| lung 24 KC | COPD | 31.27 | 100.29 | 100.29 | lung 24 | −61.80 |
| lung 28 KC | COPD | 28.64 | 511.37 | 511.37 | lung 28 | −12.12 |
| lung 23 KC | COPD | 30.52 | 159.17 | 159.17 | lung 23 | −38.94 |
| lung 25 KC | normal | 32.15 | 57.91 | 57.91 | lung 25 | |
| asthmatic lung | 29321 | 23.19 | 15086.65 | 15086.65 | asthmatic lung | 2.43 |
| ODO3112 | | | | | | |
| asthmatic lung | 29323 | 24.76 | 5706.9 | 11413.80 | asthmatic lung | 1.84 |
| ODO3433 | | | | | | |
| asthmatic lung | 29322 | 21.71 | 37760.01 | 75520.02 | asthmatic lung | 12.18 |
| ODO3397 | | | | | | |
| asthmatic lung | 29325 | 24.16 | 8255.16 | 16510.32 | asthmatic lung | 2.66 |
| ODO4928 | | | | | | |
| endo cells KC | control | 37.31 | 2.36 | 2.36 | endo cells | |
| endo VEGF KC | | 40 | 0 | 0.00 | endo VEGF | −2.36 |
| endo bFGF KC | | 35.67 | 6.54 | 6.54 | endo bFGF | 2.77 |
| heart Clontech | normal | 26.32 | 2170.24 | 4340.48 | heart | |
| heart (T-1) ischemic | 29417 | 25.87 | 2863.04 | 5726.08 | heart T-1 | 1.32 |
| heart (T-14) non- | 29422 | 24.62 | 6200.03 | 12400.06 | heart T-14 | 2.86 |
| obstructive DCM | | | | | | |
| heart (T-3399) DCM | 29426 | 24.06 | 8775.18 | 17550.36 | heart T-3399 | 4.04 |
| adenoid GW99-269 | 26162 | 29.2 | 362.88 | 725.76 | adenoid | |
| tonsil GW98-280 | 22582 | 27.24 | 1222.33 | 2444.66 | tonsil | |
| T cells PC00314 | 28453 | 28.09 | 723.06 | 1446.12 | T cells | |
| PBMNC | | 30.67 | 145.75 | 145.75 | PBMNC | |
| monocyte | | 28.42 | 587.16 | 1174.32 | monocyte | |
| B cells PC00665 | 28455 | 34.17 | 16.57 | 33.14 | B cells | |
| dendritic cells 28441 | | 31.78 | 72.95 | 145.90 | dendritic cells | |
| neutrophils | 28440 | 21.46 | 44297.23 | 44297.23 | neutrophils | |
| eosinophils | 28446 | 22.79 | 19332.21 | 38664.42 | eosinophils | |
| BM unstim | | 29.22 | 358.53 | 358.53 | BM unstim | |
| BM stim | | 31.27 | 100.39 | 100.39 | BM stim | −3.57 |
| osteo dif | | 30.14 | 202.25 | 202.25 | osteo dif | 4.97 |
| osteo undif | | 32.72 | 40.67 | 40.67 | osteo undif | |
| chondrocytes | | 27.3 | 1178.3 | 2945.75 | chondrocytes | |
| OA Synovium IP12/01 | 29462 | 23.33 | 13860.23 | 13860.23 | OA Synovium | |
| OA Synovium NP10/01 | 29461 | 25.3 | 4080.49 | 8160.98 | OA Synovium | |
| OA Synovium NP57/00 | 28464 | 25.23 | 4253.8 | 8507.60 | OA Synovium | |
| RA Synovium NP03/01 | 28466 | 25.46 | 3686.62 | 7373.24 | RA Synovium | |
| RA Synovium NP71/00 | 28467 | 23.31 | 14036.44 | 28072.88 | RA Synovium | |
| RA Synovium NP45/00 | 28475 | 24.28 | 7658.52 | 15317.04 | RA Synovium | |
| OA bone (biobank) | 29217 | 26.48 | 1958.1 | 1958.10 | OA bone | |
| | | | | | (biobank) | |
| OA bone Sample 1 | J. Emory | 25.28 | 4131.76 | 8263.52 | OA bone | |
| OA bone Sample 2 | J. Emory | 25.23 | 4242.9 | 8485.80 | OA bone | |
| Cartilage (pool) | Normal | 24.05 | 8829.67 | 17659.34 | Cartilage (pool) | |
| Cartilage (pool) | OA | 24.28 | 7685.44 | 15370.88 | Cartilage (pool) | −1.15 |
| PBL unifected | 28441 | 29.33 | 334.71 | 669.42 | PBL unifected | |
| PBL HIV IIIB | 28442 | 29.59 | 283.96 | 567.92 | PBL HIV IIIB | −1.18 |
| MRC5 uninfected | 29158 | 23.92 | 9595.12 | 19190.24 | MRC5 | |
| (100%) | | | | | uninfected | |
| | | | | | (100%) | |
| MRC5 HSV strain F | 29178 | 25.2 | 4341.36 | 8682.72 | MRC5 HSV | −2.21 |
| | | | | | strain F | |
| W12 cells | 29179 | 30.43 | 168.9 | 337.80 | W12 cells | |
| Keratinocytes | 29180 | 29.66 | 272.9 | 545.80 | Keratinocytes | |
| B-actin control | | 27.64 | 956.41 | | | |
| genomic | | 27.35 | 1143.39 | | | |
| 1.00E+05 | | 20.14 | 100000 | | | |
| 1.00E+05 | | 20.26 | 100000 | | | |
| 1.00E+04 | | 23.6 | 10000 | | | |
| 1.00E+04 | | 24.02 | 10000 | | | |
| 1.00E+03 | | 27.49 | 1000 | | | |
| 1.00E+03 | | 27.5 | 1000 | | | |
| 1.00E+02 | | 31.66 | 100 | | | |
| 1.00E+02 | | 31.01 | 100 | | | |
| 1.00E+01 | | 40 | 0 | | | |
| 1.00E+01 | | 40 | 0 | | | |
| 1.00E−00 | | 40 | 0 | | | |
| 1.00E−00 | | 40 | 0 | | | |
| NTC | | 40 | 0 | | | |

Gene Name sbg1002620TIb

| | Fold Change in Disease |
|---|---|
| | Population Relative to |
| Disease tissues | Normal |
| colon tumor | 1.24 |
| colon tumor | −2.37 |
| colon tumor | −1.55 |
| colon tumor | −2.97 |
| lung tumor | −9.28 |
| lung tumor | −2.02 |
| lung tumor | −1.54 |
| lung tumor | 2.52 |
| breast tumor | 1.84 |
| breast tumor | −1.43 |
| breast tumor | 2.40 |
| breast tumor | −1.88 |
| brain stage 5 ALZ | −2.15 |
| brain stage 5 ALZ | −1.47 |
| brain stage 5 ALZ | −1.86 |
| brain stage 5 ALZ | −1.54 |
| lung 24 | −61.80 |
| lung 28 | −12.12 |
| lung 23 | −38.94 |
| asthmatic lung | 2.43 |
| asthmatic lung | 1.84 |
| asthmatic lung | 12.18 |
| asthmatic lung | 2.66 |
| endo VEGF | −2.36 |
| endo bFGF | 2.77 |
| heart T-1 | 1.32 |
| heart T-14 | 2.86 |
| heart T-3399 | 4.04 |
| BM stim | −3.57 |
| osteo dif | 4.97 |
| Cartilage (pool) | −1.15 |
| PBL HIV IIIB | −1.18 |
| MRC5 HSV strain F | −2.21 |

Gene Name sbg102200MCTa

Moderate to low overall expression. Highest normal expression in the subcutaneous adipose tissue, whole brain, fetal brain, cerebellum, and fetal liver. Highest disease expression in 2 of 4 lung tumor samples, one of the normal lung samples, one of the normal breast samples, and the CT lung sample. Downregulation in 1 of 4 breast cancer samples implicates this gene in cancer of the breast. Downregulation in 3 of 3 COPD lung samples suggests involvement in chronic obstructive pulmonary disease. Moderate expression in the OA and RA synovium as well as the PBLs, adenoid, tonsil, T cells, B cells, and the chondrocytes indicates involvement in osteoarthritis and rheumatoid arthritis.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg102200MCTa | 1 and 2) | 1) | 2) | Copies | (ng) | (ng) | RNA |
| Subcutaneous | 36.01, 34.76 | 3.83 | 8.04 | 5.94 | 3.06 | 16.34 | 96.98 |
| Adipocytes Zenbio | | | | | | | |
| Subcutaneous Adipose | 34.85, 33.96 | 7.63 | 12.94 | 10.29 | 0.96 | 52.36 | 538.48 |
| Zenbio | | | | | | | |
| Adrenal Gland Clontech | 40, 40 | 0 | 0 | 0.00 | 0.61 | 81.97 | 0.00 |
| Whole Brain Clontech | 26.05, 26.18 | 1434.25 | 1331.2 | 1382.73 | 7.24 | 6.91 | 9549.21 |
| Fetal Brain Clontech | 40, 34.46 | 0 | 9.63 | 4.82 | 0.48 | 103.95 | 500.52 |
| Cerebellum Clontech | 31.7, 32.73 | 49.65 | 26.9 | 38.28 | 2.17 | 23.04 | 881.91 |
| Cervix | 40, 40 | 0 | 0 | 0.00 | 2.42 | 20.66 | 0.00 |
| Colon | 38.55, 38.57 | 0.84 | 0.83 | 0.84 | 2.71 | 18.45 | 15.41 |
| Endometrium | 40, 40 | 0 | 0 | 0.00 | 0.73 | 68.21 | 0.00 |
| Esophagus | 35.23, 40 | 6.09 | 0 | 3.05 | 1.37 | 36.50 | 111.13 |
| Heart Clontech | 40, 40 | 0 | 0 | 0.00 | 1.32 | 37.88 | 0.00 |
| Hypothalamus | 39.8, 40 | 0.4 | 0 | 0.20 | 0.32 | 155.28 | 31.06 |
| Ileum | 34.16, 40 | 11.53 | 0 | 5.77 | 2.58 | 19.38 | 111.72 |
| Jejunum | 32.82, 33.18 | 25.46 | 20.6 | 23.03 | 6.60 | 7.58 | 174.47 |
| Kidney | 34.23, 34.1 | 11.02 | 11.9 | 11.46 | 2.12 | 23.58 | 270.28 |
| Liver | 35.35, 37.28 | 5.65 | 1.79 | 3.72 | 1.50 | 33.33 | 124.00 |
| Fetal Liver Clontech | 29.45, 28.98 | 189.89 | 250.96 | 220.43 | 10.40 | 4.81 | 1059.74 |
| Lung | 34.99, 33.43 | 7.04 | 17.81 | 12.43 | 2.57 | 19.46 | 241.73 |
| Mammary Gland | 31.76, 31.05 | 48.02 | 73.18 | 60.60 | 13.00 | 3.85 | 233.08 |
| Clontech | | | | | | | |
| Myometrium | 34.46, 35.22 | 9.64 | 6.12 | 7.88 | 2.34 | 21.37 | 168.38 |
| Omentum | 37.94, 34.13 | 1.21 | 11.71 | 6.46 | 3.94 | 12.69 | 81.98 |
| Ovary | 34.03, 33.43 | 12.44 | 17.77 | 15.11 | 4.34 | 11.52 | 174.02 |
| Pancreas | 40, 40 | 0 | 0 | 0.00 | 0.81 | 61.80 | 0.00 |
| Head of Pancreas | 40, 40 | 0 | 0 | 0.00 | 1.57 | 31.85 | 0.00 |
| Parotid Gland | 32.56, 31.81 | 29.88 | 46.65 | 38.27 | 5.48 | 9.12 | 349.13 |
| Placenta Clontech | 40, 40 | 0 | 0 | 0.00 | 5.26 | 9.51 | 0.00 |
| Prostate | 40, 36.21 | 0 | 3.39 | 1.70 | 3.00 | 16.67 | 28.25 |
| Rectum | 40, 39.37 | 0 | 0.52 | 0.26 | 1.23 | 40.65 | 10.57 |
| Salivary Gland | 30.6, 31.77 | 95.89 | 47.76 | 71.83 | 7.31 | 6.84 | 491.28 |
| Clontech | | | | | | | |
| Skeletal Muscle | 40, 40 | 0 | 0 | 0.00 | 1.26 | 39.68 | 0.00 |
| Clontech | | | | | | | |
| Skin | 34.47, 35.32 | 9.58 | 5.75 | 7.67 | 1.21 | 41.32 | 316.74 |
| Small Intestine | 40, 40 | 0 | 0 | 0.00 | 0.98 | 51.07 | 0.00 |
| Clontech | | | | | | | |
| Spleen | 34.1, 36.49 | 11.93 | 2.87 | 7.40 | 4.92 | 10.16 | 75.20 |
| Stomach | 35.17, 36.07 | 6.3 | 3.68 | 4.99 | 2.73 | 18.32 | 91.39 |
| Testis Clontech | 37.98, 40 | 1.19 | 0 | 0.60 | 0.57 | 87.87 | 52.28 |
| Thymus Clontech | 31.28, 30.4 | 63.69 | 108.05 | 85.87 | 9.89 | 5.06 | 434.13 |
| Thyroid | 33.08, 32.96 | 21.88 | 23.47 | 22.68 | 2.77 | 18.05 | 409.30 |
| Trachea Clontech | 32.54, 31.34 | 30.14 | 61.71 | 45.93 | 9.71 | 5.15 | 236.48 |
| Urinary Bladder | 33.91, 40 | 13.32 | 0 | 6.66 | 5.47 | 9.14 | 60.88 |
| Uterus | 33.71, 32.43 | 15.04 | 32.13 | 23.59 | 5.34 | 9.36 | 220.83 |
| genomic | 26.3 | 1237.42 | | | | | |
| b-actin | 27.49 | 610.72 | | | | | |
| 1.00E+05 | 19.18 | 100000 | | | | | |
| 1.00E+05 | 19.45 | 100000 | | | | | |
| 1.00E+04 | 22.6 | 10000 | | | | | |
| 1.00E+04 | 22.53 | 10000 | | | | | |
| 1.00E+03 | 26.17 | 1000 | | | | | |
| 1.00E+03 | 26.19 | 1000 | | | | | |
| 1.00E+02 | 30.61 | 100 | | | | | |
| 1.00E+02 | 30.53 | 100 | | | | | |
| 1.00E+01 | 40 | 0 | | | | | |
| 1.00E+01 | 34.91 | 10 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies | | |
|---|---|---|---|---|---|---|
| | | | | of | | |
| | | | | mRNA | | |
| | Reg | | | detected/ | | Fold |
| | number | | Mean | 50 ng | | Change in |
| Sample | (GSK | | GOI | total | | Disease |
| sbg102200MCTa | identifier) | Ct | copies | RNA | Sample | Population |
| colon normal GW98-167 | 21941 | 28.58 | 519.72 | 1039.44 | colon normal | |
| colon tumor GW98-166 | 21940 | 30.18 | 202.86 | 405.72 | colon tumor | −2.56 |
| colon normal GW98-178 | 22080 | 31.39 | 100.15 | 200.30 | colon normal | |
| colon tumor GW98-177 | 22060 | 29.62 | 282.42 | 564.84 | colon tumor | 2.82 |
| colon normal GW98-561 | 23514 | 30.36 | 183.13 | 366.26 | colon normal | |
| colon tumor GW98-560 | 23513 | 30.45 | 173.87 | 347.74 | colon tumor | −1.05 |
| colon normal GW98-894 | 24691 | 30.23 | 196.98 | 393.96 | colon normal | |
| colon tumor GW98-893 | 24690 | 30.35 | 183.76 | 367.52 | colon tumor | −1.07 |
| lung normal GW98-3 | 20742 | 26.68 | 1575.72 | 3151.44 | lung normal | |
| lung tumor GW98-2 | 20741 | 28.8 | 456.47 | 912.94 | lung tumor | −3.45 |
| lung normal GW97-179 | 20677 | 27.94 | 754.47 | 1508.94 | lung normal | |
| lung tumor GW97-178 | 20676 | 26.27 | 2002.28 | 4004.56 | lung tumor | 2.65 |
| lung normal GW98-165 | 21922 | 26.87 | 1411.09 | 2822.18 | lung normal | |
| lung tumor GW98-164 | 21921 | 29.38 | 325.51 | 651.02 | lung tumor | −4.34 |
| lung normal GW98-282 | 22584 | 30 | 225.32 | 450.64 | lung normal | |
| lung tumor GW98-281 | 22583 | 28.64 | 502.02 | 1004.04 | lung tumor | 2.23 |
| breast normal GW00-392 | 28750 | 28.32 | 602.59 | 602.59 | breast normal | |
| breast tumor GW00-391 | 28746 | 28.05 | 709.37 | 1418.74 | breast tumor | 2.35 |
| breast normal GW00-413 | 28798 | 29.56 | 292.43 | 292.43 | breast normal | |
| breast tumor GW00-412 | 28797 | 30.05 | 218.89 | 437.78 | breast tumor | 1.50 |
| breast normal GW00- | 27592-95 | 30.96 | 128.91 | 128.91 | breast normal | |
| 235:238 | | | | | | |
| breast tumor GW00- | 27588-91 | 30.57 | 161.3 | 161.30 | breast tumor | 1.25 |
| 231:234 | | | | | | |
| breast normal GW98-621 | 23656 | 27.04 | 1275.81 | 2551.62 | breast normal | |
| breast tumor GW98-620 | 23655 | 31.35 | 102.26 | 204.52 | breast tumor | −12.48 |
| brain normal BB99-542 | 25507 | 28.44 | 564.32 | 1128.64 | brain normal | |
| brain normal BB99-406 | 25509 | 29.01 | 402.37 | 804.74 | brain normal | |
| brain normal BB99-904 | 25546 | 29.67 | 274.03 | 548.06 | brain normal | |
| brain stage 5 ALZ BB99- | 25502 | 29.6 | 284.82 | 569.64 | brain stage 5 ALZ | −1.45 |
| 874 | | | | | | |
| brain stage 5 ALZ BB99- | 25503 | 27.92 | 765.16 | 1530.32 | brain stage 5 ALZ | 1.85 |
| 887 | | | | | | |
| brain stage 5 ALZ BB99- | 25504 | 28.74 | 472.27 | 944.54 | brain stage 5 ALZ | 1.14 |
| 862 | | | | | | |
| brain stage 5 ALZ BB99- | 25542 | 29.3 | 340.25 | 680.50 | brain stage 5 ALZ | −1.22 |
| 927 | | | | | | |
| CT lung KC | normal | 26.69 | 1569.87 | 3139.74 | CT lung | |
| lung 26 KC | normal | 31.07 | 120.9 | 120.90 | lung 26 | |
| lung 27 KC | normal | 31.17 | 113.69 | 113.69 | lung 27 | |
| lung 24 KC | COPD | 31.8 | 78.78 | 78.78 | lung 24 | −10.98 |
| lung 28 KC | COPD | 32.79 | 44.02 | 44.02 | lung 28 | −19.65 |
| lung 23 KC | COPD | 31.35 | 102.33 | 102.33 | lung 23 | −8.45 |
| lung 25 KC | normal | 31.66 | 85.57 | 85.57 | lung 25 | |
| asthmatic lung ODO3112 | 29321 | 28.76 | 467.45 | 467.45 | asthmatic lung | −1.85 |
| asthmatic lung ODO3433 | 29323 | 27.73 | 851.21 | 1702.42 | asthmatic lung | 1.97 |
| asthmatic lung ODO3397 | 29322 | 27.81 | 812.68 | 1625.36 | asthmatic lung | 1.88 |
| asthmatic lung ODO4928 | 29325 | 29.42 | 317.12 | 634.24 | asthmatic lung | −1.36 |
| endo cells KC | control | 33.06 | 37.57 | 37.57 | endo cells | |
| endo VEGF KC | | 33.9 | 22.97 | 22.97 | endo VEGF | −1.64 |
| endo bFGF KC | | 33.13 | 36.03 | 36.03 | endo bFGF | −1.04 |
| heart Clontech | normal | 31.1 | 118.18 | 236.36 | heart | |
| heart (T-1) ischemic | 29417 | 31.16 | 114.67 | 229.34 | heart T-1 | −1.03 |
| heart (T-14) non- | 29422 | 30.52 | 166.47 | 332.94 | heart T-14 | 1.41 |
| obstructive DCM | | | | | | |
| heart (T-3399) DCM | 29426 | 30.14 | 208.3 | 416.60 | heart T-3399 | 1.76 |
| adenoid GW99-269 | 26162 | 29.07 | 388.9 | 777.80 | adenoid | |
| tonsil GW98-280 | 22582 | 28.29 | 614.82 | 1229.64 | tonsil | |
| T cells PC00314 | 28453 | 29.78 | 256.1 | 512.20 | T cells | |
| PBMNC | | 33.73 | 25.44 | 25.44 | PBMNC | |
| monocyte | | 33.52 | 28.71 | 57.42 | monocyte | |
| B cells PC00665 | 28455 | 28.66 | 495.36 | 990.72 | B cells | |
| dendritic cells 28441 | | 30.81 | 140.17 | 280.34 | dendritic cells | |
| neutrophils | 28440 | 30.17 | 204.92 | 204.92 | neutrophils | |
| eosinophils | 28446 | 34.19 | 19.39 | 38.78 | eosinophils | |
| BM unstim | | 35.9 | 7.11 | 7.11 | BM unstim | |
| BM stim | | 35.77 | 7.7 | 7.70 | BM stim | 1.08 |
| osteo dif | | 34.98 | 12.18 | 12.18 | osteo dif | 2.55 |
| osteo undif | | 36.59 | 4.77 | 4.77 | osteo undif | |
| chondrocytes | | 32.91 | 41.03 | 102.58 | chondrocytes | |
| OA Synovium IP12/01 | 29462 | 29.16 | 370.33 | 370.33 | OA Synovium | |
| OA Synovium NP10/01 | 29461 | 30.69 | 151.13 | 302.26 | OA Synovium | |
| OA Synovium NP57/00 | 28464 | 29.51 | 301.55 | 603.10 | OA Synovium | |
| RA Synovium NP03/01 | 28466 | 30.36 | 183.35 | 366.70 | RA Synovium | |
| RA Synovium NP71/00 | 28467 | 29.27 | 346.61 | 693.22 | RA Synovium | |
| RA Synovium NP45/00 | 28475 | 29.13 | 376.88 | 753.76 | RA Synovium | |
| OA bone (biobank) | 29217 | 30.47 | 171.6 | 171.60 | OA bone | |
| | | | | | (biobank) | |
| OA bone Sample 1 | J. Emory | 30.05 | 219.19 | 438.38 | OA bone | |
| OA bone Sample 2 | J. Emory | 31.13 | 116.62 | 233.24 | OA bone | |
| Cartilage (pool) | Normal | 30.65 | 154.56 | 309.12 | Cartilage (pool) | |
| Cartilage (pool) | OA | 32.01 | 69.39 | 138.78 | Cartilage (pool) | −2.23 |
| PBL unifected | 28441 | 27.91 | 769.91 | 1539.82 | PBL unifected | |
| PBL HIV IIIB | 28442 | 28.14 | 672.12 | 1344.24 | PBL HIV IIIB | −1.15 |
| MRC5 uninfected | 29158 | 31.66 | 85.38 | 170.76 | MRC5 uninfected | |
| (100%) | | | | | (100%) | |
| MRC5 HSV strain F | 29178 | 30.08 | 214.88 | 429.76 | MRC5 HSV strain F | 2.52 |
| W12 cells | 29179 | 33.33 | 32.15 | 64.30 | W12 cells | |
| Keratinocytes | 29180 | 30.64 | 155.16 | 310.32 | Keratinocytes | |
| B-actin control | | 27.55 | 946.64 | | | |
| genomic | | 26.82 | 1451.22 | | | |
| 1.00E+05 | | 19.71 | 100000 | | | |
| 1.00E+05 | | 19.95 | 100000 | | | |
| 1.00E+04 | | 23.43 | 10000 | | | |
| 1.00E+04 | | 23.38 | 10000 | | | |
| 1.00E+03 | | 26.88 | 1000 | | | |
| 1.00E+03 | | 26.8 | 1000 | | | |
| 1.00E+02 | | 31.99 | 100 | | | |
| 1.00E+02 | | 32.15 | 100 | | | |
| 1.00E+01 | | 34.99 | 10 | | | |
| 1.00E+01 | | 40 | 0 | | | |
| 1.00E−00 | | 40 | 0 | | | |
| 1.00E−00 | | 40 | 0 | | | |
| NTC | | 40 | 0 | | | |

Gene Name sbg102200MCTa

| | Fold Change in Disease |
|---|---|
| | Population Relative to |
| Disease tissues | Normal |
| colon tumor | −2.56 |
| colon tumor | 2.82 |
| colon tumor | −1.05 |
| colon tumor | −1.07 |
| lung tumor | −3.45 |
| lung tumor | 2.65 |
| lung tumor | −4.34 |
| lung tumor | 2.23 |
| breast tumor | 2.35 |
| breast tumor | 1.50 |
| breast tumor | 1.25 |
| breast tumor | −12.48 |
| brain stage 5 ALZ | −1.45 |
| brain stage 5 ALZ | 1.85 |
| brain stage 5 ALZ | 1.14 |
| brain stage 5 ALZ | −1.22 |
| lung 24 | −10.98 |
| lung 28 | −19.65 |
| lung 23 | −8.45 |
| asthmatic lung | −1.85 |
| asthmatic lung | 1.97 |
| asthmatic lung | 1.88 |
| asthmatic lung | −1.36 |
| endo VEGF | −1.64 |
| endo bFGF | −1.04 |
| heart T-1 | −1.03 |
| heart T-14 | 1.41 |
| heart T-3399 | 1.76 |
| BM stim | 1.08 |
| osteo dif | 2.55 |
| Cartilage (pool) | −2.23 |
| PBL HIV IIIB | −1.15 |
| MRC5 HSV strain F | 2.52 |

Gene Name sbg102200MCTb

High to moderate overall expression. Highest normal expression in the whole brain, liver, fetal liver, and thymus. Highest disease expression in one of the colon normal/tumor pairs, one of the lung normal/tumor pairs, one of the asthmatic lung samples, the dendritic cells, and the uninfected and HIV-infected PBL cells. Upregulation in 2 of 4 breast tumor samples is sufficient to make a disease claim in cancer of the breast. Upregulation in 1 of 4 AD brain samples indicates a potential role in Alzheimer's disease. Downregulation in 3 of 3 COPD lung samples suggests involvement in chronic obstructive pulmonary disease. Upregulation in 1 of 4 asthmatic lung samples indicates a potential role for this gene in lung cancer. High expression in all of the immune cells. Also high to moderate expression in the OA and RA synovium samples, the OA bone samples, and in the chondrocytes suggests an involvement in osteoarthritis and rheumatoid arthritis.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg102200MCTb | 1 and 2) | 1) | 2) | Copies | (ng) | (ng) | RNA |
| Subcutaneous | 35.66, 34.05 | 3.76 | 10.2 | 6.98 | 3.06 | 16.34 | 114.05 |
| Adipocytes Zenbio | | | | | | | |
| Subcutaneous Adipose | 40, 36 | 0.17 | 3.07 | 1.62 | 0.96 | 52.36 | 84.82 |
| Zenbio | | | | | | | |
| Adrenal Gland Clontech | 40, 40 | 0 | 0 | 0.00 | 0.61 | 81.97 | 0.00 |
| Whole Brain Clontech | 26.32, 26.41 | 1192.66 | 1124.69 | 1158.68 | 7.24 | 6.91 | 8001.90 |
| Fetal Brain Clontech | 40, 35.84 | 0 | 3.38 | 1.69 | 0.48 | 103.95 | 175.68 |
| Cerebellum Clontech | 34.51, 34.28 | 7.68 | 8.8 | 8.24 | 2.17 | 23.04 | 189.86 |
| Cervix | 40, 34.34 | 3.17 | 8.5 | 5.84 | 2.42 | 20.66 | 120.56 |
| Colon | 33.67, 35.6 | 12.86 | 3.91 | 8.39 | 2.71 | 18.45 | 154.70 |
| Endometrium | 35.32, 34.43 | 4.66 | 8.05 | 6.36 | 0.73 | 68.21 | 433.49 |
| Esophagus | 34.27, 35.14 | 8.86 | 5.19 | 7.03 | 1.37 | 36.50 | 256.39 |
| Heart Clontech | 40, 35.05 | 0 | 5.5 | 2.75 | 1.32 | 37.88 | 104.17 |
| Hypothalamus | 40, 40 | 0 | 0 | 0.00 | 0.32 | 155.28 | 0.00 |
| Ileum | 35.29, 33.68 | 4.74 | 12.8 | 8.77 | 2.58 | 19.38 | 169.96 |
| Jejunum | 31.23, 30.98 | 57.65 | 67.22 | 62.44 | 6.60 | 7.58 | 472.99 |
| Kidney | 34.67, 34.21 | 6.95 | 9.2 | 8.08 | 2.12 | 23.58 | 190.45 |
| Liver | 30.76, 30.65 | 77.12 | 82.56 | 79.84 | 1.50 | 33.33 | 2661.33 |
| Fetal Liver Clontech | 26.8, 27.1 | 885.14 | 734.31 | 809.73 | 10.40 | 4.81 | 3892.91 |
| Lung | 40, 40 | 0 | 0.17 | 0.09 | 2.57 | 19.46 | 1.65 |
| Mammary Gland | 31.28, 31.37 | 56.1 | 52.95 | 54.53 | 13.00 | 3.85 | 209.71 |
| Clontech | | | | | | | |
| Myometrium | 34.16, 36.28 | 9.48 | 2.57 | 6.03 | 2.34 | 21.37 | 128.74 |
| Omentum | 34.18, 33.42 | 9.38 | 15 | 12.19 | 3.94 | 12.69 | 154.70 |
| Ovary | 34.21, 34.18 | 9.24 | 9.39 | 9.32 | 4.34 | 11.52 | 107.32 |
| Pancreas | 40, 40 | 0 | 0.14 | 0.07 | 0.81 | 61.80 | 4.33 |
| Head of Pancreas | 40, 35.02 | 0 | 5.59 | 2.80 | 1.57 | 31.85 | 89.01 |
| Parotid Gland | 31.23, 31.9 | 57.68 | 38.33 | 48.01 | 5.48 | 9.12 | 438.00 |
| Placenta Clontech | 31.77, 33.13 | 41.33 | 17.94 | 29.64 | 5.26 | 9.51 | 281.70 |
| Prostate | 39.72, 35.03 | 0.31 | 5.56 | 2.94 | 3.00 | 16.67 | 48.92 |
| Rectum | 35.36, 34.34 | 4.53 | 8.5 | 6.52 | 1.23 | 40.65 | 264.84 |
| Salivary Gland | 30.52, 30.54 | 89.5 | 88.43 | 88.97 | 7.31 | 6.84 | 608.52 |
| Clontech | | | | | | | |
| Skeletal Muscle | 40, 40 | 0 | 0 | 0.00 | 1.26 | 39.68 | 0.00 |
| Clontech | | | | | | | |
| Skin | 40, 40 | 0 | 0 | 0.00 | 1.21 | 41.32 | 0.00 |
| Small Intestine | 40, 39.27 | 0 | 0.41 | 0.21 | 0.98 | 51.07 | 10.47 |
| Clontech | | | | | | | |
| Spleen | 34.21, 33.54 | 9.2 | 13.91 | 11.56 | 4.92 | 10.16 | 117.43 |
| Stomach | 35.05, 33.62 | 5.51 | 13.22 | 9.37 | 2.73 | 18.32 | 171.52 |
| Testis Clontech | 40, 40 | 0 | 0 | 0.00 | 0.57 | 87.87 | 0.00 |
| Thymus Clontech | 28.56, 28.44 | 299.45 | 322.02 | 310.74 | 9.89 | 5.06 | 1570.96 |
| Thyroid | 31.65, 32.3 | 44.76 | 29.81 | 37.29 | 2.77 | 18.05 | 673.01 |
| Trachea Clontech | 32.3, 31.9 | 29.9 | 38.28 | 34.09 | 9.71 | 5.15 | 175.54 |
| Urinary Bladder | 34.34, 35.02 | 8.49 | 5.59 | 7.04 | 5.47 | 9.14 | 64.35 |
| Uterus | 33.07, 34.56 | 18.62 | 7.45 | 13.04 | 5.34 | 9.36 | 122.05 |
| genomic | 25.84 | 1597.08 | | | | | |
| b-actin | 27.32 | 643.56 | | | | | |
| 1.00E+05 | 19.22 | 100000 | | | | | |
| 1.00E+05 | 19.33 | 100000 | | | | | |
| 1.00E+04 | 22.48 | 10000 | | | | | |
| 1.00E+04 | 22.95 | 10000 | | | | | |
| 1.00E+03 | 26.19 | 1000 | | | | | |
| 1.00E+03 | 26.37 | 1000 | | | | | |
| 1.00E+02 | 31.23 | 100 | | | | | |
| 1.00E+02 | 30.48 | 100 | | | | | |
| 1.00E+01 | 32.76 | 10 | | | | | |
| 1.00E+01 | 35.02 | 10 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg102200MCTb | identifier) | Ct | copies | RNA | Sample | Population |
| colon normal GW98-167 | 21941 | 26.48 | 1723.59 | 3447.18 | colon normal | |
| colon tumor GW98-166 | 21940 | 26.06 | 2195.04 | 4390.08 | colon tumor | 1.27 |
| colon normal GW98-178 | 22080 | 29.03 | 389.88 | 779.76 | colon normal | |
| colon tumor GW98-177 | 22060 | 27.39 | 1015.65 | 2031.30 | colon tumor | 2.61 |
| colon normal GW98-561 | 23514 | 26.74 | 1478.76 | 2957.52 | colon normal | |
| colon tumor GW98-560 | 23513 | 26.37 | 1831.8 | 3663.60 | colon tumor | 1.24 |
| colon normal GW98-894 | 24691 | 25.58 | 2918.02 | 5836.04 | colon normal | |
| colon tumor GW98-893 | 24690 | 25 | 4089.75 | 8179.50 | colon tumor | 1.40 |
| lung normal GW98-3 | 20742 | 24.59 | 5183.31 | 10366.62 | lung normal | |
| lung tumor GW98-2 | 20741 | 24.94 | 4232.23 | 8464.46 | lung tumor | −1.22 |
| lung normal GW97-179 | 20677 | 25.73 | 2672.73 | 5345.46 | lung normal | |
| lung tumor GW97-178 | 20676 | 25.36 | 3307.37 | 6614.74 | lung tumor | 1.24 |
| lung normal GW98-165 | 21922 | 26.13 | 2109.28 | 4218.56 | lung normal | |
| lung tumor GW98-164 | 21921 | 25.54 | 2973.82 | 5947.64 | lung tumor | 1.41 |
| lung normal GW98-282 | 22584 | 27.08 | 1212.64 | 2425.28 | lung normal | |
| lung tumor GW98-281 | 22583 | 27.45 | 979.82 | 1959.64 | lung tumor | −1.24 |
| breast normal GW00-392 | 28750 | 26.68 | 1536.57 | 1536.57 | breast normal | |
| breast tumor GW00-391 | 28746 | 26.58 | 1626.58 | 3253.16 | breast tumor | 2.12 |
| breast normal GW00-413 | 28798 | 31.71 | 81.9 | 81.90 | breast normal | |
| breast tumor GW00-412 | 28797 | 26.57 | 1632 | 3264.00 | breast tumor | 39.85 |
| breast normal GW00- | 27592-95 | 32.52 | 51.1 | 51.10 | breast normal | |
| 235:238 | | | | | | |
| breast tumor GW00- | 27588-91 | 29.67 | 268.7 | 268.70 | breast tumor | 5.26 |
| 231:234 | | | | | | |
| breast normal GW98-621 | 23656 | 26.48 | 1727.44 | 3454.88 | breast normal | |
| breast tumor GW98-620 | 23655 | 25.65 | 2793.6 | 5587.20 | breast tumor | 1.62 |
| brain normal BB99-542 | 25507 | 28.62 | 494 | 988.00 | brain normal | |
| brain normal BB99-406 | 25509 | 29.45 | 304.68 | 609.36 | brain normal | |
| brain normal BB99-904 | 25546 | 30.08 | 211.25 | 422.50 | brain normal | |
| brain stage 5 ALZ BB99- | 25502 | 28.75 | 458.64 | 917.28 | brain stage 5 ALZ | 1.36 |
| 874 | | | | | | |
| brain stage 5 ALZ BB99- | 25503 | 26.86 | 1383.71 | 2767.42 | brain stage 5 ALZ | 4.11 |
| 887 | | | | | | |
| brain stage 5 ALZ BB99- | 25504 | 28.02 | 702.59 | 1405.18 | brain stage 5 ALZ | 2.09 |
| 862 | | | | | | |
| brain stage 5 ALZ BB99- | 25542 | 29.57 | 284.31 | 568.62 | brain stage 5 ALZ | −1.18 |
| 927 | | | | | | |
| CT lung KC | normal | 26.58 | 1624.29 | 3248.58 | CT lung | |
| lung 26 KC | normal | 34.19 | 19.27 | 19.27 | lung 26 | |
| lung 27 KC | normal | 32.45 | 53.23 | 53.23 | lung 27 | |
| lung 24 KC | COPD | 33 | 38.6 | 38.60 | lung 24 | −21.75 |
| lung 28 KC | COPD | 32.24 | 59.95 | 59.95 | lung 28 | −14.01 |
| lung 23 KC | COPD | 32.87 | 41.63 | 41.63 | lung 23 | −20.17 |
| lung 25 KC | normal | 33.04 | 37.52 | 37.52 | lung 25 | |
| asthmatic lung ODO3112 | 29321 | 30.13 | 205.46 | 205.46 | asthmatic lung | −4.09 |
| asthmatic lung ODO3433 | 29323 | 27.82 | 788.82 | 1577.64 | asthmatic lung | 1.88 |
| asthmatic lung ODO3397 | 29322 | 25.17 | 3695.43 | 7390.86 | asthmatic lung | 8.80 |
| asthmatic lung ODO4928 | 29325 | 27.6 | 894.3 | 1788.60 | asthmatic lung | 2.13 |
| endo cells KC | control | 28.2 | 633.43 | 633.43 | endo cells | |
| endo VEGF KC | | 28.86 | 429.51 | 429.51 | endo VEGF | −1.47 |
| endo bFGF KC | | 28.97 | 403.08 | 403.08 | endo bFGF | −1.57 |
| heart Clontech | normal | 28.83 | 437.62 | 875.24 | heart | |
| heart (T-1) ischemic | 29417 | 28.42 | 557.54 | 1115.08 | heart T-1 | 1.27 |
| heart (T-14) non- | 29422 | 27.72 | 835.11 | 1670.22 | heart T-14 | 1.91 |
| obstructive DCM | | | | | | |
| heart (T-3399) DCM | 29426 | 28.63 | 493.01 | 986.02 | heart T-3399 | 1.13 |
| adenoid GW99-269 | 26162 | 27 | 1269.75 | 2539.50 | adenoid | |
| tonsil GW98-280 | 22582 | 26.33 | 1876.29 | 3752.58 | tonsil | |
| T cells PC00314 | 28453 | 29.15 | 363.35 | 726.70 | T cells | |
| PBMNC | | 33.05 | 37.41 | 37.41 | PBMNC | |
| monocyte | | 31.49 | 92.84 | 185.68 | monocyte | |
| B cells PC00665 | 28455 | 26.5 | 1700.87 | 3401.74 | B cells | |
| dendritic cells 28441 | | 24.2 | 6511.17 | 13022.34 | dendritic cells | |
| neutrophils | 28440 | 27.01 | 1262.74 | 1262.74 | neutrophils | |
| eosinophils | 28446 | 29.23 | 347.08 | 694.16 | eosinophils | |
| BM unstim | | 30.85 | 135.01 | 135.01 | BM unstim | |
| BM stim | | 28.68 | 478.5 | 478.50 | BM stim | 3.54 |
| osteo dif | | 31.03 | 121.2 | 121.20 | osteo dif | 3.93 |
| osteo undif | | 33.38 | 30.85 | 30.85 | osteo undif | |
| chondrocytes | | 26.63 | 1579.73 | 3949.33 | chondrocytes | |
| OA Synovium IP12/01 | 29462 | 29.11 | 371.98 | 371.98 | OA Synovium | |
| OA Synovium NP10/01 | 29461 | 29.45 | 304.55 | 609.10 | OA Synovium | |
| OA Synovium NP57/00 | 28464 | 27.83 | 784.87 | 1569.74 | OA Synovium | |
| RA Synovium NP03/01 | 28466 | 27.31 | 1063.77 | 2127.54 | RA Synovium | |
| RA Synovium NP71/00 | 28467 | 27.08 | 1217.21 | 2434.42 | RA Synovium | |
| RA Synovium NP45/00 | 28475 | 26.6 | 1606.41 | 3212.82 | RA Synovium | |
| OA bone (biobank) | 29217 | 28.65 | 485.63 | 485.63 | OA bone (biobank) | |
| OA bone Sample 1 | J. Emory | 28.78 | 451.74 | 903.48 | OA bone | |
| OA bone Sample 2 | J. Emory | 28.27 | 607.15 | 1214.30 | OA bone | |
| Cartilage (pool) | Normal | 29.42 | 310.76 | 621.52 | Cartilage (pool) | |
| Cartilage (pool) | OA | 30.09 | 209.7 | 419.40 | Cartilage (pool) | −1.48 |
| PBL unifected | 28441 | 23.85 | 7997.03 | 15994.06 | PBL unifected | |
| PBL HIV IIIB | 28442 | 24.85 | 4447.34 | 8894.68 | PBL HIV IIIB | −1.80 |
| MRC5 uninfected (100%) | 29158 | 27.02 | 1258.46 | 2516.92 | MRC5 uninfected | |
| | | | | | (100%) | |
| MRC5 HSV strain F | 29178 | 29.6 | 278.84 | 557.68 | MRC5 HSV strain F | −4.51 |
| W12 cells | 29179 | 27.21 | 1122.77 | 2245.54 | W12 cells | |
| Keratinocytes | 29180 | 25.64 | 2815.12 | 5630.24 | Keratinocytes | |
| B-actin control | | 27.78 | 807.72 | | | |
| genomic | | 27.04 | 1246.22 | | | |
| 1.00E+05 | | 19.69 | 100000 | | | |
| 1.00E+05 | | 20.01 | 100000 | | | |
| 1.00E+04 | | 23.15 | 10000 | | | |
| 1.00E+04 | | 23.2 | 10000 | | | |
| 1.00E+03 | | 27.02 | 1000 | | | |
| 1.00E+03 | | 26.76 | 1000 | | | |
| 1.00E+02 | | 31.45 | 100 | | | |
| 1.00E+02 | | 32.39 | 100 | | | |
| 1.00E+01 | | 35.72 | 10 | | | |
| 1.00E+01 | | 34.74 | 10 | | | |
| 1.00E−00 | | 40 | 0 | | | |
| 1.00E−00 | | 40 | 0 | | | |
| NTC | | 40 | 0 | | | |

Gene Name sbg102200MCTb

| | Fold Change in Disease |
|---|---|
| | Population Relative to |
| Disease tissues | Normal |
| colon tumor | 1.27 |
| colon tumor | 2.61 |
| colon tumor | 1.24 |
| colon tumor | 1.40 |
| lung tumor | −1.22 |
| lung tumor | 1.24 |
| lung tumor | 1.41 |
| lung tumor | −1.24 |
| breast tumor | 2.12 |
| breast tumor | 39.85 |
| breast tumor | 5.26 |
| breast tumor | 1.62 |
| brain stage 5 ALZ | 1.36 |
| brain stage 5 ALZ | 4.11 |
| brain stage 5 ALZ | 2.09 |
| brain stage 5 ALZ | −1.18 |
| lung 24 | −21.75 |
| lung 28 | −14.01 |
| lung 23 | −20.17 |
| asthmatic lung | −4.09 |
| asthmatic lung | 1.88 |
| asthmatic lung | 8.80 |
| asthmatic lung | 2.13 |
| endo VEGF | −1.47 |
| endo bFGF | −1.57 |
| heart T-1 | 1.27 |
| heart T-14 | 1.91 |
| heart T-3399 | 1.13 |
| BM stim | 3.54 |
| osteo dif | 3.93 |
| Cartilage (pool) | −1.48 |
| PBL HIV IIIB | −1.80 |
| MRC5 HSV strain F | −4.51 |

Gene Name sbg1020380LYG
Failed
Gene Name sbg1007026SGLT

Good to moderate overall expression. The highest normal expression is seen in the whole brain, cerebellum, hypothalamus, jejunum, fetal liver, rectum, and uterus. This gene shows system specific expression in samples representing the central nervous system, the female reproductive organs, and the GI tract. The expression seen in the disease samples confirms that seen in the normal samples with the highest levels of expression seen in the normal and Alzheimer's brain samples. Upregulation in 1 of 4 colon tumor samples and 2 of 4 breast tumor samples as well as downregulation in 1 of 4 lung tumors poses a potential role for this gene in cancers of the colon and breast. Downregulation in 2 of 4 Alzheimer's brain samples implies involvement in Alzheimer's disease. Downregulation in 3 of 3 COPD samples and upregulation in 2 of 4 asthmatic lung samples suggests a potential role for this gene in chronic obstructive pulmonary disorder. Upregulation in the VEGF-treated endothelial cell line implicates a possible role for this gene in angiogenesis. Downregulated in the stimulated bone marrow sample. High expression in the RA and OA synovium samples, the OA bone samples, and the chondrocytes with corroborating high expression in the T cells, B cells, neutrophils, and eosinophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1007026SGLT | 1 and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous Adipose | 40, 40 | 0 | 0.1 | 0.05 | 0.96 | 52.36 | 2.62 |
| Zenbio | | | | | | | |
| Adrenal Gland Clontech | 40, 38.76 | 0 | 0.31 | 0.16 | 0.61 | 81.97 | 12.70 |
| Whole Brain Clontech | 23.01, 22.63 | 3438.45 | 4301.69 | 3870.07 | 7.24 | 6.91 | 26727.00 |
| Fetal Brain Clontech | 35.91, 39.16 | 1.66 | 0.24 | 0.95 | 0.48 | 103.95 | 98.75 |
| Cerebellum Clontech | 34.55, 32.7 | 3.71 | 11.08 | 7.40 | 2.17 | 23.04 | 170.39 |
| Cervix | 34.21, 34.61 | 4.54 | 3.58 | 4.06 | 2.42 | 20.66 | 83.88 |
| Colon | 33.44, 33.7 | 7.16 | 6.14 | 6.65 | 2.71 | 18.45 | 122.69 |
| Endometrium | 34.88, 40 | 3.05 | 0.1 | 1.58 | 0.73 | 68.21 | 107.44 |
| Esophagus | 40, 40 | 0 | 0.1 | 0.05 | 1.37 | 36.50 | 1.82 |
| Heart Clontech | 39.63, 39.53 | 0.18 | 0.19 | 0.19 | 1.32 | 37.88 | 7.01 |
| Hypothalamus | 40, 35.34 | 0 | 2.33 | 1.17 | 0.32 | 155.28 | 180.90 |
| Ileum | 40, 40 | 0 | 0 | 0.00 | 2.58 | 19.38 | 0.00 |
| Jejunum | 30.1, 30.34 | 51.59 | 44.92 | 48.26 | 6.60 | 7.58 | 365.57 |
| Kidney | 33.49, 40 | 6.96 | 0 | 3.48 | 2.12 | 23.58 | 82.08 |
| Liver | 40, 33.7 | 0 | 6.15 | 3.08 | 1.50 | 33.33 | 102.50 |
| Fetal Liver Clontech | 29.58, 29.41 | 70.26 | 77.58 | 73.92 | 10.40 | 4.81 | 355.38 |
| Lung | 35.61, 37.83 | 1.98 | 0.53 | 1.26 | 2.57 | 19.46 | 24.42 |
| Mammary Gland | 33.05, 34.04 | 9.02 | 5.03 | 7.03 | 13.00 | 3.85 | 27.02 |
| Clontech | | | | | | | |
| Myometrium | 33.63, 34.13 | 6.38 | 4.77 | 5.58 | 2.34 | 21.37 | 119.12 |
| Omentum | 40, 40 | 0 | 0 | 0.00 | 3.94 | 12.69 | 0.00 |
| Ovary | 35.38, 37.6 | 2.27 | 0.61 | 1.44 | 4.34 | 11.52 | 16.59 |
| Pancreas | 40, 37.1 | 0 | 0.82 | 0.41 | 0.81 | 61.80 | 25.34 |
| Head of Pancreas | 35.45, 36.72 | 2.18 | 1.03 | 1.61 | 1.57 | 31.85 | 51.11 |
| Parotid Gland | 36.88, 40 | 0.94 | 0 | 0.47 | 5.48 | 9.12 | 4.29 |
| Placenta Clontech | 33.84, 38.46 | 5.66 | 0.37 | 3.02 | 5.26 | 9.51 | 28.66 |
| Prostate | 38.76, 37.12 | 0.31 | 0.81 | 0.56 | 3.00 | 16.67 | 9.33 |
| Rectum | 36.18, 33.82 | 1.42 | 5.7 | 3.56 | 1.23 | 40.65 | 144.72 |
| Salivary Gland Clontech | 38.36, 39.93 | 0.39 | 0.12 | 0.26 | 7.31 | 6.84 | 1.74 |
| Skeletal Muscle | 35.69, 36.23 | 1.9 | 1.38 | 1.64 | 1.26 | 39.68 | 65.08 |
| Clontech | | | | | | | |
| Skin | 39.51, 40 | 0.2 | 0.09 | 0.15 | 1.21 | 41.32 | 5.99 |
| Small Intestine Clontech | 40, 36.04 | 0.1 | 1.53 | 0.82 | 0.98 | 51.07 | 41.62 |
| Spleen | 33.51, 38.51 | 6.87 | 0.36 | 3.62 | 4.92 | 10.16 | 36.74 |
| Stomach | 34.14, 34.19 | 4.73 | 4.59 | 4.66 | 2.73 | 18.32 | 85.35 |
| Testis Clontech | 35.81, 40 | 1.76 | 0.11 | 0.94 | 0.57 | 87.87 | 82.16 |
| Thymus Clontech | 33.26, 32.49 | 7.96 | 12.55 | 10.26 | 9.89 | 5.06 | 51.85 |
| Thyroid | 40, 39.9 | 0.08 | 0.16 | 0.12 | 2.77 | 18.05 | 2.17 |
| Trachea Clontech | 34.25, 33.8 | 4.42 | 5.77 | 5.10 | 9.71 | 5.15 | 26.24 |
| Urinary Bladder | 39.95, 36.54 | 0.1 | 1.14 | 0.62 | 5.47 | 9.14 | 5.67 |
| Uterus | 33, 31.23 | 9.3 | 26.52 | 17.91 | 5.34 | 9.36 | 167.70 |
| genomic | 24.72 | 1251.19 | | | | | |
| b-actin | 25.89 | 625.04 | | | | | |
| 1.00E+05 | 17.54 | 100000 | | | | | |
| 1.00E+05 | 17.65 | 100000 | | | | | |
| 1.00E+04 | 21.03 | 10000 | | | | | |
| 1.00E+04 | 20.92 | 10000 | | | | | |
| 1.00E+03 | 24.87 | 1000 | | | | | |
| 1.00E+03 | 24.96 | 1000 | | | | | |
| 1.00E+02 | 29.1 | 100 | | | | | |
| 1.00E+02 | 29.04 | 100 | | | | | |
| 1.00E+01 | 32.05 | 10 | | | | | |
| 1.00E+01 | 33.51 | 10 | | | | | |
| 1.00E−00 | 36.41 | 1 | | | | | |
| 1.00E−00 | 37.41 | 1 | | | | | |
| NTC | 40 | 0 | | | | | |
| NTC | 40 | −1 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1007026SGLT | identifier) | Ct | copies | RNA | Sample | Population |
| colon normal GW98-167 | 21941 | 29.22 | 127.1 | 254.20 | colon normal | |
| colon tumor GW98-166 | 21940 | 31.07 | 44.11 | 88.22 | colon tumor | −2.88 |
| colon normal GW98-178 | 22080 | 38.41 | 0.65 | 1.30 | colon normal | |
| colon tumor GW98-177 | 22060 | 31.17 | 41.5 | 83.00 | colon tumor | 63.85 |
| colon normal GW98-561 | 23514 | 31.21 | 40.69 | 81.38 | colon normal | |
| colon tumor GW98-560 | 23513 | 33.06 | 14.04 | 28.08 | colon tumor | −2.90 |
| colon normal GW98-894 | 24691 | 29.63 | 100.41 | 200.82 | colon normal | |
| colon tumor GW98-893 | 24690 | 32.22 | 22.7 | 45.40 | colon tumor | −4.42 |
| lung normal GW98-3 | 20742 | 29.8 | 91.46 | 182.92 | lung normal | |
| lung tumor GW98-2 | 20741 | 34.26 | 7.02 | 14.04 | lung tumor | −13.03 |
| lung normal GW97-179 | 20677 | 29.59 | 103.13 | 206.26 | lung normal | |
| lung tumor GW97-178 | 20676 | 29.84 | 89.09 | 178.18 | lung tumor | −1.16 |
| lung normal GW98-165 | 21922 | 29.6 | 102.46 | 204.92 | lung normal | |
| lung tumor GW98-164 | 21921 | 30.8 | 51.53 | 103.06 | lung tumor | −1.99 |
| lung normal GW98-282 | 22584 | 32.53 | 18.97 | 37.94 | lung normal | |
| lung tumor GW98-281 | 22583 | 32.29 | 21.8 | 43.60 | lung tumor | 1.15 |
| breast normal GW00-392 | 28750 | 28.77 | 164.85 | 164.85 | breast normal | |
| breast tumor GW00-391 | 28746 | 30.64 | 56.21 | 112.42 | breast tumor | −1.47 |
| breast normal GW00-413 | 28798 | 34.49 | 6.17 | 6.17 | breast normal | |
| breast tumor GW00-412 | 28797 | 30.37 | 65.97 | 131.94 | breast tumor | 21.38 |
| breast normal GW00- | 27592-95 | 32.87 | 15.66 | 15.66 | breast normal | |
| 235:238 | | | | | | |
| breast tumor GW00- | 27588-91 | 29.8 | 91.07 | 91.07 | breast tumor | 5.82 |
| 231:234 | | | | | | |
| breast normal GW98-621 | 23656 | 28.95 | 149.19 | 298.38 | breast normal | |
| breast tumor GW98-620 | 23655 | 29.62 | 101.25 | 202.50 | breast tumor | −1.47 |
| brain normal BB99-542 | 25507 | 24.5 | 1917.28 | 3834.56 | brain normal | |
| brain normal BB99-406 | 25509 | 21.35 | 11736.92 | 23473.84 | brain normal | |
| brain normal BB99-904 | 25546 | 25.25 | 1248.68 | 2497.36 | brain normal | |
| brain stage 5 ALZ BB99- | 25502 | 27.29 | 386.81 | 773.62 | brain stage 5 ALZ | −12.84 |
| 874 | | | | | | |
| brain stage 5 ALZ BB99- | 25503 | 23.61 | 3196.37 | 6392.74 | brain stage 5 ALZ | −1.55 |
| 887 | | | | | | |
| brain stage 5 ALZ BB99- | 25504 | 25.56 | 1045.09 | 2090.18 | brain stage 5 ALZ | −4.75 |
| 862 | | | | | | |
| brain stage 5 ALZ BB99- | 25542 | 24.45 | 1976.24 | 3952.48 | brain stage 5 ALZ | −2.51 |
| 927 | | | | | | |
| CT lung | normal | 31.07 | 44.03 | 88.06 | CT lung Nml | |
| lung 26 | normal | 24.93 | 1496.87 | | lung 26 Nml | |
| lung 27 | normal | 34.06 | 7.92 | 7.92 | lung 27 Nml | |
| lung 24 | COPD | 34.58 | 5.87 | 5.87 | lung 24 COPD | −5.45 |
| lung 28 | COPD | 40 | 0 | 0.00 | lung 28 COPD | −31.99 |
| lung 23 | COPD | 40 | 0 | 0.00 | lung 23 COPD | −31.99 |
| lung 25 | normal | 40 | 0 | 0.00 | lung 25 Nml | |
| asthmatic lung ODO3112 | 29321 | 33.19 | 13.04 | 13.04 | asthmatic lung | −2.45 |
| asthmatic lung ODO3433 | 29323 | 30.61 | 57.38 | 114.76 | asthmatic lung | 3.59 |
| asthmatic lung ODO3397 | 29322 | 29.2 | 129.16 | 258.32 | asthmatic lung | 8.07 |
| asthmatic lung ODO4928 | 29325 | 30.32 | 67.67 | 135.34 | asthmatic lung | 4.23 |
| endo cells | control | 35.09 | 4.37 | 4.37 | endo cells | |
| endo VEGF | | 32.22 | 22.7 | 22.70 | endo VEGF | 5.19 |
| endo bFGF | | 33.23 | 12.7 | 12.70 | endo bFGF | 2.91 |
| heart Clontech | normal | 33.53 | 10.71 | 21.42 | heart | |
| heart (T-1) ischemic | 29417 | 33.43 | 11.37 | 22.74 | heart (T-1) | 1.06 |
| | | | | | ischemic | |
| heart (T-14) non- | 29422 | 34.45 | 6.32 | 12.64 | heart (T-14) non- | −1.69 |
| obstructive DCM | | | | | obstructive DCM | |
| heart (T-3399) DCM | 29426 | 31.98 | 26.02 | 52.04 | heart (T-3399) | 2.43 |
| | | | | | DCM | |
| adenoid GW99-269 | 26162 | 29.56 | 104.93 | 209.86 | adenoid | |
| tonsil GW98-280 | 22582 | 29 | 144.55 | 289.10 | tonsil | |
| T cells PC00314 | 28453 | 32.03 | 25.34 | 50.68 | T cells | |
| PBMNC | | 37.71 | 0.97 | 0.97 | PBMNC | |
| monocyte | | 37.49 | 1.1 | 2.20 | monocyte | |
| B cells PC00665 | 28455 | 27.18 | 410.49 | 820.98 | B cells | |
| dendritic cells | 28441 | 33.7 | 9.73 | 19.46 | dendritic cells | |
| neutrophils | 28440 | 32.48 | 19.6 | 19.60 | neutrophils | |
| eosinophils | 28446 | 32.44 | 20.08 | 40.16 | eosinophils | |
| BM unstim | | 33.8 | 9.17 | 9.17 | BM unstim | |
| BM stim | treated | 38.89 | 0.49 | 0.49 | BM stim | −18.71 |
| osteo dif | treated | 37.26 | 1.26 | 1.26 | osteo dif | 1.26 |
| osteo undif | | 40 | 0 | 0.00 | osteo undif | |
| chondrocytes | | 32.07 | 24.82 | 62.05 | chondrocytes | |
| OA Synovium IP12/01 | 29462 | 30.31 | 68.26 | 68.26 | OA Synovium | |
| OA Synovium NP10/01 | 29461 | 30.74 | 53.26 | 106.52 | OA Synovium | |
| OA Synovium NP57/00 | 28464 | 31.3 | 38.48 | 76.96 | OA Synovium | |
| RA Synovium NP03/01 | 28466 | 31.08 | 43.89 | 87.78 | RA Synovium | |
| RA Synovium NP71/00 | 28467 | 31.35 | 37.58 | 75.16 | RA Synovium | |
| RA Synovium NP45/00 | 28475 | 30.74 | 53.21 | 106.42 | RA Synovium | |
| OA bone (biobank) | 29217 | 30.47 | 61.99 | 61.99 | OA bone | |
| | | | | | (biobank) | |
| OA bone Sample 1 | J. Emory | 29.92 | 85.09 | 170.18 | OA bone | |
| OA bone Sample 2 | J. Emory | 30.91 | 48.27 | 96.54 | OA bone | |
| Cartilage (pool) | Normal | 31.34 | 37.68 | 75.36 | Nml Cartilage | |
| | | | | | (pool) | |
| Cartilage (pool) | OA | 31.72 | 30.35 | 60.70 | OA Cartilage | −1.24 |
| | | | | | (pool) | |
| PBL unifected | 28441 | 30.8 | 51.54 | 103.08 | PBL unifected | |
| PBL HIV IIIB | 28442 | 32.03 | 25.38 | 50.76 | PBL HIV IIIB | −2.03 |
| MRC5 uninfected | 29158 | 32.29 | 21.85 | 43.70 | MRC5 uninfected | |
| (100%) | | | | | (100%) | |
| MRC5 HSV strain F | 29178 | 31.21 | 40.73 | 81.46 | MRC5 HSV | 1.86 |
| | | | | | strain F | |
| W12 cells | 29179 | 33.12 | 13.52 | 27.04 | W12 cells | |
| Keratinocytes | 29180 | 32.35 | 21.06 | 42.12 | Keratinocytes | |
| B-actin control | | 25.63 | 1002.01 | | | |
| genomic | | 25.19 | 1290.48 | | | |
| 1.00E+05 | | 17.86 | 100000 | | | |
| 1.00E+05 | | 17.85 | 100000 | | | |
| 1.00E+04 | | 21.44 | 10000 | | | |
| 1.00E+04 | | 21.51 | 10000 | | | |
| 1.00E+03 | | 25.33 | 1000 | | | |
| 1.00E+03 | | 25.26 | 1000 | | | |
| 1.00E+02 | | 29.62 | 100 | | | |
| 1.00E+02 | | 30.55 | 100 | | | |
| 1.00E+01 | | 32.93 | 10 | | | |
| 1.00E+01 | | 33.46 | 10 | | | |
| 1.00E−00 | | 38.18 | 1 | | | |
| 1.00E−00 | | 40 | 0 | | | |
| NTC | | 38.28 | −1 | | | |

*lung 26 Normal has been omitted due to multiple amplification failures from that sample

Gene Name sbg1007026SGLT

| | Fold Change in Disease |
|---|---|
| | Population Relative to |
| Disease tissues | Normal |
| colon tumor | −2.88 |
| colon tumor | 63.85 |
| colon tumor | −2.90 |
| colon tumor | −4.42 |
| lung tumor | −13.03 |
| lung tumor | −1.16 |
| lung tumor | −1.99 |
| lung tumor | 1.15 |
| breast tumor | −1.47 |
| breast tumor | 21.38 |
| breast tumor | 5.82 |
| breast tumor | −1.47 |
| brain stage 5 ALZ | −12.84 |
| brain stage 5 ALZ | −1.55 |
| brain stage 5 ALZ | −4.75 |
| brain stage 5 ALZ | −2.51 |
| lung 24 | −5.45 |
| lung 28 | −31.99 |
| lung 23 | −31.99 |
| asthmatic lung | −2.45 |
| asthmatic lung | 3.59 |
| asthmatic lung | 8.07 |
| asthmatic lung | 4.23 |
| endo VEGF | 5.19 |
| endo bFGF | 2.91 |
| heart T-1 | 1.06 |
| heart T-14 | −1.69 |
| heart T-3399 | 2.43 |
| BM stim | −18.71 |
| osteo dif | 1.26 |
| Cartilage (pool) | −1.24 |
| PBL HIV IIIB | −2.03 |
| MRC5 HSV strain F | 1.86 |

Gene Name sbg1012732GLUT

High to moderate overall expression. This gene is expressed fairly ubiquitously in all normal samples analyzed with highest levels of expression seen in the whole brain, fetal brain cerebellum, kidney, fetal liver, and the placenta. This gene is also expressed fairly ubiquitously in the disease samples. Downregulation in 3 of 3 COPD samples suggests a potential role for this gene in chronic obstructive pulmonary disorder. Upregulation in 3 of 3 disease heart samples implies an involvement in cardiovascular diseases such as non-obstructive and obstructive DCM and ischemia. Downregulation in the HSV-infected MRC5 cells suggests that this gene may play a role in HSV. Upregulated in the differentiated osteoblasts. High expression in the RA and OA synovium samples, the OA bone samples, and the chondrocytes with corroborating high expression in the T cells, B cells, dendritic cells, neutrophils, and eosinophils implicates this gene in osteoarthritis and rheumatoid arthritis.

| | | | | | | | copies of |
|---|---|---|---|---|---|---|---|
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1012732GLUT | 1 and 2) | 1) | 2) | Copies | (ng) | (ng) | RNA |
| Subcutaneous | 30.84, 30.49 | 46.76 | 57.63 | 52.20 | 3.06 | 16.34 | 852.86 |
| Adipocytes Zenbio | | | | | | | |
| Subcutaneous Adipose | 34.89, 35.19 | 4.31 | 3.62 | 3.97 | 0.96 | 52.36 | 207.59 |
| Zenbio | | | | | | | |
| Adrenal Gland Clontech | 33.79, 32.74 | 8.27 | 15.31 | 11.79 | 0.61 | 81.97 | 966.39 |
| Whole Brain Clontech | 22.02, 21.91 | 8374.61 | 8944.05 | 8659.33 | 7.24 | 6.91 | 59802.00 |
| Fetal Brain Clontech | 32.03, 32.85 | 23.23 | 14.34 | 18.79 | 0.48 | 103.95 | 1952.70 |
| Cerebellum Clontech | 28.2, 28.26 | 221.12 | 213.07 | 217.10 | 2.17 | 23.04 | 5002.19 |
| Cervix | 32.91, 34.54 | 13.81 | 5.31 | 9.56 | 2.42 | 20.66 | 197.52 |
| Colon | 30.88, 32.48 | 45.58 | 17.87 | 31.73 | 2.71 | 18.45 | 585.33 |
| Endometrium | 36.13, 32.5 | 2.08 | 17.6 | 9.84 | 0.73 | 68.21 | 671.21 |
| Esophagus | 32.19, 33.5 | 21.15 | 9.77 | 15.46 | 1.37 | 36.50 | 564.23 |
| Heart Clontech | 31.93, 31.73 | 24.67 | 27.76 | 26.22 | 1.32 | 37.88 | 992.99 |
| Hypothalamus | 40, 32.77 | 0 | 15.04 | 7.52 | 0.32 | 155.28 | 1167.70 |
| Ileum | 30.94, 30.52 | 44.17 | 56.37 | 50.27 | 2.58 | 19.38 | 974.22 |
| Jejunum | 30.04, 29.34 | 75.03 | 113.25 | 94.14 | 6.60 | 7.58 | 713.18 |
| Kidney | 29.72, 29.18 | 90.58 | 124.19 | 107.39 | 2.12 | 23.58 | 2532.67 |
| Liver | 34.81, 32.2 | 4.52 | 21.05 | 12.79 | 1.50 | 33.33 | 426.17 |
| Fetal Liver Clontech | 26.6, 26.85 | 567.46 | 488.36 | 527.91 | 10.40 | 4.81 | 2538.03 |
| Lung | 31.61, 30.52 | 29.69 | 56.54 | 43.12 | 2.57 | 19.46 | 838.81 |
| Mammary Gland | 28.06, 27.59 | 239.58 | 316.2 | 277.89 | 13.00 | 3.85 | 1068.81 |
| Clontech | | | | | | | |
| Myometrium | 30.44, 29.88 | 59.31 | 82.52 | 70.92 | 2.34 | 21.37 | 1515.28 |
| Omentum | 31.7, 30.82 | 28.2 | 47.35 | 37.78 | 3.94 | 12.69 | 479.38 |
| Ovary | 30.92, 31.56 | 44.74 | 30.55 | 37.65 | 4.34 | 11.52 | 433.70 |
| Pancreas | 33.08, 32.66 | 12.54 | 16.03 | 14.29 | 0.81 | 61.80 | 882.88 |
| Head of Pancreas | 33.98, 34.1 | 7.36 | 6.89 | 7.13 | 1.57 | 31.85 | 226.91 |
| Parotid Gland | 29.5, 30.55 | 102.86 | 55.41 | 79.14 | 5.48 | 9.12 | 722.03 |
| Placenta Clontech | 25.63, 25.87 | 1002.18 | 869.55 | 935.87 | 5.26 | 9.51 | 8896.06 |
| Prostate | 30.23, 31.17 | 67.04 | 38.48 | 52.76 | 3.00 | 16.67 | 879.33 |
| Rectum | 31.29, 31.15 | 35.89 | 38.94 | 37.42 | 1.23 | 40.65 | 1520.93 |
| Salivary Gland Clontech | 28.82, 28.83 | 153.9 | 152.53 | 153.22 | 7.31 | 6.84 | 1047.98 |
| Skeletal Muscle | 33.23, 32.66 | 11.48 | 16.02 | 13.75 | 1.26 | 39.68 | 545.63 |
| Clontech | | | | | | | |
| Skin | 32.62, 32.57 | 16.46 | 16.96 | 16.71 | 1.21 | 41.32 | 690.50 |
| Small Intestine Clontech | 34.63, 32.82 | 5.03 | 14.62 | 9.83 | 0.98 | 51.07 | 501.79 |
| Spleen | 31.45, 32.71 | 32.66 | 15.56 | 24.11 | 4.92 | 10.16 | 245.02 |
| Stomach | 32.38, 32.43 | 18.93 | 18.41 | 18.67 | 2.73 | 18.32 | 341.94 |
| Testis Clontech | 32.32, 32.27 | 19.58 | 20.17 | 19.88 | 0.57 | 87.87 | 1746.49 |
| Thymus Clontech | 27.24, 26.75 | 388.32 | 518.55 | 453.44 | 9.89 | 5.06 | 2292.39 |
| Thyroid | 30.48, 29.44 | 57.86 | 106.5 | 82.18 | 2.77 | 18.05 | 1483.39 |
| Trachea Clontech | 29.96, 30.29 | 78.48 | 64.81 | 71.65 | 9.71 | 5.15 | 368.92 |
| Urinary Bladder | 30.59, 30.1 | 54.25 | 72.45 | 63.35 | 5.47 | 9.14 | 579.07 |
| Uterus | 30.62, 29.73 | 53.18 | 89.72 | 71.45 | 5.34 | 9.36 | 669.01 |
| genomic | 25.15 | 1330.24 | | | | | |
| b-actin | 26.01 | 800.58 | | | | | |
| 1.00E+05 | 18.01 | 100000 | | | | | |
| 1.00E+05 | 18.19 | 100000 | | | | | |
| 1.00E+04 | 21.35 | 10000 | | | | | |
| 1.00E+04 | 21.3 | 10000 | | | | | |
| 1.00E+03 | 25.59 | 1000 | | | | | |
| 1.00E+03 | 25.51 | 1000 | | | | | |
| 1.00E+02 | 29.95 | 100 | | | | | |
| 1.00E+02 | 29.37 | 100 | | | | | |
| 1.00E+01 | 34.05 | 10 | | | | | |
| 1.00E+01 | 33.22 | 10 | | | | | |
| 1.00E−00 | 37.19 | 1 | | | | | |
| 1.00E−00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1012732GLUT | identifier) | Ct | copies | RNA | Sample | Population |
| colon normal GW98-167 | 21941 | 25.38 | 1695.47 | 3390.94 | colon normal | |
| colon tumor GW98-166 | 21940 | 25.51 | 1576.91 | 3153.82 | colon tumor | −1.08 |
| colon normal GW98-178 | 22080 | 26.8 | 765.5 | 1531.00 | colon normal | |
| colon tumor GW98-177 | 22060 | 25.86 | 1297.34 | 2594.68 | colon tumor | 1.69 |
| colon normal GW98-561 | 23514 | 26.12 | 1120.64 | 2241.28 | colon normal | |
| colon tumor GW98-560 | 23513 | 26.12 | 1121.89 | 2243.78 | colon tumor | 1.00 |
| colon normal GW98-894 | 24691 | 24.85 | 2283.56 | 4567.12 | colon normal | |
| colon tumor GW98-893 | 24690 | 24.37 | 2989.5 | 5979.00 | colon tumor | 1.31 |
| lung normal GW98-3 | 20742 | 24.37 | 2984.11 | 5968.22 | lung normal | |
| lung tumor GW98-2 | 20741 | 25.38 | 1691.06 | 3382.12 | lung tumor | −1.76 |
| lung normal GW97-179 | 20677 | 25.07 | 2020.52 | 4041.04 | lung normal | |
| lung tumor GW97-178 | 20676 | 24.61 | 2607.03 | 5214.06 | lung tumor | 1.29 |
| lung normal GW98-165 | 21922 | 24.92 | 2195.85 | 4391.70 | lung normal | |
| lung tumor GW98-164 | 21921 | 25.36 | 1712.62 | 3425.24 | lung tumor | −1.28 |
| lung normal GW98-282 | 22584 | 26.24 | 1049.97 | 2099.94 | lung normal | |
| lung tumor GW98-281 | 22583 | 25.94 | 1241.8 | 2483.60 | lung tumor | 1.18 |
| breast normal GW00-392 | 28750 | 25.26 | 1813.7 | 1813.70 | breast normal | |
| breast tumor GW00-391 | 28746 | 24.87 | 2259.54 | 4519.08 | breast tumor | 2.49 |
| breast normal GW00-413 | 28798 | 25.4 | 1672.46 | 1672.46 | breast normal | |
| breast tumor GW00-412 | 28797 | 25.21 | 1864.18 | 3728.36 | breast tumor | 2.23 |
| breast normal GW00- | 27592-95 | 25.68 | 1435.2 | 1435.20 | breast normal | |
| 235:238 | | | | | | |
| breast tumor GW00- | 27588-91 | 24.08 | 3510.78 | 3510.78 | breast tumor | 2.45 |
| 231:234 | | | | | | |
| breast normal GW98-621 | 23656 | 24.16 | 3363.26 | 6726.52 | breast normal | |
| breast tumor GW98-620 | 23655 | 24.19 | 3300.23 | 6600.46 | breast tumor | −1.02 |
| brain normal BB99-542 | 25507 | 22.64 | 7880.57 | 15761.14 | brain normal | |
| brain normal BB99-406 | 25509 | 23.32 | 5357.05 | 10714.10 | brain normal | |
| brain normal BB99-904 | 25546 | 23.66 | 4436.27 | 8872.54 | brain normal | |
| brain stage 5 ALZ BB99- | 25502 | 24.7 | 2474.23 | 4948.46 | brain stage 5 ALZ | −2.38 |
| 874 | | | | | | |
| brain stage 5 ALZ BB99- | 25503 | 23.22 | 5674.88 | 11349.76 | brain stage 5 ALZ | −1.04 |
| 887 | | | | | | |
| brain stage 5 ALZ BB99- | 25504 | 23.5 | 4868.6 | 9737.20 | brain stage 5 ALZ | −1.21 |
| 862 | | | | | | |
| brain stage 5 ALZ BB99- | 25542 | 23.17 | 5843.2 | 11686.40 | brain stage 5 ALZ | −1.01 |
| 927 | | | | | | |
| CT lung | normal | 25.61 | 1486.99 | 2973.98 | CT lung Nml | |
| lung 26 | normal | 26.55 | 879.91 | | lung 26 Nml | |
| lung 27 | normal | 29.44 | 174.3 | 174.30 | lung 27 Nml | |
| lung 24 | COPD | 29.99 | 128.5 | 128.50 | lung 24 COPD | −8.67 |
| lung 28 | COPD | 29.56 | 163.34 | 163.34 | lung 28 COPD | −6.82 |
| lung 23 | COPD | 29.59 | 160.67 | 160.67 | lung 23 COPD | −6.94 |
| lung 25 | normal | 29.24 | 194.83 | 194.83 | lung 25 Nml | |
| asthmatic lung ODO3112 | 29321 | 27.22 | 604.38 | 604.38 | asthmatic lung | −1.84 |
| asthmatic lung ODO3433 | 29323 | 26.46 | 923.43 | 1846.86 | asthmatic lung | 1.66 |
| asthmatic lung ODO3397 | 29322 | 26.16 | 1094.36 | 2188.72 | asthmatic lung | 1.96 |
| asthmatic lung ODO4928 | 29325 | 25.51 | 1576.72 | 3153.44 | asthmatic lung | 2.83 |
| endo cells | control | 29.09 | 211.78 | 211.78 | endo cells | |
| endo VEGF | | 30.07 | 122.67 | 122.67 | endo VEGF | −1.73 |
| endo bFGF | | 29.93 | 132.63 | 132.63 | endo bFGF | −1.60 |
| heart Clontech | normal | 27.35 | 561.26 | 1122.52 | heart | |
| heart (T-1) ischemic | 29417 | 23.82 | 4053.65 | 8107.30 | heart (T-1) | 7.22 |
| | | | | | ischemic | |
| heart (T-14) non- | 29422 | 23.96 | 3746.25 | 7492.50 | heart (T-14) non- | 6.67 |
| obstructive DCM | | | | | obstructive DCM | |
| heart (T-3399) DCM | 29426 | 23.35 | 5282.35 | 10564.70 | heart (T-3399) | 9.41 |
| | | | | | DCM | |
| adenoid GW99-269 | 26162 | 25.71 | 1405.41 | 2810.82 | adenoid | |
| tonsil GW98-280 | 22582 | 23.97 | 3725.77 | 7451.54 | tonsil | |
| T cells PC00314 | 28453 | 25.03 | 2062.68 | 4125.36 | T cells | |
| PBMNC | | 30.16 | 116.69 | 116.69 | PBMNC | |
| monocyte | | 30.15 | 117.05 | 234.10 | monocyte | |
| B cells PC00665 | 28455 | 23.22 | 5673.63 | 11347.26 | B cells | |
| dendritic cells | 28441 | 25.74 | 1385.65 | 2771.30 | dendritic cells | |
| neutrophils | 28440 | 27.14 | 631.86 | 631.86 | neutrophils | |
| eosinophils | 28446 | 28.27 | 335.66 | 671.32 | eosinophils | |
| BM unstim | | 30.08 | 122.25 | 122.25 | BM unstim | |
| BM stim | treated | 29.57 | 162.71 | 162.71 | BM stim | 1.33 |
| osteo dif | treated | 29.07 | 214.84 | 214.84 | osteo dif | 2.91 |
| osteo undif | | 30.98 | 73.85 | 73.85 | osteo undif | |
| chondrocytes | | 25.41 | 1667.28 | 4168.20 | chondrocytes | |
| OA Synovium IP12/01 | 29462 | 24.65 | 2554.39 | 2554.39 | OA Synovium | |
| OA Synovium NP10/01 | 29461 | 25.72 | 1399 | 2798.00 | OA Synovium | |
| OA Synovium NP57/00 | 28464 | 25.24 | 1828.2 | 3656.40 | OA Synovium | |
| RA Synovium NP03/01 | 28466 | 25.69 | 1422.61 | 2845.22 | RA Synovium | |
| RA Synovium NP71/00 | 28467 | 25.25 | 1818.15 | 3636.30 | RA Synovium | |
| RA Synovium NP45/00 | 28475 | 25.22 | 1857.13 | 3714.26 | RA Synovium | |
| OA bone (biobank) | 29217 | 26.19 | 1074.74 | 1074.74 | OA bone (biobank) | |
| OA bone Sample 1 | J. Emory | 26.71 | 805.65 | 1611.30 | OA bone | |
| OA bone Sample 2 | J. Emory | 26.96 | 700.88 | 1401.76 | OA bone | |
| Cartilage (pool) | Normal | 26.38 | 968.45 | 1936.90 | Nml Cartilage | |
| | | | | | (pool) | |
| Cartilage (pool) | OA | 28.07 | 376.23 | 752.46 | OA Cartilage | −2.57 |
| | | | | | (pool) | |
| PBL unifected | 28441 | 25.09 | 1997.75 | 3995.50 | PBL unifected | |
| PBL HIV IIIB | 28442 | 25.36 | 1710.81 | 3421.62 | PBL HIV IIIB | −1.17 |
| MRC5 uninfected | 29158 | 25.28 | 1788.71 | 3577.42 | MRC5 uninfected | |
| (100%) | | | | | (100%) | |
| MRC5 HSV strain F | 29178 | 30.17 | 116.17 | 232.34 | MRC5 HSV strain F | −15.40 |
| W12 cells | 29179 | 27.6 | 489.01 | 978.02 | W12 cells | |
| Keratinocytes | 29180 | 26.4 | 959.61 | 1919.22 | Keratinocytes | |
| B-actin control | | 25.62 | 1482.86 | | | |
| genomic | | 25.42 | 1657.68 | | | |
| 1.00E+05 | | 18.49 | 100000 | | | |
| 1.00E+05 | | 18.49 | 100000 | | | |
| 1.00E+04 | | 21.94 | 10000 | | | |
| 1.00E+04 | | 21.98 | 10000 | | | |
| 1.00E+03 | | 25.34 | 1000 | | | |
| 1.00E+03 | | 25.39 | 1000 | | | |
| 1.00E+02 | | 30.59 | 100 | | | |
| 1.00E+02 | | 30.9 | 100 | | | |
| 1.00E+01 | | 32.51 | 10 | | | |
| 1.00E+01 | | 39.12 | 10 | | | |
| 1.00E−00 | | 39.07 | 1 | | | |
| 1.00E−00 | | 36.71 | 1 | | | |
| NTC | | 39.63 | −1 | | | |

*lung 26 Normal has been omitted due to multiple amplification failures from that sample

Gene Name sbg1012732GLUT

| | Fold Change in Disease |
|---|---|
| | Population Relative to |
| Disease tissues | Normal |
| colon tumor | −1.08 |
| colon tumor | 1.69 |
| colon tumor | 1.00 |
| colon tumor | 1.31 |
| lung tumor | −1.76 |
| lung tumor | 1.29 |
| lung tumor | −1.28 |
| lung tumor | 1.18 |
| breast tumor | 2.49 |
| breast tumor | 2.23 |
| breast tumor | 2.45 |
| breast tumor | −1.02 |
| brain stage 5 ALZ | −2.38 |
| brain stage 5 ALZ | −1.04 |
| brain stage 5 ALZ | −1.21 |
| brain stage 5 ALZ | −1.01 |
| lung 24 | −8.67 |
| lung 28 | −6.82 |
| lung 23 | −6.94 |
| asthmatic lung | −1.84 |
| asthmatic lung | 1.66 |
| asthmatic lung | 1.96 |
| asthmatic lung | 2.83 |
| endo VEGF | −1.73 |
| endo bFGF | −1.60 |
| heart T-1 | 7.22 |
| heart T-14 | 6.67 |
| heart T-3399 | 9.41 |
| BM stim | 1.33 |
| osteo dif | 2.91 |
| Cartilage (pool) | −2.57 |
| PBL HIV IIIB | −1.17 |
| MRC5 HSV strain F | −15.40 |

Gene Name sbg1012732GLUTb
The same as sbg1012732GLUT.
Gene Name sbg1018172CSP

Moderate to low overall expression. Highest normal expression is seen in the whole brain, kidney, thyroid, and uterus. This gene is expressed in all of the samples representing the female reproductive system. Highest disease expression is seen in many of the normal/tumor lung samples and the asthmatic lung samples. Downregulation in 2 of 4 lung tumor samples and upregulation in 2 of 4 breast tumor samples suggests an involvement in cancers of the lung and breast. Downregulation in 3 of 3 COPD samples suggests a potential role for this gene in chronic obstructive pulmonary disorder. Upregulation in 2 of 4 asthmatic lung samples implies an involvement in asthma. Upregulation in 1 of 3 disease heart samples implies an involvement in cardiovascular disease such as obstructive DCM. Downregulation in the OA cartilage pool with corroborating low expression in the immune cells (T and B cells in particular) implicates this gene in osteoarthritis and rheumatoid arthritis. Upregulation in the HSV-infected MRC5 cells suggests that this gene may be a host factor in HSV.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1018172CSP | 1 and 2) | 1) | 2) | Copies | (ng) | (ng) | RNA |
| Subcutaneous | 35.54, 34.87 | 7.65 | 11.21 | 9.43 | 3.06 | 16.34 | 154.08 |
| Adipocytes Zenbio | | | | | | | |
| Subcutaneous Adipose | 40, 40 | 0.4 | 0 | 0.20 | 0.96 | 52.36 | 10.47 |
| Zenbio | | | | | | | |
| Adrenal Gland Clontech | 40, 40 | 0 | 0 | 0.00 | 0.61 | 81.97 | 0.00 |
| Whole Brain Clontech | 25.71, 25.78 | 1950.98 | 1867.58 | 1909.28 | 7.24 | 6.91 | 13185.64 |
| Fetal Brain Clontech | 40, 40 | 0 | 0 | 0.00 | 0.48 | 103.95 | 0.00 |
| Cerebellum Clontech | 36.32, 35.16 | 4.95 | 9.48 | 7.22 | 2.17 | 23.04 | 166.24 |
| Cervix | 36.76, 36.16 | 3.85 | 5.42 | 4.64 | 2.42 | 20.66 | 95.76 |
| Colon | 36.52, 36.41 | 4.41 | 4.7 | 4.56 | 2.71 | 18.45 | 84.04 |
| Endometrium | 36.4, 35.92 | 4.73 | 6.19 | 5.46 | 0.73 | 68.21 | 372.44 |
| Esophagus | 40, 40 | 0 | 0 | 0.00 | 1.37 | 36.50 | 0.00 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 40, 40 | 0 | 0 | 0.00 | 2.58 | 19.38 | 0.00 |
| Jejunum | 36.53, 33.22 | 4.38 | 28.28 | 16.33 | 6.60 | 7.58 | 123.71 |
| Kidney | 32.81, 32.45 | 35.74 | 43.67 | 39.71 | 2.12 | 23.58 | 936.44 |
| Liver | 35.92, 36.2 | 6.19 | 5.29 | 5.74 | 1.50 | 33.33 | 191.33 |
| Fetal Liver Clontech | 31.57, 30.46 | 71.69 | 134.34 | 103.02 | 10.40 | 4.81 | 495.26 |
| Lung | 33.35, 36.86 | 26.32 | 3.65 | 14.99 | 2.57 | 19.46 | 291.54 |
| Mammary Gland | 32.99, 32.17 | 32.23 | 51.27 | 41.75 | 13.00 | 3.85 | 160.58 |
| Clontech | | | | | | | |
| Myometrium | 34.99, 40 | 10.44 | 0 | 5.22 | 2.34 | 21.37 | 111.54 |
| Omentum | 35.61, 40 | 7.37 | 0 | 3.69 | 3.94 | 12.69 | 46.76 |
| Ovary | 35.83, 35.54 | 6.53 | 7.67 | 7.10 | 4.34 | 11.52 | 81.80 |
| Pancreas | 35.86, 40 | 6.39 | 0 | 3.20 | 0.81 | 61.80 | 197.47 |
| Head of Pancreas | 40, 40 | 0 | 0 | 0.00 | 1.57 | 31.85 | 0.00 |
| Parotid Gland | 34.26, 34.03 | 15.73 | 17.96 | 16.85 | 5.48 | 9.12 | 153.70 |
| Placenta Clontech | 32.64, 33.16 | 39.34 | 29.26 | 34.30 | 5.26 | 9.51 | 326.05 |
| Prostate | 35.71, 40 | 6.95 | 0 | 3.48 | 3.00 | 16.67 | 57.92 |
| Rectum | 33.84, 34.42 | 19.99 | 14.41 | 17.20 | 1.23 | 40.65 | 699.19 |
| Salivary Gland | 40, 40 | 0 | 0 | 0.00 | 7.31 | 6.84 | 0.00 |
| Clontech | | | | | | | |
| Skeletal Muscle | 34.2, 40 | 16.33 | 0 | 8.17 | 1.26 | 39.68 | 324.01 |
| Clontech | | | | | | | |
| Skin | 35.02, 40 | 10.31 | 0.48 | 5.40 | 1.21 | 41.32 | 222.93 |
| Small Intestine | 40, 40 | 0 | 0 | 0.29 | 0.98 | 51.07 | 14.81 |
| Clontech | | | | | | | |
| Spleen | 40, 35.31 | 0 | 8.71 | 4.36 | 4.92 | 10.16 | 44.26 |
| Stomach | 40, 35.4 | 0 | 8.3 | 4.15 | 2.73 | 18.32 | 76.01 |
| Testis Clontech | 40, 37.31 | 0 | 2.82 | 1.41 | 0.57 | 87.87 | 123.90 |
| Thymus Clontech | 30.9, 31.1 | 104.45 | 93.52 | 98.99 | 9.89 | 5.06 | 500.43 |
| Thyroid | 31.62, 31.57 | 69.89 | 71.93 | 70.91 | 2.77 | 18.05 | 1279.96 |
| Trachea Clontech | 34.19, 34.08 | 16.41 | 17.49 | 16.95 | 9.71 | 5.15 | 87.28 |
| Urinary Bladder | 40, 34.4 | 0 | 14.55 | 7.28 | 5.47 | 9.14 | 66.50 |
| Uterus | 30.63, 30.6 | 122.13 | 123.57 | 122.85 | 5.34 | 9.36 | 1150.28 |
| genomic | 26.58 | 1190.6 | | | | | |
| b-actin | 27.38 | 758.43 | | | | | |
| 1.00E+05 | 19.07 | 100000 | | | | | |
| 1.00E+05 | 19.35 | 100000 | | | | | |
| 1.00E+04 | 22.57 | 10000 | | | | | |
| 1.00E+04 | 22.59 | 10000 | | | | | |
| 1.00E+03 | 26.24 | 1000 | | | | | |
| 1.00E+03 | 26.31 | 1000 | | | | | |
| 1.00E+02 | 30.18 | 100 | | | | | |
| 1.00E+02 | 31.64 | 100 | | | | | |
| 1.00E+01 | 35.9 | 10 | | | | | |
| 1.00E+01 | 40 | 0 | | | | | |
| 1.00E−00 | 40 | 0 | | | | | |
| 1.00E−00 | 40 | 0 | | | | | |
| NTC | 40 | 0 | | | | | |
| NTC | 40 | 0 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1018172CSP | identifier) | Ct | copies | RNA | Sample | Population |
| colon normal GW98-167 | 21941 | 27.29 | 1064.89 | 2129.78 | colon normal | |
| colon tumor GW98-166 | 21940 | 26.18 | 2023.11 | 4046.22 | colon tumor | 1.90 |
| colon normal GW98-178 | 22080 | 30.45 | 168.68 | 337.36 | colon normal | |
| colon tumor GW98-177 | 22060 | 29.33 | 324.49 | 648.98 | colon tumor | 1.92 |
| colon normal GW98-561 | 23514 | 30.36 | 177.62 | 355.24 | colon normal | |
| colon tumor GW98-560 | 23513 | 32.27 | 58.7 | 117.40 | colon tumor | −3.03 |
| colon normal GW98-894 | 24691 | 30.71 | 145.57 | 291.14 | colon normal | |
| colon tumor GW98-893 | 24690 | 32.3 | 57.43 | 114.86 | colon tumor | −2.53 |
| lung normal GW98-3 | 20742 | 24.82 | 4478.67 | 8957.34 | lung normal | |
| lung tumor GW98-2 | 20741 | 33.61 | 26.86 | 53.72 | lung tumor | −166.74 |
| lung normal GW97-179 | 20677 | 26.31 | 1874.25 | 3748.50 | lung normal | |
| lung tumor GW97-178 | 20676 | 24.52 | 5311.72 | 10623.44 | lung tumor | 2.83 |
| lung normal GW98-165 | 21922 | 24.99 | 4042.28 | 8084.56 | lung normal | |
| lung tumor GW98-164 | 21921 | 27.19 | 1127.26 | 2254.52 | lung tumor | −3.59 |
| lung normal GW98-282 | 22584 | 25.51 | 2990.53 | 5981.06 | lung normal | |
| lung tumor GW98-281 | 22583 | 26.67 | 1522.51 | 3045.02 | lung tumor | −1.96 |
| breast normal GW00-392 | 28750 | 32.25 | 59.17 | 59.17 | breast normal | |
| breast tumor GW00-391 | 28746 | 30.48 | 165.82 | 331.64 | breast tumor | 5.60 |
| breast normal GW00-413 | 28798 | 34.58 | 15.31 | 15.31 | breast normal | |
| breast tumor GW00-412 | 28797 | 30.05 | 213.4 | 426.80 | breast tumor | 27.88 |
| breast normal GW00- | 27592-95 | 34.41 | 16.85 | 16.85 | breast normal | |
| 235:238 | | | | | | |
| breast tumor GW00- | 27588-91 | 33.52 | 28.31 | 28.31 | breast tumor | 1.68 |
| 231:234 | | | | | | |
| breast normal GW98-621 | 23656 | 28.22 | 618.19 | 1236.38 | breast normal | |
| breast tumor GW98-620 | 23655 | 32.02 | 67.94 | 135.88 | breast tumor | −9.10 |
| brain normal BB99-542 | 25507 | 29.11 | 367.88 | 735.76 | brain normal | |
| brain normal BB99-406 | 25509 | 28.05 | 682.39 | 1364.78 | brain normal | |
| brain normal BB99-904 | 25546 | 29.06 | 379.07 | 758.14 | brain normal | |
| brain stage 5 ALZ BB99- | 25502 | 30.06 | 211.81 | 423.62 | brain stage 5 ALZ | −2.25 |
| 874 | | | | | | |
| brain stage 5 ALZ BB99- | 25503 | 26.97 | 1280.13 | 2560.26 | brain stage 5 ALZ | 2.69 |
| 887 | | | | | | |
| brain stage 5 ALZ BB99- | 25504 | 29.85 | 239.03 | 478.06 | brain stage 5 ALZ | −1.99 |
| 862 | | | | | | |
| brain stage 5 ALZ BB99- | 25542 | 28.13 | 652.56 | 1305.12 | brain stage 5 ALZ | 1.37 |
| 927 | | | | | | |
| CT lung | normal | 26.97 | 1280.81 | 2561.62 | CT lung Nml | |
| lung 26 | normal | 32.21 | 60.75 | | lung 26 Nml | |
| lung 27 | normal | 34 | 21.39 | 21.39 | lung 27 Nml | |
| lung 24 | COPD | 32.11 | 64.11 | 64.11 | lung 24 COPD | −13.87 |
| lung 28 | COPD | 33.01 | 38.18 | 38.18 | lung 28 COPD | −23.29 |
| lung 23 | COPD | 32.84 | 42.15 | 42.15 | lung 23 COPD | −21.10 |
| lung 25 | normal | 31.63 | 84.78 | 84.78 | lung 25 Nml | |
| asthmatic lung ODO3112 | 29321 | 29.4 | 310.75 | 310.75 | asthmatic lung | −2.86 |
| asthmatic lung ODO3433 | 29323 | 27.02 | 1242.79 | 2485.58 | asthmatic lung | 2.80 |
| asthmatic lung ODO3397 | 29322 | 25.97 | 2289.74 | 4579.48 | asthmatic lung | 5.15 |
| asthmatic lung ODO4928 | 29325 | 26.84 | 1380.5 | 2761.00 | asthmatic lung | 3.10 |
| endo cells | control | 40 | 0 | 0.00 | endo cells | |
| endo VEGF | | 40 | 0 | 0.00 | endo VEGF | 0.00 |
| endo bFGF | | 40 | 1.01 | 1.01 | endo bFGF | 1.01 |
| heart Clontech | normal | 33.02 | 37.93 | 75.86 | heart | |
| heart (T-1) ischemic | 29417 | 34.34 | 17.51 | 35.02 | heart (T-1) | −2.17 |
| | | | | | ischemic | |
| heart (T-14) non- | 29422 | 34.85 | 13.07 | 26.14 | heart (T-14) non- | −2.90 |
| obstructive DCM | | | | | obstructive DCM | |
| heart (T-3399) DCM | 29426 | 29.74 | 254.69 | 509.38 | heart (T-3399) | 6.71 |
| | | | | | DCM | |
| adenoid GW99-269 | 26162 | 35.07 | 11.5 | 23.00 | adenoid | |
| tonsil GW98-280 | 22582 | 40 | 0 | 0.00 | tonsil | |
| T cells PC00314 | 28453 | 36.12 | 6.22 | 12.44 | T cells | |
| PBMNC | | 40 | 0 | 0.00 | PBMNC | |
| monocyte | | 40 | 0 | 0.00 | monocyte | |
| B cells PC00665 | 28455 | 40 | 0 | 0.00 | B cells | |
| dendritic cells | 28441 | 40 | 0 | 0.00 | dendritic cells | |
| neutrophils | 28440 | 35.43 | 9.3 | 9.30 | neutrophils | |
| eosinophils | 28446 | 40 | 1.32 | 2.64 | eosinophils | |
| BM unstim | | 40 | 0 | 0.00 | BM unstim | |
| BM stim | treated | 40 | 0 | 0.00 | BM stim | 0.00 |
| osteo dif | treated | 40 | 0 | 0.00 | osteo dif | 0.00 |
| osteo undif | | 40 | 0 | 0.00 | osteo undif | |
| chondrocytes | | 33.23 | 33.49 | 83.73 | chondrocytes | |
| OA Synovium IP12/01 | 29462 | 34.9 | 12.68 | 12.68 | OA Synovium | |
| OA Synovium NP10/01 | 29461 | 28.55 | 510.63 | 1021.26 | OA Synovium | |
| OA Synovium NP57/00 | 28464 | 34 | 21.41 | 42.82 | OA Synovium | |
| RA Synovium NP03/01 | 28466 | 40 | 0 | 0.00 | RA Synovium | |
| RA Synovium NP71/00 | 28467 | 36.2 | 5.96 | 11.92 | RA Synovium | |
| RA Synovium NP45/00 | 28475 | 36.38 | 5.34 | 10.68 | RA Synovium | |
| OA bone (biobank) | 29217 | 35.58 | 8.52 | 8.52 | OA bone | |
| | | | | | (biobank) | |
| OA bone Sample 1 | J. Emory | 33.54 | 27.91 | 55.82 | OA bone | |
| OA bone Sample 2 | J. Emory | 34.92 | 12.54 | 25.08 | OA bone | |
| Cartilage (pool) | Normal | 33.88 | 22.98 | 45.96 | Nml Cartilage | |
| | | | | | (pool) | |
| Cartilage (pool) | OA | 40 | 0 | 0.00 | OA Cartilage | −45.96 |
| | | | | | (pool) | |
| PBL unifected | 28441 | 30.74 | 142.65 | 285.30 | PBL unifected | |
| PBL HIV IIIB | 28442 | 32.47 | 52.13 | 104.26 | PBL HIV IIIB | −2.74 |
| MRC5 uninfected | 29158 | 40 | 0 | 0.00 | MRC5 uninfected | |
| (100%) | | | | | (100%) | |
| MRC5 HSV strain F | 29178 | 30.06 | 211.88 | 423.76 | MRC5 HSV | 423.76 |
| | | | | | strain F | |
| W12 cells | 29179 | 39.65 | 0.8 | 1.60 | W12 cells | |
| Keratinocytes | 29180 | 33.76 | 24.58 | 49.16 | Keratinocytes | |
| B-actin control | | 27.17 | 1140.82 | | | |
| genomic | | 26.81 | 1405.46 | | | |
| 1.00E+05 | | 19.68 | 100000 | | | |
| 1.00E+05 | | 19.63 | 100000 | | | |
| 1.00E+04 | | 23.15 | 10000 | | | |
| 1.00E+04 | | 23.27 | 10000 | | | |
| 1.00E+03 | | 27.1 | 1000 | | | |
| 1.00E+03 | | 27.33 | 1000 | | | |
| 1.00E+02 | | 31.34 | 100 | | | |
| 1.00E+02 | | 32.04 | 100 | | | |
| 1.00E+01 | | 35.09 | 10 | | | |
| 1.00E+01 | | 40 | 10 | | | |
| 1.00E−00 | | 40 | 0 | | | |
| 1.00E−00 | | 40 | 0 | | | |
| NTC | | 40 | 0 | | | |

*lung 26 Normal has been omitted due to multiple amplification failures from that sample

Gene Name sbg1018172CSP

| | Fold Change in Disease |
|---|---|
| | Population Relative to |
| Disease tissues | Normal |
| colon tumor | 1.90 |
| colon tumor | 1.92 |
| colon tumor | −3.03 |
| colon tumor | −2.53 |
| lung tumor | −166.74 |
| lung tumor | 2.83 |
| lung tumor | −3.59 |
| lung tumor | −1.96 |
| breast tumor | 5.60 |
| breast tumor | 27.88 |
| breast tumor | 1.68 |
| breast tumor | −9.10 |
| brain stage 5 ALZ | −2.25 |
| brain stage 5 ALZ | 2.69 |
| brain stage 5 ALZ | −1.99 |
| brain stage 5 ALZ | 1.37 |
| lung 24 | −13.87 |
| lung 28 | −23.29 |
| lung 23 | −21.10 |
| asthmatic lung | −2.86 |
| asthmatic lung | 2.80 |
| asthmatic lung | 5.15 |
| asthmatic lung | 3.10 |
| endo VEGF | 0.00 |
| endo bFGF | 1.01 |
| heart T-1 | −2.17 |
| heart T-14 | −2.90 |
| heart T-3399 | 6.71 |
| BM stim | 0.00 |
| osteo dif | 0.00 |
| Cartilage (pool) | −45.96 |
| PBL HIV IIIB | −2.74 |
| MRC5 HSV strain F | 423.76 |

Gene Name sbg1004570ERGIC

Moderate to low overall expression. This gene is expressed fairly ubiquitously in all normal samples analyzed with highest levels of expression seen in the whole brain, hypothalamus, pancreas, and head of pancreas. This pattern of expression suggests that this gene may be involved in diabetes or other metabolic diseases. Highest disease expression is seen in the colon, breast, and lung normal/tumor pairs as well as the Alzheimer's brain samples and the T cells, B cells, dendritic cells, and eosinophils. Upregulation in 2 of 4 breast tumor samples suggests a role for this gene in breast cancer. Upregulation in 2 of 4 Alzheimer's brain samples implies an involvement in Alzheimer's disease. Downregulation in 3 of 3 COPD samples and 4 of 4 asthmatic lung samples suggests a potential role for this gene in chronic obstructive pulmonary disorder and asthma. Upregulated in the stimulated bone marrow sample.

| | | | | | | | copies |
|---|---|---|---|---|---|---|---|
| | | | | | | | of |
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1004570ERGIC | 1 and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous Adipose | 39.46, 35.22 | 0.08 | 1.37 | 0.73 | 0.96 | 52.36 | 37.96 |
| Zenbio | | | | | | | |
| Adrenal Gland Clontech | 36.91, 36.21 | 0.43 | 0.7 | 0.57 | 0.61 | 81.97 | 46.31 |
| Whole Brain Clontech | 26.46, 30.31 | 547.02 | 39.49 | 293.26 | 7.24 | 6.91 | 2025.24 |
| Fetal Brain Clontech | 36.04, 36.66 | 0.78 | 0.51 | 0.65 | 0.48 | 103.95 | 67.05 |
| Cerebellum Clontech | 35.46, 34.63 | 1.16 | 2.06 | 1.61 | 2.17 | 23.04 | 37.10 |
| Cervix | 35.63, 36.28 | 1.04 | 0.67 | 0.86 | 2.42 | 20.66 | 17.67 |
| Colon | 35.38, 34.57 | 1.23 | 2.13 | 1.68 | 2.71 | 18.45 | 31.00 |
| Endometrium | 40, 35.24 | 0.06 | 1.35 | 0.71 | 0.73 | 68.21 | 48.09 |
| Esophagus | 35.02, 36.31 | 1.57 | 0.65 | 1.11 | 1.37 | 36.50 | 40.51 |
| Heart Clontech | 37.16, 35.48 | 0.36 | 1.15 | 0.76 | 1.32 | 37.88 | 28.60 |
| Hypothalamus | 35.15, 36.01 | 1.44 | 0.8 | 1.12 | 0.32 | 155.28 | 173.91 |
| Ileum | 35.04, 35.5 | 1.55 | 1.13 | 1.34 | 2.58 | 19.38 | 25.97 |
| Jejunum | 35.14, 34.88 | 1.45 | 1.73 | 1.59 | 6.60 | 7.58 | 12.05 |
| Kidney | 35.81, 37.16 | 0.91 | 0.36 | 0.64 | 2.12 | 23.58 | 14.98 |
| Liver | 36.19, 34.39 | 0.71 | 2.42 | 1.57 | 1.50 | 33.33 | 52.17 |
| Fetal Liver Clontech | 32.94, 33.1 | 6.51 | 5.85 | 6.18 | 10.40 | 4.81 | 29.71 |
| Lung | 34.54, 35.16 | 2.18 | 1.43 | 1.81 | 2.57 | 19.46 | 35.12 |
| Mammary Gland | 34.45, 34.76 | 2.33 | 1.88 | 2.11 | 13.00 | 3.85 | 8.10 |
| Clontech | | | | | | | |
| Myometrium | 34.08, 34.61 | 2.98 | 2.09 | 2.54 | 2.34 | 21.37 | 54.17 |
| Omentum | 35.22, 36.18 | 1.37 | 0.71 | 1.04 | 3.94 | 12.69 | 13.20 |
| Ovary | 34.52, 34.83 | 2.21 | 1.78 | 2.00 | 4.34 | 11.52 | 22.98 |
| Pancreas | 34.45, 33.99 | 2.32 | 3.18 | 2.75 | 0.81 | 61.80 | 169.96 |
| Head of Pancreas | 33.24, 33.63 | 5.32 | 4.06 | 4.69 | 1.57 | 31.85 | 149.36 |
| Parotid Gland | 33.22, 33.08 | 5.38 | 5.9 | 5.64 | 5.48 | 9.12 | 51.46 |
| Placenta Clontech | 36.02, 35.39 | 0.79 | 1.22 | 1.01 | 5.26 | 9.51 | 9.55 |
| Prostate | 35.98, 35.07 | 0.81 | 1.51 | 1.16 | 3.00 | 16.67 | 19.33 |
| Rectum | 36.71, 37.13 | 0.49 | 0.37 | 0.43 | 1.23 | 40.65 | 17.48 |
| Salivary Gland Clontech | 33.51, 34.22 | 4.41 | 2.71 | 3.56 | 7.31 | 6.84 | 24.35 |
| Skeletal Muscle Clontech | 35.53, 34.52 | 1.11 | 2.21 | 1.66 | 1.26 | 39.68 | 65.87 |
| Skin | 36.02, 36.07 | 0.79 | 0.77 | 0.78 | 1.21 | 41.32 | 32.23 |
| Small Intestine Clontech | 35.02, 37.21 | 1.57 | 0.35 | 0.96 | 0.98 | 51.07 | 49.03 |
| Spleen | 35.64, 35.27 | 1.03 | 1.33 | 1.18 | 4.92 | 10.16 | 11.99 |
| Stomach | 35.08, 35.41 | 1.51 | 1.2 | 1.36 | 2.73 | 18.32 | 24.82 |
| Testis Clontech | 35.48, 38.1 | 1.15 | 0.19 | 0.67 | 0.57 | 87.87 | 58.88 |
| Thymus Clontech | 32.15, 31.72 | 11.16 | 14.98 | 13.07 | 9.89 | 5.06 | 66.08 |
| Thyroid | 35.61, 35.09 | 1.05 | 1.49 | 1.27 | 2.77 | 18.05 | 22.92 |
| Trachea Clontech | 35.04, 34.75 | 1.55 | 1.89 | 1.72 | 9.71 | 5.15 | 8.86 |
| Urinary Bladder | 36.11, 36.24 | 0.74 | 0.68 | 0.71 | 5.47 | 9.14 | 6.49 |
| Uterus | 35.59, 35.68 | 1.06 | 1 | 1.03 | 5.34 | 9.36 | 9.64 |
| genomic | 24.29 | 2416.83 | | | | | |
| b-actin | 26.09 | 706.6 | | | | | |
| 1.00E+05 | 20.09 | 100000 | | | | | |
| 1.00E+05 | 19.53 | 100000 | | | | | |
| 1.00E+04 | 21.72 | 10000 | | | | | |
| 1.00E+04 | 21.68 | 10000 | | | | | |
| 1.00E+03 | 24.13 | 1000 | | | | | |
| 1.00E+03 | 24.18 | 1000 | | | | | |
| 1.00E+02 | 29.13 | 100 | | | | | |
| 1.00E+02 | 30.16 | 100 | | | | | |
| 1.00E+01 | 31.7 | 10 | | | | | |
| 1.00E+01 | 33.16 | 10 | | | | | |
| 1.00E−00 | 36.93 | 1 | | | | | |
| 1.00E−00 | 34.75 | 1 | | | | | |
| NTC | 36 | −1 | | | | | |
| NTC | 35.85 | −1 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1004570ERGIC | identifier) | Ct | copies | RNA | Sample | Population |
| colon normal GW98-167 | 21941 | 30.51 | 38.25 | 76.50 | colon normal | |
| colon tumor GW98-166 | 21940 | 31.28 | 23.13 | 46.26 | colon tumor | −1.65 |
| colon normal GW98-178 | 22080 | 31.79 | 16.57 | 33.14 | colon normal | |
| colon tumor GW98-177 | 22060 | 31.3 | 22.86 | 45.72 | colon tumor | 1.38 |
| colon normal GW98-561 | 23514 | 30.71 | 33.5 | 67.00 | colon normal | |
| colon tumor GW98-560 | 23513 | 31.18 | 24.73 | 49.46 | colon tumor | −1.35 |
| colon normal GW98-894 | 24691 | 30.16 | 48.2 | 96.40 | colon normal | |
| colon tumor GW98-893 | 24690 | 29.96 | 55 | 110.00 | colon tumor | 1.14 |
| lung normal GW98-3 | 20742 | 30.1 | 50.19 | 100.38 | lung normal | |
| lung tumor GW98-2 | 20741 | 32.86 | 8.15 | 16.30 | lung tumor | −6.16 |
| lung normal GW97-179 | 20677 | 31.65 | 18.14 | 36.28 | lung normal | |
| lung tumor GW97-178 | 20676 | 31.05 | 26.89 | 53.78 | lung tumor | 1.48 |
| lung normal GW98-165 | 21922 | 30.44 | 40.16 | 80.32 | lung normal | |
| lung tumor GW98-164 | 21921 | 30.72 | 33.36 | 66.72 | lung tumor | −1.20 |
| lung normal GW98-282 | 22584 | 31.83 | 16.13 | 32.26 | lung normal | |
| lung tumor GW98-281 | 22583 | 32.09 | 13.61 | 27.22 | lung tumor | −1.19 |
| breast normal GW00-392 | 28750 | 32.76 | 8.73 | 8.73 | breast normal | |
| breast tumor GW00-391 | 28746 | 30.68 | 34.38 | 68.76 | breast tumor | 7.88 |
| breast normal GW00-413 | 28798 | 37.11 | 0.5 | 0.50 | breast normal | |
| breast tumor GW00-412 | 28797 | 30.8 | 31.72 | 63.44 | breast tumor | 126.88 |
| breast normal GW00- | 27592-95 | 38.8 | 0.17 | 0.17 | breast normal | |
| 235:238 | | | | | | |
| breast tumor GW00- | 27588-91 | 37.36 | 0.43 | 0.43 | breast tumor | 2.53 |
| 231:234 | | | | | | |
| breast normal GW98-621 | 23656 | 31.67 | 17.86 | 35.72 | breast normal | |
| breast tumor GW98-620 | 23655 | 32.59 | 9.8 | 19.60 | breast tumor | −1.82 |
| brain normal BB99-542 | 25507 | 33.66 | 4.83 | 9.66 | brain normal | |
| brain normal BB99-406 | 25509 | 33.24 | 6.37 | 12.74 | brain normal | |
| brain normal BB99-904 | 25546 | 33.2 | 6.54 | 13.08 | brain normal | |
| brain stage 5 ALZ BB99- | 25502 | 33.1 | 6.97 | 13.94 | brain stage 5 ALZ | 1.18 |
| 874 | | | | | | |
| brain stage 5 ALZ BB99- | 25503 | 30.93 | 29.17 | 58.34 | brain stage 5 ALZ | 4.93 |
| 887 | | | | | | |
| brain stage 5 ALZ BB99- | 25504 | 31.44 | 20.73 | 41.46 | brain stage 5 ALZ | 3.51 |
| 862 | | | | | | |
| brain stage 5 ALZ BB99- | 25542 | 32.93 | 7.83 | 15.66 | brain stage 5 ALZ | 1.32 |
| 927 | | | | | | |
| CT lung | normal | 32.62 | 9.6 | 19.20 | CT lung Nml | |
| lung 26 | normal | | | | lung 26 Nml | |
| lung 27 | normal | 40 | 0 | 0.00 | lung 27 Nml | |
| lung 24 | COPD | 39.15 | 0.13 | 0.13 | lung 24 COPD | −49.46 |
| lung 28 | COPD | 40 | 0.08 | 0.08 | lung 28 COPD | −80.38 |
| lung 23 | COPD | 38.59 | 0.19 | 0.19 | lung 23 COPD | −33.84 |
| lung 25 | normal | 40 | 0.09 | 0.09 | lung 25 Nml | |
| asthmatic lung ODO3112 | 29321 | 40 | 0 | 0.00 | asthmatic lung | −6.43 |
| asthmatic lung ODO3433 | 29323 | 38.47 | 0.2 | 0.40 | asthmatic lung | −16.08 |
| asthmatic lung ODO3397 | 29322 | 38.01 | 0.28 | 0.56 | asthmatic lung | −11.48 |
| asthmatic lung ODO4928 | 29325 | 38.13 | 0.26 | 0.52 | asthmatic lung | −12.37 |
| endo cells | control | 36.24 | 0.89 | 0.89 | endo cells | |
| endo VEGF | | 39.8 | 0.09 | 0.09 | endo VEGF | −9.89 |
| endo bFGF | | 37.19 | 0.47 | 0.47 | endo bFGF | −1.89 |
| heart Clontech | normal | 35.52 | 1.43 | 2.86 | heart | |
| heart (T-1) ischemic | 29417 | 33.79 | 4.43 | 8.86 | heart (T-1) | 3.10 |
| | | | | | ischemic | |
| heart (T-14) non- | 29422 | 34.81 | 2.27 | 4.54 | heart (T-14) non- | 1.59 |
| obstructive DCM | | | | | obstructive DCM | |
| heart (T-3399) DCM | 29426 | 34.11 | 3.59 | 7.18 | heart (T-3399) | 2.51 |
| | | | | | DCM | |
| adenoid GW99-269 | 26162 | 34.97 | 2.05 | 4.10 | adenoid | |
| tonsil GW98-280 | 22582 | 33.05 | 7.23 | 14.46 | tonsil | |
| T cells PC00314 | 28453 | 31.09 | 26.2 | 52.40 | T cells | |
| PBMNC | | 38.01 | 0.28 | 0.28 | PBMNC | |
| monocyte | | 36.29 | 0.86 | 1.72 | monocyte | |
| B cells PC00665 | 28455 | 32.13 | 13.23 | 26.46 | B cells | |
| dendritic cells | 28441 | 31.94 | 14.96 | 29.92 | dendritic cells | |
| neutrophils | 28440 | 34.08 | 3.66 | 3.66 | neutrophils | |
| eosinophils | 28446 | 32.23 | 12.37 | 24.74 | eosinophils | |
| BM unstim | | 39.73 | 0.09 | 0.09 | BM unstim | |
| BM stim | treated | 37.03 | 0.53 | 0.53 | BM stim | 5.89 |
| osteo dif | treated | 36.8 | 0.61 | 0.61 | osteo dif | 0.61 |
| osteo undif | | 40 | 0 | 0.00 | osteo undif | |
| chondrocytes | | 31.85 | 15.9 | 39.75 | chondrocytes | |
| OA Synovium IP12/01 | 29462 | 38.61 | 0.19 | 0.19 | OA Synovium | |
| OA Synovium NP10/01 | 29461 | 33.11 | 6.96 | 13.92 | OA Synovium | |
| OA Synovium NP57/00 | 28464 | 33.81 | 4.39 | 8.78 | OA Synovium | |
| RA Synovium NP03/01 | 28466 | 33.11 | 6.96 | 13.92 | RA Synovium | |
| RA Synovium NP71/00 | 28467 | 32.03 | 14.14 | 28.28 | RA Synovium | |
| RA Synovium NP45/00 | 28475 | 32.47 | 10.55 | 21.10 | RA Synovium | |
| OA bone (biobank) | 29217 | 35.25 | 1.7 | 1.70 | OA bone (biobank) | |
| OA bone Sample 1 | J. Emory | 34.54 | 2.72 | 5.44 | OA bone | |
| OA bone Sample 2 | J. Emory | 36.28 | 0.87 | 1.74 | OA bone | |
| Cartilage (pool) | Normal | 35.24 | 1.71 | 3.42 | Nml Cartilage | |
| | | | | | (pool) | |
| Cartilage (pool) | OA | 34.45 | 2.87 | 5.74 | OA Cartilage | 1.68 |
| | | | | | (pool) | |
| PBL unifected | 28441 | 32.53 | 10.19 | 20.38 | PBL unifected | |
| PBL HIV IIIB | 28442 | 31.77 | 16.79 | 33.58 | PBL HIV IIIB | 1.65 |
| MRC5 uninfected | 29158 | 33.12 | 6.87 | 13.74 | MRC5 uninfected | |
| (100%) | | | | | (100%) | |
| MRC5 HSV strain F | 29178 | 33.76 | 4.54 | 9.08 | MRC5 HSV strain F | −1.51 |
| W12 cells | 29179 | 33.1 | 6.96 | 13.92 | W12 cells | |
| Keratinocytes | 29180 | 32.67 | 9.29 | 18.58 | Keratinocytes | |
| B-actin control | | 26.03 | 726.55 | | | |
| genomic | | 24.63 | 1825.58 | | | |
| 1.00E+05 | | 19.96 | 100000 | | | |
| 1.00E+05 | | 19.27 | 100000 | | | |
| 1.00E+04 | | 21.83 | 10000 | | | |
| 1.00E+04 | | 21.45 | 10000 | | | |
| 1.00E+03 | | 23.86 | 1000 | | | |
| 1.00E+03 | | 23.84 | 1000 | | | |
| 1.00E+02 | | 28.42 | 100 | | | |
| 1.00E+02 | | 29.35 | 100 | | | |
| 1.00E+01 | | 33.3 | 10 | | | |
| 1.00E+01 | | 35.09 | 10 | | | |
| 1.00E−00 | | 35.16 | 1 | | | |
| 1.00E−00 | | 36.05 | 1 | | | |
| NTC | | 38.24 | −1 | | | |

*lung 26 Normal has been omitted due to multiple amplification failures from that sample

Gene Name sbg1004570ERGIC

| | Fold Change in Disease |
|---|---|
| | Population Relative to |
| Disease tissues | Normal |
| colon tumor | −1.65 |
| colon tumor | 1.38 |
| colon tumor | −1.35 |
| colon tumor | 1.14 |
| lung tumor | −6.16 |
| lung tumor | 1.48 |
| lung tumor | −1.20 |
| lung tumor | −1.19 |
| breast tumor | 7.88 |
| breast tumor | 126.88 |
| breast tumor | 2.53 |
| breast tumor | −1.82 |
| brain stage 5 ALZ | 1.18 |
| brain stage 5 ALZ | 4.93 |
| brain stage 5 ALZ | 3.51 |
| brain stage 5 ALZ | 1.32 |
| lung 24 | −49.46 |
| lung 28 | −80.38 |
| lung 23 | −33.84 |
| asthmatic lung | −6.43 |
| asthmatic lung | −16.08 |
| asthmatic lung | −11.48 |
| asthmatic lung | −12.37 |
| endo VEGF | −9.89 |
| endo bFGF | −1.89 |
| heart T-1 | 3.10 |
| heart T-14 | 1.59 |
| heart T-3399 | 2.51 |
| BM stim | 5.89 |
| osteo dif | 0.61 |
| Cartilage (pool) | 1.68 |
| PBL HIV IIIB | 1.65 |
| MRC5 HSV strain F | −1.51 |

Gene Name sbg1016995 IGBrecpt

Moderate to low overall expression. Highest normal expression is seen in the whole brain in lung with slightly lower levels of expression in the endometrium, ileum, rectum, and skin. High level of expression in the skin may suggest a possible role for this gene in psoriasis and Lupus. The patterns of expression in the samples on the disease plate indicate that this gene is highly specific to the adenoid and tonsil. Downregulation in 2 of 4 lung tumor samples and upregulation in 2 of 4 breast tumor samples suggests an involvement in cancers of the lung and breast. Downregulation in 3 of 3 COPD samples suggests a potential role for this gene in chronic obstructive pulmonary disorder. Upregulated in the stimulated bone marrow sample. Downregulated in the differentiated osteoblast. Upregulated in the HIV-infected PBL cells suggests that this gene may be a host factor in HIV.

| | | | | | | | copies of |
|---|---|---|---|---|---|---|---|
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1016995IGBrecpt | 1 and 2) | 1) | 2) | Copies | (ng) | (ng) | RNA |
| Subcutaneous | 40, 40 | 0.61 | 0.61 | 0.61 | 3.06 | 16.34 | 9.97 |
| Adipocytes Zenbio | | | | | | | |
| Subcutaneous Adipose | 40, 40 | 0.59 | 0.56 | 0.58 | 0.96 | 52.36 | 30.10 |
| Zenbio | | | | | | | |
| Adrenal Gland | 39.89, 39.79 | 0.54 | 0.57 | 0.56 | 0.61 | 81.97 | 45.49 |
| Clontech | | | | | | | |
| Whole Brain Clontech | 30.69, 30.76 | 108.67 | 104.35 | 106.51 | 7.24 | 6.91 | 735.57 |
| Fetal Brain Clontech | 39.41, 40 | 0.71 | 0.62 | 0.67 | 0.48 | 103.95 | 69.13 |
| Cerebellum Clontech | 39.33, 37.82 | 0.74 | 1.78 | 1.26 | 2.17 | 23.04 | 29.03 |
| Cervix | 36.12, 36.58 | 4.73 | 3.62 | 4.18 | 2.42 | 20.66 | 86.26 |
| Colon | 35.23, 38.35 | 7.9 | 1.31 | 4.61 | 2.71 | 18.45 | 84.96 |
| Endometrium | 35.51, 40 | 6.73 | 0 | 3.37 | 0.73 | 68.21 | 229.54 |
| Esophagus | 37.22, 38.18 | 2.5 | 1.45 | 1.98 | 1.37 | 36.50 | 72.08 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 34.65, 34.11 | 11.04 | 15.09 | 13.07 | 2.58 | 19.38 | 253.20 |
| Jejunum | 34.84, 34.04 | 9.91 | 15.72 | 12.82 | 6.60 | 7.58 | 97.08 |
| Kidney | 38.2, 39.11 | 1.43 | 0.84 | 1.14 | 2.12 | 23.58 | 26.77 |
| Liver | 34.47, 38.59 | 12.26 | 1.14 | 6.70 | 1.50 | 33.33 | 223.33 |
| Fetal Liver Clontech | 33.51, 33.07 | 21.26 | 27.43 | 24.35 | 10.40 | 4.81 | 117.04 |
| Lung | 27.32, 37.11 | 755.31 | 2.68 | 379.00 | 2.57 | 19.46 | 7373.44 |
| Mammary Gland | 36.31, 36.4 | 4.24 | 4.03 | 4.14 | 13.00 | 3.85 | 15.90 |
| Clontech | | | | | | | |
| Myometrium | 40, 38.7 | 0.7 | 1.07 | 0.89 | 2.34 | 21.37 | 18.91 |
| Omentum | 35.44, 36.14 | 6.98 | 4.68 | 5.83 | 3.94 | 12.69 | 73.98 |
| Ovary | 38.76, 35.49 | 1.03 | 6.82 | 3.93 | 4.34 | 11.52 | 45.22 |
| Pancreas | 40, 38.56 | 0.48 | 1.16 | 0.82 | 0.81 | 61.80 | 50.68 |
| Head of Pancreas | 40, 40 | 0 | 0 | 0.00 | 1.57 | 31.85 | 0.00 |
| Parotid Gland | 36.8, 35.45 | 3.2 | 6.98 | 5.09 | 5.48 | 9.12 | 46.44 |
| Placenta Clontech | 35.63, 35.11 | 6.27 | 8.47 | 7.37 | 5.26 | 9.51 | 70.06 |
| Prostate | 37.4, 37.5 | 2.26 | 2.14 | 2.20 | 3.00 | 16.67 | 36.67 |
| Rectum | 35.45, 35.25 | 6.94 | 7.81 | 7.38 | 1.23 | 40.65 | 299.80 |
| Salivary Gland | 37.3, 37.06 | 2.4 | 2.75 | 2.58 | 7.31 | 6.84 | 17.61 |
| Clontech | | | | | | | |
| Skeletal Muscle | 40, 39.34 | 0 | 0.74 | 0.37 | 1.26 | 39.68 | 14.68 |
| Clontech | | | | | | | |
| Skin | 38.84, 34.56 | 0.98 | 11.63 | 6.31 | 1.21 | 41.32 | 260.54 |
| Small Intestine | 40, 40 | 0 | 0.63 | 0.32 | 0.98 | 51.07 | 16.09 |
| Clontech | | | | | | | |
| Spleen | 34.37, 34.89 | 13 | 9.6 | 11.30 | 4.92 | 10.16 | 114.84 |
| Stomach | 39.73, 35.52 | 0.59 | 6.67 | 3.63 | 2.73 | 18.32 | 66.48 |
| Testis Clontech | 38.91, 40 | 0.94 | 0 | 0.47 | 0.57 | 87.87 | 41.30 |
| Thymus Clontech | 31.96, 32.96 | 52.16 | 29.2 | 40.68 | 9.89 | 5.06 | 205.66 |
| Thyroid | 35.53, 40 | 6.66 | 0 | 3.33 | 2.77 | 18.05 | 60.11 |
| Trachea Clontech | 37.99, 37.69 | 1.61 | 1.91 | 1.76 | 9.71 | 5.15 | 9.06 |
| Urinary Bladder | 39.69, 39.02 | 0.6 | 0.89 | 0.75 | 5.47 | 9.14 | 6.81 |
| Uterus | 34.41, 33.56 | 12.67 | 20.75 | 16.71 | 5.34 | 9.36 | 156.46 |
| genomic | 26.31 | 1359.1 | | | | | |
| b-actin | 27.2 | 812.88 | | | | | |
| 1.00E+05 | 19.24 | 100000 | | | | | |
| 1.00E+05 | 19.38 | 100000 | | | | | |
| 1.00E+04 | 22.67 | 10000 | | | | | |
| 1.00E+04 | 22.67 | 10000 | | | | | |
| 1.00E+03 | 26.31 | 1000 | | | | | |
| 1.00E+03 | 26.28 | 1000 | | | | | |
| 1.00E+02 | 30.17 | 100 | | | | | |
| 1.00E+02 | 31.02 | 100 | | | | | |
| 1.00E+01 | 36.17 | 10 | | | | | |
| 1.00E+01 | 34.46 | 10 | | | | | |
| 1.00E−00 | 40 | 0 | | | | | |
| 1.00E−00 | 40 | 1 | | | | | |
| NTC | 40 | −1 | | | | | |
| NTC | 40 | −1 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1016995IGBrecpt | identifier) | Ct | copies | RNA | Sample | Population |
| colon normal GW98-167 | 21941 | 29.45 | 174.86 | 349.72 | colon normal | |
| colon tumor GW98-166 | 21940 | 32.1 | 33.44 | 66.88 | colon tumor | −5.23 |
| colon normal GW98-178 | 22080 | 31.77 | 41.07 | 82.14 | colon normal | |
| colon tumor GW98-177 | 22060 | 32.66 | 23.5 | 47.00 | colon tumor | −1.75 |
| colon normal GW98-561 | 23514 | 29.15 | 211.24 | 422.48 | colon normal | |
| colon tumor GW98-560 | 23513 | 31.25 | 56.95 | 113.90 | colon tumor | −3.71 |
| colon normal GW98-894 | 24691 | 30.68 | 81.3 | 162.60 | colon normal | |
| colon tumor GW98-893 | 24690 | 31.33 | 54.12 | 108.24 | colon tumor | −1.50 |
| lung normal GW98-3 | 20742 | 31.86 | 38.92 | 77.84 | lung normal | |
| lung tumor GW98-2 | 20741 | 34.55 | 7.25 | 14.50 | lung tumor | −5.37 |
| lung normal GW97-179 | 20677 | 28.38 | 342.07 | 684.14 | lung normal | |
| lung tumor GW97-178 | 20676 | 32.1 | 33.52 | 67.04 | lung tumor | −10.20 |
| lung normal GW98-165 | 21922 | 32.2 | 31.46 | 62.92 | lung normal | |
| lung tumor GW98-164 | 21921 | 30.5 | 90.8 | 181.60 | lung tumor | 2.89 |
| lung normal GW98-282 | 22584 | 29.82 | 138.8 | 277.60 | lung normal | |
| lung tumor GW98-281 | 22583 | 32.72 | 22.64 | 45.28 | lung tumor | −6.13 |
| breast normal GW00-392 | 28750 | 31.5 | 48.65 | 48.65 | breast normal | |
| breast tumor GW00-391 | 28746 | 31.9 | 37.84 | 75.68 | breast tumor | 1.56 |
| breast normal GW00-413 | 28798 | 34.37 | 8.07 | 8.07 | breast normal | |
| breast tumor GW00-412 | 28797 | 29.97 | 126.73 | 253.46 | breast tumor | 31.41 |
| breast normal GW00- | 27592-95 | 35.08 | 5.2 | 5.20 | breast normal | |
| 235:238 | | | | | | |
| breast tumor GW00- | 27588-91 | 32.3 | 29.54 | 29.54 | breast tumor | 5.68 |
| 231:234 | | | | | | |
| breast normal GW98-621 | 23656 | 31.11 | 61.96 | 123.92 | breast normal | |
| breast tumor GW98-620 | 23655 | 31.27 | 56.22 | 112.44 | breast tumor | −1.10 |
| brain normal BB99-542 | 25507 | 33.3 | 15.82 | 31.64 | brain normal | |
| brain normal BB99-406 | 25509 | 33.02 | 18.83 | 37.66 | brain normal | |
| brain normal BB99-904 | 25546 | 33.93 | 10.62 | 21.24 | brain normal | |
| brain stage 5 ALZ BB99- | 25502 | 32.36 | 28.38 | 56.76 | brain stage 5 ALZ | 1.88 |
| 874 | | | | | | |
| brain stage 5 ALZ BB99- | 25503 | 31.79 | 40.66 | 81.32 | brain stage 5 ALZ | 2.69 |
| 887 | | | | | | |
| brain stage 5 ALZ BB99- | 25504 | 32.04 | 34.76 | 69.52 | brain stage 5 ALZ | 2.30 |
| 862 | | | | | | |
| brain stage 5 ALZ BB99- | 25542 | 31.79 | 40.51 | 81.02 | brain stage 5 ALZ | 2.68 |
| 927 | | | | | | |
| CT lung | normal | 33.32 | 15.63 | 31.26 | CT lung Nml | |
| lung 26 | normal | 29.8 | 140.4 | | lung 26 Nml | |
| lung 27 | normal | 38.71 | 0.54 | 0.54 | lung 27 Nml | |
| lung 24 | COPD | 39.31 | 0.37 | 0.37 | lung 24 COPD | −29.00 |
| lung 28 | COPD | 37.09 | 1.48 | 1.48 | lung 28 COPD | −7.25 |
| lung 23 | COPD | 38.02 | 0.83 | 0.83 | lung 23 COPD | −12.93 |
| lung 25 | normal | 39.22 | 0.39 | 0.39 | lung 25 Nml | |
| asthmatic lung ODO3112 | 29321 | 37.96 | 0.86 | 0.86 | asthmatic lung | −12.48 |
| asthmatic lung ODO3433 | 29323 | 31.15 | 60.54 | 121.08 | asthmatic lung | 11.28 |
| asthmatic lung ODO3397 | 29322 | 33.74 | 12.01 | 24.02 | asthmatic lung | 2.24 |
| asthmatic lung ODO4928 | 29325 | 31.59 | 46.09 | 92.18 | asthmatic lung | 8.59 |
| endo cells | control | 36.98 | 1.58 | 1.58 | endo cells | |
| endo VEGF | | 39.28 | 0.38 | 0.38 | endo VEGF | −4.16 |
| endo bFGF | | 37.3 | 1.3 | 1.30 | endo bFGF | −1.22 |
| heart Clontech | normal | 35.73 | 3.45 | 6.90 | heart | |
| heart (T-1) ischemic | 29417 | 34.7 | 6.58 | 13.16 | heart (T-1) | 1.91 |
| | | | | | ischemic | |
| heart (T-14) non- | 29422 | 37.5 | 1.15 | 2.30 | heart (T-14) non- | −3.00 |
| obstructive DCM | | | | | obstructive DCM | |
| heart (T-3399) DCM | 29426 | 35.15 | 4.96 | 9.92 | heart (T-3399) | 1.44 |
| | | | | | DCM | |
| adenoid GW99-269 | 26162 | 25.98 | 1528.07 | 3056.14 | adenoid | |
| tonsil GW98-280 | 22582 | 24.6 | 3626.43 | 7252.86 | tonsil | |
| T cells PC00314 | 28453 | 34.49 | 7.5 | 15.00 | T cells | |
| PBMNC | | 37.58 | 1.09 | 1.09 | PBMNC | |
| monocyte | | 37.4 | 1.22 | 2.44 | monocyte | |
| B cells PC00665 | 28455 | 31.68 | 43.59 | 87.18 | B cells | |
| dendritic cells | 28441 | 35.05 | 5.28 | 10.56 | dendritic cells | |
| neutrophils | 28440 | 35.68 | 3.57 | 3.57 | neutrophils | |
| eosinophils | 28446 | 35.07 | 5.23 | 10.46 | eosinophils | |
| BM unstim | | 38.19 | 0.75 | 0.75 | BM unstim | |
| BM stim | treated | 34.27 | 8.61 | 8.61 | BM stim | 11.48 |
| osteo dif | treated | 40 | 0.09 | 0.09 | osteo dif | −5.78 |
| osteo undif | | 40 | 0.52 | 0.52 | osteo undif | |
| chondrocytes | | 32.86 | 20.79 | 51.98 | chondrocytes | |
| OA Synovium IP12/01 | 29462 | 31.85 | 38.99 | 38.99 | OA Synovium | |
| OA Synovium NP10/01 | 29461 | 34.76 | 6.33 | 12.66 | OA Synovium | |
| OA Synovium NP57/00 | 28464 | 31.39 | 51.96 | 103.92 | OA Synovium | |
| RA Synovium NP03/01 | 28466 | 31.1 | 62.3 | 124.60 | RA Synovium | |
| RA Synovium NP71/00 | 28467 | 31.95 | 36.76 | 73.52 | RA Synovium | |
| RA Synovium NP45/00 | 28475 | 32.43 | 27.14 | 54.28 | RA Synovium | |
| OA bone (biobank) | 29217 | 35.84 | 3.22 | 3.22 | OA bone (biobank) | |
| OA bone Sample 1 | J. Emory | 35.43 | 4.18 | 8.36 | OA bone | |
| OA bone Sample 2 | J. Emory | 34.86 | 5.95 | 11.90 | OA bone | |
| Cartilage (pool) | Normal | 34.79 | 6.21 | 12.42 | Nml Cartilage | |
| | | | | | (pool) | |
| Cartilage (pool) | OA | 36.55 | 2.07 | 4.14 | OA Cartilage | −3.00 |
| | | | | | (pool) | |
| PBL unifected | 28441 | 30.02 | 122.76 | 245.52 | PBL unifected | |
| PBL HIV IIIB | 28442 | 28.17 | 388.59 | 777.18 | PBL HIV IIIB | 3.17 |
| MRC5 uninfected | 29158 | 34.6 | 7.01 | 14.02 | MRC5 uninfected | |
| (100%) | | | | | (100%) | |
| MRC5 HSV strain F | 29178 | 33.27 | 16.13 | 32.26 | MRC5 HSV strain F | 2.30 |
| W12 cells | 29179 | 34.43 | 7.8 | 15.60 | W12 cells | |
| Keratinocytes | 29180 | 35.04 | 5.31 | 10.62 | Keratinocytes | |
| B-actin control | | 27 | 808.77 | | | |
| genomic | | 26.18 | 1353.11 | | | |
| 1.00E+05 | | 19.37 | 100000 | | | |
| 1.00E+05 | | 19.59 | 100000 | | | |
| 1.00E+04 | | 22.75 | 10000 | | | |
| 1.00E+04 | | 22.8 | 10000 | | | |
| 1.00E+03 | | 26.43 | 1000 | | | |
| 1.00E+03 | | 26.17 | 1000 | | | |
| 1.00E+02 | | 30.09 | 100 | | | |
| 1.00E+02 | | 30.21 | 100 | | | |
| 1.00E+01 | | 35.27 | 10 | | | |
| 1.00E+01 | | 35.55 | 10 | | | |
| 1.00E−00 | | 39.31 | 1 | | | |
| 1.00E−00 | | 34.53 | 1 | | | |
| NTC | | 40 | −1 | | | |

*lung 26 Normal has been omitted-due to multiple amplification failures from that sample

Gene Name sbg1016995 IGBrecpt

| | Fold Change in Disease |
|---|---|
| | Population Relative to |
| Disease tissues | Normal |
| colon tumor | −5.23 |
| colon tumor | −1.75 |
| colon tumor | −3.71 |
| colon tumor | −1.50 |
| lung tumor | −5.37 |
| lung tumor | −10.20 |
| lung tumor | 2.89 |
| lung tumor | −6.13 |
| breast tumor | 1.56 |
| breast tumor | 31.41 |
| breast tumor | 5.68 |
| breast tumor | −1.10 |
| brain stage 5 ALZ | 1.88 |
| brain stage 5 ALZ | 2.69 |
| brain stage 5 ALZ | 2.30 |
| brain stage 5 ALZ | 2.68 |
| lung 24 | −29.00 |
| lung 28 | −7.25 |
| lung 23 | −12.93 |
| asthmatic lung | −12.48 |
| asthmatic lung | 11.28 |
| asthmatic lung | 2.24 |
| asthmatic lung | 8.59 |
| endo VEGF | −4.16 |
| endo bFGF | −1.22 |
| heart T-1 | 1.91 |
| heart T-14 | −3.00 |
| heart T-3399 | 1.44 |
| BM stim | 11.48 |
| osteo dif | −5.78 |
| Cartilage (pool) | −3.00 |
| PBL HIV IIIB | 3.17 |
| MRC5 HSV strain F | 2.30 |

Gene Name sbg1151bSREC

Highest overall expression in normal and disease samples. Fairly ubiquitously expressed but highest normal expression in adipocytes, adipose, whole brain, fetal brain, and endometrium. Highest disease expression in one of the colon tumor samples, one of the normal lung samples, chondrocytes, and the uninfected MRC5. There are no significant changes in brains from patients with Alzheimer's disease. Downregulation in 1 of 4 lung tumors suggests possible implication in lung cancer. Upregulation in 1 of 4 breast tumor samples is sufficient to claim a role in cancer of the breast. Upregulation in 1 of 4 asthma lungs implies a role in asthma. Downregulation in HSV implicates involvement in herpes simplex virus as a potential host factor. High expression in immune cells. High expression in cartilage and bone samples from patients with OA as well as high expression in chondrocytes possible involvement in osteoarthritis and rheumatoid arthritis. Additionally, the corroborating expression in immune cells (particularly B and T cells) provides additional evidence for a role in RA/OA.

| | | | | | | | copies of |
|---|---|---|---|---|---|---|---|
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1151bSREC | 1 and 2) | 1) | 2) | Copies | (ng) | (ng) | RNA |
| Subcutaneous | 28.31, 28.35 | 477.04 | 466.02 | 471.53 | 3.06 | 16.34 | 7704.74 |
| Adipocytes Zenbio | | | | | | | |
| Subcutaneous Adipose | 30.79, 30.22 | 122.3 | 166.95 | 144.63 | 0.96 | 52.36 | 7571.99 |
| Zenbio | | | | | | | |
| Adrenal Gland Clontech | 33.96, 33.47 | 21.39 | 27.97 | 24.68 | 0.61 | 81.97 | 2022.95 |
| Whole Brain Clontech | 24.07, 23.98 | 4889.28 | 5123.8 | 5006.54 | 7.24 | 6.91 | 34575.55 |
| Fetal Brain Clontech | 31.1, 32.29 | 103.16 | 53.55 | 78.36 | 0.48 | 103.95 | 8145.01 |
| Cerebellum Clontech | 31.03, 31.99 | 107.02 | 63.18 | 85.10 | 2.17 | 23.04 | 1960.83 |
| Cervix | 31.22, 30.06 | 96.6 | 182.64 | 139.62 | 2.42 | 20.66 | 2884.71 |
| Colon | 30.68, 30.53 | 129.52 | 140.99 | 135.26 | 2.71 | 18.45 | 2495.48 |
| Endometrium | 30.59, 30.44 | 136.06 | 147.8 | 141.93 | 0.73 | 68.21 | 9681.45 |
| Esophagus | 33.11, 32.17 | 34.08 | 57.32 | 45.70 | 1.37 | 36.50 | 1667.88 |
| Heart Clontech | 33.19, 32.41 | 32.68 | 50.11 | 41.40 | 1.32 | 37.88 | 1567.99 |
| Hypothalamus | 34.34, 40 | 17.4 | 0 | 8.70 | 0.32 | 155.28 | 1350.93 |
| Ileum | 31.29, 30.13 | 92.84 | 174.99 | 133.92 | 2.58 | 19.38 | 2595.25 |
| Jejunum | 29.7, 29.48 | 221.9 | 251.05 | 236.48 | 6.60 | 7.58 | 1791.48 |
| Kidney | 31.03, 30.17 | 107.15 | 171.45 | 139.30 | 2.12 | 23.58 | 3285.38 |
| Liver | 32.89, 33.16 | 38.61 | 33.17 | 35.89 | 1.50 | 33.33 | 1196.33 |
| Fetal Liver Clontech | 28.05, 28.15 | 550.64 | 518.95 | 534.80 | 10.40 | 4.81 | 2571.13 |
| Lung | 29.39, 28.63 | 263.85 | 398.99 | 331.42 | 2.57 | 19.46 | 6447.86 |
| Mammary Gland | 27.56, 27.39 | 717.67 | 789.94 | 753.81 | 13.00 | 3.85 | 2899.25 |
| Clontech | | | | | | | |
| Myometrium | 29.08, 28.93 | 312.86 | 339.46 | 326.16 | 2.34 | 21.37 | 6969.23 |
| Omentum | 30.72, 29.32 | 126.7 | 273.04 | 199.87 | 3.94 | 12.69 | 2536.42 |
| Ovary | 28.89, 28.68 | 346.91 | 388.02 | 367.47 | 4.34 | 11.52 | 4233.47 |
| Pancreas | 35.24, 35.75 | 10.59 | 8.02 | 9.31 | 0.81 | 61.80 | 575.09 |
| Head of Pancreas | 35.25, 33.21 | 10.57 | 32.34 | 21.46 | 1.57 | 31.85 | 683.28 |
| Parotid Gland | 28.46, 27.84 | 438.62 | 615.36 | 526.99 | 5.48 | 9.12 | 4808.30 |
| Placenta Clontech | 28.67, 28.66 | 391.9 | 393.15 | 392.53 | 5.26 | 9.51 | 3731.23 |
| Prostate | 30.55, 31.46 | 139.05 | 84.64 | 111.85 | 3.00 | 16.67 | 1864.08 |
| Rectum | 31.28, 31.43 | 93.33 | 85.92 | 89.63 | 1.23 | 40.65 | 3643.29 |
| Salivary Gland | 31.13, 30.57 | 101.46 | 138.16 | 119.81 | 7.31 | 6.84 | 819.49 |
| Clontech | | | | | | | |
| Skeletal Muscle | 34.05, 35.24 | 20.38 | 10.59 | 15.49 | 1.26 | 39.68 | 614.48 |
| Clontech | | | | | | | |
| Skin | 31.53, 31.2 | 81.49 | 97.36 | 89.43 | 1.21 | 41.32 | 3695.25 |
| Small Intestine | 34.81, 33.82 | 13.41 | 23.18 | 18.30 | 0.98 | 51.07 | 934.37 |
| Clontech | | | | | | | |
| Spleen | 31.01, 30.44 | 108.41 | 147.9 | 128.16 | 4.92 | 10.16 | 1302.39 |
| Stomach | 32.01, 31.1 | 62.6 | 102.97 | 82.79 | 2.73 | 18.32 | 1516.21 |
| Testis Clontech | 31.74, 32.29 | 72.49 | 53.45 | 62.97 | 0.57 | 87.87 | 5533.39 |
| Thymus Clontech | 28.84, 28.53 | 356.64 | 421.44 | 389.04 | 9.89 | 5.06 | 1966.84 |
| Thyroid | 30.12, 30.04 | 176.76 | 184.5 | 180.63 | 2.77 | 18.05 | 3260.47 |
| Trachea Clontech | 28.48, 28.38 | 434.3 | 459.42 | 446.86 | 9.71 | 5.15 | 2301.03 |
| Urinary Bladder | 29.63, 29.55 | 230.25 | 241.15 | 235.70 | 5.47 | 9.14 | 2154.48 |
| Uterus | 28.69, 28.37 | 387.47 | 461.07 | 424.27 | 5.34 | 9.36 | 3972.57 |
| genomic | 26.24 | 1487.44 | | | | | |
| b-actin | 27.28 | 839.2 | | | | | |
| 1.00E+05 | 18.96 | 100000 | | | | | |
| 1.00E+05 | 19.34 | 100000 | | | | | |
| 1.00E+04 | 22.64 | 10000 | | | | | |
| 1.00E+04 | 22.84 | 10000 | | | | | |
| 1.00E+03 | 26.22 | 1000 | | | | | |
| 1.00E+03 | 26.04 | 1000 | | | | | |
| 1.00E+02 | 31.04 | 100 | | | | | |
| 1.00E+02 | 30.1 | 100 | | | | | |
| 1.00E+01 | 33.33 | 10 | | | | | |
| 1.00E+01 | 39.08 | 10 | | | | | |
| 1.00E−00 | 40 | 0 | | | | | |
| 1.00E−00 | 40 | 1 | | | | | |
| NTC | 40 | 0 | | | | | |
| NTC | 40 | 0 | | | | | |

| | | | | copies of | | |
|---|---|---|---|---|---|---|
| | | | | mRNA | | |
| | Reg | | | detected/ | | Fold |
| | number | | Mean | 50 ng | | Change |
| Sample | (GSK | | GOI | total | | in Disease |
| sbg1151bSREC | identifier) | Ct | copies | RNA | Sample | Population |
| colon normal GW98-167 | 21941 | 24.55 | 5357.25 | 10714.50 | colon normal | |
| colon tumor GW98-166 | 21940 | 22.61 | 19769.94 | 39539.88 | colon tumor | 3.69 |
| colon normal GW98-178 | 22080 | 26.71 | 1252.3 | 2504.60 | colon normal | |
| colon tumor GW98-177 | 22060 | 26.13 | 1854.49 | 3708.98 | colon tumor | 1.48 |
| colon normal GW98-561 | 23514 | 26.82 | 1165.06 | 2330.12 | colon normal | |
| colon tumor GW98-560 | 23513 | 25.75 | 2390.26 | 4780.52 | colon tumor | 2.05 |
| colon normal GW98-894 | 24691 | 26.06 | 1948.57 | 3897.14 | colon normal | |
| colon tumor GW98-893 | 24690 | 26.59 | 1362.55 | 2725.10 | colon tumor | −1.43 |
| lung normal GW98-3 | 20742 | 22.77 | 17753 | 35506.00 | lung normal | |
| lung tumor GW98-2 | 20741 | 26.17 | 1803.8 | 3607.60 | lung tumor | −9.84 |
| lung normal GW97-179 | 20677 | 25.24 | 3370.88 | 6741.76 | lung normal | |
| lung tumor GW97-178 | 20676 | 24.14 | 7057.92 | 14115.84 | lung tumor | 2.09 |
| lung normal GW98-165 | 21922 | 23.87 | 8442.49 | 16884.98 | lung normal | |
| lung tumor GW98-164 | 21921 | 24.08 | 7339.83 | 14679.66 | lung tumor | −1.15 |
| lung normal GW98-282 | 22584 | 25.51 | 2804.42 | 5608.84 | lung normal | |
| lung tumor GW98-281 | 22583 | 24.2 | 6787.31 | 13574.62 | lung tumor | 2.42 |
| breast normal GW00-392 | 28750 | 25.7 | 2480.5 | 2480.50 | breast normal | |
| breast tumor GW00-391 | 28746 | 25.77 | 2364.2 | 4728.40 | breast tumor | 1.91 |
| breast normal GW00-413 | 28798 | 26.06 | 1948.1 | 1948.10 | breast normal | |
| breast tumor GW00-412 | 28797 | 27.21 | 894.11 | 1788.22 | breast tumor | −1.09 |
| breast normal GW00- | 27592-95 | 26.64 | 1317.83 | 1317.83 | breast normal | |
| 235:238 | | | | | | |
| breast tumor GW00- | 27588-91 | 23.91 | 8225.11 | 8225.11 | breast tumor | 6.24 |
| 231:234 | | | | | | |
| breast normal GW98-621 | 23656 | 24.46 | 5693.73 | 11387.46 | breast normal | |
| breast tumor GW98-620 | 23655 | 23.91 | 8218.73 | 16437.46 | breast tumor | 1.44 |
| brain normal BB99-542 | 25507 | 26.39 | 1553.13 | 3106.26 | brain normal | |
| brain normal BB99-406 | 25509 | 26.63 | 1325.63 | 2651.26 | brain normal | |
| brain normal BB99-904 | 25546 | 27.05 | 1001.6 | 2003.20 | brain normal | |
| brain stage 5 ALZ BB99- | 25502 | 26.97 | 1052.15 | 2104.30 | brain stage 5 ALZ | −1.23 |
| 874 | | | | | | |
| brain stage 5 ALZ BB99- | 25503 | 25.28 | 3289.99 | 6579.98 | brain stage 5 ALZ | 2.54 |
| 887 | | | | | | |
| brain stage 5 ALZ BB99- | 25504 | 26.24 | 1725.06 | 3450.12 | brain stage 5 ALZ | 1.33 |
| 862 | | | | | | |
| brain stage 5 ALZ BB99- | 25542 | 26.12 | 1864.26 | 3728.52 | brain stage 5 ALZ | 1.44 |
| 927 | | | | | | |
| CT lung KC | normal | 24.74 | 4711.99 | 9423.98 | CT lung | |
| lung 26 KC | normal | 27.78 | 611.36 | 611.36 | lung 26 | |
| lung 27 KC | normal | 28.27 | 439.19 | 439.19 | lung 27 | |
| lung 24 KC | COPD | 26.92 | 1091.11 | 1091.11 | lung 24 | −2.56 |
| lung 28 KC | COPD | 26.93 | 1085.65 | 1085.65 | lung 28 | −2.57 |
| lung 23 KC | COPD | 27.19 | 909.68 | 909.68 | lung 23 | −3.07 |
| lung 25 KC | normal | 27.62 | 678.79 | 678.79 | lung 25 | |
| asthmatic lung ODO3112 | 29321 | 25.33 | 3173.52 | 3173.52 | asthmatic lung | 1.14 |
| asthmatic lung ODO3433 | 29323 | 25.36 | 3106.89 | 6213.78 | asthmatic lung | 2.23 |
| asthmatic lung ODO3397 | 29322 | 23.81 | 8809.42 | 17618.84 | asthmatic lung | 6.32 |
| asthmatic lung ODO4928 | 29325 | 24.76 | 4649.98 | 9299.96 | asthmatic lung | 3.34 |
| endo cells KC | control | 26 | 2021.13 | 2021.13 | endo cells | |
| endo VEGF KC | | 25.78 | 2343.21 | 2343.21 | endo VEGF | 1.16 |
| endo bFGF KC | | 26.7 | 1264.03 | 1264.03 | endo bFGF | −1.60 |
| heart Clontech | normal | 26.62 | 1330.64 | 2661.28 | heart | |
| heart (T-1) ischemic | 29417 | 27.07 | 984.33 | 1968.66 | heart T-1 | −1.35 |
| heart (T-14) non- | 29422 | 26.11 | 1877.75 | 3755.50 | heart T-14 | 1.41 |
| obstructive DCM | | | | | | |
| heart (T-3399) DCM | 29426 | 26.34 | 1608.79 | 3217.58 | heart T-3399 | 1.21 |
| adenoid GW99-269 | 26162 | 27.64 | 670.25 | 1340.50 | adenoid | |
| tonsil GW98-280 | 22582 | 27.61 | 684.15 | 1368.30 | tonsil | |
| T cells PC00314 | 28453 | 25.95 | 2098.64 | 4197.28 | T cells | |
| PBMNC | | 31.16 | 63.19 | 63.19 | PBMNC | |
| monocyte | | 31.32 | 56.63 | 113.26 | monocyte | |
| B cells PC00665 | 28455 | 26.34 | 1609.52 | 3219.04 | B cells | |
| dendritic cells 28441 | | 28.25 | 444.68 | 889.36 | dendritic cells | |
| neutrophils | 28440 | 26.11 | 1874.13 | 1874.13 | neutrophils | |
| eosinophils | 28446 | 26.39 | 1553.82 | 3107.64 | eosinophils | |
| BM unstim | | 31.45 | 51.76 | 51.76 | BM unstim | |
| BM stim | | 31.28 | 58.37 | 58.37 | BM stim | 1.13 |
| osteo dif | | 24.62 | 5118.74 | 5118.74 | osteo dif | 1.70 |
| osteo undif | | 25.41 | 3015.6 | 3015.60 | osteo undif | |
| chondrocytes | | 22.12 | 27351.89 | 68379.73 | chondrocytes | |
| OA Synovium IP12/01 | 29462 | 24.5 | 5551.61 | 5551.61 | OA Synovium | |
| OA Synovium NP10/01 | 29461 | 25.1 | 3711.29 | 7422.58 | OA Synovium | |
| OA Synovium NP57/00 | 28464 | 24.5 | 5537.1 | 11074.20 | OA Synovium | |
| RA Synovium NP03/01 | 28466 | 26.45 | 1492.95 | 2985.90 | RA Synovium | |
| RA Synovium NP71/00 | 28467 | 24.44 | 5783.96 | 11567.92 | RA Synovium | |
| RA Synovium NP45/00 | 28475 | 25.94 | 2112.54 | 4225.08 | RA Synovium | |
| OA bone (biobank) | 29217 | 26.16 | 1811.72 | 1811.72 | OA bone | |
| | | | | | (biobank) | |
| OA bone Sample 1 | J. Emory | 24.54 | 5399.31 | 10798.62 | OA bone | |
| OA bone Sample 2 | J. Emory | 26.07 | 1931.94 | 3863.88 | OA bone | |
| Cartilage (pool) | Normal | 25.09 | 3730.42 | 7460.84 | Cartilage (pool) | |
| Cartilage (pool) | OA | 25.79 | 2328.66 | 4657.32 | Cartilage (pool) | −1.60 |
| PBL unifected | 28441 | 26.95 | 1068.16 | 2136.32 | PBL unifected | |
| PBL HIV IIIB | 28442 | 28.41 | 401.86 | 803.72 | PBL HIV IIIB | −2.66 |
| MRC5 uninfected | 29158 | 22.28 | 24694.87 | 49389.74 | MRC5 uninfected | |
| (100%) | | | | | (100%) | |
| MRC5 HSV strain F | 29178 | 29.07 | 256.64 | 513.28 | MRC5 HSV strain F | −96.22 |
| W12 cells | 29179 | 28.37 | 410.68 | 821.36 | W12 cells | |
| Keratinocytes | 29180 | 29.12 | 249.25 | 498.50 | Keratinocytes | |
| B-actin control | | 27.53 | 721.15 | | | |
| genomic | | 26.92 | 1091.74 | | | |
| 1.00E+05 | | 19.96 | 100000 | | | |
| 1.00E+05 | | 20.19 | 100000 | | | |
| 1.00E+04 | | 23.43 | 10000 | | | |
| 1.00E+04 | | 23.34 | 10000 | | | |
| 1.00E+03 | | 26.64 | 1000 | | | |
| 1.00E+03 | | 26.8 | 1000 | | | |
| 1.00E+02 | | 31.34 | 100 | | | |
| 1.00E+02 | | 31.48 | 100 | | | |
| 1.00E+01 | | 34.9 | 10 | | | |
| 1.00E+01 | | 34.19 | 10 | | | |
| 1.00E−00 | | 40 | 0 | | | |
| 1.00E−00 | | 35.53 | 1 | | | |
| NTC | | 40 | 0 | | | |

Gene Name sbg1151bSREC

| | Fold Change in Disease |
|---|---|
| | Population Relative to |
| Disease tissues | Normal |
| colon tumor | 3.69 |
| colon tumor | 1.48 |
| colon tumor | 2.05 |
| colon tumor | −1.43 |
| lung tumor | −9.84 |
| lung tumor | 2.09 |
| lung tumor | −1.15 |
| lung tumor | 2.42 |
| breast tumor | 1.91 |
| breast tumor | −1.09 |
| breast tumor | 6.24 |
| breast tumor | 1.44 |
| brain stage 5 ALZ | −1.23 |
| brain stage 5 ALZ | 2.54 |
| brain stage 5 ALZ | 1.33 |
| brain stage 5 ALZ | 1.44 |
| lung 24 | −2.56 |
| lung 28 | −2.57 |
| lung 23 | −3.07 |
| asthmatic lung | 1.14 |
| asthmatic lung | 2.23 |
| asthmatic lung | 6.32 |
| asthmatic lung | 3.34 |
| endo VEGF | 1.16 |
| endo bFGF | −1.60 |
| heart T-1 | −1.35 |
| heart T-14 | 1.41 |
| heart T-3399 | 1.21 |
| BM stim | 1.13 |
| osteo dif | 1.70 |
| Cartilage (pool) | −1.60 |
| PBL HIV IIIB | −2.66 |
| MRC5 HSV strain F | −96.22 |

Gene Name sbg1399854ANK

Low overall expression. Highest normal expression is seen in the whole brain, fetal brain, and liver. Good levels of expression are seen in all of the samples representing the female reproductive system. Highest disease expression is seen in the normal and Alzheimer's brain samples as well as in the dendritic cells. Upregulation in 2 of 4 colon tumor samples and in 2 of 4 breast tumor samples as well as downregulation in 2 of 4 lung tumor samples implicates this gene in cancers of the colon, breast, and lung. Downregulation in 3 of 3 COPD samples and in 2 of 4 asthmatic lung samples suggests a potential role for this gene in chronic obstructive pulmonary disorder and asthma. Downregulation in the OA cartilage sample as well as corroborating low expression in the normal chondrocytes and many of the immune cells suggests involvement in osteoarthritis. Upregulation in the HSV-infected MRC5 cells suggests that this gene may be a host factor in HSV.

| | | | | | | | copies of |
|---|---|---|---|---|---|---|---|
| | | Mean | Mean | | | | mRNA |
| | | GOI | GOI | | | 50 ng/ | detected/ |
| | | copies | copies | Average | 18S | 18S | 50 ng |
| Sample | Ct (sample | (sample | (sample | GOI | rRNA | rRNA | total |
| sbg1399854ANK | 1 and 2) | 1) | 2) | Copies | (ng) | (ng) | RNA |
| Subcutaneous | 40, 40 | 0 | 0 | 0.00 | 3.06 | 16.34 | 0.00 |
| Adipocytes Zenbio | | | | | | | |
| Subcutaneous Adipose | 40, 40 | 1.83 | 1.77 | 1.80 | 0.96 | 52.36 | 94.24 |
| Zenbio | | | | | | | |
| Adrenal Gland Clontech | 40, 40 | 1.75 | 0 | 0.88 | 0.61 | 81.97 | 71.72 |
| Whole Brain Clontech | 28.57, 28.35 | 944.29 | 1058.09 | 1001.19 | 7.24 | 6.91 | 6914.30 |
| Fetal Brain Clontech | 36.57, 34.65 | 14.38 | 39.28 | 26.83 | 0.48 | 103.95 | 2788.98 |
| Cerebellum Clontech | 37.21, 36.71 | 10.3 | 13.37 | 11.84 | 2.17 | 23.04 | 272.70 |
| Cervix | 36.3, 40 | 16.52 | 0 | 8.26 | 2.42 | 20.66 | 170.66 |
| Colon | 36.17, 35.71 | 17.73 | 22.5 | 20.12 | 2.71 | 18.45 | 371.13 |
| Endometrium | 40, 36.1 | 0 | 18.41 | 9.21 | 0.73 | 68.21 | 627.90 |
| Esophagus | 40, 40 | 0 | 1.58 | 0.79 | 1.37 | 36.50 | 28.83 |
| Heart Clontech | 40, 40 | 0 | 0 | 0.00 | 1.32 | 37.88 | 0.00 |
| Hypothalamus | 40, 40 | 0 | 0 | 0.00 | 0.32 | 155.28 | 0.00 |
| Ileum | 40, 40 | 0 | 0 | 0.00 | 2.58 | 19.38 | 0.00 |
| Jejunum | 34.07, 34.37 | 53.18 | 45.33 | 49.26 | 6.60 | 7.58 | 373.14 |
| Kidney | 37.55, 40 | 8.58 | 0 | 4.29 | 2.12 | 23.58 | 101.18 |
| Liver | 36.53, 35.62 | 14.69 | 23.59 | 19.14 | 1.50 | 33.33 | 638.00 |
| Fetal Liver Clontech | 34.56, 34.61 | 41.04 | 40.04 | 40.54 | 10.40 | 4.81 | 194.90 |
| Lung | 40, 40 | 0 | 0 | 0.00 | 2.57 | 19.46 | 0.00 |
| Mammary Gland | 39.07, 39.45 | 3.89 | 3.18 | 3.54 | 13.00 | 3.85 | 13.60 |
| Clontech | | | | | | | |
| Myometrium | 39.5, 35.37 | 3.1 | 26.93 | 15.02 | 2.34 | 21.37 | 320.83 |
| Omentum | 40, 36.18 | 0 | 17.61 | 8.81 | 3.94 | 12.69 | 111.74 |
| Ovary | 35.87, 34.34 | 20.75 | 46.22 | 33.49 | 4.34 | 11.52 | 385.77 |
| Pancreas | 40, 40 | 0 | 0 | 0.00 | 0.81 | 61.80 | 0.00 |
| Head of Pancreas | 40, 39.79 | 0 | 2.66 | 1.33 | 1.57 | 31.85 | 42.36 |
| Parotid Gland | 35.2, 38.6 | 29.46 | 4.97 | 17.22 | 5.48 | 9.12 | 157.07 |
| Placenta Clontech | 40, 38.14 | 0 | 6.32 | 3.16 | 5.26 | 9.51 | 30.04 |
| Prostate | 40, 40 | 0 | 0 | 0.00 | 3.00 | 16.67 | 0.00 |
| Rectum | 40, 39.2 | 0 | 3.63 | 1.82 | 1.23 | 40.65 | 73.78 |
| Salivary Gland | 40, 39.19 | 2.19 | 3.64 | 2.92 | 7.31 | 6.84 | 19.94 |
| Clontech | | | | | | | |
| Skeletal Muscle | 40, 39.37 | 0 | 3.32 | 1.66 | 1.26 | 39.68 | 65.87 |
| Clontech | | | | | | | |
| Skin | 40, 40 | 0 | 2.09 | 1.05 | 1.21 | 41.32 | 43.18 |
| Small Intestine | 40, 40 | 1.44 | 0 | 0.72 | 0.98 | 51.07 | 36.77 |
| Clontech | | | | | | | |
| Spleen | 35.36, 40 | 27.05 | 1.84 | 14.45 | 4.92 | 10.16 | 146.80 |
| Stomach | 40, 37.03 | 1.93 | 11.29 | 6.61 | 2.73 | 18.32 | 121.06 |
| Testis Clontech | 40, 37.99 | 0 | 6.82 | 3.41 | 0.57 | 87.87 | 299.65 |
| Thymus Clontech | 38.47, 35.55 | 5.32 | 24.52 | 14.92 | 9.89 | 5.06 | 75.43 |
| Thyroid | 40, 40 | 0 | 2.15 | 1.08 | 2.77 | 18.05 | 19.40 |
| Trachea Clontech | 35.37, 36.67 | 26.97 | 13.66 | 20.32 | 9.71 | 5.15 | 104.61 |
| Urinary Bladder | 39.07, 40 | 3.89 | 1.42 | 2.66 | 5.47 | 9.14 | 24.27 |
| Uterus | 36.01, 33.41 | 19.29 | 75.06 | 47.18 | 5.34 | 9.36 | 441.71 |
| genomic | 29.57 | 558.84 | | | | | |
| b-actin | 27.57 | 1592.66 | | | | | |
| 1.00E+05 | 19.91 | 100000 | | | | | |
| 1.00E+05 | 20.08 | 100000 | | | | | |
| 1.00E+04 | 23.79 | 10000 | | | | | |
| 1.00E+04 | 24.06 | 10000 | | | | | |
| 1.00E+03 | 27.72 | 1000 | | | | | |
| 1.00E+03 | 28.29 | 1000 | | | | | |
| 1.00E+02 | 31.95 | 100 | | | | | |
| 1.00E+02 | 33.62 | 100 | | | | | |
| 1.00E+01 | 39.75 | 10 | | | | | |
| 1.00E+01 | 35.41 | 10 | | | | | |
| 1.00E−00 | 40 | 0 | | | | | |
| 1.00E−00 | 40 | 0 | | | | | |
| NTC | 40 | −1 | | | | | |
| NTC | 40 | −1 | | | | | |

| | | | | copies of | | |
|---|---|---|---|---|---|---|
| | | | | mRNA | | Fold |
| | Reg | | | detected/ | | Change |
| | number | | Mean | 50 ng | | in |
| Sample | (GSK | | GOI | total | | Disease |
| sbg1399854ANK | identifier) | Ct | copies | RNA | Sample | Population |
| colon normal GW98-167 | 21941 | 35.32 | 22.34 | 44.68 | colon normal | |
| colon tumor GW98-166 | 21940 | 34.14 | 47.5 | 95.00 | colon tumor | 2.13 |
| colon normal GW98-178 | 22080 | 36.16 | 13.07 | 26.14 | colon normal | |
| colon tumor GW98-177 | 22060 | 35.58 | 18.93 | 37.86 | colon tumor | 1.45 |
| colon normal GW98-561 | 23514 | 36.58 | 10.03 | 20.06 | colon normal | |
| colon tumor GW98-560 | 23513 | 32.61 | 126.48 | 252.96 | colon tumor | 12.61 |
| colon normal GW98-894 | 24691 | 35.61 | 18.62 | 37.24 | colon normal | |
| colon tumor GW98-893 | 24690 | 33.24 | 84.75 | 169.50 | colon tumor | 4.55 |
| lung normal GW98-3 | 20742 | 34.77 | 31.8 | 63.60 | lung normal | |
| lung tumor GW98-2 | 20741 | 33.63 | 66.03 | 132.06 | lung tumor | 2.08 |
| lung normal GW97-179 | 20677 | 34.76 | 32.04 | 64.08 | lung normal | |
| lung tumor GW97-178 | 20676 | 34.44 | 39.23 | 78.46 | lung tumor | 1.22 |
| lung normal GW98-165 | 21922 | 35.18 | 24.44 | 48.88 | lung normal | |
| lung tumor GW98-164 | 21921 | 37.99 | 4.06 | 8.12 | lung tumor | −6.02 |
| lung normal GW98-282 | 22584 | 33.64 | 65.37 | 130.74 | lung normal | |
| lung tumor GW98-281 | 22583 | 37.3 | 6.34 | 12.68 | lung tumor | −10.31 |
| breast normal GW00-392 | 28750 | 36.29 | 12.08 | 12.08 | breast normal | |
| breast tumor GW00-391 | 28746 | 36.14 | 13.29 | 26.58 | breast tumor | 2.20 |
| breast normal GW00-413 | 28798 | 37.08 | 7.29 | 7.29 | breast normal | |
| breast tumor GW00-412 | 28797 | 33.26 | 83.58 | 167.16 | breast tumor | 22.93 |
| breast normal GW00- | 27592-95 | 38.93 | 2.24 | 2.24 | breast normal | |
| 235:238 | | | | | | |
| breast tumor GW00- | 27588-91 | 36.57 | 10.08 | 10.08 | breast tumor | 4.50 |
| 231:234 | | | | | | |
| breast normal GW98-621 | 23656 | 34.9 | 29.32 | 58.64 | breast normal | |
| breast tumor GW98-620 | 23655 | 36.11 | 13.51 | 27.02 | breast tumor | −2.17 |
| brain normal BB99-542 | 25507 | 29.6 | 866.9 | 1733.80 | brain normal | |
| brain normal BB99-406 | 25509 | 31.93 | 194.87 | 389.74 | brain normal | |
| brain normal BB99-904 | 25546 | 30.38 | 526.58 | 1053.16 | brain normal | |
| brain stage 5 ALZ BB99- | 25502 | 32.7 | 119.57 | 239.14 | brain stage 5 ALZ | −4.43 |
| 874 | | | | | | |
| brain stage 5 ALZ BB99- | 25503 | 30.08 | 634.97 | 1269.94 | brain stage 5 ALZ | 1.20 |
| 887 | | | | | | |
| brain stage 5 ALZ BB99- | 25504 | 29.7 | 809.22 | 1618.44 | brain stage 5 ALZ | 1.53 |
| 862 | | | | | | |
| brain stage 5 ALZ BB99- | 25542 | 29.93 | 700.82 | 1401.64 | brain stage 5 ALZ | 1.32 |
| 927 | | | | | | |
| CT lung | normal | 35.1 | 25.82 | 51.64 | CT lung Nml | |
| lung 26 | normal | 36.74 | 9.07 | | lung 26 Nml | |
| lung 27 | normal | 40 | 0 | 0.00 | lung 27 Nml | |
| lung 24 | COPD | 40 | 2.5 | 2.50 | lung 24 COPD | −7.11 |
| lung 28 | COPD | 40 | 0 | 0.00 | lung 28 COPD | −17.77 |
| lung 23 | COPD | 40 | 0 | 0.00 | lung 23 COPD | −17.77 |
| lung 25 | normal | 39.39 | 1.67 | 1.67 | lung 25 Nml | |
| asthmatic lung ODO3112 | 29321 | 40 | 0 | 0.00 | asthmatic lung | −17.77 |
| asthmatic lung ODO3433 | 29323 | 37 | 7.68 | 15.36 | asthmatic lung | −1.16 |
| asthmatic lung ODO3397 | 29322 | 36.08 | 13.8 | 27.60 | asthmatic lung | 1.55 |
| asthmatic lung ODO4928 | 29325 | 40 | 0 | 0.00 | asthmatic lung | −17.77 |
| endo cells | control | 40 | 0 | 0.00 | endo cells | |
| endo VEGF | | 40 | 0 | 0.00 | endo VEGF | 0.00 |
| endo bFGF | | 35.68 | 17.77 | 17.77 | endo bFGF | 17.77 |
| heart Clontech | normal | 35.03 | 26.95 | 53.90 | heart | |
| heart (T-1) ischemic | 29417 | 36.36 | 11.53 | 23.06 | heart (T-1) | −2.34 |
| | | | | | ischemic | |
| heart (T-14) non- | 29422 | 34.57 | 36.11 | 72.22 | heart (T-14) non- | 1.34 |
| obstructive DCM | | | | | obstructive DCM | |
| heart (T-3399) DCM | 29426 | 36.25 | 12.37 | 24.74 | heart (T-3399) | −2.18 |
| | | | | | DCM | |
| adenoid GW99-269 | 26162 | 38.51 | 2.92 | 5.84 | adenoid | |
| tonsil GW98-280 | 22582 | 35.05 | 26.54 | 53.08 | tonsil | |
| T cells PC00314 | 28453 | 35.5 | 19.98 | 39.96 | T cells | |
| PBMNC | | 40 | 0 | 0.00 | PBMNC | |
| monocyte | | 40 | 0 | 0.00 | monocyte | |
| B cells PC00665 | 28455 | 33.78 | 59.82 | 119.64 | B cells | |
| dendritic cells | 28441 | 29.33 | 1026.14 | 2052.28 | dendritic cells | |
| neutrophils | 28440 | 31.3 | 292.56 | 292.56 | neutrophils | |
| eosinophils | 28446 | 35.97 | 14.79 | 29.58 | eosinophils | |
| BM unstim | | 35.56 | 19.16 | 19.16 | BM unstim | |
| BM stim | treated | 34.79 | 31.48 | 31.48 | BM stim | 1.64 |
| osteo dif | treated | 40 | 2.59 | 2.59 | osteo dif | 2.59 |
| osteo undif | | 40 | 0 | 0.00 | osteo undif | |
| chondrocytes | | 37.11 | 7.15 | 17.88 | chondrocytes | |
| OA Synovium IP12/01 | 29462 | 35.95 | 14.93 | 14.93 | OA Synovium | |
| OA Synovium NP10/01 | 29461 | 35.74 | 17.17 | 34.34 | OA Synovium | |
| OA Synovium NP57/00 | 28464 | 39.09 | 2.02 | 4.04 | OA Synovium | |
| RA Synovium NP03/01 | 28466 | 38.03 | 3.97 | 7.94 | RA Synovium | |
| RA Synovium NP71/00 | 28467 | 35.08 | 26.03 | 52.06 | RA Synovium | |
| RA Synovium NP45/00 | 28475 | 37.11 | 7.13 | 14.26 | RA Synovium | |
| OA bone (biobank) | 29217 | 33.76 | 60.54 | 60.54 | OA bone | |
| | | | | | (biobank) | |
| OA bone Sample 1 | J. Emory | 33.35 | 78.68 | 157.36 | OA bone | |
| OA bone Sample 2 | J. Emory | 34.15 | 47.2 | 94.40 | OA bone | |
| Cartilage (pool) | Normal | 35.05 | 26.63 | 53.26 | Nml Cartilage | |
| | | | | | (pool) | |
| Cartilage (pool) | OA | 37.42 | 5.87 | 11.74 | OA Cartilage | −4.54 |
| | | | | | (pool) | |
| PBL unifected | 28441 | 33.95 | 53.63 | 107.26 | PBL unifected | |
| PBL HIV IIIB | 28442 | 33.3 | 81.2 | 162.40 | PBL HIV IIIB | 1.51 |
| MRC5 uninfected | 29158 | 39.41 | 1.64 | 3.28 | MRC5 uninfected | |
| (100%) | | | | | (100%) | |
| MRC5 HSV strain F | 29178 | 35.73 | 17.22 | 34.44 | MRC5 HSV | 10.50 |
| | | | | | strain F | |
| W12 cells | 29179 | 35.08 | 26.08 | 52.16 | W12 cells | |
| Keratinocytes | 29180 | 36.69 | 9.33 | 18.66 | Keratinocytes | |
| B-actin control | | 28.13 | 2213.67 | | | |
| genomic | | 29.03 | 1240.79 | | | |
| 1.00E+05 | | 22.03 | 100000 | | | |
| 1.00E+05 | | 22.36 | 100000 | | | |
| 1.00E+04 | | 25.68 | 10000 | | | |
| 1.00E+04 | | 25.78 | 10000 | | | |
| 1.00E+03 | | 29.01 | 1000 | | | |
| 1.00E+03 | | 28.67 | 1000 | | | |
| 1.00E+02 | | 33.46 | 100 | | | |
| 1.00E+02 | | 40 | 100 | | | |
| 1.00E+01 | | 38.75 | 10 | | | |
| 1.00E+01 | | 40 | 10 | | | |
| 1.00E−00 | | 40 | 0 | | | |
| 1.00E−00 | | 38.6 | 1 | | | |
| NTC | | 40 | 0 | | | |

*lung 26 Normal has been omitted due to multiple amplification failures from that sample

Gene Name sbg1399854ANK

| | Fold Change in Disease |
|---|---|
| | Population Relative to |
| Disease tissues | Normal |
| colon tumor | 2.13 |
| colon tumor | 1.45 |
| colon tumor | 12.61 |
| colon tumor | 4.55 |
| lung tumor | 2.08 |
| lung tumor | 1.22 |
| lung tumor | −6.02 |
| lung tumor | −10.31 |
| breast tumor | 2.20 |
| breast tumor | 22.93 |
| breast tumor | 4.50 |
| breast tumor | −2.17 |
| brain stage 5 ALZ | −4.43 |
| brain stage 5 ALZ | 1.20 |
| brain stage 5 ALZ | 1.53 |
| brain stage 5 ALZ | 1.32 |
| lung 24 | −7.11 |
| lung 28 | −17.77 |
| lung 23 | −17.77 |
| asthmatic lung | −17.77 |
| asthmatic lung | −1.16 |
| asthmatic lung | 1.55 |
| asthmatic lung | −17.77 |
| endo VEGF | 0.00 |
| endo bFGF | 17.77 |
| heart T-1 | −2.34 |
| heart T-14 | 1.34 |
| heart T-3399 | −2.18 |
| BM stim | 1.64 |
| osteo dif | 2.59 |
| Cartilage (pool) | −4.54 |
| PBL HIV IIIB | 1.51 |
| MRC5 HSV strain F | 10.50 |

TABLE V
Additional diseases based on mRNA expression in specific tissues

| Tissue | |
|---|---|
| Expression | Additional Diseases |
| Brain | Neurological and psychiatric diseases, including Alzheimers, parasupranuclear |
| | palsey, Huntington's disease, myotonic dystrophy, anorexia, depression, |
| | schizophrenia, headache, amnesias, anxiety disorders, sleep disorders, multiple |
| | sclerosis |
| Heart | Cardiovascular diseases, including congestive heart failure, dilated |
| | cardiomyopathy, cardiac arrhythmias, Hodgson's Disease, myocardial |
| | infarction, cardiac arrhythmias |
| Lung | Respiratory diseases, including asthma, Chronic Obstructive Pulmonary |
| | Disease, cystic fibrosis, acute bronchitis, adult respiratory distress syndrome |
| Liver | Dyslipidemia, hypercholesterolemia, hypertriglyceridemia, cirrhosis, hepatic |
| | encephalopathy, fatty hepatocirrhosis, viral and nonviral hepatitis, Type II |
| | Diabetes Mellitis, impaired glucose tolerance |
| Kidney | Renal diseases, including acute and chronic renal failure, acute tubular necrosis, |
| | cystinuria, Fanconi's Syndrome, glomerulonephritis, renal cell carcinoma, |
| | renovascular hypertension |
| Skeletal | Eulenburg's Disease, hypoglycemia, obesity, tendinitis, periodic paralyses, |
| muscle | malignant hyperthermia, paramyotonia congenita, myotonia congenita |
| Intestine | Gastrointestinal diseases, including Myotonia congenita, Ileus, Intestinal |
| | Obstruction, Tropical Sprue, Pseudomembranous Enterocolitis |
| Spleen/lymph | Lymphangiectasia, hypersplenism, angiomas, ankylosing spondylitis, Hodgkin's |
| | Disease, macroglobulinemia, malignant lymphomas, rheumatoid arthritis |
| Placenta | Choriocarcinoma, hydatidiform mole, placenta previa |
| Testis | Testicular cancer, male reproductive diseases, including low testosterone and |
| | male infertility |
| Pancreas | Diabetic ketoacidosis, Type 1 & 2 diabetes, obesity, impaired glucose tolerance |

1-7. (canceled)

8. An isolated polypeptide selected from the group consisting of:
(a) an isolated polypeptide encoded by a polynucleotide of SEQ ID NO: 11;
(b) an isolated polypeptide comprising a polypeptide of SEQ ID NO: 55; and
(c) a polypeptide of SEQ ID 55.

9. An isolated polynucleotide selected from the group consisting of:
(a) an isolated polynucleotide comprising a polynucleotide SEQ ID NO: 11;
(b) an isolated polynucleotide of SEQ ID NO: 11;
(c) an isolated polynucleotide encoding a polypeptide of SEQ ID NO: 55;
(e) a polynucleotide which is an RNA equivalent of the polynucleotide of (a) to (c);
or a polynucleotide sequence complementary to said isolated polynucleotide.

10. An expression vector comprising a polynucleotide capable of producing a polypeptide of claim 8 when said expression vector is present in a compatible host cell.

11. A process for producing a recombinant host cell which comprises the step of introducing an expression vector comprising a polynucleotide capable of producing a polypeptide of claim 8 into a cell such that the host cell, under appropriate culture conditions, produces said polypeptide.

12. A recombinant host cell produced by the process of claim 11.

13. A process for producing a polypeptide which comprises culturing a host cell of claim 12 under conditions sufficient for the production of said polypeptide and recovering said polypeptide from the culture.
